SEQUENCE LISTING (115 sequences)

SEQ ID NO: 1 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 14 aa, synthetic construct
TEELRVSLASHLRK

SEQ ID NO: 3 - protein, 14 aa, synthetic construct
TEELRVRLASHLRK

SEQ ID NO: 4 - protein, 15 aa, synthetic construct
KASQSVDYDGDSYMN

SEQ ID NO: 5 - protein, 7 aa, synthetic construct
AASNLES

SEQ ID NO: 6 - protein, 9 aa, synthetic construct
QQSNEDPWT

SEQ ID NO: 7 - protein, 5 aa, synthetic construct
SYTMS

SEQ ID NO: 8 - protein, 17 aa, synthetic construct
KIRNGGGITYYLDTLKG

SEQ ID NO: 9 - protein, 11 aa, synthetic construct
HYYGSEDYFDY

SEQ ID NO: 10 - DNA, 393 bp, synthetic construct
  source 1..393: mol_type = other DNA
atggagacagacacaatcctgctatgggtgctgctgctctgggttccaggctccactggtgacaatgtgctgacccaatctccagcttctttggctgtgtctctagggcagagggccaccatctcctgcaaggccagccaaagtgttgattatgatggtgatagttatatgaactggtaccaacagaaaccaggacagccacccaaagtcttcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 11 - DNA, 417 bp, synthetic construct
  source 1..417: mol_type = other DNA
atgaatttcgggctcagcttgattttccttgtccttgttttaaaaggtgtcctgtgtgaagtgaagctggtggaatctgggggaggtgtggtgcagcctggagggtccctgaaactctcctgtgcagcctctggattcactttcagtagctataccatgtcttgggttcgtcagactccagagaagaggctggagtgggtcgcaaaaattcgtaatggtggtggtatcacctactatttagacactttaaagggccgattcaccatctccagagacaacgccaagaacaccctatacctgcaaatgagcagtctgaagtctgaagacacggccatttatttctgtgcaagacattactacggtagcgaggactactttgactactggggccaaggcaccactctcacagtctcctca

SEQ ID NO: 12 - protein, 131 aa, synthetic construct
METDTILLWVLLLWVPGSTGDNVLTQSPASLAVSLGQRATISCKASQSVDYDGDSYMNWYQQKPGQPPKVFIYAASNLESGIPARFSGSGSGTDFTLNIHPVEEEDAATYYCQQSNEDPWTFGGGTKLEIK

SEQ ID NO: 13 - protein, 139 aa, synthetic construct
MNFGLSLIFLVLVLKGVLCEVKLVESGGGVVQPGGSLKLSCAASGFTFSSYTMSWVRQTPEKRLEWVAKIRNGGGITYYLDTLKGRFTISRDNAKNTLYLQMSSLKSEDTAIYFCARHYYGSEDYFDYWGQGTTLTVSS

SEQ ID NO: 14 - protein, 15 aa, synthetic construct
KASQSVDYDGDSYMN

SEQ ID NO: 15 - protein, 7 aa, synthetic construct
AASNLES

SEQ ID NO: 16 - protein, 9 aa, synthetic construct
QQSNEDPWT

SEQ ID NO: 17 - protein, 5 aa, synthetic construct
RYTMS

SEQ ID NO: 18 - protein, 17 aa, synthetic construct
KIRNVGGITYYPDTVKG

SEQ ID NO: 19 - protein, 11 aa, synthetic construct
HYYGSEDYFDY

SEQ ID NO: 20 - DNA, 417 bp, synthetic construct
  source 1..417: mol_type = other DNA
atgaatttcgggctcagcgtgattttccttgtccttgttttaaaaggtgtcctgtgtgaagtgaagctggtggagtctgggggaggtttagtgcagcctggagggtccctgaaactctcctgtgcagcctctggattcactttcagtaggtataccatgtcttgggttcggcagactccagagaagaggctggagtgggtcgcaaaaattcgtaatgttggtggtatcacctactatccagacactgtaaagggccgattcaccatctccagagacaacgccaagaacaccctttacctgcaaatgagcagtctgaagtctgaagacacggccatgtattactgtgcaagacattattacggtagcgaggactactttgactactggggccaaggcaccactctcacagtctcctca

SEQ ID NO: 21 - DNA, 393 bp, synthetic construct
  source 1..393: mol_type = other DNA
atggagacagacacaatcctgctatgggtgctgctgctctgggttccaggctccactggtgacaatgtgctgacccaatctccagcttctttggctgtgtctctagggcagagggccaccatctcctgcaaggccagccaaagtgttgattatgatggtgatagttatatgaactggtaccaacagaaaccaggacagccacccaaagtcttcatctatgctgcatccaatctagaatctgggatcccagccaggtttagtggcagtgggtctgggacaaacttcaccctcaacatccatcctgtggaggaggaggatgctgcaacctattactgtcagcaaagtaatgaggatccgtggacgttcggtggaggcaccaagctggaaatcaaa

SEQ ID NO: 22 - protein, 139 aa, synthetic construct
MNFGLSVIFLVLVLKGVLCEVKLVESGGGLVQPGGSLKLSCAASGFTFSRYTMSWVRQTPEKRLEWVAKIRNVGGITYYPDTVKGRFTISRDNAKNTLYLQMSSLKSEDTAMYYCARHYYGSEDYFDYWGQGTTLTVSS

SEQ ID NO: 23 - protein, 131 aa, synthetic construct
METDTILLWVLLLWVPGSTGDNVLTQSPASLAVSLGQRATISCKASQSVDYDGDSYMNWYQQKPGQPPKVFIYAASNLESGIPARFSGSGSGTNFTLNIHPVEEEDAATYYCQQSNEDPWTFGGGTKLEIK

SEQ ID NO: 24 - protein, 15 aa, synthetic construct
KASQSVDYDGDSYMN

SEQ ID NO: 25 - protein, 7 aa, synthetic construct
AASNLES

SEQ ID NO: 26 - protein, 9 aa, synthetic construct
QQSNVDPWT

SEQ ID NO: 27 - protein, 5 aa, synthetic construct
DYHMH

SEQ ID NO: 28 - protein, 17 aa, synthetic construct
WIDPENGNTMYDPKFQG

SEQ ID NO: 29 - protein, 9 aa, synthetic construct
GTARASFDY

SEQ ID NO: 30 - DNA, 393 bp, synthetic construct
  source 1..393: mol_type = other DNA
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaattggtaccaacagaaatcaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgtggatccgtggacgttcggtggaggcaccaagctggaaatcaaa

SEQ ID NO: 31 - DNA, 411 bp, synthetic construct
  source 1..411: mol_type = other DNA
atgaaatgcagctgggtcatcttcttcctgatggcagtggttacaggggtcaattcagaggttcagctgcagcagtctggggctgagcttgtgaggccaggggccttagtcaagttgtcctgcaaagcttctggcttcaacattaaagactaccatatgcactgggtgaaggagaggcctgaacagggcctggagtggattggatggattgatcctgagaatggtaatactatgtatgacccgaagttccagggcaaggccagtataacagcagacacatcctccaacacagcctacctgcagctcagcagcctgacatctgaggacactgccgtctattactgtgttagggggacagctcgggcttcctttgactactggggccaaggcaccactctcacagtctcctca

SEQ ID NO: 32 - protein, 131 aa, synthetic construct
METDTILLWVLLLWVPGSTGDIVLTQSPASLAVSLGQRATISCKASQSVDYDGDSYMNWYQQKSGQPPKLLIYAASNLESGIPARFSGSGSGTDFTLNIHPVEEEDAATYYCQQSNVDPWTFGGGTKLEIK

SEQ ID NO: 33 - protein, 137 aa, synthetic construct
MKCSWVIFFLMAVVTGVNSEVQLQQSGAELVRPGALVKLSCKASGFNIKDYHMHWVKERPEQGLEWIGWIDPENGNTMYDPKFQGKASITADTSSNTAYLQLSSLTSEDTAVYYCVRGTARASFDYWGQGTTLTVSS

SEQ ID NO: 34 - protein, 15 aa, synthetic construct
KASQSVDYDGDTYMN

SEQ ID NO: 35 - protein, 7 aa, synthetic construct
TASNLES

SEQ ID NO: 36 - protein, 9 aa, synthetic construct
QQSNEDPWT

SEQ ID NO: 37 - protein, 5 aa, synthetic construct
DYHIH

SEQ ID NO: 38 - protein, 17 aa, synthetic construct
WIDPEIDKTLYDPKFQG

SEQ ID NO: 39 - protein, 9 aa, synthetic construct
GTARASFDY

SEQ ID NO: 40 - DNA, 393 bp, synthetic construct
  source 1..393: mol_type = other DNA
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatacttatatgaactggtaccaacagaaaccaggacagccacccaaactcctcatctatactgcatccaatctagaatctgggatcccagccaggtttagtggcagtgggtctgggacagacttcaccctcaacatccatcctgtggaggaggtggatgctgcaacctattactgtcagcaaagtaatgaggatccatggacgttcggtggaggcaccaagctggaaatcaaa

SEQ ID NO: 41 - DNA, 411 bp, synthetic construct
  source 1..411: mol_type = other DNA
atgaaatgcagctgggtcatcttcttcctgatggcagtggttacaggggtcaattcagaggttcagctgcagcagtctggggctgagcttgtgaggccaggggccttagtcaagtggtcctgcaaagcttctggcttcaacattaaagactaccatatacactgggtgaaacagaggcctgaacagggcctggactggattggatggattgatcctgagattgataaaactctatatgacccgaagtttcagggcaaggccagaataacagcagacacatcctccaatacagcctacctgcagctcagcagcctgacatctgaagacactgccgtctattactgtgccagggggacagctcgggcttcctttgactactggggccaaggcaccactctcacagtctcctca

SEQ ID NO: 42 - protein, 131 aa, synthetic construct
METDTILLWVLLLWVPGSTGDIVLTQSPASLAVSLGQRATISCKASQSVDYDGDTYMNWYQQKPGQPPKLLIYTASNLESGIPARFSGSGSGTDFTLNIHPVEEVDAATYYCQQSNEDPWTFGGGTKLEIK

SEQ ID NO: 43 - protein, 137 aa, synthetic construct
MKCSWVIFFLMAVVTGVNSEVQLQQSGAELVRPGALVKWSCKASGFNIKDYHIHWVKQRPEQGLDWIGWIDPEIDKTLYDPKFQGKARITADTSSNTAYLQLSSLTSEDTAVYYCARGTARASFDYWGQGTTLTVSS

SEQ ID NO: 44 - protein, 15 aa, synthetic construct
KASQSVDYDGENYMN

SEQ ID NO: 45 - protein, 7 aa, synthetic construct
VASNLES

SEQ ID NO: 46 - protein, 9 aa, synthetic construct
QQSNLDPWT

SEQ ID NO: 47 - protein, 7 aa, synthetic construct
GFNIKDY

SEQ ID NO: 48 - protein, 6 aa, synthetic construct
DPENGN

SEQ ID NO: 49 - protein, 9 aa, synthetic construct
GTARASFDY

SEQ ID NO: 50 - protein, 317 aa, synthetic construct
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 51 - skipped

SEQ ID NO: 52 - protein, 218 aa, synthetic construct
DIVLTQSPASLAVSLGQRATISCKASQSVDYDGENYMNWYQQKPGQSPKLLIYVASNLESGIPARFSGSGSGTDFTLNIHPVEEEDAATYYCQQSNLDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 442 aa, synthetic construct
EVQLQQSGAELVRPGALVKLSCKASGFNIKDYHLHWVKQRPEQGLEWIGWIDPENGNVIYDPKFQGKATMTVVTSSNTAYLQLRSLTSEDTAVYFCTRGTARASFDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 15 aa, synthetic construct
CTEELRVRLASHLRK

SEQ ID NO: 55 - protein, 15 aa, synthetic construct
CTEELRVSLASHLRK

SEQ ID NO: 56 - DNA, 954 bp, synthetic construct
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 57 - protein, 29 aa, Homo sapiens
STEELRVRLASHLRKLRKRLLRDADDLQK

SEQ ID NO: 58 - protein, 29 aa, synthetic construct
STEELRVSLASHLRKLRKRLLRDADDLQK

SEQ ID NO: 59 - protein, 31 aa, Homo sapiens
RLVQYRGEVQAMLGQSTEELRVRLASHLRKL

SEQ ID NO: 60 - protein, 31 aa, synthetic construct
RLVQYRGEVQAMLGQSTEELRVSLASHLRKL

SEQ ID NO: 61 - protein, 7 aa, unidentified
  REGION 1..7: note = Description of Unknown: nuclear localization sequence
PKKKRKV

SEQ ID NO: 62 - protein, 7 aa, Simian immunodeficiency virus
PKKKRRV

SEQ ID NO: 63 - protein, 16 aa, unidentified
  REGION 1..16: note = Description of Unknown: nucleoplasmin NLS sequence
KRPAATKKAGQAKKKK

SEQ ID NO: 64 - DNA, 60 bp, synthetic construct
  source 1..60: mol_type = other DNA
tagcaataaaggatcgtttattttcattggaagcgtgtgttggttttttgatcaggcgcg

SEQ ID NO: 65 - DNA, 127 bp, synthetic construct
  source 1..127: mol_type = other DNA
cgcctggtgcagtaccgcggcgaggtgcaggccatgctcggccagagcacagaggagctccgcgtgagtctcgcaagccacctgcgcaagctgcgtaagcggctcctccgcgatgccgatgacctgc

SEQ ID NO: 66 - DNA, 264 bp, Homo sapiens
  source 1..264: mol_type = genomic DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 67 - DNA, 25 bp, synthetic construct
  source 1..25: mol_type = other DNA
agcccttctccccgcctcccactgt

SEQ ID NO: 68 - DNA, 25 bp, synthetic construct
  source 1..25: mol_type = other DNA
ctccgccacctgctccttcacctcg

SEQ ID NO: 69 - DNA, 20 bp, synthetic construct
  source 1..20: mol_type = other DNA
cttacgcagcttgcgcaggt

SEQ ID NO: 70 - DNA, 20 bp, synthetic construct
  source 1..20: mol_type = other DNA
gcttgcgcaggtgggaggcg

SEQ ID NO: 71 - DNA, 20 bp, synthetic construct
  source 1..20: mol_type = other DNA
acgcagcttgcgcaggtggg

SEQ ID NO: 72 - DNA, 20 bp, synthetic construct
  source 1..20: mol_type = other DNA
ccagagcaccgaggagctgc

SEQ ID NO: 73 - DNA, 20 bp, synthetic construct
  source 1..20: mol_type = other DNA
gccagagcaccgaggagctg

SEQ ID NO: 74 - DNA, 20 bp, synthetic construct
  source 1..20: mol_type = other DNA
gaggcgcacccgcagctcct

SEQ ID NO: 75 - DNA, 20 bp, synthetic construct
  source 1..20: mol_type = other DNA
agctgcgccagcagaccgag

SEQ ID NO: 76 - DNA, 20 bp, synthetic construct
  source 1..20: mol_type = other DNA
ccaggccaaggtggagcaag

SEQ ID NO: 77 - DNA, 20 bp, synthetic construct
  source 1..20: mol_type = other DNA
cacaggatgccaggccaagg

SEQ ID NO: 78 - DNA, 20 bp, synthetic construct
  source 1..20: mol_type = other DNA
acagtgtctgcacccagcgc

SEQ ID NO: 79 - DNA, 20 bp, synthetic construct
  source 1..20: mol_type = other DNA
ggccaaggtggagcaagcgg

SEQ ID NO: 80 - DNA, 20 bp, synthetic construct
  source 1..20: mol_type = other DNA
gaggcgcacccgcagctcct

SEQ ID NO: 81 - DNA, 4290 bp, synthetic construct
  source 1..4290: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgcgcctggtgcagtaccgcggcgaggtgcaggccatgctcggccagagcaccgaggagctgcgggtgcgcctcgcctcccacctgcgcaagctgtgatatctcgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 82 - protein, 284 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTRLVQYRGEVQAMLGQSTEELRVRLASHLRKL

SEQ ID NO: 83 - DNA, 4290 bp, synthetic construct
  source 1..4290: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgcgcctggtgcagtaccgcggcgaggtgcaggccatgctcggccagagcaccgaggagctgcgggtgagcctcgcctcccacctgcgcaagctgtgatatctcgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 84 - protein, 284 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTRLVQYRGEVQAMLGQSTEELRVSLASHLRKL

SEQ ID NO: 85 - DNA, 4284 bp, synthetic construct
  source 1..4284: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatacgcctggtgcagtaccgcggcgaggtgcaggccatgctcggccagagtactgaggagctgcgggtgcgcctcgcctcccacctgcgcaagctgatatcggccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 86 - protein, 282 aa, synthetic construct
MYRMQLLSCIALSLALVTNSIRLVQYRGEVQAMLGQSTEELRVRLASHLRKLISAMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - DNA, 4284 bp, synthetic construct
  source 1..4284: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatacgcctggtgcagtaccgcggcgaggtgcaggccatgctcggccagagtactgaggagctgcgggtgagcctcgcctcccacctgcgcaagctgatatcggccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 88 - protein, 282 aa, synthetic construct
MYRMQLLSCIALSLALVTNSIRLVQYRGEVQAMLGQSTEELRVSLASHLRKLISAMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 89 - DNA, 4284 bp, synthetic construct
  source 1..4284: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgagcaccgaggagctgcgggtgcgcctcgcctcccacctgcgcaagctgcgtaagcggctcctccgcgatgccgatgacctgcagaagtgatatctcgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 90 - protein, 282 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTSTEELRVRLASHLRKLRKRLLRDADDLQK

SEQ ID NO: 91 - DNA, 4284 bp, synthetic construct
  source 1..4284: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgagcaccgaggagctgcgggtgagcctcgcctcccacctgcgcaagctgcgtaagcggctcctccgcgatgccgatgacctgcagaagtgatatctcgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 92 - protein, 282 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTSTEELRVSLASHLRKLRKRLLRDADDLQK

SEQ ID NO: 93 - DNA, 4278 bp, synthetic construct
  source 1..4278: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgataagcaccgaggagctgcgggtgcgcctcgcctcccacctgcgcaagctgcgtaagcggctcctccgcgatgccgatgacctgcagaagatatcggccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 94 - protein, 280 aa, synthetic construct
MYRMQLLSCIALSLALVTNSISTEELRVRLASHLRKLRKRLLRDADDLQKISAMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 95 - DNA, 4278 bp, synthetic construct
  source 1..4278: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgataagcaccgaggagctgcgggtgagcctcgcctcccacctgcgcaagctgcgtaagcggctcctccgcgatgccgatgacctgcagaagatatcggccatggttagatctgtggagtgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 96 - protein, 280 aa, synthetic construct
MYRMQLLSCIALSLALVTNSISTEELRVSLASHLRKLRKRLLRDADDLQKISAMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - DNA, 6773 bp, synthetic construct
  source 1..6773: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcagacaacgtgctgacacagagccctgccagcctggctgtttctctgggacagagagccaccatcagctgcaaggccagccagagcgttgactacgacggcgacagctacatgaactggtatcagcagaagcccggccagccacctaaggtgttcatctacgccgccagcaacctggaaagcggcatccctgccagattttctggctctggcagcggcaccgacttcaccctgaatatccatcctgtggaagaagaggacgccgccacctactactgccagcagagcaatgaggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 98 - DNA, 7452 bp, synthetic construct
  source 1..7452: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcggaagtgaagctggtggaaagcggcggaggtgttgttcagcctggcggatctctgaagctgagctgtgccgccagcggcttcacctttagcagctacacaatgagctgggtccgacagacccctgagaagagactggaatgggtcgccaagatccggaacggcggaggcatcacctactacctggataccctgaagggcagattcaccatcagccgggacaacgccaagaacaccctgtacctgcagatgagcagcctgaagtccgaggacaccgccatctacttttgcgccagacactactacggcagcgaggactacttcgactattggggccagggcaccacactgaccgttagctctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgacctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 99 - DNA, 6773 bp, synthetic construct
  source 1..6773: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcagacatcgtgctgacacagagccctgccagcctggctgtttctctgggacagagagccaccatcagctgcaaggccagccagagcgttgactacgacggcgagaactacatgaactggtatcagcagaagcccggacagagccccaagctgctgatctacgtggccagcaatctggaaagcggcatccccgccagattttctggcagcggaagcggcaccgacttcaccctgaatatccatcctgtggaagaagaggacgccgccacctactactgccagcagtccaatctggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 100 - DNA, 7446 bp, synthetic construct
  source 1..7446: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcggaggtccagctgcagcagtctggcgccgaacttgttagacctggcgctctggtcaagctgagctgtaaagccagcggcttcaacatcaaggactaccatctgcactgggtcaagcagaggcctgagcagggactcgagtggatcggctggatcgaccccgagaacggcaacgtgatctacgaccccaagttccagggcaaagccaccatgaccgtggtcaccagcagcaacacagcctacctgcagctgagaagcctgaccagcgaagataccgccgtgtacttctgcaccagaggcacagccagagccagcttcgattattggggccagggcaccagcctgaccgtttcttctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgacctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 101 - DNA, 6773 bp, synthetic construct
  source 1..6773: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcagacatcgtgctgacacagagccctgccagcctggctgtttctctgggacagagagccaccatcagctgcaaggccagccagagcgttgactacgacggcgacagctacatgaactggtatcagcagaagtccggccagcctcctaagctgctgatctacgccgccagcaatctggaaagcggcatccctgccagattttccggctctggcagcggcaccgacttcaccctgaatatccatcctgtggaagaagaggacgccgccacctactactgccagcagagcaatgtggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 102 - DNA, 7449 bp, synthetic construct
  source 1..7449: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcggaggtccagctgcagcagtctggcgccgaacttgttagacctggcgctctggtcaagctgagctgtaaagccagcggcttcaacatcaaggactaccacatgcactgggtcaaagagcggcctgagcagggactcgagtggatcggatggatcgaccccgagaacggcaacactatgtacgaccccaagttccagggcaaagccagcatcaccgccgacacctctagcaacacagcctacctgcagctgagcagcctgacctctgaagataccgccgtgtactactgcgtgcggggaacagccagagccagctttgattattggggccagggcaccacactgaccgtgtcatctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataacctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 103 - DNA, 6773 bp, synthetic construct
  source 1..6773: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcagacatcgtgctgacacagagccctgccagcctggctgtttctctgggacagagagccaccatcagctgcaaggccagccagagcgttgactacgacggcgacacctacatgaactggtatcagcagaagcccggccagccacctaagctgctgatctacacagccagcaacctggaaagcggcatccccgccagattttctggcagcggaagcggcaccgacttcaccctgaatatccatcctgtggaagaggtggacgccgccacctactactgccagcagagcaatgaggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 104 - DNA, 7449 bp, synthetic construct
  source 1..7449: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcggaggtccagctgcagcagtctggcgccgaacttgttagacctggcgctctggtcaagtggtcctgtaaagccagcggcttcaacatcaaggactaccacatccactgggtcaagcagaggcctgagcagggcctcgattggatcggctggatcgaccccgagatcgacaagaccctgtacgaccccaagttccagggcaaagccagaatcaccgccgacaccagcagcaacacagcctacctgcaactgagcagcctgaccagcgaagataccgccgtgtactactgcgccagaggaacagccagagccagcttcgattattggggccagggcaccacactgaccgtgtcatctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataacctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 105 - DNA, 6773 bp, synthetic construct
  source 1..6773: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcagacaacgtgctgacacagagccctgccagcctggctgtttctctgggacagagagccaccatcagctgcaaggccagccagagcgttgactacgacggcgacagctacatgaactggtatcagcagaagcccggccagccacctaaggtgttcatctacgccgccagcaacctggaaagcggcatccctgccagattttctggctctggcagcggcaccaacttcaccctgaacattcaccccgtggaagaagaggacgccgccacctactactgccagcagagcaatgaggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 106 - DNA, 7452 bp, synthetic construct
  source 1..7452: mol_type = other DNA
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagcttgccaccatgtacaggatgcaactcctgtcttgcattgcactaagtcttgcacttgtcacgaattcggaagtgaagctggtggaaagcggcggaggactggttcaacctggcggatctctgaagctgagctgtgccgccagcggcttcaccttcagcagatacacaatgagctgggtccgacagacccctgagaagagactggaatgggtcgccaagatcagaaacgtcggcggcatcacctactatcccgacaccgtgaagggcagattcaccatctccagagacaacgccaagaacaccctgtacctgcagatgagcagcctgaagtccgaggacaccgccatgtactactgcgccagacactactacggcagcgaggactacttcgactattggggccagggcaccacactgaccgttagctctgctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgacctagcgtggcatctagacactctcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 107 - DNA, 15 bp, synthetic construct
  source 1..15: mol_type = other DNA
atggaggacgtgtgc

SEQ ID NO: 108 - DNA, 15 bp, synthetic construct
  source 1..15: mol_type = other DNA
atggaggacgtgtgc

SEQ ID NO: 109 - DNA, 15 bp, synthetic construct
  source 1..15: mol_type = other DNA
atggaggacgtgtgc

SEQ ID NO: 110 - DNA, 15 bp, synthetic construct
  source 1..15: mol_type = other DNA
gagctgcgggtgcgc

SEQ ID NO: 111 - DNA, 15 bp, synthetic construct
  source 1..15: mol_type = other DNA
gagctgcgggtgagc

SEQ ID NO: 112 - DNA, 15 bp, synthetic construct
  source 1..15: mol_type = other DNA
gagctgcgggtgagc

SEQ ID NO: 113 - DNA, 15 bp, synthetic construct
  source 1..15: mol_type = other DNA
gacctgcagaagcgc

SEQ ID NO: 114 - DNA, 15 bp, synthetic construct
  source 1..15: mol_type = other DNA
gacctgcagaagcgc

SEQ ID NO: 115 - DNA, 15 bp, synthetic construct
  source 1..15: mol_type = other DNA
gacctgcanaagcgc

(19) United States
(12) Patent Application Publication
(10) Pub. No.: US 2024/0301044 A1
(43) Pub. Date: Sep. 12, 2024

(54) APOE ANTIBODIES, FUSION PROTEINS AND USES THEREOF

(71) Applicants: The General Hospital Corporation, Boston, MA (US); Banner Health, Phoenix, AZ (US); The Schepens Eye Research Institute, Inc., Boston, MA (US)

(72) Inventors: Yakeel T. Quiroz, Charlestown, MA (US); Joseph F. Arboleda-Velasquez, Newton, MA (US); Eric Reiman, Phoenix, AZ (US); Francisco Lopera, Medellin (CO)

(21) Appl. No.: 18/611,374

(22) Filed: Mar. 20, 2024

Related U.S. Application Data
(63) Continuation of application No. 17/614,008, filed on Nov. 24, 2021.
(60) Provisional application No. 62/873,019, filed on Jul. 11, 2019.
(60) Provisional application No. 62/853,676, filed on May 28, 2019.

(51) Int. Cl. C07K 16/18; A61K 39/00; A61P 25/28; C07K 14/775

(52) U.S. Cl. CPC C07K 16/18; A61P 25/28; C07K 14/775; A61K 2039/505; C07K 2317/24; C07K 2317/34; C07K 2317/92; C07K 2319/30

(57) ABSTRACT

Methods and compositions for preventing or treating cognitive decline associated with dementia and/or mild cognitive impairment and/or neurodegeneration using antibodies, peptides, fusion proteins, or genome editing systems that modulate HSPG/heparin binding affinities of ApoE.

### CLAIM OF PRIORITY

This application is a continuation of U.S. patent application Ser. No. 17/614,008, filed Nov. 24, 2021, which is a § 371 National Stage Application of PCT/US2020/034978, filed May 28, 2020, which claims the benefit of U.S. Provisional Application No. 62/853,676, filed May 28, 2019, and U.S. Provisional Application No. 62/873,019, filed Jul. 11, 2019. The entire contents of the foregoing are incorporated by reference herein.

### FEDERALLY SPONSORED RESEARCH OR DEVELOPMENT

This invention was made with Government support under Grant No. OD019833 awarded by the National Institutes of Health, Grant Nos. AG054671, AG031581, and AG19610 awarded by the National Institute on Aging, and Grant Nos. NS100121 and NS110048 awarded by the National Institute of Neurological Disorders and Stroke. The Government has certain rights in the invention.

### SEQUENCE LISTING

This application contains a Sequence Listing that has been submitted electronically as an XML file named “29539-0386003_SL_ST26.XML.” The XML file, created on Mar. 20, 2024, is 215,484 bytes in size. The material in the XML file is hereby incorporated by reference in its entirety.

### TECHNICAL FIELD

Described herein are methods and compositions for preventing or treating cognitive decline associated with dementia and/or mild cognitive impairment by modulating the heparan sulfate proteoglycans (HSPG)/glycosaminoglycan (GAG) heparin-binding affinity of Apolipoprotein E (ApoE).

### BACKGROUND

Alzheimer's disease (AD) is a chronic neurodegenerative disease that usually starts slowly and gradually worsens over time. It is the cause of 60-70% of cases of dementia. The disease process is associated with plaques and neurofibrillary tangles in the brain. There are presently no treatments to stop or reverse its progression, though some may temporarily improve symptoms. The accumulation, aggregation and deposition of amyloid-β (Aβ) peptides in the brain are central to the pathogenesis of Alzheimer's disease (AD). Growing evidence has demonstrated that ApoE strongly influences AD pathogenesis by controlling Aβ aggregation and metabolism (Fu et al., Mol Neurodegener 11:37, 2016). APOE impacts amyloid production, aggregation, and clearance, is a component of amyloid plaques, and exacerbates tau-mediated neurodegeneration. ApoE is 299 amino acids long and is polymorphic with three major alleles (epsilon 2, epsilon 3, and epsilon 4) which differ from each other by only one or two amino acids at positions 112 and 158: ApoE2 (cys112, cys158), ApoE3 (cys112, arg158), and ApoE4 (arg112, arg158). Accordingly, there is a need of therapeutics targeting and modulating the function of ApoE proteins for treating or preventing AD and cognitive decline associated with dementia or mild cognitive impairment.

### SUMMARY

In one aspect, this disclosure features an isolated monoclonal antibody that specifically binds to one or more (e.g. 1, 2, 3 or 4) HSPG-binding sites or one or more (e.g. 1, 2, 3, or 4) sites of allosteric modulation of HSPG binding of a wild type or mutant Apolipoprotein E (ApoE). In some embodiments, the antibody binds to a polypeptide having an amino acid sequence at least 95% (e.g. 96%, 97%, 98%, 99% or 100%) identical to TEELRVRLASHLRK (SEQ ID NO:3). In some embodiments, the antibody binds to a polypeptide having an amino acid sequence at least 95% (e.g. 96%, 97%, 98%, 99% or 100%) identical to TEELRVSLASHLRK (SEQ ID NO:2). In some embodiments, the antibody binds to one or more (e.g. 1, 2, 3 or 4) HSPG-binding sites of a wild type or mutant ApoE2, ApoE3, or ApoE4.

In some embodiments, the antibody competes with and/or binds the same epitope as a reference anti-ApoE antibody comprising a heavy chain variable region (VH) and a light chain variable region (VL), wherein the VH and VL of the reference antibody comprise: (i) the amino acid sequence set forth in SEQ ID NO: 13 and the amino acid sequence set forth in SEQ ID NO: 12, respectively; (ii) the amino acid sequence set forth in SEQ ID NO:23 and the amino acid sequence set forth in SEQ ID NO:22, respectively; (iii) the amino acid sequence set forth in SEQ ID NO:33 and the amino acid sequence set forth in SEQ ID NO:32, respectively; or (iv) the amino acid sequence set forth in SEQ ID NO:43 and the amino acid sequence set forth in SEQ ID NO:42, respectively. In some embodiments of any of the antibodies described herein, the antibody competes with and/or binds the same epitope as a reference anti-ApoE antibody comprising a heavy chain and a light chain, wherein the heavy chain and light chain of the reference antibody comprise the amino acid sequence set forth in SEQ ID NO: 53 and the amino acid sequence set forth in SEQ ID NO: 52.

In another aspect, provided herein are anti-ApoE antibodies comprising a VH comprising VHCDR1, VHCDR2, and VHCDR3, and a VL comprising VLCDR1, VLCDR2, and VLCDR3, wherein VHCDR1, VHCDR2, VHCDR3, VLCDR1, VLCDR2, and VLCDR3 comprise: (i) SEQ ID Nos: 7, 8, 9, 4, 5, 6, respectively; (ii) SEQ ID Nos: 17, 18, 19, 14, 15, 16, respectively; (iii) SEQ ID Nos: 27, 28, 29, 24, 25, 26, respectively; (iv) SEQ ID Nos: 37, 38, 39, 34, 35, 36, respectively; or (v) SEQ ID Nos: 47, 48, 49, 44, 45, 46, respectively. In some embodiments of any of the antibodies described herein, (i) the VH and the VL comprise an amino acid sequence that is at least 75%, 80%, 85%, 90%, 95%, or 100% identical to the amino acid sequences set forth in SEQ ID NOs: 13 and 12, respectively; (ii) the VH and the VL comprise an amino acid sequence that is at least 75%, 80%, 85%, 90%, 95%, or 100% identical to the amino acid sequences set forth in SEQ ID NOs:23 and 22, respectively; (iii) the VH and the VL comprise an amino acid sequence that is at least 75%, 80%, 85%, 90%, 95%, or 100% identical to the amino acid sequences set forth in SEQ ID NOs:33 and 32, respectively; or (iv) the VH and the VL comprise an amino acid sequence that is at least 75%, 80%, 85%, 90%, 95%, or 100% identical to the amino acid sequences set forth in SEQ ID NOs:43 and 42, respectively. In some embodiments of any of the antibodies described herein, the antibody comprises a heavy chain and a light chain comprising an amino acid sequence that is at least 70%, 75%, 80%, 85%, 90%, 95%, or 100% identical to the amino acid sequence set forth in SEQ ID Nos: 53 and 52, respectively. In some embodiments, the antibody includes a mouse IgG1, IgG2a, IgG2b, IgG2c, or IgG3 heavy chain constant region. In some embodiments, the antibody includes a human IgG1, IgG2, IgG3, or IgG4 heavy chain constant region. In some embodiments, the antibody includes a human kappa or human lambda light chain constant region. In some embodiments, the antibody is a whole antibody, a single domain antibody, a humanized antibody, a chimeric antibody, a bispecific antibody, a Fv, a scFv, an sc(Fv)2, a diabody, a nanobody, an Fab, or an F(ab′)2. In some embodiments, the antibody further includes a half-life extending moiety. In some embodiments, the antibody further includes a blood-brain barrier penetrating moiety. In some embodiments, the antibody further includes a detectable label. In some embodiments, provided herein are pharmaceutical compositions comprising any of the antibodies described herein. In some embodiments, provided herein are polynucleotide or polynucleotides encoding any of the antibodies described herein. In some embodiments, provided herein are vector or vectors comprising the polynucleotide or polynucleotides described herein. In some embodiments provided herein are host cells comprising the polynucleotide or polynucleotides described herein, or the vector or vectors described herein. In another aspect, provided herein are methods of making an anti-ApoE antibody, the methods include: (a) culturing any of the host cells described herein under conditions that permit expression of the antibody; and (b) isolating the antibody. In some embodiments, the methods further include formulating the antibody as a sterile formulation suitable for administration to a human.

In another aspect, provided herein is an Fc-fusion protein that includes: a HSPG-binding domain of a wild type ApoE or mutant ApoE comprising an amino acid sequence at least 95% identical to an amino acid sequence selected from the group consisting of STEELRVRLASHLRKLRKRLLRDADDLQK (SEQ ID NO:57), STEELRVSLASHLRKLRKRLLRDADDLQK (SEQ ID NO:58), RLVQYRGEVQAMLGQSTEELRVRLASHLRKL (SEQ ID NO:59), and RLVQYRGEVQAMLGQSTEELRVSLASHLRKL (SEQ ID NO:60). In some embodiments, the Fc-fusion protein includes an Fc region of a human antibody. In some embodiments, the human antibody is selected from the group consisting of a human IgG1, IgG2, IgG3 and IgG4 molecule. In some embodiments, provided herein are pharmaceutical compositions comprising any of the Fc-fusion proteins described herein. In some embodiments, provided herein are polynucleotide or polynucleotides encoding the Fc-fusion proteins described herein. In some embodiments, provided herein are vector or vectors comprising any of the polynucleotide or polynucleotides described herein. In some embodiments, provided herein are host cells comprising the polynucleotide or polynucleotides described herein, or the vector or vectors described herein.

In another aspect, provided herein are pharmaceutical composition for eliciting an immune response that include: (i) a HSPG-binding domain of a wild type ApoE or mutant ApoE comprising an amino acid sequence at least 95% identical to an amino acid sequence selected from the group consisting of STEELRVRLASHLRKLRKRLLRDADDLQK (SEQ ID NO:57), STEELRVSLASHLRKLRKRLLRDADDLQK (SEQ ID NO:58), RLVQYRGEVQAMLGQSTEELRVRLASHLRKL (SEQ ID NO:59), and RLVQYRGEVQAMLGQSTEELRVSLASHLRKL (SEQ ID NO:60); and (ii) a pharmaceutically acceptable adjuvant.

In another aspect, provided herein is a pharmaceutical composition comprising a human cell expressing any of the antibodies described herein or any of the Fc-fusion proteins described herein.

In another aspect, provided herein is a method of improving, slowing down, delaying the onset of, preventing or reversing cognitive decline associated with dementia and/or mild cognitive impairment and/or neurodegeneration in a human subject in need thereof, comprising administering to the human subject a therapeutically effective amount of any of the antibody, the Fc-fusion protein, or the pharmaceutical compositions described herein.

In another aspect, provided herein is a method of improving, slowing down, delaying the onset, preventing or reversing cognitive decline associated with dementia and/or mild cognitive impairment and/or neurodegeneration in a human subject in need thereof, the method comprising administering to said subject: (i) a viral vector comprising a nucleotide sequence encoding a gRNA molecule comprising a targeting domain complementary with a target domain from the APOE gene; (ii) a viral vector comprising a nucleotide sequence encoding a Cas9 molecule; and (iii) a viral vector comprising a template nucleic acid, wherein the template nucleic acid comprises an Adenine to replace the Cytosine at position 19:g.45412013C>A in the APOE gene, wherein said administration results in the generation of one or more ApoE R136S alleles in one or more cells of said subject. In some embodiments, the targeting domain of the gRNA molecule includes a sequence that is the same as, or differs by no more than 3 nucleotides from, a sequence from Table 7.

As used herein, “prevent” means to reduce risk of developing the disorder.

In some embodiments, the human subject is diagnosed with or is at risk for developing Alzheimer's disease. In some embodiments, the human subject carries one or more copies of the APOE4 allele. In some embodiments, the human subject carries one or more mutations in at least one gene selected from the group consisting of: APP, PSEN1, and PSEN2. In some embodiments, the human subject carries one or more mutations in additional genes that cause autosomal-dominant Alzheimer's disease (e.g. those described in Bateman et al., Alzheimer's Research & Therapy 3(1): 1, 2011). In some embodiments, the human subject carries all or a portion of a third copy of chromosome 21. In some embodiments, the human subject is diagnosed with Alzheimer's disease by established biomarkers, such as those obtained via brain imaging, or blood or CSF samples. In some embodiments, the human subject is over the age of 50 (e.g. over the age of 55, 60, 65, 70, 75, 80, 85, 90, or 95).

In some embodiments, the human subject is diagnosed with or is at risk of developing a disorder selected from the group consisting of: vascular cognitive impairment, vascular dementia, cerebral autosomal dominant arteriopathy with subcortical infarcts and leukoencephalopathy (CADASIL), cerebral autosomal recessive arteriopathy with subcortical infarcts and leukoencephalopathy (CARASIL), Lewy body dementia, frontotemporal dementia, amyotrophic lateral sclerosis, multiple sclerosis, Parkinson's disease, Huntington's disease, neurodegenerative diseases, cerebrovascular diseases, brain injury, chronic traumatic encephalopathy, tauopathies, amyloidopathies, synucleinopathies, Creutzfeldt-Jakob disease, retinal degeneration, glaucoma, retinal injury, optic nerve degeneration, and aging.

In yet another aspect, provided herein is a method of identifying a human subject less susceptible to developing an early onset neurodegenerative disease, comprising: obtaining a biological sample from the subject; detecting the presence of at least one mutant allele of APOE3, or the presence of a mutant ApoE3 gene product, in the biological sample; and identifying a subject as being less susceptible to developing an early onset neurodegenerative disease, based on the presence of a mutant ApoE3 allele or gene product in the biological sample. In some embodiments, the biological sample is blood, cerebrospinal fluid, saliva, urine, tears, vitreous humor, aqueous humor, or a tissue specimen. In some embodiments, the neurodegenerative disease is Alzheimer's disease, Parkinson's disease, Huntington's disease, amyotrophic lateral sclerosis, retinal degeneration, or glaucoma. In some embodiments, the retinal degeneration is age-related macular degeneration. In some embodiments, the detecting comprises determining the sequence of an APOE3 allele in the subject. In some embodiments, the detecting comprises determining the presence or absence of an APOE3 sequence that encodes an ApoE3 protein with a mutation at R136 as compared to a wild type ApoE3 protein. In some embodiments, the mutation at R136 is R136S, R136H, or R136C. In some embodiments of any of the methods of identifying a human subject less susceptible to developing an early onset neurodegenerative disease described herein, the methods further include selecting a subject for inclusion in a clinical trial, and optionally administering an experimental treatment, or excluding the subject from the clinical trial, if the subject does not have a mutant APOE3 allele. In some embodiments of any of the methods of identifying a human subject less susceptible to developing an early onset neurodegenerative disease described herein, the methods further include selecting a subject for inclusion in a clinical trial, and optionally administering an experimental treatment, or excluding the subject from the clinical trial, if the subject has a mutant APOE3 allele.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows a model of the structure of the wild-type APOE3 protein. N-terminal (residues 1-191) and C-terminal (residues 201-299) domains are shown. The amino acid positions for APOE4 (C112R), APOE3ch (R136S) and APOE2 (R158C) variants are shown.

FIG. 2 shows representative Sanger sequencing results of APOE from control, proband and descendant's samples.

FIG. 3 depicts the subject's genealogy, with circles representing females, squares representing males, diamonds representing individuals whose gender has been masked for privacy, arrowhead depicts proband individual with MCI, and shading indicates individual with history of dementia. Deceased individuals are marked with a crossed bar. The individual APOE and PSEN1 genotypes are indicated as appropriate to preserve anonymity.

FIGS. 4A and 4B are fundus photographs of the right and left eyes, respectively. FIG. 4C shows an infrared image of the right eye that depicts the cross section of the retina (line) seen in FIG. 4D. FIG. 4D shows results of optical coherence tomography (OCT) of the right eye. FIG. 4E shows an infrared image of the left eye. FIG. 4F shows results of OCT imaging of the left eye.

FIG. 5 shows brain imaging results showing the amyloid plaque burden and PHF Tau burden in the brains of the PSEN1 mutation carrier with late onset MCI (mild cognitive impairment) and a PSEN1 mutation carrier with MCI onset at a typical age for this mutation (44 years).

FIG. 6 shows measurements of amyloid burden, tau burden, hippocampal volume, and the levels of glucose metabolism in PSEN1 E280A mutation carriers. Red dots represent the measurements for the carrier with two APOE3ch alleles and an exceptionally late onset of MCI. Black dots represent PSEN1 E280A mutation carriers with MCI at the kindred's typical, younger age at MCI onset. Gray dots represent PSEN1 E280A mutation carriers who have not yet developed MCI.

FIG. 7 shows the rate of Aβ₄₂ fibril formation in the presence of APOE3 wild-type, APOE3ch, or in the absence of APOE as detected by Thioflavin T fluorescence. Changes in relative fluorescence units (RFU) were plotted for time in minutes (min). (***P<0.001, ****P<0.0001)

FIG. 8 shows schematic of the split-luciferase complementation triggered by amyloid oligomerization (top) and percentage of luminescence obtained by split-luciferase complementation assay after 24 hours in culture medium from 293T cells transfected with ApoE3ch or ApoE3 wild type.

FIG. 9 shows ELISA results of APOE2 and APOE4 heparin-binding affinity.

FIG. 10 shows western blot analysis of the heparin-binding affinity of ApoE2, ApoE3, ApoE4 and ApoE3ch.

FIGS. 11A and 11B show ELISA results of the heparin-binding affinity of ApoE2, ApoE3, ApoE4 and ApoE3ch.

FIG. 12A is a schematic showing the experimental setup for testing the specificity of the monoclonal ApoE3 antibody in blocking ApoE3/heparin binding.

FIGS. 12B and 12C are schematics showing the process of passing the ApoE3 protein pre-incubated with the monoclonal antibody through a heparin binding column followed by washing and eluting.

FIGS. 13A-13B show results from BCA assays performed on various fractions from the heparin binding column. FIG. 13A shows the amount of ApoE3 in various fractions in the absence of the ApoE3 antibody. FIG. 13B shows the amount of ApoE3 in various fractions in the presence of the ApoE3 antibody.

FIG. 14 shows western blot results showing the amount of ApoE3 in various fractions with or without pre-incubation with the monoclonal ApoE3 antibody.

FIGS. 15A-15G show ELISA analysis of the 19G10-2, 23B2, 2H79-1, 30E1-2, 16H8, 25F1-2, and 29G10-1 antibody, respectively.

FIG. 16 shows western blot analysis of the heparin-binding affinity of ApoE3 treated with the wild type ApoE3 peptide and the ApoE3ch mutant peptide.

FIGS. 17A-17D show modeling of the interaction of ApoE fragments with heparin. FIG. 17A shows a model of the wild type ApoE fragment containing amino acids 129-157 interacting with heparin. FIG. 17B shows a model of the fragment of ApoE R136S that contains amino acids 129-157 interacting with heparin. FIG. 17C shows a model of the wild type ApoE fragment containing amino acids 114-144 interacting with heparin. FIG. 17D shows a model of the fragment of ApoE R136S that contains amino acids 114-144 interacting with heparin.

FIGS. 18A-18B show heparin-affinity chromatography and western blot analysis of antibody 1H4.

FIGS. 19A-19D show ELISA results of 1H4-2 serum tested with ApoE3 WT full-length protein (A), ApoE3 WT peptide (B), ApoE3ch full length protein (C), and ApoE3ch peptide (D).

FIG. 20 shows ELISA results of 1H4-2 serum tested with ApoE3 WT full-length protein, ApoE3 WT peptide, ApoE3ch full length protein, and ApoE3ch peptide.

FIG. 21 shows representative ELISA profiles of serial dilutions of the antibody 1H4 incubated either with human recombinant ApoE3 or mouse recombinant ApoE3.

FIG. 22 shows ELISA results for the monoclonal 1H4 antibody purified from cloned hybridoma.

FIGS. 23A and 23B show heparin-affinity chromatography and western blot analysis of antibody 7C11.

FIGS. 24A-24D show ELISA results from testing the 7C11-1 serum with ApoE3 WT full-length protein (A), ApoE3 WT peptide (B), ApoE3ch full length protein (C), or ApoE3ch peptide (D).

FIG. 25 shows ELISA results from testing the 7C11-1 serum with ApoE3 WT full-length protein, ApoE3 WT peptide, ApoE3ch full length protein, or ApoE3ch peptide.

FIG. 26 shows ELISA results for the monoclonal 7C11-1 antibody purified from cloned hybridoma.

FIG. 27 shows results from ELISA screening of the 19G10-2 antibody against the heparin binding domain of APOE3 Wild Type (WT) and APOE3ch Mutant recombinant protein.

FIGS. 28A and 28B show heparin-affinity chromatography and western blot analysis of antibody 19G10-2.

FIG. 29 shows western blotting of ApoE3 WT incubated with 19G10-2 serum antibody.

FIG. 30 is representative ELISA showing the differences in binding of both serum and monoclonal antibody hybridoma supernatant 19G10-2 for ApoE3WT or ApoE3ch.

FIG. 31 is an enlargement of the Y axes in FIG. 30.

FIG. 32 shows ELISA results for the monoclonal 19G10-2 antibody purified from cloned hybridoma.

FIG. 33 shows results from ELISA screening of the 25F1-2 antibody against the heparin binding domain of APOE3 Wild Type (WT) and APOE3ch Mutant recombinant protein.

FIGS. 34A and 34B show heparin-affinity chromatography and western blot analysis of antibody 25F1-2.

FIG. 35 shows western blotting of ApoE3 WT incubated with the 25F1-2 monoclonal antibody.

FIG. 36 is representative ELISA showing the differences in binding of both 25F1-2 serum and monoclonal antibody hybridoma supernatant 25F1-2 for ApoE3WT or ApoE3ch.

FIG. 37 is an enlargement of the Y axes of FIG. 36.

FIG. 38 shows ELISA results for the monoclonal 25F1-2 antibody purified from cloned hybridoma.

FIG. 39 shows ELISA screening of the 1343 antibody against the heparin binding domain of APOE3 Wild Type (WT) and APOE3ch Mutant recombinant protein.

FIG. 40 shows ELISA screening of the 1343 antibody against the heparin binding domain of APOE3 Wild Type (WT) and APOE3ch Mutant recombinant protein.

FIG. 41A shows western blot analysis of ApoE in protein fractions eluted from heparin columns in the presence or absence of the 1343 antibody. FIG. 41B shows ELISA analysis of the fractions.

FIG. 42A shows an exemplary experimental outline for an intraocular model of inducible APOE-dependent Tau hyperphosphorylation. FIG. 42B shows PHF tau in control retina injected with PBS. FIG. 42C shows retina injected with recombinant human APOE3.

FIGS. 43A-43I show PHF tau in control retina as compared to retina injected with either the mouse 1H4-2 antibody or the humanized 1343 Ah antibody.

FIG. 44 shows representative binding measurements of increasing concentrations (nM) of ApoE3 protein to 1H4 on the protein A biosensor.

### DETAILED DESCRIPTION

The present disclosure uncovers that homozygosity for APOE3ch (having two copies of the APOE3 Christchurch (R136S) mutation) is associated with a profound resistance to the clinical onset of Alzheimer's disease, and that the R136S mutation significantly diminishes the ability of ApoE to bind heparan sulfate proteoglycans (HSPG)/heparin. Accordingly, the present disclosure is related to antibodies that bind to wild type ApoE and/or ApoE isoform(s) containing the R136S mutation (e.g. antibodies that block the interaction and/or reduces binding between ApoE and HSPG/GAG/heparin). Fusion proteins containing peptide fragments (e.g. HSPG/GAG/heparin-binding domain) of wild type and mutant ApoE containing the R136S mutation are also contemplated. These proteins may be administered via human cells expressing such compositions. The present disclosure is further related to small molecules that block the interaction between ApoE and HSPG/heparin, and methods of screening for small molecules of the same. Also provided are compositions and methods of editing the ApoE locus with a genome editing system. The antibodies, fusion proteins, small molecules, and genome editing systems described herein are useful in the treatment or prevention of cognitive decline associated with dementia and/or mild cognitive impairment (MCI) and/or neurodegeneration, e.g. Alzheimer's disease, vascular dementia, Lewy body dementia, frontotemporal dementia, Parkinson's disease, or Huntington's disease. The antibodies, fusion proteins, small molecules, and genome editing systems described herein are also useful in the treatment or prevention of neurodegenerative diseases, cerebrovascular conditions, brain injuries, retinal degeneration, optic nerve degeneration, or retinal injury.

### Apolipoprotein E (ApoE)

Apolipoprotein E variants are the major genetic modifier of AD contributing to the susceptibility to late-onset AD. APOE4 allele leads to a change of cysteine to arginine at position 112 and is associated with a 5 fold increase in AD risk in single allele carriers, reaching a 20 fold increase in homozygote carriers. APOE2 leads to an amino acid change of arginine to cysteine at position 158 and is protective for AD whereas the APOE3 allele is thought to be neutral (Corder et al., Nat Genet (7) 180-184, 1994; Hauser et al., Cure Alzheimer Res (10); 808-817, 2013). APOE impacts amyloid production, aggregation, and clearance, is a component of amyloid plaques, and exacerbates tau-mediated neurodegeneration. APOE alleles also regulate lipid metabolism and cardiovascular risk. About 5-10% of APOE2 homozygote individuals develop hyperlipoproteinemia type III (HLP III), whereas other APOE rare variants are linked to autosomal dominant HLP III. HLP III is characterized by increased plasma cholesterol and triglycerides levels and by the presence of tuberous or striated palmar xanthomas. The mechanisms by which APOE alleles modify AD risk and cause HLP III are not completely understood. Salient APOE properties impacted by specific mutations include differences in 1) binding affinities to lipids and the LDL receptor; 2) nature of interdomain interactions between its N-terminus (amino acids 1 to 199) and C-terminus domains (216 to 299); and, 3) ability to form homo-oligomers mediated by the C-terminus domain (Frieden et al., PNAS (109):8913-8918, 2012; Georgiadou et al., PLOS One (6)e27037, 2011; Lalazar et al., J Biol Chem (263)3542-3545, 1988).

Heparan sulfate (HS) is a linear polysaccharide found in all animal tissues, which occurs as a proteoglycan (HSPG) in which two or three HS chains are attached in close proximity to cell surface or extracellular matrix proteins. HSPG moieties are present in hundreds of proteins located in the plasma membrane and in the extracellular matrix. Protein-protein interactions mediated via HSPG play a critical role in a multitude of processes relevant to Alzheimer's pathology including amyloid and tau pathology. Heparan sulfate is a member of the glycosaminoglycan family of carbohydrates and is very closely related in structure to heparin. Both consist of a variably sulfated repeating disaccharide unit. Heparan sulfate binds with a large number of extracellular proteins. These are often collectively called the “heparin interactome” or “heparin-binding proteins”, because they are isolated by affinity chromatography on the related polysaccharide heparin.

An exemplary amino acid sequence of the human ApoE3 protein (Uniprot Accession No. P02649) is shown below:

(SEQ ID NO: 1)
KVEQAVETEPEPELRQQTEWQSGQRWELALGRFWDYLRWVQTLSEQVQEE
LLSSQVTQELRALMDETMKELKAYKSELEEQLTPVAEETRARLSKELQAA
QARLGADMEDVCGRLVQYRGEVQAMLGQSTEELRVRLASHLRKLRKRLLR
DADDLQKRLAVYQAGAREGAERGLSAIRERLGPLVEQGRVRAATVGSLAG
QPLQERAQAWGERLRARMEEMGSRTRDRLDEVKEQVAEVRAKLEEQAQQI
RLQAEAFQARLKSWEEPLVEDMQRQWAGLVEKVQAAVGTSAAPVPSDNH

At least two HSPG/heparin-binding domains have been identified in human ApoE, one located in the N-terminal domain and one in the C-terminal domain (Weisgraber et al. J Biol Chem, 261(5):2068-76, 1986; Saito et al., J Biol Chem, 278(17): 14782-7, 2003). The HSPG/heparin-binding domain near arginine 136 (R136) (the N-terminal HSPG/heparin-binding domain) is functional in the full-length lipidated and delipidated ApoE, while the HSPG/heparin-binding domain in the C-terminal domain is functional only in the absence of the N-terminal domain and in delipidated ApoE. The N-terminal HSPG/heparin-binding domain is well-characterized, and comprises the amino acid residues 142 to 147 of SEQ ID NO: 1 (bolded). The C-terminal HSPG/heparin-binding domain is less well-characterized, and comprises the lysine (K) at position 233 and other charged amino acids in the vicinity including amino acid residues 211 to 218 and 243 to 272 of SEQ ID NO: 1. The present inventors show that the arginine at position 136 of ApoE plays a critical role in heparin binding of ApoE. Without wishing to be bound by theory, a potential mechanism is the allosteric modulation of heparin binding mediated by the arginine at position 136. Allosteric modulation as used herein is related to the modulation of ligand binding through the binding of allosteric modulators at one or more sites of allosteric modulation, which may be different from the binding site(s) of the ligand. In some embodiments, the one or more sites of allosteric modulation for ApoE and HSPG/heparin binding comprise the arginine at position 136 of ApoE as shown in SEQ ID NO: 1. “HSPG/heparin-binding domain(s)”, “HSPG/heparin-binding site(s)”, “HSPG-binding domain(s)”, and “HSPG-binding site(s)” are used interchangeably herein.

### Anti-ApoE Antibodies

Provided are anti-ApoE antibodies that bind to a wild type or a mutant ApoE protein (e.g., a human ApoE protein). In some instances, the antibodies described herein bind to a wild type ApoE protein (e.g. ApoE2, ApoE3 or ApoE4), but not to a mutant ApoE protein (e.g. ApoEch). In some instances, the antibodies described herein bind to a mutant ApoE protein (e.g. ApoEch), but not to a wild type ApoE protein (e.g. ApoE2, ApoE3 or ApoE4). In some instances, the antibodies described herein bind to both a mutant ApoE protein (e.g. ApoEch), and a wild type ApoE protein (e.g. ApoE2, ApoE3 or ApoE4).

In some instances, the antibodies provided herein block the interaction between a wild type ApoE protein (e.g. ApoE2, ApoE3 or ApoE4) and HSPG. The antibodies provided herein may reduce or modulate the binding affinity of an ApoE protein (e.g. ApoE2, ApoE3 or ApoE4) to HSPG. In some instances, the antibodies provided herein bind to the HSPG-binding domain of a wild type ApoE protein. In some instances, the antibodies provided herein bind to one or more sites of allosteric modulation of HSPG/ApoE binding (e.g., amino acid position 136 of ApoE). In some instances, the antibodies described herein reduces fibril formation and/or amyloid oligomerization.

In some instances, the antibodies provided herein bind to an amino acid sequence in a wild type or mutant ApoE that comprises or consists of TEELRVSLASHLRK (SEQ ID NO:2). In some instances, the antibodies provided herein bind to an amino acid sequence in a wild type or mutant ApoE that comprises or consists of TEELRVRLASHLRK (SEQ ID NO:3). In some instances, the amino acid sequence TEELRVSLASHLRK (SEQ ID NO:2) comprises or consists of an epitope for the antibodies provided herein. In some instances, the amino acid sequence TEELRVRLASHLRK (SEQ ID NO:3) comprises or consists of an epitope for the antibodies provided herein. Variants of these sequences can also be used, e.g., those that are at least 80%, 85%, 90%, or 95% identical to these sequences.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 70% (e.g., at least 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the Needleman and Wunsch ((1970) J. Mol. Biol. 48:444-453) algorithm, which has been incorporated into the GAP program in the GCG software package (available at gcg.com), using either a Blossum 62 matrix, a PAM250 matrix, a NWSgapdna.CMP matrix. In some embodiments, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

Usage of the term “antibody” in this disclosure is meant to cover a whole antibody (as opposed to a minibody, nanobody or antibody fragment), a bispecific antibody, a tertravalent antibody, a multispecific antibody, a minibody, a nanobody, and antibody fragments. In some instances, the anti-ApoE antibody of this disclosure is a whole antibody. In some instances, the anti-ApoE antibody of this disclosure is a chimeric, human, or humanized antibody. In certain instances, the heavy chain constant region of the anti-ApoE antibody is a human IgG1, human IgG2, human IgG3, or human IgG4 constant region. In certain instances, the light constant region is a human kappa constant region. In other instances, the light constant region is a human lambda constant region. In some instances, the antibodies of this disclosure are designed to have low effector functionality (e.g., by Fc modifications such as N297Q, T299A, etc. Sec, also, Wang, X., Mathicu, M. & Brezski, R. J. Protein Cell (2018) 9: 63. doi.org/10.1007/s13238-017-0473-8 (incorporated by reference herein)). In some cases, the Fc moiety of the antibody is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc (in this format—that minimizes effector function—the CH1 and CH2 domains are IgG4 with a ‘fixed’ hinge (S228P) and is aglycosylated. The CH3 domain is hIgG1, or a hIgG4(S228P) agly Fc). In one case, the antibody has one of the following three scaffolds with reduced effector function: hIgG1 agly (N297Q); hIgG2 SAA (see, Vafa et al. Methods, 65(1): 114-26 (2014); and hIgG4P/G1 agly (see, US 2012/0100140 A1).

In some embodiments, an antibody or ApoE-binding fragment thereof described herein demonstrates the binding characteristics and/or biological properties as outlined for the antibodies 1H4-2, 7C11-1, 19G10-2, 23B2 (1343), 2H79-1, 30E1-2, 16H8, 25F1-2, and 29G10-1 illustrated in the Examples section below.

In some embodiments, the present disclosure provides an antibody that binds to wild type human ApoE or a portion thereof and has one or more of the following properties: (i) binds with high affinity of KD≤20 nM to wild type human ApoE; (ii) competes with wild type human ApoE for binding to heparin; and (iii) reduces Paired Helical Filament (PHF) Tau formation in retinal cells.

In some embodiments, the present disclosure provides an antibody that binds to a mutant human ApoE (e.g., those having a mutation at amino acid position 136 of the human ApoE, such as ApoEch) or a portion thereof and has one or more of the following properties: (i) binds with high affinity of KD≤20 nM to mutant human ApoE (e.g., mutation at amino acid position 136 of the human ApoE, such as ApoEch); (ii) competes with wild type human ApoE for binding to heparin; and (iii) reduces Paired Helical Filament (PHF) Tau formation in retinal cells.

Any of the anti-ApoE antibodies described herein are useful for treating or preventing disorders associated with dementia or mild cognitive impairment (MCI) (e.g. Alzheimer's disease, vascular dementia, Lewy body dementia, frontotemporal dementia, Parkinson's disease, or Huntington's disease), neurodegenerative diseases, cerebrovascular diseases, brain injury, retinal degeneration, or retinal injury.

### Exemplary Antibody 1H4-2

Antibody 1H4-2 was generated by immunizing with APOE: KLH-CTEELRVRLASHLRK-CONH2. The amino acid sequences of the complementarity determining regions (CDRs) and the heavy chain variable region and light chain variable regions of 1H4-2 are provided below.

| Variable | Chain | | | |
|---|---|---|---|---|
| region | type | CDR-1 | CDR-2 | CDR-3 |
| 1H4-2 VL | Light | KASQSVDY | AASNLES | QQSNEDPWT |
| | chain | DGDSYMN | (SEQ ID | (SEQ ID |
| | | (SEQ ID | NO: 5) | NO: 6) |
| | | NO: 4) | | |
| 1H4-2 VH | Heavy | SYTMS | KIRNGGGIT | HYYGSEDYFDY |
| | chain | (SEQ ID | YYLDTLKG | (SEQ ID |
| | | NO: 7) | (SEQ ID | NO: 9) |
| | | | NO: 8) | |

### Variable Light Chain:

Nucleotide sequence:
Signal sequence-FR1-CDR1-FR2-CDR2-FR3 -CDR3-FR4
(SEQ ID NO: 10)
ATGGAGACAGACACAATCCTGCTATGGGTGC
TGCTGCTCTGGGTTCCAGGCTCCACTGGT
GACAATGTGCTGACCCAATCTCCAGCTTCTTTGGCTGTGT
CTCTAGGGCAGAGGGCCACCATCTCCTGCAAGGCCAGCCAAAGTGTTGAT
TATGATGGTGATAGTTATATGAACTGGTACCAACAGAAACCAGGACAGCC
ACCCAAAGTCTTCATCTATGCTGCATCCAATCTAGAATCTGGGATCCCAG
CCAGGTTTAGTGGCAGTGGGTCTGGGACAGACTTCACCCTCAACATCCAT
CCTGTGGAGGAGGAGGATGCTGCAACCTATTACTGTCAGCAAAGTAATGA
GGATCCGTGGACGTTCGGTGGAGGCACCAAGCTGGAAATCAAA
Amino acid sequence:
Signal peptide-FR1-CDR1-FR2-CDR2-FR3-CDR3-FR4
(SEQ ID NO: 12)
METDTILLWVLLLWVPGSTGDNVLTQ
SPASLAVSLGQRATISCKASQSVDYDGDSYMN
WYQQKPGQPPKVFIYAASNLESGIPARFSGSGSGTDFTLNIH
PVEEEDAATYYCQQSNEDPWTFGGGTKLEIK
Variable Heavy chain:
Nucleotide sequence
Signal sequence-FR1-CDR1-FR2-CDR2-FR3-CDR3-FR4
(SEQ ID NO: 11)
ATGAATTTCGGGCTCAGCTTGATTTTCCTT
GTCCTTGTTTTAAAAGGTGTCCTGTGT
GAAGTGAAGCTGGTGGAATCTGGGGGAGGTGTGGTGCAGCCTG
GAGGGTCCCTGAAACTCTCCTGTGCAGCCTCTGGATTCACTTTCAGT
AGCTATACCATGTCT
TGGGTTCGTCAGACTCCAGAGAAGAGGCTGGAGTGGGTCGCA
AAAATTCGTAATGGTGGTGGTATCACCTACTATTTAGACACTTTAA
AGGGCCGATTCACCATCTCCAGAGACAACGCCAAGAACACCCTATACCTG
CAAATGAGCAGTCTGAAGTCTGAAGACACGGCCATTTATTTCTGTGCAAG
ACATTACTACGGTAGCGAGGACTACTTTGACTACTGGGGCCAAGGCACCA
CTCTCACAGTCTCCTCA
Amino acid sequence:
Signal peptide-FR1-CDR1-FR2-CDR2-FR3-CDR3-FR4
(SEQ ID NO: 13)
MNFGLSLIFLVLVLKGVLCEVK
LVESGGGVVQPGGSLKLSCAASGFTFSSYTMS
WVRQTPEKRLEWVAKIRNGGGITYYLDTLKG
RFTISRDNAKNTLYLQMSSLKSEDTAIYFCAR
HYYGSEDYFDYWGQGTTLTVSS

In some instances, the anti-ApoE antibody comprises a VH comprising the three VH CDRs and a VL comprising the three VL CDRs of antibody 1H4-2. The six CDRs can be based on any definition known in the art such as, but not limited to, Kabat, Chothia, enhanced Chothia, contact, IMGT, or Honegger definitions. These CDRs can be determined, e.g., by using the AbYsis database (bioinf.org.uk/abysis/sequence_input/key_annotation/key_annotation.cgi).

In one instance, an anti-ApoE antibody of this disclosure comprises (i) a VH comprising a VHCDR1 comprising the amino acid sequence set forth in SEQ ID NO: 7, a VHCDR2 comprising the amino acid sequence set forth in SEQ ID NO: 8, and a VHCDR3 comprising the amino acid sequence set forth in SEQ ID NO: 9; and (ii) a VL comprising a VLCDR1 comprising the amino acid sequence set forth in SEQ ID NO: 4, a VLCDR2 comprising the amino acid sequence set forth in SEQ ID NO: 5, and a VLCDR3 comprising the amino acid sequence set forth in SEQ ID NO: 6.

In some instances, the anti-ApoE antibody comprises a VH that is at least 75%, 80%, 85%, 90%, 91%, 92%, 93%, 94%, 95%, 96%, 97%, 98%, or 99% identical to the amino acid sequence set forth in SEQ ID NO: 13. In some instances, the anti-ApoE antibody comprises a VL that is at least 75%, 80%, 85%, 90%, 91%, 92%, 93%, 94%, 95%, 96%, 97%, 98%, or 99% identical to the amino acid sequence set forth in SEQ ID NO: 12. In one instance, the anti-ApoE antibody comprises a VH that is at least 85% identical to the amino acid sequence set forth in SEQ ID NO: 13 and a VL that is at least 85% identical to the amino acid sequence set forth in SEQ ID NO: 12. In another instance, the anti-ApoE antibody comprises a VH that is at least 90% identical to the amino acid sequence set forth in SEQ ID NO: 13 and a VL that is at least 90% identical to the amino acid sequence set forth in SEQ ID NO: 12. In yet another instance, the anti-ApoE antibody comprises a VH that is identical to the amino acid sequence set forth in SEQ ID NO: 13 and a VL that is identical to the amino acid sequence set forth in SEQ ID NO: 12.

In certain instances, an antibody of this disclosure that binds to ApoE is one that competes with or binds to the same epitope as a reference antibody with a VH having the amino acid sequence set forth in SEQ ID NO: 13 and a VL having the amino acid sequence set forth in SEQ ID NO: 12.

### Exemplary Antibody 7C11-1

Antibody 7C11-1 generated by immunizing with APOE: KLH-CTEELRVRLASHLRK-CONH2 (SEQ ID NO: 54). The amino acid sequences of the complementarity determining regions (CDRs) and the heavy chain variable region and light chain variable regions of 7C11-1 are provided below.

| Variable | Chain | | | |
|---|---|---|---|---|
| region | type | CDR-1 | CDR-2 | CDR-3 |
| 7C11-1 | Light | KASQSVDY | AASNLES | QQSNEDPWT |
| VL | chain | DGDSYMN | (SEQ ID | (SEQ ID |
| | | (SEQ ID | NO: 15) | NO: 16) |
| | | NO: 14) | | |
| 7C11-1 | Heavy | RYTMS | KIRNVGGIT | HYYGSEDYFDY |
| VH | chain | (SEQ ID | YYPDTVKG | (SEQ ID |
| | | NO: 17) | (SEQ ID | NO: 19) |
| | | | NO: 18) | |

### Variable Heavy Chain:

Nucleotide sequence
Signal sequence-FR1-CDR1-FR2-CDR2-FR3-CDR3-FR4
(SEQ ID NO: 20)
ATGAATTTCGGGCTCAGCGTGATTTTCCTTG
TCCTTGTTTTAAAAGGTGTCCTGTGT
GAAGTGAAGCTGGTGGAGTCTGGGGGAGGTTTAGTGCAGCCTG
GAGGGTCCCTGAAACTCTCCTGTGCAGCCTCTGGATTCACTTTCAGTAGG
TATACCATGTCTTGGGTTCGGCAGACTCCAGAGAAGAGGCTGGAGTGGGT
CGCAAAAATTCGTAATGTTGGTGGTATCACCTACTATCCAGACACTGTAA
AGGGCCGATTCACCATCTCCAGAGACAACGCCAAGAACACCCTTTACCTG
CAAATGAGCAGTCTGAAGTCTGAAGACACGGCCATGTATTACTGTGCAAG
ACATTATTACGGTAGCGAGGACTACTTTGACTACTGGGGCCAAGGCACCA
CTCTCACAGTCTCCTCA
Amino acid sequence:
Signal peptide-FR1-CDR1-FR2-CDR2-FR3-CDR3-FR4
(SEQ ID NO: 22)
MNFGLSVIFLVLVLKGVLCEVKL
VESGGGLVQPGGSLKLSCAASGFTFSR
YTMSWVRQTPEKRLEWVAKIRNVGGITYYPDTVKGRFTISRDNAKNTLYL
QMSSLKSEDTAMYYCARHYYGSEDYFDYWGQGTTLTVSS
Variable Light chain:
Nucleotide sequence:
Signal sequence-FR1-CDR1-FR2-CDR2-FR3-CDR3-FR4
(SEQ ID NO: 21)
ATGGAGACAGACACAATCCTGCTATGGGTG
CTGCTGCTCTGGGTTCCAGGCTCCACTGGT
GACAATGTGCTGACCCAATCTCCAGCTTCTTTGGCTGTGT
CTCTAGGGCAGAGGGCCACCATCTCCTGCAAGGCCAGCCAAAGTGTTGAT
TATGATGGTGATAGTTATATGAACTGGTACCAACAGAAACCAGGACAGCC
ACCCAAAGTCTTCATCTATGCTGCATCCAATCTAGAATCTGGGATCCCAG
CCAGGTTTAGTGGCAGTGGGTCTGGGACAAACTTCACCCTCAACATCCAT
CCTGTGGAGGAGGAGGATGCTGCAACCTATTACTGTCAGCAAAGTAATGA
GGATCCGTGGACGTTCGGTGGA GGCACCAAGCTGGAAATCAAA
Amino acid sequence:
Signal sequence-FR1-CDR1-FR2-CDR2-FR3-CDR3-FR4
(SEQ ID NO: 23)
METDTILLWVLLLWVPGSTGDNVLT
QSPASLAVSLGQRATISCKASQSVD
YDGDSYMNWYQQKPGQPPKVFIYAASNLESGIPARFSGSGSGTNFTLNIH
PVEEEDAATYYCQQSNEDPWTFGG GTKLEIK

In some instances, the anti-ApoE antibody comprises a VH comprising the three VH CDRs and a VL comprising the three VL CDRs of antibody 7C11-1. The six CDRs can be based on any definition known in the art such as, but not limited to, Kabat, Chothia, enhanced Chothia, contact, IMGT, or Honegger definitions. These CDRs can be determined, e.g., by using the AbYsis database (bioinf.org.uk/abysis/sequence_input/key_annotation/key_annotation.cgi).

In one instance, an anti-ApoE antibody of this disclosure comprises (i) a VH comprising a VHCDR1 comprising the amino acid sequence set forth in SEQ ID NO: 17, a VHCDR2 comprising the amino acid sequence set forth in SEQ ID NO: 18, and a VHCDR3 comprising the amino acid sequence set forth in SEQ ID NO: 19; and (ii) a VL comprising a VLCDR1 comprising the amino acid sequence set forth in SEQ ID NO: 14, a VLCDR2 comprising the amino acid sequence set forth in SEQ ID NO: 15, and a VLCDR3 comprising the amino acid sequence set forth in SEQ ID NO: 16.

In some instances, the anti-ApoE antibody comprises a VH that is at least 75%, 80%, 85%, 90%, 91%, 92%, 93%, 94%, 95%, 96%, 97%, 98%, or 99% identical to the amino acid sequence set forth in SEQ ID NO: 23. In some instances, the anti-ApoE antibody comprises a VL that is at least 75%, 80%, 85%, 90%, 91%, 92%, 93%, 94%, 95%, 96%, 97%, 98%, or 99% identical to the amino acid sequence set forth in SEQ ID NO: 22. In one instance, the anti-ApoE antibody comprises a VH that is at least 85% identical to the amino acid sequence set forth in SEQ ID NO: 23 and a VL that is at least 85% identical to the amino acid sequence set forth in SEQ ID NO: 22. In another instance, the anti-ApoE antibody comprises a VH that is at least 90% identical to the amino acid sequence set forth in SEQ ID NO: 23 and a VL that is at least 90% identical to the amino acid sequence set forth in SEQ ID NO: 22. In yet another instance, the anti-ApoE antibody comprises a VH that is identical to the amino acid sequence set forth in SEQ ID NO: 23 and a VL that is identical to the amino acid sequence set forth in SEQ ID NO: 22.

In certain instances, an antibody of this disclosure that binds to ApoE is one that competes with or binds to the same epitope as a reference antibody with a VH having the amino acid sequence set forth in SEQ ID NO: 23 and a VL having the amino acid sequence set forth in SEQ ID NO: 22.

### Exemplary Antibody 19G10-2

Antibody 19G10-2 generated by immunizing with KLH-CTEELRVSLASHLRK-CONH2. The amino acid sequences of the complementarity determining regions (CDRs) and the heavy chain variable region and light chain variable regions of 19G10-2 are provided below.

| Variable | Chain | | | |
|---|---|---|---|---|
| region | type | CDR-1 | CDR-2 | CDR-3 |
| 19G10- | Light | KASQSVDY | AASNLES | QQSNVDPWT |
| 2 VL | chain | DGDSYMN | (SEQ ID | (SEQ ID |
| | | (SEQ ID | NO: 25) | NO: 26) |
| | | NO: 24) | | |
| 19G10- | Heavy | DYHMH | WIDPENGNT | GTARASFDY |
| 2 VH | chain | (SEQ ID | MYDPKFQG | (SEQ ID |
| | | NO: 27) | (SEQ ID | NO: 29) |
| | | | NO: 28) | |

### Variable Light Chain:

Nucleotide sequence:
Signal sequence-FR1-CDR1-FR2-CDR2-FR3-CDR3-FR4
(SEQ ID NO: 30)
ATGGAGACAGACACAATCCTGCTATGGGTG
CTGCTGCTCTGGGTTCCAGGCTCCACTGGT
GACATTGTGCTGACCCAATCTCCAGCTTCTTTGGCTGTGT
CTCTAGGGCAGAGGGCCACCATCTCCTGCAAGGCCAGCCAAAGTGTTGAT
TATGATGGTGATAGTTATATGAATTGGTACCAACAGAAATCAGGACAGCC
ACCCAAACTCCTCATCTATGCTGCATCCAATCTAGAATCTGGGATCCCAG
CCAGGTTTAGTGGCAGTGGGTCTGGGACAGACTTCACCCTCAACATCCAT
CCTGTGGAGGAGGAGGATGCTGCAACCTATTACTGTCAGCAAAGTAATGT
GGATCCGTGGACGTTCGGTGGAGGCACCAAGCTGGAAATCAAA
Amino acid sequence:
Signal sequence-FR1-CDR1-FR2-CDR2-FR3-CDR3-FR4
(SEQ ID NO: 32)
METDTILLWVLLLWVPGSTGDIVLTQ
SPASLAVSLGQRATISCKASQSVD
YDGDSYMNWYQQKSGQPPKLLIYAASNLESGIPARFSGSGSGTDFTLNIH
PVEEEDAATYYCQQSNVDPWTFGGGTKLEIK
Variable Heavy chain analysis:
Nucleotide sequence
Signal sequence-FR1-CDR1-FR2-CDR2-FR3-CDR3-FR4
(SEQ ID NO: 31)
ATGAAATGCAGCTGGGTCATCTTCTTCCTG
ATGGCAGTGGTTACAGGGGTCAATTCA
GAGGTTCAGCTGCAGCAGTCTGGGGCTGAGCTTGTGAGGCCAG
GGGCCTTAGTCAAGTTGTCCTGCAAAGCTTCTGGCTTCAACATTAAAGAC
TACCATATGCACTGGGTGAAGGAGAGGCCTGAACAGGGCCTGGAGTGGAT
TGGATGGATTGATCCTGAGAATGGTAATACTATGTATGACCCGAAGTTCC
AGGGCAAGGCCAGTATAACAGCAGACACATCCTCCAACACAGCCTACCTG
CAGCTCAGCAGCCTGACATCTGAGGACACTGCCGTCTATTACTGTGTTAG
GGGGACAGCTCGGGCTTCCTTTGACTACTGGGGCCAAGGCACCACTCTCA
CAGTCTCCTCA
Amino acid sequence:
Signal sequence-FR1-CDR1-FR2-CDR2-FR3-CDR3-FR4
(SEQ ID NO: 33)
MKCSWVIFFLMAVVTGVNSEVQLQQSGA
ELVRPGALVKLSCKASGFNIKD
YHMHWVKERPEQGLEWIGWIDPENGNTMYDPKFQGKASITADTSSNTAYL
QLSSLTSEDTAVYYCVRGTARASFDYWGQGTTLTVSS

In some instances, the anti-ApoE antibody comprises a VH comprising the three VH CDRs and a VL comprising the three VL CDRs of antibody 19G10-2. The six CDRs can be based on any definition known in the art such as, but not limited to, Kabat, Chothia, enhanced Chothia, contact, IMGT, or Honegger definitions. These CDRs can be determined, e.g., by using the AbYsis database (bioinf.org.uk/abysis/sequence_input/key_annotation/key_annotation.cgi).

In one instance, an anti-ApoE antibody of this disclosure comprises (i) a VH comprising a VHCDR1 comprising the amino acid sequence set forth in SEQ ID NO: 27, a VHCDR2 comprising the amino acid sequence set forth in SEQ ID NO: 28, and a VHCDR3 comprising the amino acid sequence set forth in SEQ ID NO: 29; and (ii) a VL comprising a VLCDR1 comprising the amino acid sequence set forth in SEQ ID NO: 24, a VLCDR2 comprising the amino acid sequence set forth in SEQ ID NO: 25, and a VLCDR3 comprising the amino acid sequence set forth in SEQ ID NO: 26.

In some instances, the anti-ApoE antibody comprises a VH that is at least 75%, 80%, 85%, 90%, 91%, 92%, 93%, 94%, 95%, 96%, 97%, 98%, or 99% identical to the amino acid sequence set forth in SEQ ID NO: 33. In some instances, the anti-ApoE antibody comprises a VL that is at least 75%, 80%, 85%, 90%, 91%, 92%, 93%, 94%, 95%, 96%, 97%, 98%, or 99% identical to the amino acid sequence set forth in SEQ ID NO: 32. In one instance, the anti-ApoE antibody comprises a VH that is at least 85% identical to the amino acid sequence set forth in SEQ ID NO: 33 and a VL that is at least 85% identical to the amino acid sequence set forth in SEQ ID NO: 32. In another instance, the anti-ApoE antibody comprises a VH that is at least 90% identical to the amino acid sequence set forth in SEQ ID NO: 33 and a VL that is at least 90% identical to the amino acid sequence set forth in SEQ ID NO: 32. In yet another instance, the anti-ApoE antibody comprises a VH that is identical to the amino acid sequence set forth in SEQ ID NO: 33 and a VL that is identical to the amino acid sequence set forth in SEQ ID NO: 32.

In certain instances, an antibody of this disclosure that binds to ApoE is one that competes with or binds to the same epitope as a reference antibody with a VH having the amino acid sequence set forth in SEQ ID NO: 33 and a VL having the amino acid sequence set forth in SEQ ID NO: 32.

### Exemplary Antibody 25F1-2

Antibody 25F1-2 was generated by immunizing with KLH-CTEELRVSLASHLRK-CONH2. The amino acid sequences of the complementarity determining regions (CDRs) and the heavy chain variable region and light chain variable regions of 25F1-2 are provided below.

| Variable | Chain | | | |
|---|---|---|---|---|
| region | type | CDR-1 | CDR-2 | CDR-3 |
| 25F1- | Light | KASQSVDY | TASNLES | QQSNEDPWT |
| 2 VL | chain | DGDTYMN | (SEQ ID | (SEQ ID |
| | | (SEQ ID | NO: 35) | NO: 36) |
| | | NO: 34) | | |
| 25F1- | Heavy | DYHIH | WIDPEIDK | GTARASFDY |
| 2 VH | chain | (SEQ ID | TLYDPKFQG | (SEQ ID |
| | | NO: 37) | (SEQ ID | NO: 39) |
| | | | NO: 38) | |

### Variable Light Chain Analysis:

Nucleotide sequence:
Signal sequence-FR1-CDR1-FR2-CDR2-FR3-CDR3-FR4
(SEQ ID NO: 40)
ATGGAGACAGACACAATCCTGCTATGGGTG
CTGCTGCTCTGGGTTCCAGGCTCCACTGGT
GACATTGTGCTGACCCAATCTCCAGCTTCTTTGGCTGTGT
CTCTAGGGCAGAGGGCCACCATCTCCTGCAAGGCCAGCCAAAGTGTTGAT
TATGATGGTGATACTTATATGAACTGGTACCAACAGAAACCAGGACAGCC
ACCCAAACTCCTCATCTATACTGCATCCAATCTAGAATCTGGGATCCCAG
CCAGGTTTAGTGGCAGTGGGTCTGGGACAGACTTCACCCTCAACATCCAT
CCTGTGGAGGAGGTGGATGCTGCAACCTATTACTGTCAGCAAAGTAATGA
GGATCCATGGACGTTCGGTGGAGGCACCAAGCTGG AAATCAAA
Amino acid sequence:
Signal sequence-FR1-CDR1-FR2-CDR2-FR3-CDR3-FR4
(SEQ ID NO: 42)
METDTILLWVLLLWVPGSTGDIVLTQSPA
SLAVSLGQRATISCKASQSVD
YDGDTYMNWYQQKPGQPPKLLIYTASNLESGIPARFSGSGSGTDFTLNIH
PVEEVDAATYYCQQSNEDPWTFGGGTKLEIK
Variable Heavy chain analysis:
Nucleotide sequence
Signal sequence-FR1-CDR1-FR2-CDR2-FR3-CDR3-FR4
(SEQ ID NO: 41)
ATGAAATGCAGCTGGGTCATCTTCTTCCTG
ATGGCAGTGGTTACAGGGGTCAATTCA
GAGGTTCAGCTGCAGCAGTCTGGGGCTGAGCTTGTGAGGCCAG
GGGCCTTAGTCAAGTGGTCCTGCAAAGCTTCTGGCTTCAACATTAAAGAC
TACCATATACACTGGGTGAAACAGAGGCCTGAACAGGGCCTGGACTGGAT
TGGATGGATTGATCCTGAGATTGATAAAACTCTATATGACCCGAAGTTTC
AGGGCAAGGCCAGAATAACAGCAGACACATCCTCCAATACAGCCTACCTG
CAGCTCAGCAGCCTGACATCTGAAGACACTGCCGTCTATTACTGTGCCAG
GGGGACAGCTCGGGCTTCCTTTGACTACTGGGGCCAAGGCACCACTCTCA
CAGTCTCCTCA
Amino acid sequence:
Signal sequence-FR1-CDR1-FR2-CDR2-FR3-CDR3-FR4
(SEQ ID NO: 43)
MKCSWVIFFLMAVVTGVNSEVQLQQSGAELVR
PGALVKWSCKASGFNIKD
YHIHWVKQRPEQGLDWIGWIDPEIDKTLYDPKFQGKARITADTSSNTAYL
QLSSLTSEDTAVYYCARGTARASFDYWGQGTTLTVSS

In some instances, the anti-ApoE antibody comprises a VH comprising the three VH CDRs and a VL comprising the three VL CDRs of antibody 25F1-2. The six CDRs can be based on any definition known in the art such as, but not limited to, Kabat, Chothia, enhanced Chothia, contact, IMGT, or Honegger definitions. These CDRs can be determined, e.g., by using the AbYsis database (bioinf.org.uk/abysis/sequence_input/key_annotation/key_annotation.cgi).

In one instance, an anti-ApoE antibody of this disclosure comprises (i) a VH comprising a VHCDR1 comprising the amino acid sequence set forth in SEQ ID NO: 37, a VHCDR2 comprising the amino acid sequence set forth in SEQ ID NO: 38, and a VHCDR3 comprising the amino acid sequence set forth in SEQ ID NO: 39; and (ii) a VL comprising a VLCDR1 comprising the amino acid sequence set forth in SEQ ID NO: 34, a VLCDR2 comprising the amino acid sequence set forth in SEQ ID NO: 35, and a VLCDR3 comprising the amino acid sequence set forth in SEQ ID NO: 36.

In some instances, the anti-ApoE antibody comprises a VH that is at least 75%, 80%, 85%, 90%, 91%, 92%, 93%, 94%, 95%, 96%, 97%, 98%, or 99% identical to the amino acid sequence set forth in SEQ ID NO: 43. In some instances, the anti-ApoE antibody comprises a VL that is at least 75%, 80%, 85%, 90%, 91%, 92%, 93%, 94%, 95%, 96%, 97%, 98%, or 99% identical to the amino acid sequence set forth in SEQ ID NO: 42. In one instance, the anti-ApoE antibody comprises a VH that is at least 85% identical to the amino acid sequence set forth in SEQ ID NO: 43 and a VL that is at least 85% identical to the amino acid sequence set forth in SEQ ID NO: 42. In another instance, the anti-ApoE antibody comprises a VH that is at least 90% identical to the amino acid sequence set forth in SEQ ID NO: 43 and a VL that is at least 90% identical to the amino acid sequence set forth in SEQ ID NO: 42. In yet another instance, the anti-ApoE antibody comprises a VH that is identical to the amino acid sequence set forth in SEQ ID NO: 43 and a VL that is identical to the amino acid sequence set forth in SEQ ID NO: 42.

In certain instances, an antibody of this disclosure that binds to ApoE is one that competes with or binds to the same epitope as a reference antibody with a VH having the amino acid sequence set forth in SEQ ID NO: 43 and a VL having the amino acid sequence set forth in SEQ ID NO: 42.

### Exemplary Antibody 1343ab

Antibody 1343ab was generated by immunizing with KLH-CTEELRVSLASHLRK-CONH2. The amino acid sequences of the complementarity determining regions (CDRs) and the full length heavy and light chains are provided below.

| Variable | Chain | | | |
|---|---|---|---|---|
| region | type | CDR-1 | CDR-2 | CDR-3 |
| 1343 VL | Light | KASQSVDY | VASNLES | QQSNLDPWT |
| | chain | DGENYMN | (SEQ ID | (SEQ ID |
| | | (SEQ ID | NO: 45) | NO: 46) |
| | | NO: 44) | | |
| 1343 VH | Heavy | GFNIKDY | DPENGN | GTARASFDY |
| | chain | (SEQ ID | (SEQ ID | (SEQ ID |
| | | NO: 47) | NO: 48) | NO: 49) |

Full length heavy chain
(SEQ ID NO: 53)
EVQLQQSGAELVRPGALVKLSCKASGFNIKDYHLHWVKQRPEQGLE
WIGWIDPENGNVIYDPKFQGKATMTV
VTSSNTAYLQLRSLTSEDTAVYFCTRGTARASFDYWGQGTSLTVSSAK
TTPPSVYPLAPGSAAQTNSMVTLG
CLVKGYFPEPVTVTWNSGSLSSGVHTFPAVLQSDLYTLSSSVTVPSST
WPSETVTCNVAHPASSTKVDKKIV
PRDCGCKPCICTVPEVSSVFIFPPKPKDVLTITLTPKVTCVVVDISKDD
PEVQFSWFVDDVEVHTAQTQPRE
EQFNSTFRSVSELPIMHQDWLNGKEFKCRVNSAAFPAPIEKTISKTKGR
PKAPQVYTIPPPKEQMAKDKVSL
TCMITDFFPEDITVEWQWNGQPAENYKNTQPIMDTDGSYFVYSKLNV
QKSNWEAGNTFTCSVLHEGLHNHHT
EKSLSHSPGK
Full length light chain
(SEQ ID NO: 52)
DIVLTQSPASLAVSLGQRATISCKASQSVDYDGENYMNWYQQKPGQS
PKLLIYVASNLESGIPARFSGSGSG
TDFTLNIHPVEEEDAATYYCQQSNLDPWTFGGGTKLEIKRADAAPTVS
IFPPSSEQLTSGGASVVCFLNNFY
PKDINVKWKIDGSERQNGVLNSWTDQDSKDSTYSMSSTLTLTKDEYE
RHNSYTCEATHKTSTSPIVKSFNRN
EC

N-linked glycosylation was detected on heavy chain constant region N at 292. Loss of C-terminal lysine observed on heavy chain.

In some instances, the anti-ApoE antibody comprises a VH comprising the three VH CDRs and a VL comprising the three VL CDRs of antibody 1343ab. The six CDRs can be based on any definition known in the art such as, but not limited to, Kabat, Chothia, enhanced Chothia, contact, IMGT, or Honegger definitions. These CDRs can be determined, e.g., by using the AbYsis database (bioinf.org.uk/abysis/sequence_input/key_annotation/key_annotation.cgi).

In one instance, an anti-ApoE antibody of this disclosure comprises (i) a VH comprising a VHCDR1 comprising the amino acid sequence set forth in SEQ ID NO: 47, a VHCDR2 comprising the amino acid sequence set forth in SEQ ID NO: 48, and a VHCDR3 comprising the amino acid sequence set forth in SEQ ID NO: 49; and (ii) a VL comprising a VLCDR1 comprising the amino acid sequence set forth in SEQ ID NO: 44, a VLCDR2 comprising the amino acid sequence set forth in SEQ ID NO: 45, and a VLCDR3 comprising the amino acid sequence set forth in SEQ ID NO: 46.

In some instances, the anti-ApoE antibody comprises a heavy chain that is at least 70%, 71%, 72%, 73%, 74%, 75%, 80%, 85%, 90%, 91%, 92%, 93%, 94%, 95%, 96%, 97%, 98%, or 99% identical to the amino acid sequence set forth in SEQ ID NO: 53. In some instances, the anti-ApoE antibody comprises a light chain that is at least 70%, 71%, 72%, 73%, 74%, 75%, 80%, 85%, 90%, 91%, 92%, 93%, 94%, 95%, 96%, 97%, 98%, or 99% identical to the amino acid sequence set forth in SEQ ID NO: 52. In one instance, the anti-ApoE antibody comprises a heavy chain that is at least 80% identical to the amino acid sequence set forth in SEQ ID NO: 53 and a light chain that is at least 80% identical to the amino acid sequence set forth in SEQ ID NO: 52. In another instance, the anti-ApoE antibody comprises a heavy chain that is at least 85% identical to the amino acid sequence set forth in SEQ ID NO: 53 and a light chain that is at least 85% identical to the amino acid sequence set forth in SEQ ID NO: 52. In yet another instance, the anti-ApoE antibody comprises a heavy chain that is identical to the amino acid sequence set forth in SEQ ID NO: 53 and a light chain that is identical to the amino acid sequence set forth in SEQ ID NO: 52.

In certain instances, an antibody of this disclosure that binds to ApoE is one that competes with or binds to the same epitope as a reference antibody with a heavy chain having the amino acid sequence set forth in SEQ ID NO: 53 and a light chain having the amino acid sequence set forth in SEQ ID NO: 52.

Chimeric, human, or humanized antibodies having the CDR sequences of any of the above antibodies can be generated based on methods described herein.

### Antibody Fragments

Antibody fragments (e.g., Fab, Fab′, F(ab′)2, Facb, and Fv) can be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Conjugated Antibodies

The antibodies disclosed herein can be conjugated antibodies that are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. 90Y, 1311), fluorescent substances, luminescent substances, haptens, enzymes, metal chelates, and drugs.

In some embodiments, the antibodies described herein are modified with a moiety that improves its stabilization and/or retention in circulation, e.g., in blood, serum, or other tissues, including the brain, e.g., by at least 1.5, 2, 5, 10, 15, 20, 25, 30, 40, or 50-fold. For example, the antibodies described herein can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the antibodies described herein can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. In some embodiments, the antibodies described herein are modified with a moiety that improves its penetration of the blood-brain barrier (such as those described in Pardridge, J Cereb Blood Flow Metab 32(11): 1959-1972, 2012). Exemplary blood-brain barrier penetrating moieties include, but are not limited to, glucose transporter type 1 (GLUT1), cationic amino-acid transporter type 1 (CAT1), monocarboxylic acid transporter type 1 (MCT1), concentrative nucleoside transporter type 2 (CNT2), active efflux transporter (AET) (e.g., p-glycoprotein, and those described in Pardridge, J Cereb Blood Flow Metab 32(11): 1959-1972, 2012), Additional blood-brain barrier penetrating moieties are known in the art.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

The anti-ApoE antibodies can be in the form of full length (or whole) antibodies, or in the form of low molecular weight forms (e.g., biologically active antibody fragments or minibodies) of the anti-ApoE antibodies, e.g., Fab, Fab′, F(ab′)2, Fv, Fd, dAb, scFv, and sc(Fv)2. Other anti-ApoE antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1): 13-22 (2007); U.S. 2005/0079574 and Davies et al. (1996) Protein Eng., 9(6):531-7. Like a whole antibody, a sdAb is able to bind selectively to a specific antigen (e.g., ApoE2, ApoE3, ApoE4, or ApoEch). With a molecular weight of only 12-15 kDa, sdAbs are much smaller than common antibodies and even smaller than Fab fragments and single-chain variable fragments.

In certain embodiments, an anti-ApoE antibody or antigen-binding fragment thereof or low molecular weight antibodies thereof specifically binds to the HSPG/heparin-binding domain of ApoE and reduces the severity of symptoms when administered to human patients having one or more of, or animal models of: dementia and/or mild cognitive impairment (MCI) (e.g. those associated with Alzheimer's disease, vascular dementia, Lewy body dementia, frontotemporal dementia, Parkinson's disease, Huntington's disease, or neurodegeneration). In certain embodiments, an anti-ApoE antibody or antigen-binding fragment thereof or low molecular weight antibodies thereof specifically binds to the HSPG/heparin-binding domain of ApoE and reduces the severity of symptoms when administered to human patients having one or more of, or animal models of: neurodegenerative diseases, cerebrovascular diseases (e.g. stroke, carotid stenosis, vertebral stenosis, or aneurysms), brain injuries (e.g. traumatic brain injury, acquired brain injury), retinal degeneration, glaucoma, or retinal injury. These features of an anti-ApoE antibody or low molecular weight antibodies thereof can be measured according to methods known in the art.

### Nucleic Acids, Vector, Host Cells

This disclosure also features nucleic acids encoding the antibodies disclosed herein. Provided herein are nucleic acids encoding the VH CDR1, VH CDR2, and VH CDR3 of the anti-ApoE antibodies described herein. Also provided are nucleic acids encoding the VL CDR1, VL CDR2, and VL CDR3 of the anti-ApoE antibodies described herein. Provided herein are nucleic acids encoding the VH CDR1, VH CDR2, VH CDR3, VL CDR1, VL CDR2, and VL CDR3 of the anti-ApoE antibodies described herein. Also provided are nucleic acids encoding the heavy chain variable region (VH) of the anti-ApoE antibodies described herein, and/or nucleic acids encoding the light chain variable region (VL) of the anti-ApoE antibodies described herein. In certain instances, provided herein are nucleic acids encoding the VH and/or VL of the anti-ApoE antibodies described herein, linked to human heavy and/or human light chain constant regions, respectively. Also provided herein are nucleic acids encoding both VH and VL of the anti-ApoE antibodies described herein. In some instances, the nucleic acids described herein include a nucleic acid encoding the Fc region of a human antibody (e.g., human IgG1, IgG2, IgG3, or IgG4). In certain instances, the nucleic acids include a nucleic acid encoding the Fc region of a human antibody that has been modified to reduce or eliminate effector function (e.g., a N297Q or T299A substitution in a human IgG1 Fc region (numbering according to EU numbering)). In some cases, the nucleic acids include a nucleic acid encoding an Fc moiety that is a hIgG1 Fc, a hIgG2 Fc, a hIgG3 Fc, a hIgG4 Fc, a hIgG1agly Fc, a hIgG2 SAA Fc, a hIgG4(S228P) Fc, or a hIgG4(S228P)/G1 agly Fc.

Also disclosed herein are vectors (e.g. expression vectors) containing any of the nucleic acids described above.

Furthermore, this disclosure relates to host cells (e.g. bacterial cells, yeast cells, insect cells, or mammalian cells) containing the vector(s) or the nucleic acid(s) described above.

### Methods of Obtaining Anti-ApoE Antibodies

Also provided herein are methods for making anti-ApoE antibodies useful in the present methods. General methods for making antibodies, e.g., monospecific, polyclonal, or monoclonal antibodies, are known in the art. For monoclonal antibodies, the process involves obtaining antibody-secreting immune cells (lymphocytes) from the spleen of a mammal (e.g., mouse) that has been previously immunized with the antigen of interest (e.g., a peptide antigen as described herein) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975).

Mammalian lymphocytes can be immunized by in vivo immunization of the animal (e.g., a mouse) with a peptide antigen, e.g., a peptide antigen that is at least 80%, 85%, 90%, or 95% identical to KLH-CTEELRVRLASHLRK-CONH2 (SEQ ID NO:54) or KLH-CTEELRVSLASHLRK-CONH2 (SEQ ID NO:55), optionally with one or more substitutions or deletions, e.g., of up to 20% of the residues. For example, the methods can include immunizing the animal with a peptide comprising a sequence that is at least 80% identical to at least 10 consecutive amino acids from: the heparin-binding domain of APOE, e.g., a peptide comprising TEELRVRLASHLRK (SEQ ID NO: 3) or TEELRVSLASHLRK (SEQ ID NO: 2). Such immunizations are repeated as necessary at intervals of up to several weeks to obtain a sufficient titer of antibodies. Following the last antigen boost, the animals are sacrificed, and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected, e.g., at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

The method described herein comprises any one of the step(s) of producing a chimeric antibody, humanized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor (1988). When derivatives of said antibodies are obtained by the phage display technique, surface plasmon resonance as employed in the BIAcore system can be used to increase the efficiency of phage antibodies which bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the production of humanized antibodies are described in, e.g., European application EP-A1 0 239 400 and international application WO90/07861. A further source of antibodies to be utilized in accordance with the present invention are so-called xenogeneic antibodies. The general principle for the production of xenogeneic antibodies such as human-like antibodies in mice is described in, e.g., international applications WO91/10741, WO94/02602, WO96/34096 and WO 96/33735. As discussed above, the antibody described herein may exist in a variety of forms besides complete antibodies; including, for example, Fv, Fab and F(ab)2, as well as in single chains; see e.g. international application WO88/09344.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981), said references incorporated by reference in their entireties. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology.

In the known hybridoma process (Kohler et al., Nature 256 (1975), 495) the relatively short-lived, or mortal, lymphocytes from a mammal, e.g., B cells derived from a murine subject as described herein, are fused with an immortal tumor cell line (e.g., a myeloma cell line), thus, producing hybrid cells or “hybridomas” which are both immortal and capable of producing the genetically coded antibody of the B cell. The resulting hybrids are segregated into single genetic strains by selection, dilution, and re-growth with each individual strain comprising specific genes for the formation of a single antibody. They produce antibodies, which are homogeneous against a desired antigen and, in reference to their pure genetic parentage, are termed “monoclonal”.

Hybridoma cells thus prepared are seeded and grown in a suitable culture medium that contain one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. The binding specificity of the monoclonal antibodies produced by hybridoma cells is determined by in vitro assays such as immunoprecipitation, radioimmunoassay (RIA) or enzyme-linked immunoabsorbent assay (ELISA) as described herein. After hybridoma cells are identified that produce antibodies of the desired specificity, affinity and/or activity, the clones may be subcloned by limiting dilution procedures and grown by standard methods; see, e.g., Goding, Monoclonal Antibodies: Principles and Practice, Academic Press, pp 59-103 (1986). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, lymphocytes can be selected by micromanipulation and the variable genes isolated. For example, peripheral blood mononuclear cells can be isolated from an immunized or naturally immune mammal, e.g., a human, and cultured for about 7 days in vitro. The cultures can be screened for specific immunoglobulins that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies, such as those described above, can be made, for example, by preparing and expressing synthetic genes that encode the recited amino acid sequences. Methods of generating variants (e.g., comprising amino acid substitutions) of any of the anti-ApoE antibodies are well known in the art. These methods include, but are not limited to, preparation by site-directed (or oligonucleotide-mediated) mutagenesis, PCR mutagenesis, and cassette mutagenesis of a prepared DNA molecule encoding the antibody or any portion thereof (e.g., a framework region, a CDR, a constant region). Site-directed mutagenesis is well known in the art (see, e.g., Carter et al., Nucl. Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nucl. Acids Res. 17:723-733 (1989). Another method for preparing sequence variants, cassette mutagenesis, is based on the technique described by Wells et al., Gene, 34:315-323 (1985).

Antibodies can be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS, Hela). In addition, antibodies (e.g., scFv's) can be expressed in a yeast cell such as Pichia (see, e.g., Powers et al., J Immunol Methods. 251: 123-35 (2001)), Hanseula, or Saccharomyces. To produce the antibody or antigen binding fragments thereof of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, POPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The ApoE-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to wild type ApoE or mutant ApoE (e.g. ApoEch) using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

### Mutant ApoE Proteins, Peptides and Fusion Proteins Thereof

The present disclosure provides mutant ApoE proteins or fragments thereof containing amino acid substitutions at one or more positions in the HSPG-binding domain as compared to a wild type ApoE protein. In some embodiments, the mutant ApoE protein or fragments thereof includes an amino acid other than Arginine at position 136. In some embodiments, the mutant ApoE protein or fragments thereof contains Serine, Histidine, or Cysteine at position 136. Also provided are nucleic acid (e.g., DNA or RNA) sequences encoding the mutant ApoE proteins or fragments thereof, and vectors containing the nucleic acid sequences. The mutant ApoE proteins or fragments thereof, nucleic acids encoding such proteins or fragments, and vectors containing the nucleic acid sequences are useful for treating or preventing disorders associated with dementia or mild cognitive impairment (MCI) (e.g. Alzheimer's disease, vascular dementia, Lewy body dementia, frontotemporal dementia, Parkinson's disease, or Huntington's disease), neurodegenerative diseases, cerebrovascular diseases, brain injury, retinal degeneration, or retinal injury.

In some embodiments, the mutant ApoE protein is an ApoEch protein (e.g. ApoE2ch, ApoE3ch, or ApoE4ch protein). Fragments of the ApoEch protein that includes the amino acid position 136 are also contemplated here. Exemplary sequence of a full-length ApoE3ch protein is shown below. The mutation from arginine to serine is bolded and double underlined.

(SEQ ID NO: 45)
MKVLWAALLVTFLAGCQAKVEQAVETEPEPELRQQTEWQSGQRWELALG
RFWDYLRWVQTLSEQVQEELLSSQVTQELRALMDETMKELKAYKSELEEQ
LTPVAEETRARLSKELQAAQARLGADMEDVCGRLVQYRGEVQAMLGQST
EELRV [symbol] LASHLRKLRKRLLRDADDLQKRLAVYQAGAREGAERGLSAIRER
LGPLVEQGRVRAATVGSLAGQPLQERAQAWGERLRARMEEMGSRTRDRLD
EVKEQVAEVRAKLEEQAQQIRLQAEAFQARLKSWFEPLVEDMQRQWAGL
VEKVQAAVGTSAAPVPSDNH [symbol]
indicates data missing or illegible when filed

In some embodiments, the methods disclosed herein allows the mutant ApoE protein or fragments thereof to cross the blood-brain barrier. The mutant ApoE protein or fragments thereof may be delivered using nanocarriers, including but not limited to, polymeric nanoparticles, lipid based nanoparticles, liposome, micelle, dendrimer, a human cell expressing the protein, and nanotube (See, Dominguez et al. J Nanosci nanotechnol. 14(1):766-79, 2014). In some embodiments, the mutant ApoE protein or fragments thereof is delivered intranasally, via intracarotid or transmucosal delivery (e.g. intracarotid infusion of hypertonic solutions (arabinose or mannitol); see, Sanchez-Covarrubias et al., Curr Pharm Des. 20(10): 1422-49, 2014 and Miyake et al., World J Otorhinolaryngol Head Neck Surg. 1(1): 11-16, 2015), or via the use of chlorotoxin (See, McCall et al., Tissue Barriers 2(4):e944449, 2014). Hypothermia techniques, receptor-mediated transport, cell-penetrating peptides, and cell-mediated delivery can also be used to facilitate the ApoE3ch protein to cross the blood brain barrier (See, Pandey et al., Tissue Barriers 4(1): e1129476, 2016). For example, immunocytes and stem cells (e.g., neural stem cells, induced pluripotent cells, and mesenchymal stem cells) can be used to carry therapeutic payloads across the BBB. Nanoparticle-loaded mesenchymal stem cells can be used for this purpose (See e.g., Roger et al. Biomaterials 31:8393-401, 2010). Genetically modified stem cells (e.g. genetically modified mesenchymal stem cells) can also be used (See e.g., Ebrahimi and Lalvand Hygeia. J. D. Med. vol. 5 (1): 90-104, 2013). Chemical drug delivery systems (CDDS), such as those described in He et al., Cells, 7(4):24, 2018, can also be used. Additional methods of transporting proteins across the blood-brain barrier are known in the art.

Nucleic acids (e.g., DNA or mRNA) encoding the mutant ApoE protein (e.g., any of the mutant ApoE proteins described herein, e.g. ApoEch) or fragments thereof are contemplated herein. In some embodiments, mRNA encoding the ApoEch protein may be modified to increase stability (such as those described in Zangi et al., Nat Biotechnol. 31(10):898-907, 2013 and developed by Moderna, Inc.; and those described in Alberer et al., Lancet 390(10101): 1511-1520, 2017 and developed by Curevac and BioNTech).

Viral vectors containing DNA sequences encoding the mutant ApoE or fragments thereof are contemplated herein. An exemplary cDNA sequence encoding the full-length ApoE3ch protein (including the signal peptide region) is shown below. The mutation from Cytosine to Adenine is bolded and double underlined.

(SEQ ID NO: 56)
ATGAAGGTTCTGTGGGCTGCGTTGCTGGTCACATTCCTGGCAGGATGCCA
GGCCAAGGTGGAGCAAGCGGTGGAGACAGAGCCGGAGCCCGAGCTGCGCC
AGCAGACCGAGTGGCAGAGCGGCCAGCGCTGGGAACTGGCACTGGGTCGC
TTTTGGGATTACCTGCGCTGGGTGCAGACACTGTCTGAGCAGGTGCAGGA
GGAGCTGCTCAGCTCCCAGGTCACCCAGGAACTGAGGGCGCTGATGGACG
AGACCATGAAGGAGTTGAAGGCCTACAAATCGGAACTGGAGGAACAACTG
ACCCCGGTGGCGGAGGAGACGCGGGCACGGCTGTCCAAGGAGCTGCAGGC
GGCGCAGGCCCGGCTGGGCGCGGACATGGAGGACGTGTGCGGCCGCCTGG
TGCAGTACCGCGGCGAGGTGCAGGCCATGCTCGGCCAGAGCACCGAGGAG
CTGCGGGTG [symbol] GCCTCGCCTCCCACCTGCGCAAGCTGCGTAAGCGGCTCCT
CCGCGATGCCGATGACCTGCAGAAGCGCCTGGCAGTGTACCAGGCCGGGG
CCCGCGAGGGCGCCGAGCGCGGCCTCAGCGCCATCCGCGAGCGCCTGGGG
CCCCTGGTGGAACAGGGCCGCGTGCGGGCCGCCACTGTGGGCTCCCTGGC
CGGCCAGCCGCTACAGGAGCGGGCCCAGGCCTGGGGCGAGCGGCTGCGCG
CGCGGATGGAGGAGATGGGCAGCCGGACCCGCGACCGCCTGGACGAGGTG
AAGGAGCAGGTGGCGGAGGTGCGCGCCAAGCTGGAGGAGCAGGCCCAGCA
GATACGCCTGCAGGCCGAGGCCTTCCAGGCCCGCCTCAAGAGCTGGTTCG
AGCCCCTGGTGGAAGACATGCAGCGCCAGTGGGCCGGGCTGGTGGAGAAG
GTGCAGGCTGCCGTGGGCACCAGCGCCGCCCCTGTGCCCAGCGACAATCA
CTGA [symbol]
indicates data missing or illegible when filed

Suitable vectors are known in the art. In some embodiments, the viral vector is an AAV vector (such as those described in Rosenberg et al., Hum Gene Ther Clin Dev 29(1):24-47, 2018). cDNA sequences encoding an ApoE protein containing a mutation at the R136 position other than R136S are also included. In some embodiments, the mutation is R136H or R136C.

### Peptides and Fusion Proteins

In some embodiments, provided herein are peptides that comprise or consist of the HSPG/heparin-binding domain of a wild type or mutant ApoE (e.g., any of the mutant ApoE proteins described herein). In some instances, the amino acid sequence of the peptides provided herein comprise or consist of the sequences selected from the group consisting of STEELRVRLASHLRKLRKRLLRDADDLQK (SEQ ID NO:57), STEELRVSLASHLRKLRKRLLRDADDLQK (SEQ ID NO:58), RLVQYRGEVQAMLGQSTEELRVRLASHLRKL (SEQ ID NO:59), and RLVQYRGEVQAMLGQSTEELRVSLASHLRKL (SEQ ID NO:60). Variants having at least 80%, 85%, 90%, 95%, 96%, 97%, 98%, 99% or greater sequence identity with these sequences can also be used. Also disclosed are fusion proteins comprising the peptides provided above. In some embodiments, the fusion proteins further include an Fc region of a human antibody (e.g., human IgG1, IgG2, IgG3, or IgG4). In some instances, the fusion protein comprises an Fc region of a human antibody at the C-terminal of the HSPG/heparin-binding domain of a wild type or mutant ApoE. In some instances, the fusion protein comprises an Fc region of a human antibody at the N-terminal of the HSPG/heparin-binding domain of a wild type or mutant ApoE.

In some instances, the peptides and fusion proteins provided herein competes with a wild type ApoE protein for binding to HSPG/heparin. In some instances, the peptides and fusion proteins provided herein reduce or modulate the binding between a wild type ApoE protein and HSPG/heparin. In certain embodiments, the peptides and fusion proteins provided herein inhibits and/or reduces HSPG/heparin-binding of a wild type ApoE protein, and reduces the severity of symptoms when administered to human patients having one or more of, or animal models of: disorders associated with dementia or mild cognitive impairment (MCI) (e.g. Alzheimer's disease, vascular dementia, Lewy body dementia, frontotemporal dementia, Parkinson's disease, Huntington's disease), neurodegenerative diseases, cerebrovascular diseases, brain injury, retinal degeneration, or retinal injury. These features of the peptides and fusion proteins provided herein can be measured according to methods known in the art.

Also provided herein are anti-ApoE vaccines, which can be used to elicit a protective immune response against ApoE. In some embodiments, the anti-ApoE vaccines include one or more of the ApoE peptides provided herein (e.g., and a pharmaceutically acceptable adjuvant. Pharmaceutically acceptable adjuvants are known in the art.

### Pharmaceutical Compositions and Methods of Administration

The methods described herein include the use of pharmaceutical compositions comprising any of the antibodies, peptides or fusion proteins described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intracranial, intranasal, intra-carotid, intravenous, intradermal, subcutaneous, oral (e.g., inhalation), and transmucosal.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal. For transmucosal administration, penetrants appropriate to the barrier to be permeated are used in the formulation. Such penetrants are generally known in the art, and include, for example, detergents, bile salts, and fusidic acid derivatives. Transmucosal administration can be accomplished through the use of nasal sprays or suppositories or injection.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### CRISPR/Cas9-Mediated Gene Editing of APOE

Included herein are methods for treating or preventing disorders associated with dementia and/or mild cognitive impairment (MCI) (e.g. Alzheimer's disease, vascular dementia, Lewy body dementia, frontotemporal dementia, Parkinson's disease, or Huntington's disease), neurodegenerative diseases, cerebrovascular diseases, brain injury, retinal degeneration, or retinal injury, by editing the APOE gene using a genome editing system. Generally, the methods include administering a therapeutically effective amount of a genome editing system as described herein, to a subject who is in need of, or who has been determined to be in need of, such treatment. The term “genome editing system” refers to any system having RNA-guided DNA editing activity. Genome editing systems of the present disclosure include at least two components adapted from naturally occurring CRISPR systems: a gRNA and an RNA-guided nuclease. These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution. See, e.g., WO2018/026976 for a full description of genome editing systems. In certain aspects, the present disclosure provides AAV vectors encoding CRISPR/Cas9 genome editing systems, and on the use of such vectors to treat or prevent disorders as described herein.

### RNA-Guided Nucleases/Cas9

Various RNA-guided nucleases can be used in the present methods, e.g., as described in WO 2018/026976. In some embodiments, the RNA-guided nuclease used in the present methods and compositions is a S. aureus Cas9 or a S. pyogenes cas9. Exemplary Cas9 proteins of the disclosure may be isolated or derived from any species, including, but not limited to, a bacteria or an archaca. In some embodiments of this disclosure a Cas9 sequence is modified to include two nuclear localization sequences (NLSs) (e.g., PKKKRKV (SEQ ID NO:61) at the C- and N-termini of the Cas9 protein, and a mini-polyadenylation signal (or Poly-A sequence). An exemplary NLS is SV40 large T antigen NLS (PKKKRRV (SEQ ID NO:62)) and nucleoplasmin NLS (KRPAATKKAGQAKKKK (SEQ ID NO:63)). Other NLSs are known in the art; see, e.g., Cokol et al., EMBO Rep. 2000 Nov. 15; 1(5):411-415; Freitas and Cunha, Curr Genomics. 2009 December; 10(8): 550-557. An exemplary polyadenylation signal is TAGCAATAAAGGATCGTTTATTTTCATTGGAAGCGTGTGTTGGTTTTTTGATCA GGCGCG (SEQ ID NO:64). In some embodiments, the RNA-guided nuclease is a nuclease-dead Cas protein (e.g., dCas9).

### Guide RNAs

Provided herein are guide RNAs (gRNAs) designed to target one or more sites in the HSPG binding domain of a wild type ApoE. In some embodiments, the gRNAs are designed to introduce a mutation in the wild type ApoE that results in a mutation at amino acid position 136. In some embodiments, the guide RNAs provided herein are designed to introduce an R136S mutation in a wild type APOE gene (e.g. APOE2, APOE3, or APOE4), where exemplary guide RNAs can be found in Table 7. In some embodiments, also provided are templates for repairing the double stranded break and introducing an R136S mutation. An exemplary template sequence is as follows:

(SEQ ID NO: 65)
CGCCTGGTGCAGTACCGCGGCGAGGTGCAGGCCATGCTCGGCCAGAGCAC
aGAGGAGCTcCGcGTGaGtCTCGCaagtCCACCTGCGCAAGCTGCGTAAG
CGGCTCCTCCGCGATGCCGATGACCTGC

where silent mutations to abolish PAM motifs are double underlined, the codon corresponding to the R136S mutation is bolded, and silent mutation to generate SacI site for cleaving PCR products from clones that received the template is italicized.

In some embodiments, the guide RNAs provided herein are designed to target exon 3 (amino acids 1-61) of a wild type APOE gene, or of a variant present in a subject (the methods can thus include determining the sequence of the APOE gene in a subject, and using that sequence to determine the sequence of a suitable guideRNA for targeting exon 3 in that subject). In some embodiments, a double stranded break repair through non-homologous end joining (NHEJ) results in short insertions or deletions leading to ApoE knockout. Exemplary guide RNA sequences for ApoE knockout are shown in Table 8.

### Base Editing

In some embodiments, the APOE gene is edited using the base editing technique (e.g. those described in Rees and Liu, Nature Reviews Genetics 19, 770-788, 2018; Komor et al., Nature 533, 420-424). In some embodiments, guide RNAs are designed to introduce an R136H mutation in a wild type APOE gene (e.g. APOE2, APOE3, or APOE4) using base editing, where exemplary guide RNAs can be found in Table 6. Base editors that convert C/G to A/T and adenine base editors that convert A/T to G/C can be used to introduce point mutations. Exemplary base editors include those described in Komor et al., Nature 533, 420-424 and Gaudelli et al., Nature 551, 464-471).

### AAV Delivery Systems

The methods include delivery of a CRISPR/Cas9 genome editing system, including a Cas9 nuclease and one or two guide RNAs, to a subject in need thereof. The delivery methods can include, e.g., viral delivery, e.g., preferably using an adeno-associated virus (AAV) vector that comprises sequences encoding the Cas9 and guide RNA(s). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., Pang et al., Vision Research 2008, 48(3):377-385; Khani et al., Invest Ophthalmol Vis Sci. 2007 September; 48(9):3954-61; Allocca et al., J. Virol. 2007 81(20): 11372-11380). In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and a Cas9 sequence and guide RNA sequence as described herein. In some embodiments, the AAV capsid polypeptide is an Anc80 polypeptide, e.g., Anc80L27; Anc80L59; Anc80L60; Anc80L62; Anc80L65; Anc80L33; Anc80L36; or Anc80L44. In some embodiments, the AAV incorporates inverted terminal repeats (ITRs) derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

Cas9 expression is driven by a promoter known in the art. In some embodiments, expression is driven by one of three promoters: cytomegalovirus (CMV), elongation factor-1 (EFS), or human g-protein receptor coupled kinase-1 (hGRKI), which is specifically expressed in retinal photoreceptor cells. Nucleotide sequences for each of these promoters are provided in Table 5 of WO 2018/026976. Modifications of these sequences may be possible or desirable in certain applications, and such modifications are within the scope of this disclosure.

Expression of the gRNAs in the AAV vector is driven by a promoter known in the art. In some embodiments, a polymerase III promoter, such as a human U6 promoter. An exemplary U6 promoter sequence is presented below:

(SEQ ID NO: 66)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACC.

In some embodiments, the nucleic acid or AAV vector shares at least 80%, 85%, 90%, 95%, 96%, 97%, 98%, 99% or greater sequence identity with one of the nucleic acids or AAV vectors recited above.

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection (e.g., intracranial injection) and intranasal delivery. The concentration of AAV vector within the composition is selected to ensure, among other things, that a sufficient AAV dose is administered to the brain of the subject, taking account of dead volume within the injection apparatus and the relatively limited volume that can be safely administered. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal or cochlear space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Explants are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

### Methods of Screening (Test Compounds)

Included herein are methods for screening test compounds, e.g., polypeptides, polynucleotides, inorganic or organic large or small molecule test compounds, to identify agents useful in the treatment or prevention of disorders associated with dementia and/or mild cognitive impairment (e.g., Alzheimer's disease, vascular dementia, Lewy body dementia, frontotemporal dementia, Parkinson's disease, or Huntington's disease), neurodegenerative diseases, cerebrovascular diseases, brain injury, retinal degeneration, or retinal injury. In some embodiments, the test compounds modulate the HSPG/heparin-binding properties of an ApoE protein (e.g. a wild type ApoE protein). In some embodiments, the test compounds reduce the HSPG/heparin-binding properties of an ApoE protein (e.g. a wild type ApoE protein).

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a sample containing one or more ApoE protein(s), and one or more effects of the test compound (e.g. HSPG/heparin binding affinity of the ApoE protein(s)) is evaluated. The ability of test compounds to modify the HSPG/heparin binding affinity of the ApoE protein(s) can be evaluated, e.g. using heparin sepharose columns, or antibodies that specifically recognize the HSPG-binding domain of ApoE as described herein. In some embodiments, methods for screening test compounds as described herein include evaluating the ability of a test compound to modify (e.g. inhibit or reduce) binding of antibodies as described herein that bind to one or more HSPG-binding sites or one or more sites of allosteric modulation of HSPG binding of a wild type or mutant ApoE. In some embodiments, a test compound competes with the antibodies as described herein for ApoE binding.

In some embodiments, the test sample is, or is derived from (e.g., a sample taken from) an in vivo model of a disorder as described herein. For example, an animal model, e.g., a rodent such as a rat, can be used.

A test compound that has been screened by a method described herein and determined to reduce or modify ApoE and HSPG/heparin binding, can be considered a candidate compound. A candidate compound that has been screened, e.g., in an in vivo model of a disorder, e.g., dementia and/or mild cognitive impairment (such as those associated with Alzheimer's disease, vascular dementia, Lewy body dementia, frontotemporal dementia, Parkinson's disease or Huntington's disease), neurodegenerative diseases, cerebrovascular diseases, brain injury, retinal degeneration, or retinal injury, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that have a desirable effect on the disorder) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating, preventing, or delaying of development or progression of disorders associated with dementia and/or mild cognitive impairment, as described herein, e.g., Alzheimer's disease, vascular dementia, Lewy body dementia, frontotemporal dementia, Parkinson's disease or Huntington's disease, and useful in treating, preventing, or delaying of development or progression of neurodegenerative diseases, cerebrovascular diseases, brain injury, retinal degeneration, or retinal injury. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with any of the disorders as described herein. The animal can be monitored for a change in the disorder, e.g., for an improvement in a parameter of the disorder, e.g., a parameter related to clinical outcome.

### Methods of Treatment

The methods described herein include methods for the treatment, prevention, or delay of development or progression of disorders associated with dementia and/or mild cognitive impairments, neurodegenerative diseases, cerebrovascular diseases, brain injury, retinal degeneration, or retinal injury. In some embodiments, the disorder associated with dementia and/or mild cognitive impairments is Alzheimer's disease, vascular dementia, Lewy body dementia, frontotemporal dementia, Parkinson's disease, or Huntington's disease. Additional non-limiting examples of neurodegenerative diseases include prion disease, motor neuron disease, and amyotrophic lateral sclerosis (ALS). Non-limiting examples of cerebrovascular diseases include stroke, carotid stenosis, vertebral stenosis, and aneurysms. Non-limiting examples of brain injuries include traumatic brain injury and acquired brain injury. Retinal degeneration such as glaucoma, age-related macular degeneration, may involve amyloid-beta and neurofibrillary tangle toxicity, establishing a link between retinal degeneration and neurodegeneration (e.g., Alzheimer's disease) (See, e.g. Mckinnon, Frontiers in Bioscience 8, s1140-1156, 2003; Johnson et al. PNAS 99(18) 11830-11835, 2002; and Sivak, Investigative Ophthalmology & Visual Science, 54(1) 871-880, 2013). Accordingly, treatments for neurodegeneration can be used to treat retinal or optic nerve degeneration.

The methods include administering a therapeutically effective amount of any of the antibodies, peptides, fusion proteins, or genome editing system as described herein, to a subject who is in need of, or who has been determined to be in need of, such treatment.

The methods described herein are also useful for subjects at risk for developing any of the disorders described herein. Subjects at risk for developing Alzheimer's disease may include those that are homozygous or heterozygous for the APOE4 allele, carriers of autosomal dominant Alzheimer's disease-causing mutations (e.g. mutations in the amyloid beta precursor (APP) gene, PSEN1 gene, or PSEN2 gene), trisomy 21 (e.g. subjects whose cognitive impairment is developmental only). Subjects at risk for developing Alzheimer's disease may also include those that have polygenic risk scores associated with increased risk of developing the disease, and those with brain imaging or other biomarker (e.g. biomarker in the body fluids) evidence of Alzheimer's disease. The methods for preventing or delaying the development of disorders described herein may also be useful for subjects that are not at risk for developing the above disorders, such as any subjects over the age of 50 (e.g. over the age of 55, 60, 65, 70, 75, 80, 85, 90, or 95).

As used in this context, to “treat” means to ameliorate at least one symptom of the disorder associated with the disorders as described herein. Often, Alzheimer's disease results in fibril formation, amyloid aggregation, and reduced cognitive performance; thus, a treatment can result in a reduction in fibril formation and/or amyloid aggregation in the brain, reduced tau formation of tangles, improved brain metabolism, improved neurocognitive functions and/or cognitive performance.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Identification of the APOE3 Christchurch R136S Mutation in a PSEN1 Mutation Carrier

### Materials and Methods:

Clinical assessments: Institutional review boards from the University of Antioquia, Massachusetts General Hospital, and the Schepens Eye Research Institute of Massachusetts Eye and Ear approved this study. Like all of the research participants, the proband case provided her written informed consent. Clinical ratings and neuropsychological tests were performed as noted in Table 1. PSEN1 E280A genotyping was conducted as previously described. 1

All clinical measures were undertaken at the University of Antioquia (Medellín, Colombia) and were conducted in Spanish by physicians and psychologists trained in assessment. Neurocognitive testing included a comprehensive multi-domain assessment. Some of the test administered were the Spanish versions of the Mini-Mental State Examination (MMSE), the Clinical Dementia Rating (CDR), and the Consortium to Establish a Registry for Alzheimer's disease battery, which have been adapted to this Colombian population.2 Additional testing consisted of the Yesavage Geriatric Depression Scale 3 and the Functional Assessment Staging test 4, which were done within six months of brain imaging.

A detailed ophthalmic evaluation was performed. It included visual acuity assessment, slit-lamp and indirect ophthalmoscopy examination. Ultra-widefield fundus and fundus autofluorescence images using Optos Panoramic 200Tx imaging system (Optos PLC, Dunfermline, Scotland, UK) were obtained.

Additionally, Spectralis SD-OCT (Heidelberg Engineering, Heidelberg, Germany) and OCT angiography with Cirrus HD-OCT with AngioPlex (Carl Zeiss Meditec, Dublin, CA) were also done.

Additional studies were conducted after the PSEN1 E280A mutation carrier was discovered to have two copies of the APOE3ch variant. A fasting serum lipid panel was performed to explore the possibility of hyperliproteinemia type III, a condition found in 5-10% of persons homozygous for the relatively AD protective APOE2 allele and in most but not all APOE3ch carriers.5

Finally, an analysis of data from clinically and neuropathologically verified AD cases and controls from the AD Genetics Consortium was used to clarify whether homozygosity for the APOE2 allele was associated with an exceptionally low risk of late-onset AD dementia.

Whole exome sequencing: Whole-exome capture and sequencing were performed using Illumina chemistry for variant discovery; rare variants with less than 1% frequency in genes previously associated with AD were considered in the search for candidate risk modifiers. Specifically, rare DNA variants (minor allele frequency <1%) within exonic regions and splice-site junctions (5 bp into introns) of genes were identified using bioinformatics tools. Whole exome libraries were constructed and sequenced on an Illumina HiSeq 4000 sequencer with the use of 151 bp paired-end reads. Library construction was performed using a previously described protocol 6 modified as follows. Genomic DNA input was reduced from 3 μg to 50 ng in 10 μL of solution and enzymatically sheared. Dual-indexed Illumina paired end adapters were replaced with palindromic forked adapters with unique 8 base index sequences embedded within the adapter and added to each end for adapter ligation. In-solution hybrid selection was performed using the Illumina Rapid Capture Exome enrichment kit with 38 Mb target territory (29 Mb baited). The targeted region included 98.3% of the intervals in the Refseq exome database. Dual-indexed libraries were pooled into groups of up to 96 samples prior to hybridization. The enriched library pools were quantified via PicoGreen after elution from streptavadin beads and then normalized. For cluster amplification and sequencing, the libraries prepared using forked, indexed adapters were quantified using quantitative PCR (KAPA biosystems), normalized to 2 nM using Hamilton Starlet Liquid Handling system, and pooled with equal volume using the Hamilton Starlet Liquid Handling system. Pools were then denatured in 0.1 N NaOH. Denatured samples were diluted into strip tubes using the Hamilton Starlet Liquid Handling system. Cluster amplification of the templates was performed according to the manufacturer's protocol (Illumina) using the Illumina cBot. Flowcells were sequenced on HiSeq 4000 Sequencing-by-Synthesis Kits, then analyzed using RTA2.7.3.

Exome sequencing data was processed and analyzed with the bioinformatics pipeline of the Center's Clinical Exome Sequencing of the Center for Personalized Medicine (CPM) Clinical Genomics Laboratory and the Translational Genomics Research Institute. Briefly, Edico Genome's Dragen Genome Pipeline with default parameters was used to perform sequence alignment and variant calling. The open source software samtools and bcftools (samtools.github.io/) were used along with a set of custom scripts to perform coverage determination and initial variant filtering based on ExAC (Exome Aggregation Consortium, exac.broadinstitute.org/) allele frequencies.7 Sequence alignment was done against the Human hs37d5 decoy genome.8 To identify the potential modifier variants, a primary gene list of 15 genes was generated based on two HPO terms: HP:0002511, Alzheimer disease; HP:0003584, Late onset. These genes were AAGAB, ABCC8, AKT2, APOE, APP, BEAN1, GATA1, GCK, HMGA1, HNF1B, HNF4A, LDB3, PAX₄, PSEN1, and PSEN2.9 Rare DNA variants (minor allele frequency <1%) within exonic regions and splice-site junctions (5 bp into introns) of these genes were further annotated and analyzed using a commercial tool (Cartagenia v5.0). Sequence alterations were reported according to the Human Genome Variation Society (HGVS v2.0) nomenclature guidelines.

Whole genome sequencing: Whole-genome sequencing (WGS) and a Genomizer analysis (v 10.1.0) were used to conduct a comprehensive and unbiased ranking of other potential genetic risk modifiers, including those associated with a lower risk of Alzheimer's dementia, helping to exclude other potentially protective genetic factors. 10 For processing the WGS data, the same dragen pipeline described above was used. The data was aligned to the GRCh37 decoy genome (hs37d5). Variants that were called at a depth of <10× were filtered out and then were annotated using Ensembl's Variant Effect Predictor (VEP) tool. The version of VEP using was v93. The filtered and annotated set of variants was then compiled for Genomizer analysis.

APOE structure display: Image was obtained and modified from the RCSB PDB (rcsb.org) of PDB 2L7B a previously published structure¹¹ using NGL Viewer¹².

APOE Genotyping by Sanger sequencing: Reaction mixture for the amplification process was performed in a 50 μL volume that included the following components: 1×PfuUltra II Hostart Master Mix, 1 μL of each primer (10 μmol/L)(Forward primer: 5′-AGCCCTTCTCCCCGCCTCCCACTGT-3′ (SEQ ID NO:67) and Reverse primer: 5′-CTCCGCCACCTGCTCCTTCACCTCG-3′ (SEQ ID NO:68)), 5% DMSO and 1 μL of genomic DNA (100 ng/μL). 13 PCR cycling was run with initial denaturation at 95° C. for 2 min followed by 35 cycles with denaturation at 95° C. for 20 seconds, annealing at 60° C. for 30 seconds, extension at 72° C. for 40 seconds, and a final extension at 72° C. for 5 min. PCR products were purified using QIAquick Gel Extraction kit from Qiagen and sequenced by MGH CCIB DNA core using the 3730xl sequencer from Applied Biosystems.

MRI and PET imaging: Pittsburgh Compound B (PiB), flortaucipir (FTP) positron emission tomography (PET) and structural magnetic resonance imaging (MRI) measurements were acquired at Massachusetts General Hospital and analyzed at Massachusetts General Hospital and Banner Alzheimer's Institute as previously described.14 Fluorodeoxyglucose PET images were acquired at the University of Antioquia, Colombia, and analyzed as previously described 15. Imaging data from the case were compared to those from younger PSEN1 E280A mutation carriers who developed MCI at the kindred's expected age at clinical onset, and from mutation carriers who were cognitively unimpaired.

MRI was performed on a 3T Tim Trio (Siemens) and included a magnetization-prepared rapid gradient-echo (MPRAGE) processed with Freesurfer (FS) to identify grey white and pial surfaces to permit regions of interest (ROI) parcellation as follows: cerebellar grey, hippocampus, and the following Braak Stage related cortices: entorhinal, parahippocampal, inferior temporal, fusiform, posterior cingulate, as described previously 16-19.

18F-Flortaucipir (FTP) was prepared at MGH with a radiochemical yield of 14±3% and specific activity of 216±60 GBq/umol at the end of synthesis (60 min), and validated for human use (Shoup et al., 2013). 11C-Pittsburgh Compound B was prepared and PET images were acquired as previously described. 16 All PET images were acquired using a Siemens/CTI (Knoxville, TN) ECAT HR+ scanner (3D mode; 63 image planes; 15.2 cm axial field of view; 5.6 mm transaxial resolution and 2.4 mm slice interval. 11C PiB PET was acquired with a 8.5 to 15 mCi bolus injection followed immediately by a 60-minute dynamic acquisition in 69 frames (12×15 seconds, 57×60 seconds)). 18F FTP was acquired from 80-100 minutes after a 9.0 to 11.0 mCi bolus injection in 4×5-minute frames. PET images were reconstructed and attenuation-corrected, and each frame was evaluated to verify adequate count statistics and absence of head motion.

18F FTP specific binding was expressed in FS ROIs as the standardized uptake value ratio (SUVR) to cerebellum, similar to a previous report 19, using the FS cerebellar grey ROI as reference. For voxelwise analyses, each subject's MPRAGE was registered to the template MR in SPM8 (SPM), and the spatially transformed SUVR PET data was smoothed with a 8 mm Gaussian kernel to account for individual anatomic differences 20. To account for possible 18F FTP off-target binding in choroid plexus, which may confound hippocampal signal, we used a linear regression to regress the choroid plexus, as previously reported 21.

11C PiB PET data were expressed as the distribution volume ratio (DVR) with cerebellar grey as reference tissue; regional time-activity curves were used to compute regional DVRs for each ROI using the Logan graphical method applied to data from 40 to 60 minutes after injection 16,22. 11C PiB retention was assessed using a large cortical ROI aggregate that included frontal, lateral temporal and retrosplenial cortices (FLR) as described previously 23,24

18F-fludeoxyglucose PET was performed on a 64-section PET/computed tomography imaging system (Biograph mCT; Siemens) using intravenous administration of 5 mCi (185 million Bq) of 18F-fludeoxyglucose after a 30-minute radiotracer uptake period when resting in a darkened room, followed by a 30-minute dynamic emission scan (six 5-minute frames). Images were reconstructed with computed tomographic attenuation correction. Precuneus to whole-brain cerebral metabolic rate for glucose (CMRgl) ratios were characterized from a bilateral region of interest (ROI) in each participant's 18F-fludeoxyglucose PET image using an automated brain mapping algorithm (SPM8; fil.ion.ucl.ac.uk/spm/software/spm8). Hippocampal to total intracranial volume ratios were characterized from bilateral ROIs in each participant's T1-weighted MR image using FreeSurfer (surfer.nmr.mgh.harvard.edu). All images were visually inspected to verify ROI characterization.

Amyloid aggregation studies: Human ApoE3 protein fragments (including the carboxyl-terminus domain plus a histidine tag) with and without the Christchurch variant were synthesized in bacteria, purified (Innovagen), and used to assess the differential effects of these proteins on Aβ₄₂ aggregation in vitro using Thioflavin T (SensoLyte® ThT β-Amyloid (1-42) Aggregation kit, cat. #AS-72214). For this assay, 55 μM of Aβ₄₂ was added to solutions of either 10 μM Wild Type apoE3 protein or Mutant 136 Arg→Ser ApoE3 protein in a transparent, no-binding 96-well plate. Samples were then mixed with 2 mM Thioflavin T dye and fluorescence was read at Ex/Em=440/484 at intermittent time intervals over 2 hours. The plate was kept at 37° C. with 15 seconds shaking between reads.

Full-length ApoE3 proteins with and without the Christchurch mutation were also expressed in Flp-In™ T-REX™ 293 (Thermo Fisher Scientific) mammalian cells via transient transfection to confirm the impact of these proteins on Aβ₄₂ aggregation using a previously published split-luciferase complementation assay.26 The latter analysis were conducted using the human APOE3 expression from Addgene (Plasmid #8708627) as the WT or APOE3 with the Christchurch variant introduced via site-directed mutagenesis. Reagents for luciferase assay were purchased from Promega.

### Results

About 1,200 Colombian Presenilin 1 (PSEN1) E280A mutation carriers and 4,600 non-carriers were identified, who together compose the world's largest known kindred with autosomal dominant Alzheimer's disease (ADAD).^{28,29} The mutation carriers usually develop mild cognitive impairment (MCI) and dementia at the respective median ages of 44 (95% CI, 43-45) and 49 (95% CI, 49-50) years.^{30,31} Studying autosomal dominant AD (ADAD) mutation carriers who remain cognitively unimpaired until older ages could help in the discovery of risk-reducing gene variants.³² Characterizing AD biomarkers in these individuals could help inform the potentially targetable mechanisms by which these genes exert their relative protective effects. We identified a PSEN1 E280A mutation carrier who did not develop MCI until her seventies, nearly three decades after the median age at onset.

This study was conducted with the participant's written informed consent following Institutional Review Board guidelines (her exact age and other identifying information are omitted to protect her anonymity and confidentiality). The participant was confirmed to carry the amyloid-β₄₂ (Aβ₄₂)-overproducing PSEN1 E280A mutation, confirmed by report of family informants to be cognitively unimpaired until her seventies, and subsequently met criteria for MCI³³ during a 24-month period of annual assessments. She remained fully independent for basic and instrumental activities of daily living, without evident signs of worsening of her abilities to perform these activities. At intake assessment, her memory deficits were limited to recent events and her neurological exams were normal. Her age and education-adjusted neuropsychological test scores indicated a preferential impairment in recall memory, relatively preserved recognition memory, initial learning, naming, visuospatial abilities and verbal fluency skills, and relatively stable cognitive performance during the 24-month assessment period (Table 1).

TABLE 1
Cognitive Test Scores and Percentiles

| | | | | Mean |
|---|---|---|---|---|
| Cognitive Tests | Raw Scores (Percentiles**) | | | (SD)** |
| MMSE/30* | 18 (1^{st}) | 16 (<1^{st}) | 19 (4^{th}) | 22 (1.7) |
| Naming/15 | 9 (7^{th}) | 9 (7^{th}) | 8 (3^{rd}) | 12.03 (2.1) |
| CERAD Word List | 10 (5^{th}) | 6 (<1^{st}) | 8 (1^{st}) | 15.64 (3.44) |
| Learning/30 | | | | |
| CERAD Word | 0 (<1^{st}) | 0 (<1^{st}) | 0 (<1^{st}) | 5.77 (1.98) |
| List Delayed | | | | |
| Recall/10 | | | | |
| CERAD Word List | 9 (27^{th}) | 8 (5^{th}) | 7 (1^{st}) | 9.58 (0.98) |
| Recognition/10 | | | | |
| CERAD Praxis- | 8 (18^{th}) | 9 (39^{th}) | 6 (1^{st}) | 9.42 (1.57) |
| Copy/11 | | | | |
| CERAD Praxis- | 0 (<1^{st}) | 2 (3^{rd}) | 0 (<1^{st}) | 7.52 (2.84) |
| Recall/11 | | | | |
| Semantic Fluency | 12 (13^{th}) | 12 (13^{th}) | 12 (13^{th}) | 16.97 (4.3) |
| (Animals) | | | | |
| Phonemic Fluency | 10 (13^{th}) | 11(14^{th}) | 10 (13^{th}) | 23.75 (11.89) |
| (“F” Words) | | | | |
| Raven's Matrices | 7 (27^{th}) | 7 (27^{th}) | 8 (47^{th}) | 8.21 (2.13) |
| Test, Form | | | | |
| A/12 | | | | |
| GDS/15 | 9 | 2 | 9 | |
| EDG/7 | 3 | 3 | 4 | |

MMSE: Mini-Mental State Examination
CERAD: Consortium to Establish a Registry for Alzheimer's
Disease Test Battery.
GDS: Geriatric Depression Scale
EDG: Global Deterioration Scale
*MMSE subtests that require reading and writing skills were not administered due to her limited literacy skills. Her maximum possible score was 23 (instead of 30).
**Percentiles were calculated using norms for this Colombian population.
Percentiles between 25 and 75 place her performance in the average range for her age and education.
Percentiles between 9 and 25 classify her performance as below average. Percentiles between 2 and 8 classify her performance as low. Percentiles below 1 classify her performance as extremely low.
#Brain imaging described in this report was acquired three months after the initial cognitive testing.

Whole exome sequencing corroborated her PSEN1 E280A mutation and discovered that she also had two copies of the rare APOE3 Christchurch R136S (APOEch) mutation. Sanger sequencing confirmed the latter finding. Whole genome sequencing and a Genomizer analysis were used to comprehensibly identify and rank all potentially significant rare and common variants.³⁴ Using this approach, the PSEN1 E280A mutation was confirmed to be the participant's primary risk factor and APOE3ch homozygosity was confirmed to be her primary resistance factor.

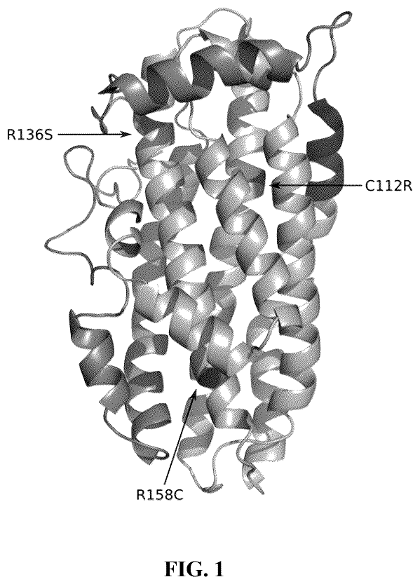

APOE, the major susceptibility gene for late-onset AD, has three common alleles (APOE2, 3, and 4). Compared to the most common APOE3/3 genotype, APOE2 is associated with a lower AD risk and older age at dementia onset,³⁵ and each additional copy of APOE4 is associated with a higher risk and younger age at onset.^{36,37} The APOEch variant, an arginine-to-serine substitution at amino acid 136 (136Arg→Ser), corresponding to codon 154,³⁸ can reside on any of the common APOE alleles,³⁹ including this participant's two APOE3 alleles. FIG. 1 shows a model of the structure of the wild type ApoE3 protein. N-terminal (residues 1-191) and C-terminal (residues 201-299) domains are highlighted. The amino acid positions for APOE4 (C112R), APOE3ch (R136S) and APOE2 (R158C) variants are shown.

The APOE3ch variant was absent from AlzAD or ExAC databases reporting on about 180,000 exomes. The R136S was previously identified in APOE2 individuals with HLP III but its potential effect in the progression of AD has not been previously reported.⁴⁰ We sequenced DNA samples from two other PSEN1 E280A carriers with delayed age-at-onset (age at onset 62 and 70 years) via whole genome sequencing. None of these individuals had the APOE3 R136S variant nor APOE2; the latter was previously shown to delay disease onset in this kindred.⁴¹

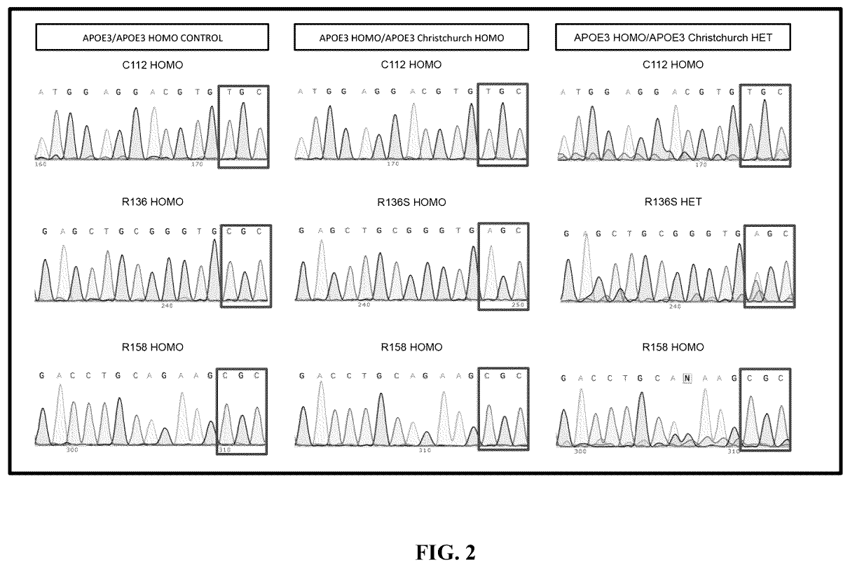

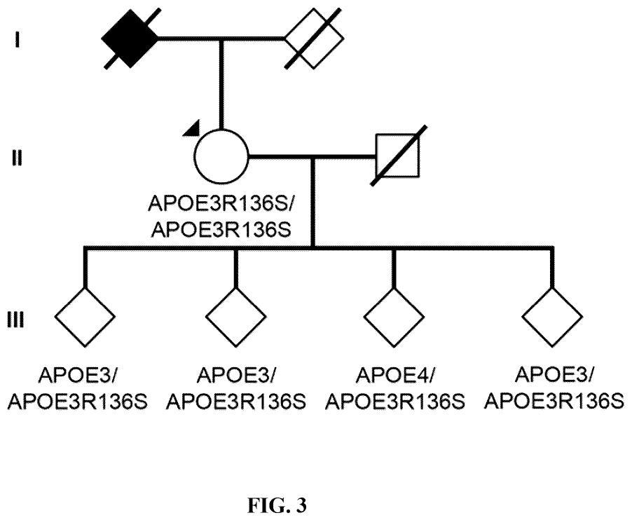

To confirm a potential association between the APOE3 R136S mutation and delayed age at onset of AD, we conducted whole genome sequencing, neurological, and neuropsychological testing in the four descendants of the proband case, which were all older than fifty years, and expected to carry the APOE3 R136S. FIG. 2 shows representative Sanger sequencing results of APOE from control, proband and descendant's samples. Upper row: C112 homozygous sequences are shown in all cases. Middle row: R136 homozygous sequence is shown in left panel from a control individual. Middle panel shows homozygous change resulting in R136S mutation. Right panel shows a R136S heterozygous mutation example of a descendant of the proband. Low row: R158 homozygous sequences are shown in all cases. FIG. 3 shows the proband's genealogy, with circles representing females, squares representing males, diamonds representing individuals whose gender has been masked for privacy, arrowhead depicts proband individual with MCI, and shading indicates individual with history of dementia. Deceased individuals are marked with a crossed bar. The individual APOE and PSEN1 genotypes are indicated as appropriate to preserve anonymity. Although other unknown genetic or epigenetic factors may have contributed to late age at onset of cognitive impairment in these two related PSEN1 E280A carriers, we suggest that the APOE3 R136S variant modifies the AD phenotype by buffering the effects of amyloid-β accumulation in the brain and subsequently delaying the emergence of tau pathology, neurodegeneration (i.e. brain atrophy), and symptoms onset.

Carriers of APOEch and other rare mutations in APOE's low density lipoprotein receptor (LDLR) binding region commonly have hyperlipoproteinemia type III (HLP-III), similar to that observed in 5-10% of APOE2 homozygotes.^{43,44} The participant in this report was confirmed to have HLP-III, including APOEch and elevated triglyceride and total cholesterol levels (See, Table 2).

TABLE 2
Dyslipidemia workup

| Test | | |
|---|---|---|
| Lipid panel | Subject | Normal range* |
| Triglycerides (mg/dl) | 691.88 | <250 |
| Total Cholesterol (mg/dl) | 511.76 | 150-199 |
| VLDL-C (mg/dl) | (−) | <30 |
| LDL-C (mg/dl) | (−) | ≤130 |
| Direct LDL-C (mg/dl) | 147 | ≤130 |
| HDL-C (mg/dl) | 55.74 | ≥40 |
| Cholesterol/HDL ratio | 9.18 | <5 |
| Apolipoprotein A-I (mg/dl) | 177 | F: 98-210 |
| Apolipoprotein B (mg/dl) | 217 | F: 44-148 |
| Apo B/Apo-I ratio | 1.23 | F: 0.35-1.15 |
| Lipoprotein A (mg/dl) | 86.6 | ≤30 |

HDL-C: High-density lipoprotein cholesterol,
VLDL-C: Very low-density lipoprotein cholesterol,
LDL-C: Low-density lipoprotein cholesterol.
*Normal range according to Merck Manual and Laboratory values.

Detailed laboratory workup showed abnormal lipid profile in our proband individual and three of the four descendants carrying the APOE3 R136S (Table 3). These four subjects had high level of total cholesterol and triglycerides. Very low-density lipoprotein (VLDL) and low-density lipoprotein (LDL) were higher in two of the descendants and not measurable in the proband individual and one of the descendants, which had triglyceride levels higher 400 mg/dL (the threshold for accuracy of the indirect method of lipid profiling) (Table 3). Further analyses using direct enzymatic tests showed higher than normal LDL in these two individuals. One of the descendants had a lipid profile within normal limits despite carrying the APOE3 R136S and the APOE4. Incomplete penetrance of HLP III has been previously reported for APOE2 and for R136S mutation carriers.⁴⁵ We ruled out secondary causes of lipid disorders as diabetes, obesity, alcoholism, renal disorders or thyroid diseases in these subjects. None of the mutation carrier individuals had xanthomas, which are diagnostic of HLP III, or cardiovascular diseases. The combination of the abnormal lipid profile and APOE3 R136S mutation in these subjects is consistent with a diagnosis of familial HLP III.

TABLE 3
Dementia and dyslipidemia workup in study population

| | Patient No. (Sex-Age) | | | | | |
|---|---|---|---|---|---|---|
| Test | 1 (F-75) | 2 (F-51) | 3 (F-53) | 4 (M-49) | 5 (M-54) | Normal range* |
| CBC | | | | | | |
| RBC count (×10⁶ mm³) | 4.7 | 4.73 | 4.47 | 5.26 | 5.37 | 4.2-5.9 |
| Hemoglobin (g/dl) | 13 | 14.1 | 12.9 | 15.1 | 15.1 | M: 14-17 F: 12-16 |
| Hematocrit (%) | 38.5 | 42.3 | 39.2 | 46.3 | 45.7 | M: 41-51 F: 36-47 |
| MCV (fL) | 81.9 | 89.4 | 87.8 | 88.1 | 85 | 80-100 |
| MCH (pg) | 27.6 | 29.8 | 28.8 | 28.6 | 28.1 | 28-32 |
| MCHC (g/dl) | 33.7 | 33.4 | 32.8 | 32.5 | 33 | 32-36 |
| RDW (%) | 13.7 | 12.5 | 13 | 13.3 | 13.5 | 11.5-14.5 |
| WBC count (×10³ mm³) | 5.51 | 10.45 | 9.61 | 12.78 | 20.18 | 4.5-11 |
| Platelets (×10³ mm³) | 264 | 289 | 334 | 256 | 295 | 150-350 |
| MPV (fL) | 7 | 8.2 | 8.8 | 8.3 | 7.8 | 6.5-13.5 |
| ESR (mm/h) | 30 | 20 | 51 | 18 | 2 | M: 0-15 F: 0-20 |
| BMP | | | | | | |
| Glucose (mg/dl) | 107.1 | 128.28 | 107.82 | 112.15 | 131.19 | 70-105 |
| BUN (mg/dl) | 11.98 | 13.03 | 9.71 | 15.65 | 14.39 | 8-20 |
| Creatinine (mg/dl) | 0.57 | 0.59 | 0.67 | 1.04 | 0.95 | 0.7-1.3 |
| Sodium (mmol/l) | 139.3 | 141.4 | 140 | 137.3 | 136.6 | 136-145 |
| Potassium (mmol/l) | 3.79 | 4.03 | 4.92 | 4.76 | 4.29 | 3.5-5 |
| Chloride (mmol/l) | 102 | 106.5 | 108.3 | 106.6 | 107.1 | 98-106 |
| LPP | | | | | | |
| Total Cholesterol (mg/dl) | 511.76 | 192.6 | 434.25 | 336.21 | 323.2 | 150-199 |
| HDL-C (mg/dl) | 55.74 | 60.52 | 60.56 | 53.76 | 33.78 | ≥40 |
| Cholesterol/HDL ratio | 9.18 | 3.18 | 7.17 | 6.25 | 9.57 | <5 |
| Triglycerides (mg/dl) | 691.88 | 50.32 | 321.3 | 282.49 | 437.92 | <250 |
| VLDL-C (mg/dl) | (—) | 10.06 | 64.26 | 56.5 | (—) | <30 |
| LDL-C (mg/dl) | (—) | 122.02 | 309.43 | 225.95 | (—) | ≤130 |
| Additional tests | | | | | | |
| Apolipoprotein A-I | 177 | 106 | 150 | 126 | 133 | M: 88-180 F: 98-210 |
| (mg/dl) | | | | | | |
| Apolipoprotein B (mg/dl) | 217 | 75 | 179 | 173 | 160 | M: 55-151 F: 44-148 |
| Apo B/Apo-I ratio | 1.23 | 0.71 | 1.19 | 1.37 | 1.2 | M: 0.45-1.25 F: 0.35-1.15 |
| Hemolytic Complement | 50.8 | 58 | 58.6 | 56.1 | 68.8 | 31.6-57.6 |
| 50% (U/ml) | | | | | | |
| Plasmatic homocysteine | 9.54 | 6.47 | 7.48 | 15.34 | 13.55 | M: 4-16 F: 3-14 |
| (μmol/l) | | | | | | |
| Lipoprotein A (mg/dl) | 86.6 | 6 | 86.1 | 6.5 | 62 | ≤30 |
| Serum complement C3 | 130.1 | 1202.6 | 1314.5 | 1281.9 | 1292.6 | 55-120 |
| (mg/dl) | | | | | | |
| Serum complement C4 | 20.6 | 177.9 | 131.7 | 146.5 | 245.5 | 20-59 |
| (mg/dl) | | | | | | |
| Complement Clq (mg/dl) | 24 | 23 | 28 | 28 | 33 | 10-25 |
| Free thyroxine (ng/dl) | 1.38 | 1.13 | 1.12 | 1.26 | 1.09 | 0.9-2.4 |
| TSH (μIU/ml) | 4.5 | 0.78 | 4.08 | 3.56 | 3.72 | 0.50-5.0 |
| Vitamin B12 (pg/ml) | 517 | 382 | 294 | 263 | 352 | 200-800 |
| Folate (ng/ml) | 16.14 | 7.52 | 8.38 | 2.14 | 6.6 | 2.5-20 |

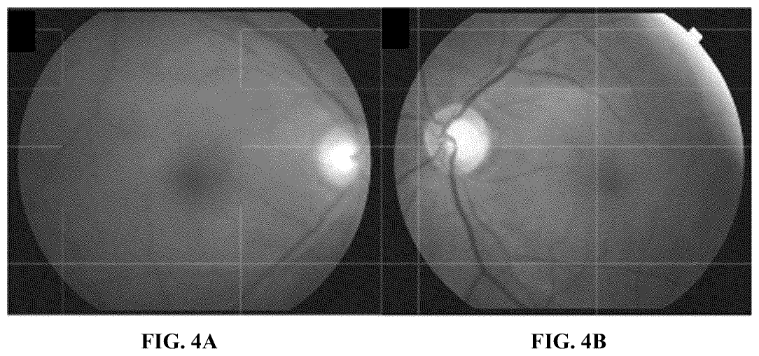

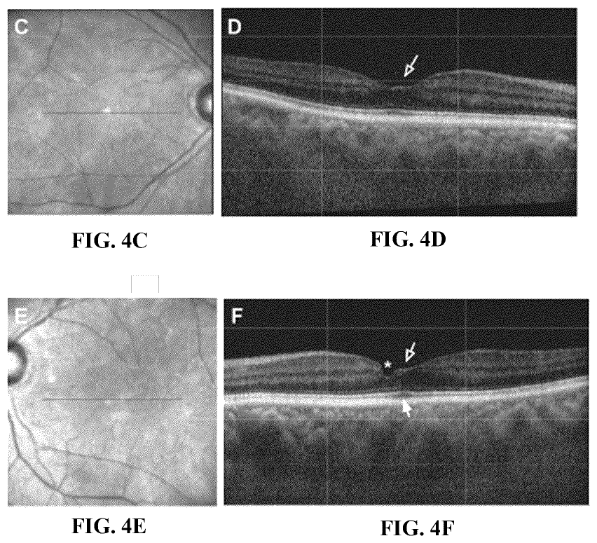

A detailed ophthalmic evaluation of the PSEN1 E280A mutation carrier with two APOE3ch alleles was performed. This carrier had a vision of 20/70 in the right eye and 20/40 in the left eye. The anterior segment exam was notable for a posterior chamber intraocular lens in the right eye with a dense posterior capsular opacification. Anterior segment of the left eye was notable for a nuclear sclerotic cataract (FIGS. 4A and 4B). The posterior segment examination of both eyes was normal, with a clear vitreous cavity, normal appearing optic nerve, macula, and peripheral retina. Further testing via optical coherence tomography (OCT) of the right eye was normal except for a small area of hyper-reflectivity overlying the fovea (FIG. 4D). FIG. 4C shows an infrared image of the right eye that depicts the cross section of the retina (line) seen in FIG. 4D. Further, OCT imaging of the left eye revealed a degenerative lamellar hole (denoted by the * in FIG. 4F) with a small defect in the external limiting membrane and ellipsoid layer (arrow in FIG. 4F).

While several mechanisms have been proposed to account for the impact of APOE variants on AD risk, most studies have focused on their differential effects (APOE2<3<4) on Aβ₄₂ aggregation and plaque burden.⁴⁷ In the present study, neuroimaging measurements were used to clarify whether the participant's resistance to the clinical onset of AD was associated with a) relatively little Aβ plaque burden despite more than seventy years of Aβ⁴² overproduction or with b) relatively high Aβ plaque burden but limited downstream measurements of paired helical filament (PHF) tau (neurofibrillary tangle burden) and neurodegeneration.

The participant's neuroimaging findings are shown in FIG. 5. The positron emission tomography (PET) images are superimposed onto the medial and lateral surfaces of the left hemisphere. The top row shows PET measurements of amyloid plaque burden (PiB DVRs). The bottom row shows PET measurements of paired helical filament (PHF) tau (i.e., neurofibrillary tangle) burden. The person with late-onset of MCI is in her seventies, and the person with the typical age at MCI onset is 44 years old.

As shown in FIG. 5, the person with late onset of MCI had unusually high PET measurements of Aβ plaque burden, as indicated by a higher mean cortical-to-cerebellar Pittsburgh Compound B (PiB) distribution volume ratio (DVR=1.96) than in PSEN1 E280A carriers who developed MCI in their forties (DVRs of 1.49-1.60). Despite her high Aβ plaque burden, the magnitude and/or spatial extent of her PHF tau burden and neurodegeneration were relatively limited: Her flortaucipir (tau) PET measurements were restricted to medial temporal and less commonly affected occipital regions with relative sparing of other regions that are characteristically affected in the clinical stages of AD (FIG. 5). Her fluorodeoxyglucose PET measurements of the cerebral metabolic rate for glucose were preserved in brain regions that are known to be preferentially affected by AD, including higher precuneus-to-whole brain measurements than in PSEN1 E280A mutation carriers who developed MCI at younger ages and many younger, cognitively unimpaired mutation carriers.

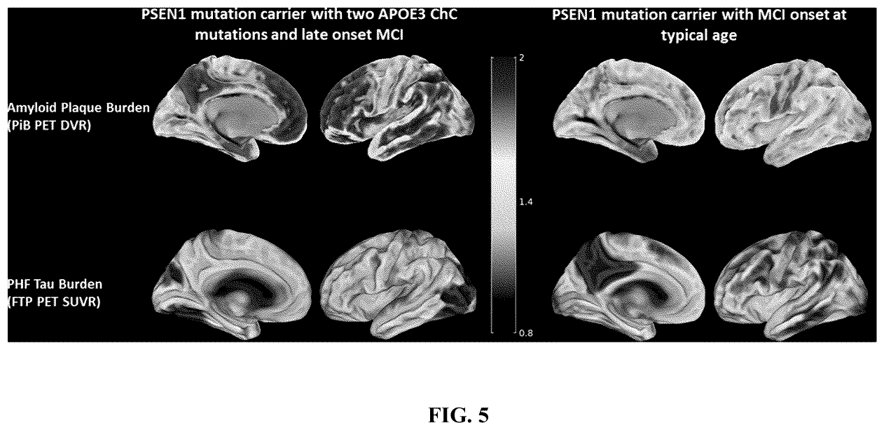

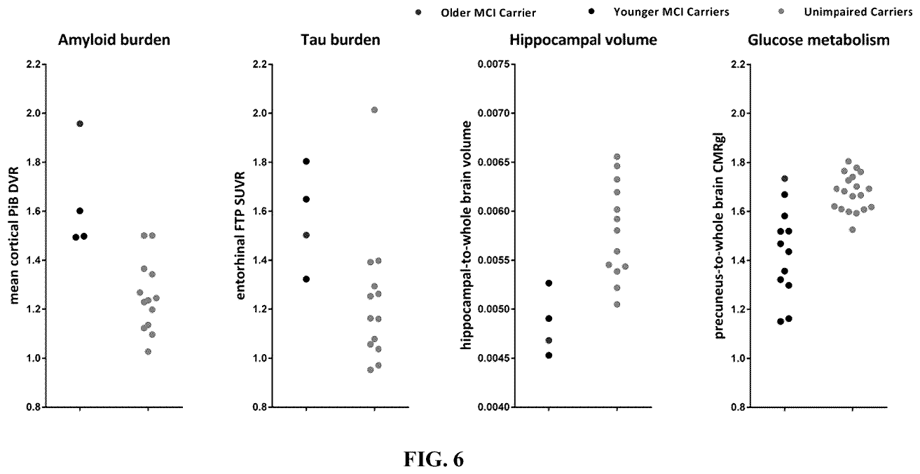

FIG. 6 shows measurements of mean cortical amyloid plaque burden, entorhinal cortex PHF tau burden, hippocampal volume, and precuneus glucose metabolism. These measurements were based on brain imaging results obtain from the PSEN1 E280A mutation carrier with two APOE3ch alleles and exceptionally late-onset of MCI (red dots), PSEN1 E280A mutation carriers with MCI at the kindred's typical, younger age at MCI onset (black dots), and PSEN1 E280A mutation carriers who have not yet developed MCI (gray dots). Amyloid plaque burden is expressed as mean cortical-to-cerebellar distribution volume ratios (DVRs). Paired helical filament (PHF) tau burden is expressed as entorhinal cortex-to-cerebellar flortaucipir (FTP) standard uptake value ratios (SUVRs). Hippocampal volumes, which may be reduced by hippocampal atrophy, are expressed as hippocampal-to-whole brain volume ratios. Cerebral glucose metabolism, which is reduced in AD-affected brain regions with synaptic dysfunction and loss, is reflected as precuneus-to-whole brain cerebral metabolic rate for glucose (CMRgl) ratios. As shown FIG. 6, while the PSEN1 E280A mutation carrier with two APOE3ch alleles had by far the highest amyloid plaque burden, she did not have comparably severe PHF tau burden or hippocampal atrophy, and she had no evidence of precuneus glucose hypometabolism. Her MRI-based hippocampal-to-whole brain volume, a hippocampal atrophy measurement that can be affected by AD and/or normal aging, was within the range of mutation carriers who developed MCI in their forties. Without wishing to be bound by theory, these results suggest that this APOE3ch homozygote's resistance to the clinical onset of AD is mediated through a mechanism that limits tau pathology and neurodegeneration even in the face of high Aβ plaque burden.

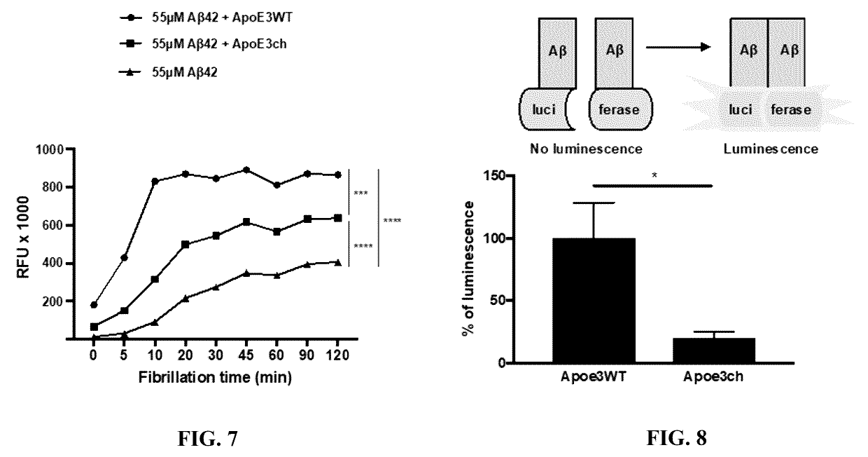

To study functional consequences of the APOE3ch variant, Aβ₄₂ aggregation in vitro in the presence of the bacteria-derived wild type human ApoE3 protein, presence of the mutant ApoE3ch protein, or in the absence of any ApoE protein were compared. The rate of Aβ₄₂ fibril formation was detected by Thioflavin T fluorescence. Aβ₄₂ aggregation was highest in the presence of wild type human ApoE3 protein (C-terminus domain), lower in the presence of human ApoE3ch (similar to that observed in the presence of ApoE2⁴⁸), and lowest in the absence of any ApoE (FIG. 7).

This finding was confirmed using a sensitive split-luciferase complementation assay in which luciferase signal is reconstituted once amyloid forms oligomers,⁴⁸ some of the most toxic amyloid species.⁴⁹ Full-length ApoE3ch expression in mammalian cells triggered significantly less oligomerization of Aβ₄₂ compared to wild type ApoE3, as luciferase luminescence by oligomer formation was significantly reduced in ApoE3ch compared to wild type ApoE3 (FIG. 8). These results provide validation of the genetic analysis and suggest that the protective effects of the ApoEch protein may result, at least in part, from its limited ability to promote Aβ₄₂ aggregation. It remains possible that the research participant may have had even greater Aβ plaque deposition had she survived to her seventies without the APOEch/3ch genotype and that the ApoE3ch protein altered the morphology of Aβ aggregates in ways that limited downstream neuroinflammation, tau pathology, neurodegeneration and cognitive decline.

A small percentage of Colombian kindred members were found to carry one copy of the APOE3ch mutation,⁵⁰ including four PSEN1 E280A mutation carriers who progressed to MCI at the median age of 45. For this reason, it was postulated that APOE3ch homozygosity may be required to dramatically lower the risk and postpone the clinical onset of autosomal dominant AD. Because the sample size was small, it remains possible that APOEch heterozygote individuals may have partial protection against autosomal dominant AD-related cognitive decline and substantial protection against sporadic late onset AD and/or neurodegeneration.

These results suggest that APOE variants differ in the extent of their pathogenic functions (APOEch and APOE2<3<4) and APOE3ch/3ch and APOE2/2 are associated with greatest functional loss. Interventions that safely and sufficiently edit APOE, lower its expression, or inhibit its pathogenic functions could have a profound impact on the treatment and prevention of AD. Interestingly, suppression of APOE expression in brain using an anti-sense oligonucleotide in Aβ-overproducing mice led to altered Aβ plaque morphology and fewer dystrophic neurites.⁵¹ This approach may be feasible because absence of APOE expression was tolerated in a middle-aged man who was homozygous for a frame-shift variant⁵² and availability of statins to treat HPL-III support the potential tolerability of ApoE-lowering treatments. See, e.g. Reiman et al. Nat Commun 1191):667, 2020.

Without wishing to be bound by theory, these results further suggest that homozygosity for APOE3ch—and APOE2—is associated with a profound resistance to the clinical onset of AD; that these genotypes exert their beneficial effects by directly or indirectly limiting downstream tau pathology and neurodegeneration; and that these effects are not based solely on the magnitude of Aβ plaque burden despite relative reductions in ApoE-mediated Aβ aggregation. These findings have implications for APOE's roles in the understanding, treatment, and prevention of AD, and may galvanize interest in developing APOE-modifying genetic and drug therapies for this disorder.

### Example 2: Heparin Binding Properties of the APOE3ch Mutant Protein

### Materials and Methods

Heparin column protocol: The heparin binding affinity of ApoE2, ApoE3, ApoE3ch and ApoE4 protein isoforms were compared using 1 ml Heparin Columns (BioVision-6554-1). The columns were acclimatized to room temperature for 1 hour prior to use. The columns were washed with 5 mL of 20 mM TRIS-HCL (pH7.5). 1 mL sample containing 50 μg/mL of APOE recombinant protein in 20 mM TRIS-HCL (pH7.5) was then recycled through the column 5 times. The column was then washed through 5 times with 20 mM TRIS-HCL (pH7.5). An increasing NaCl gradient (0.025-1M) in 20 mM TRIS-HCL was passed through the column and 1 mL fractions were collected and subsequently prepared for western blotting.

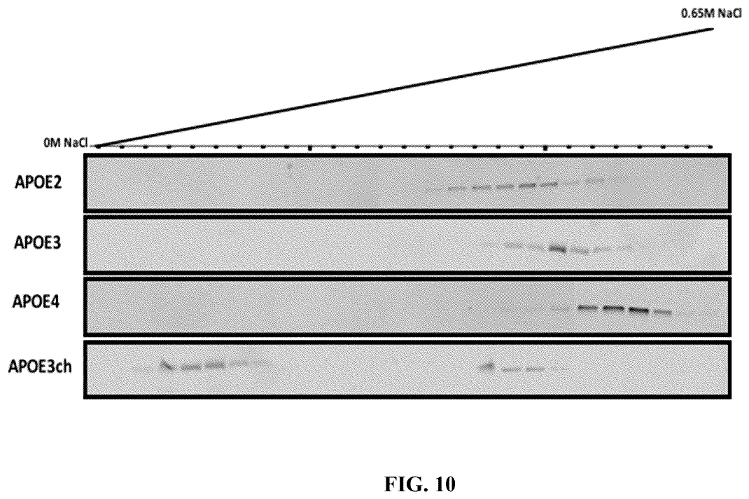

Western blotting: Western Blotting confirmed the elution of ApoE isoforms within fractions collected from the heparin binding columns. Fractions were diluted in 10 μl RIPA buffer (Cell Signaling Technology), 4 μl DTT (1M) and 10 μl Laemmli buffer to a final volume of 40 μl. Samples were separated on a 4-20% Mini-PROTEAN®TGX™ Precast Protein Gels (Bio-Rad), transferred to nitrocellulose membranes (VWR; 27376-991), blocked with Odyssey Blocking Buffer (LI-COR Biosciences, Lincoln, NE), and probed with mouse anti-his tag (Novus biologicals.), and IRDye 800CW donkey anti-rabbit (LI-COR Biosciences) antibodies. Immunoreactive bands were visualized using the Odyssey Infrared Imaging System and visualized on the Image Studio version 2.1 (LI-COR Biosciences). Individual gels were stitched together to generate FIG. 10.

Heparin plate ELISA protocol: An ELISA was carried out using heparin microplates (Bioworld; 50-197-531). These were blocked for one hour using sample preparation reagent (DY008). Heparin plates were incubated with 0.1 μg/well of each of the recombinant ApoE protein isoforms (ApoE2, ApoE3, ApoE3ch and ApoE4) for 2 hours, the plate was then washed five times in PBS containing a gradient of NaCl (0-0.5M) and then washed three times in the Wash Buffer (DY008). Anti-His tag antibody was incubated overnight at 1:10,000 (Novus biologicals; NBP2-61482). The plate was then washed five times to ensure removal of unbound primary antibody, incubated with donkey anti-rabbit-HRP (1:10000) for 45 minutes, and then washed five times to ensure removal of secondary antibody. Sulfuric acid from the ELISA reagent kit (DY008) was warmed to 37° C. prior to addition of 100 μl of tetramethylbenzidine (Millipore) initiating the detection phase of the reaction. After a 5-mins incubation, sulfuric acid was added to terminate the reaction. The plate was then read using a SPECTRAmax plus 384 (Molecular Devices). The wavelength of the read was 450 nm. For calculating the amount of antigen present in the samples, a standard curve was plotted using Prism 6 (GraphPad Software) based on the serial diluted recombinant Notch3 protein.

### Results

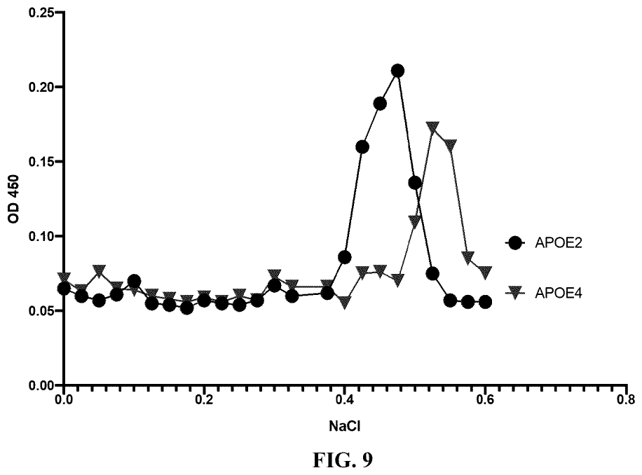

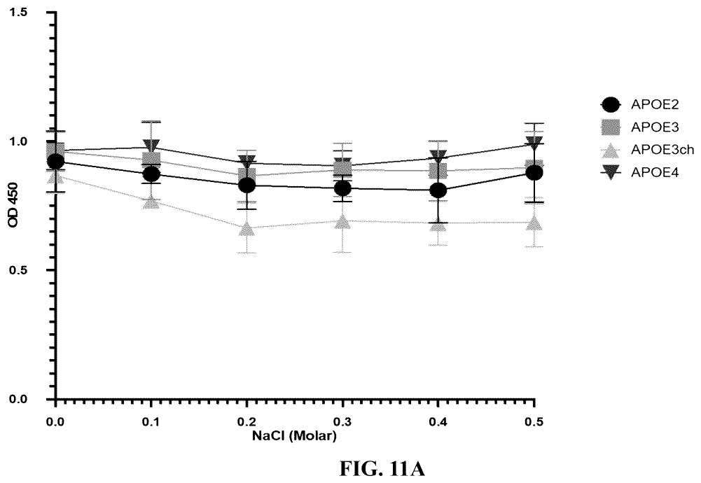

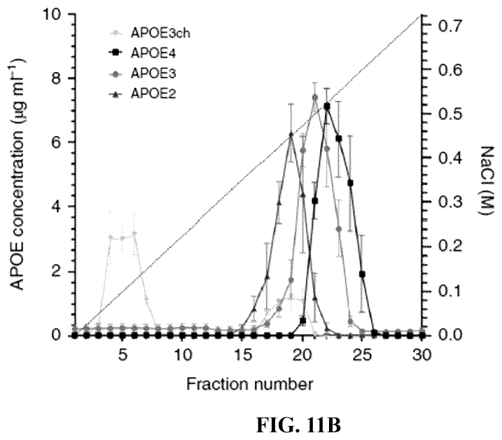

Heparin sulfate proteoglycans (HSPG) moieties are a type of glycosaminoglycans present in hundreds of proteins located in the plasma membrane and in the extracellular matrix. Protein-protein interactions mediated via HSPG play a critical role in a multitude of processes relevant to Alzheimer's pathology including amyloid and tau pathology and neurodegeneration. The ability of various ApoE isoforms, including ApoEch, to bind to heparin, a glycosaminoglycan commonly used to model HSPG-protein interactions, was investigated. Briefly, fractions containing ApoE isoforms ApoE2 and ApoE4 eluted from heparin columns under an increasing NaCl gradient (0-0.65M) were analyzed using ELISA. As shown in FIG. 9, the ApoE variant associated with higher risk of Alzheimer's disease, ApoE4, has higher affinity for heparin compared to the variant ApoE2, which is known to be protective. Next, fractions that contain His-tagged ApoE2, ApoE3, ApoE4 and ApoE3ch which were eluted from heparin columns under an increasing NaCl gradient (0-0.65M) were analyzed using western blot. As shown in FIG. 10, ApoE3ch had impaired heparin binding, which is much lower than that of ApoE2. The affinity of the ApoE isoforms for heparin were also analyzed using the heparin plate ELISA protocol as described in Materials and Methods. As shown in FIGS. 11A-11B, ApoE3ch showed remarkably low level of heparin binding, as ApoE3ch was released from the heparin column at much lower concentrations of NaCl compared to those required for ApoE4 release.

### Example 3: Generation of Antibodies Against Wild Type ApoE and ApoEch Mutant Protein

### Materials and Methods

Antibody competition assay: Antibodies were incubated with an ApoE3 recombinant protein (50 μg/ml in 20 mM Tris-HCL) at a 1:10 ratio and incubated for 3 hours at room temperature. A negative control containing the media only, and a positive control containing the recombinant protein ApoE3 only were used. The antibody/ApoE3 recombinant protein solution and controls were passed through heparin columns and exposed to an increasing NaCl gradient (as described in Example 2 above). Fractions were collected and assessed by ELISA and western blotting.

BCA Assay: Fractions collected from the heparin columns were first screened using the bicinchoninic acid assay (BCA assay) (Pierce BCA Protein Assay Kit). The assay was preformed using 200 μl of Reagent A and B Mix and 25 μl of each fraction. The 96 well plate was incubated for 30 minutes at 37° C., and read at 562 nm. The plate was read using Synery 2 microplate reader (BioTek Instrument. Inc) and the Gen5 version1.11 software).

Western Blotting: Western Blotting confirmed the elution of ApoE3 recombinant protein within fractions collected from the heparin binding columns. Fractions were diluted in 10 μl using RIPA buffer (Cell Signaling Technology), 10× (DTT 1M) and 4× Laemmli buffer for a final volume of 40 μl. Samples were separated on a 4-20% Mini-PROTEAN® TGX™ Precast Protein Gels (Bio-Rad), transferred to nitrocellulose membranes (VWR; 27376-991), blocked with Odyssey Blocking Buffer (LI-COR Biosciences, Lincoln, NE), and probed with mouse anti-his tag (Novus biologicals) and IRDye 800CW donkey anti-rabbit (LI-COR Biosciences) antibodies. Immunoreactive bands were visualized using the Odyssey Infrared Imaging System and visualized on the Image Studio version 2.1 (LI-COR Biosciences).

ELISA: Antibodies designed against the heparin-binding domain of ApoE were tested for their affinity to ApoE3 and ApoEch mutant recombinant proteins using ELISA. The Ni-NTA HisSorb Plates (Qiagen) were washed 3 times with wash buffer 1 (DY008). The ApoE recombinant proteins were suspended in buffer (DY008) to give a final concentration of 0.5 μg/ml. The plates were incubated with 200 μl of ApoE recombinant proteins for 2 hours, and washed 5 times with 1× wash buffer (DY008). The plates were then incubated with antibodies at a serial dilution of from 1:1,000 to 1:32,000 for overnight at 4° C. The plate was then washed 5 times in 1× wash buffer (DY008), and incubated with anti-mouse HRP (Abcam; ab97046, 1:10,000) for 45 minutes, followed by 5 washes in 1× wash buffer to ensure complete removal of unbound secondary antibody. The sulfuric acid from the ELISA reagent kit (DY008) was warmed to 37° C. 100 μl of tetramethylbenzidine (Millipore) was added to initiate the detection phase of the reaction. After a 5-min incubation, sulfuric acid was added to terminate the reaction. The plate was then read using Synery 2 microplate reader (BioTek Instrument. Inc) and the Gen5 version1.11 software).

### Results

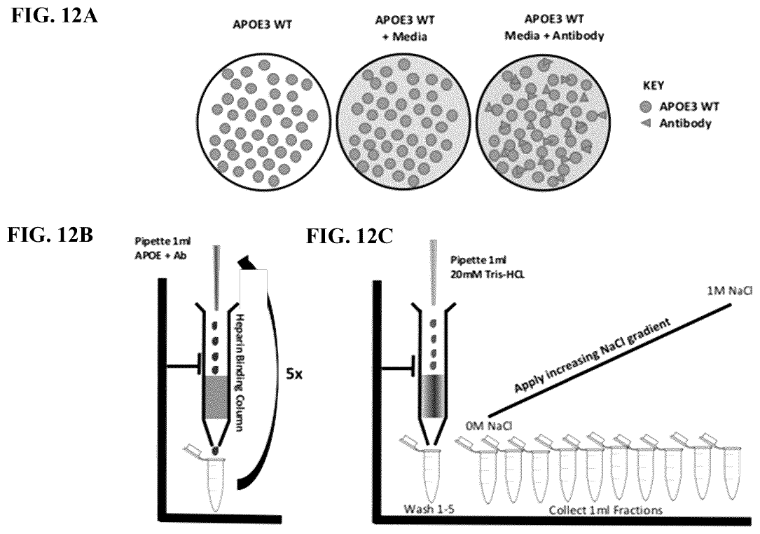

A monoclonal antibody against amino acids 130 to 143 of ApoE was generated and tested for its effect on the binding between full-length ApoE3 protein and heparin. Briefly, full-length wild type ApoE3 protein or those pre-incubated with the monoclonal antibody was passed through a heparin column and recycled five times to ensure maximal ApoE3 binding. The column was then washed five times with 20 mM Tris-HCl (pH=7.5) and exposed to an increasing gradient of NaCl (0 to 1M) in 20 mM Tris-HCl (pH=7.5). The elution from the Tris-HCl washes and from various NaCl concentrations were collected (FIGS. 12A-12C).

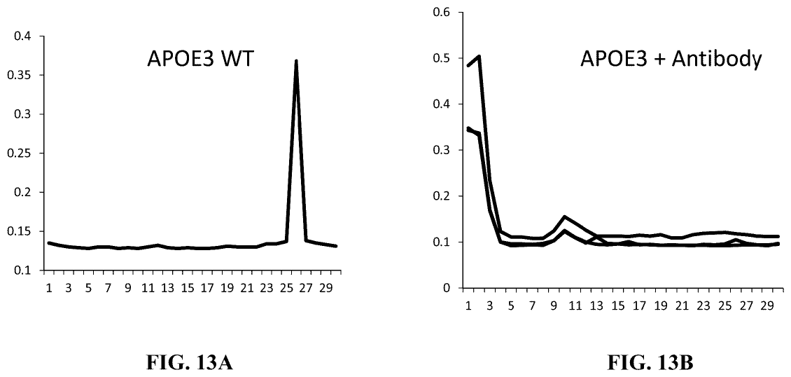

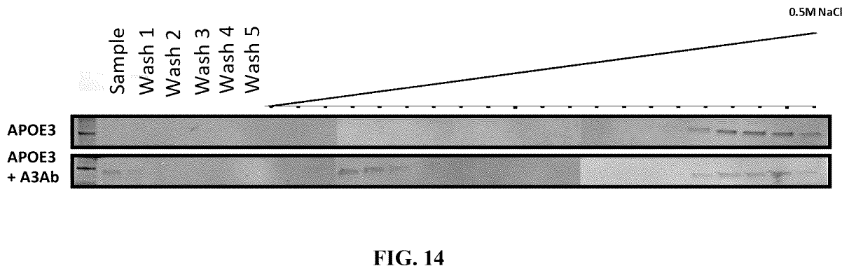

The fractions collected from the column were first screened by the bicinchoninic acid (BCA) assay. As shown in FIG. 13A, protein signal was detected for wild type ApoE3 in fractions to the right of the curve, indicating strong binding of ApoE3 to heparin. In contrast, strong signal was observed in early fractions with low ionic strength when ApoE3 was pre-incubated with the monoclonal antibody (FIG. 13B). To verify the results, western blotting was used to analyze the column washes and NaCl gradient fractions collected from the heparin column. As shown in FIG. 14, pre-incubation of wild type ApoE3 with the monoclonal antibody (A3Ab)(this antibody was named 1343 in Arboleda-Velasquez et al., Nature Medicine, 25, pages 1680-1683(2019)) reduced its ability to bind to heparin, to a level similar to that of an ApoE3ch mutant protein. Individual gels were stitched together to generate FIG. 14. These results suggest that an antibody may be used to modify the binding properties of ApoE to heparin, thereby preventing or treating Alzheimer's disease or related dementias or neurodegeneration.

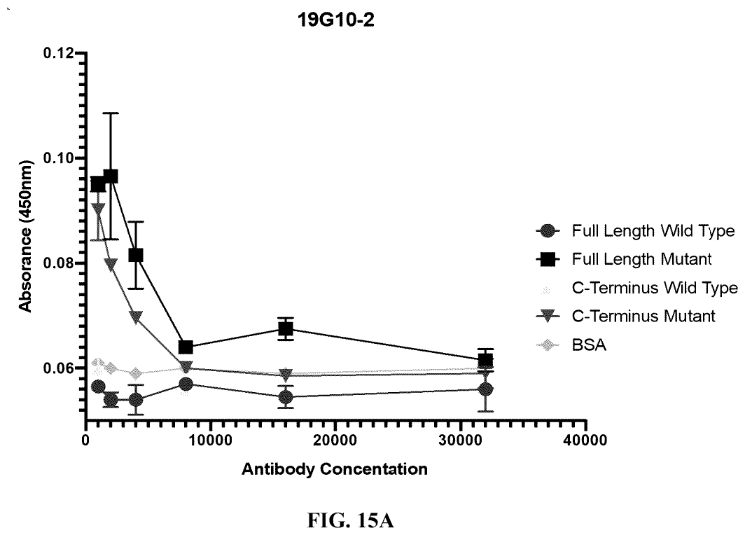

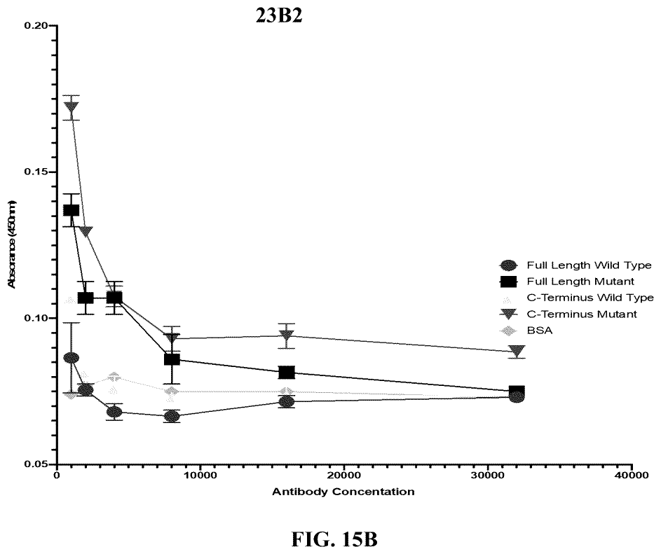

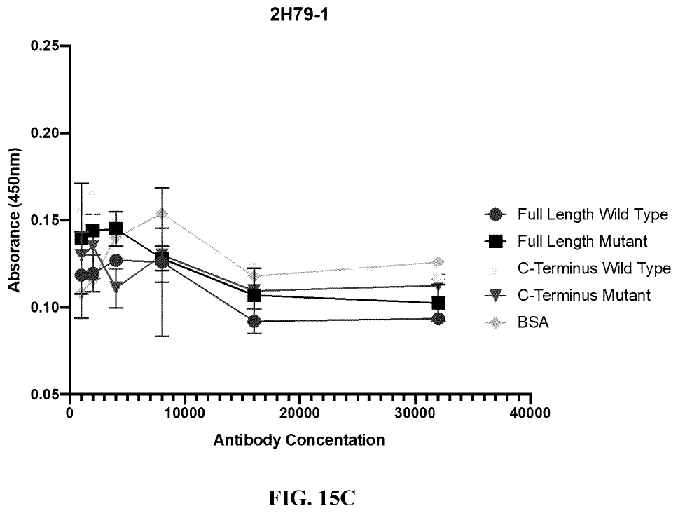

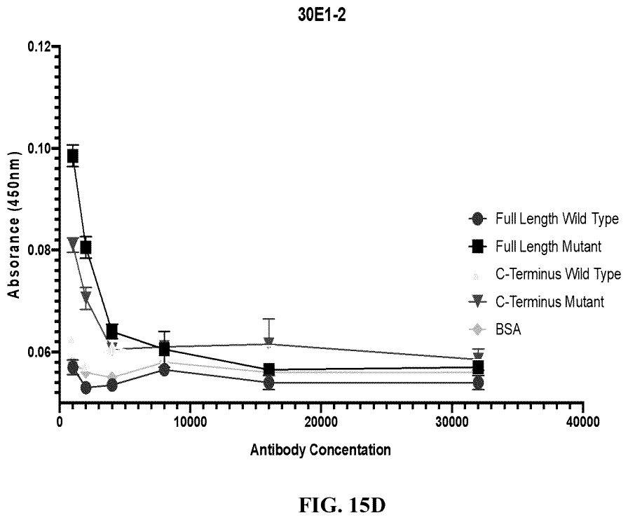

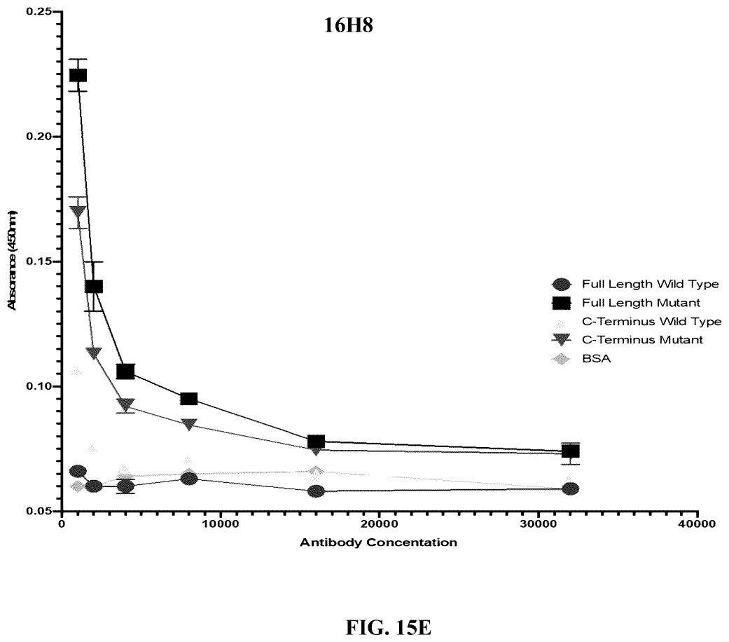

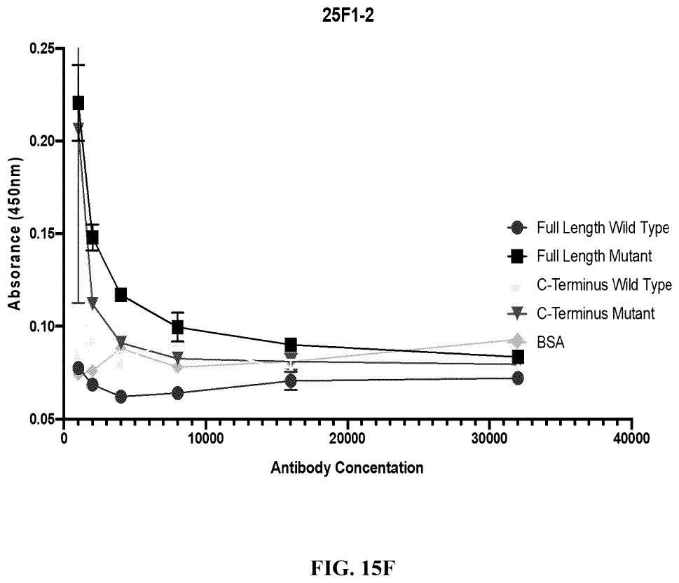

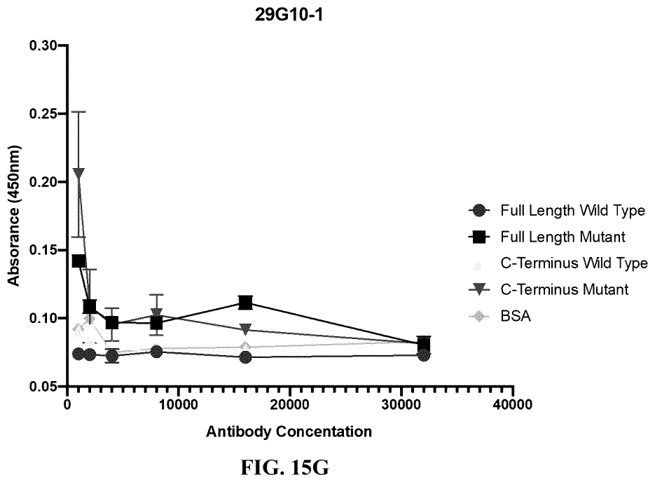

To generate monoclonal antibodies against the heparin-binding domain of ApoE, mice were immunized using the wild type ApoE peptide: KLH-CTEELRVRLASHLRK-CONH2 (SEQ ID NO:54), and the ApoEch peptide: KLH-CTEELRVSLASHLRK-CONH2 (SEQ ID NO:55). A cysteine residue was added at the N-terminus to facilitate conjugation of the peptides. Cell fusions were obtained from positive clones and cell supernatants tested for activity against the wild type and mutant peptides and proteins. Seven antibodies generated were analyzed by ELISA as examples, as described in the materials and methods section. The 19G10-2 antibody serum displayed specificity towards both the full-length and C-terminal of the APOE3ch mutant protein and some interaction with the wild type APOE3 protein (FIG. 15A). The 23B2 antibody displayed reactivity to both the wild type ApoE3 and ApoE3ch mutant C-terminal and full-length recombinant proteins (FIG. 15B). The 2H79-1 antibody displayed non-specific binding to bovine serum albumin (BSA) and showed no affinity for either wild type ApoE3 or ApoE3ch mutant (FIG. 15C). Both the 30E1-2 and 16H8 antibodies showed reactivity to the full-length and C-terminal ApoE3ch mutant proteins and the C-terminal form of wild type ApoE3, but did not react with the full-length wild type APOE3 protein (FIGS. 15D and 15E). The 25F1-2 antibody serum showed high affinity for the full-length and C-terminal ApoE3ch mutant proteins, and appeared to have variable binding to the C-terminus of the wild type ApoE3 protein and some interaction with the full-length wild type ApoE3 protein (FIG. 15F). The 29G10-2 antibody showed high affinity for both the full-length and C-terminal of the ApoE3ch mutant proteins, and also showed reactivity to the C-terminal wild type ApoE3 and BSA. Lastly, the 29G10-2 antibody did not interact with the full-length wild type ApoE3 protein (FIG. 15G).

The variable heavy chain (VH), variable light chain (VL) and complementarity determining region (CDR) sequences of 25F1-2 and 19G10-2 are described herein.

Further, the following parental clones with specificity for wild type ApoE were generated, and the specificity for the wild type ApoE peptide (WT peptide), the wild type ApoE protein (WT protein), the mutant ApoE peptide (ApoEch; Mut peptide) and the mutant ApoE3ch protein (Mut protein) were tested as shown in Table 6. The values indicate levels of absorbance as detected by ELISA. The bolded clones showed specificity for the wild type ApoE peptide (KLH-CTEELR VRLASHLRK-CONH2 (SEQ ID NO:54) and the wild type ApoE protein).

TABLE 6

| | WT peptide | WT protein | Mut peptide | Mut protein |
|---|---|---|---|---|
| 1D5 | 1.742 | 1.086 | 0.060 | 0.062 |
| 1H4 | 2.578 | 2.113 | 0.070 | 0.056 |
| 3A6 | 2.412 | 0.733 | 0.059 | 0.056 |
| 7C3 | 1.698 | 1.245 | 0.064 | 0.051 |
| 7C4 | 2.097 | 0.586 | 0.057 | 0.056 |
| 7C11 | 1.282 | 0.689 | 0.058 | 0.055 |
| 16G6 | 0.739 | 0.449 | 0.076 | 0.067 |
| Pos. ctrl | 1.980 | 1.171 | 0.254 | 0.144 |
| Neg. ctrl | 0.068 | 0.051 | 0.056 | 0.048 |

### Example 4: Generation of Fusion Proteins Containing the Heparin-Binding Domain of APOE

### Materials and Methods

Peptide Competition Assay: Wild type ApoE3 and ApoE3ch mutant peptides (50 ug/ml) were incubated with ApoE3 recombinant protein (50 ug/ml prepared in 20 mM Tris-HCL) for 3 hours at room temperature. The peptide/ApoE3 recombinant protein solution were then passed through heparin columns and exposed to an increasing NaCl gradient (as described in Example 2 and 3 above). Fractions were collected and assessed by western blotting.

Western Blotting: Western blotting confirmed the elution of ApoE3 within fractions collected from the heparin binding columns. Fractions were diluted in 10 μl RIPA buffer (Cell Signaling Technology), 10× (DTT 1M) and 4× Laemmli buffer to a final volume of 40 μl. Samples were separated on a 4-20% Mini-PROTEAN® TGX™ Precast Protein Gels (Bio-Rad), transferred to nitrocellulose membranes (VWR; 27376-991), blocked with Odyssey Blocking Buffer (LI-COR Biosciences, Lincoln, NE), and probed with mouse anti-his tag (Novus biologicals) and IRDye 800CW donkey anti-rabbit (LI-COR Biosciences) antibodies. Immunoreactive bands were visualized using the Odyssey Infrared Imaging System and visualized on the Image Studio version 2.1 (LI-COR Biosciences).

### Results

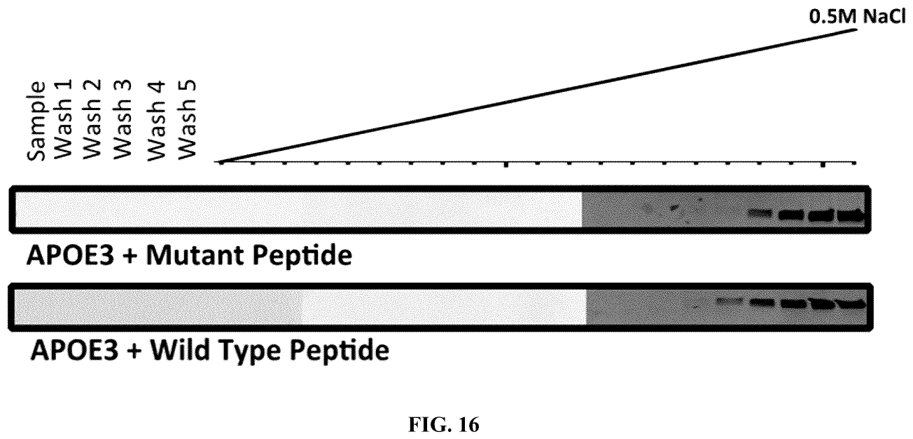

Peptides containing amino acids 130-143 of the wild type ApoE protein and ApoEch mutant protein, respectively, were generated. To examine the effect of these peptides on the binding between wild type ApoE3 recombinant protein and heparin sepharose, a peptide competition assay was carried out as described in materials and methods. As shown in FIG. 16, the wild type ApoE peptide resulted in a one-fraction shift of wild type ApoE3 recombinant protein binding, suggesting that this peptide can compete with wild type full-length ApoE3 for binding to heparin. These results suggest that ApoE fragments containing amino acids 130-143 of the wild type ApoE protein may be used to change the binding properties of ApoE to heparin, thereby preventing or treating Alzheimer's disease or related dementias or neurodegeneration.

To increase protein stability of the peptides, C-terminal and N-terminal fusion proteins containing the heparin-binding domain of human ApoE (wild type and the R136S mutant version) or a site of allosteric modulation of the heparin-binding domain were generated using the backbone Fc IgG2 constructs pfuse-hfc1 and pfen-hg2 (Invivogen). The human ApoE fragments excluded the sites for APOE2 and APOE4 variants. Administration of ApoE fragments with an R at position 136 may compete with endogenous ApoE for interaction with binding partners including HSPG leading to protection against neurodegeneration. Administration of ApoE fragments with an S at position 136 may bind molecules that do not bind to the wild type ApoE, leading to protection against neurodegeneration. The amino acid sequences for the fragments from the wild type and the R136S mutant ApoE used to generate the fusion proteins are shown below (the R136 position is bolded and double underlined).

### Downstream of R136 Fragment

WT
(SEQ ID NO: 57)
STEELRV [symbol] LASHLRKLRKRLLRDADDLQK
Mutant
(SEQ ID NO: 58)
STEELRV [symbol] LASHLRKLRKRLLRDADDLQK [symbol]
indicates data missing or illegible when filed

### Upstream of R136 Fragment

WT
(SEQ ID NO: 59)
RLVQYRGEVQAMLGQSTEELRVRLASHLRKL
Mutant
(SEQ ID NO: 60)
RLVQYRGEVQAMLGQSTEELRVSLASHLRKL

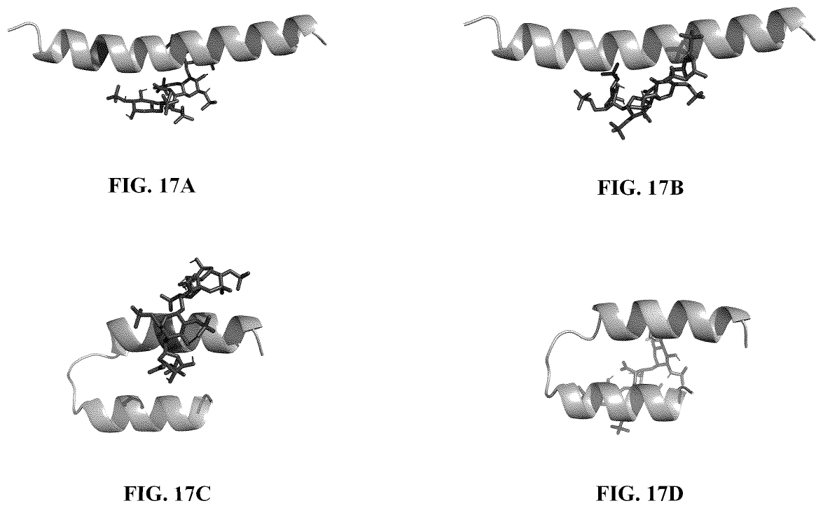

FIGS. 17A-17D show models of the interaction of ApoE fragments with heparin. FIG. 17A shows a model of the wild type ApoE fragment (downstream of R136 fragment, helix) interacting with heparin. FIG. 17B shows a model of the fragment of ApoE R136S (downstream of R136 fragment, helix) interacting with heparin. FIG. 17C shows a model of the wild type ApoE fragment (upstream of R136 fragment, helix) interacting with heparin. FIG. 17D shows a model of the fragment of ApoE R136S (upstream of R136 fragment, helix) interacting with heparin.

The nucleic acid and amino acid sequences for exemplary fusion protein constructs containing either the downstream of R136 fragment or the upstream of R136 fragment are shown below.

| 184Q pfcn-hg2 ApoE 114-144 | |
|---|---|
| Nucleic acid sequence | |
| (SEQ ID NO: 81) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCACCGGCGA AGGAGGGCCA CCATGTACAG GATGCAACTC CTGTCTTGCA |
| 601 | TTGCACTAAG TCTTGCACTT GTCACGAATT CGGCACCTCT CGAGCGCAAA TCTAGTGTCG |
| 661 | AGTGCCCACC GTGCCCAGCA CCACCTGTGG CAGGACCGTC AGTCTTCCTC TTCCCCCCAA |
| 721 | AACCCAAGGA CACCCTCATG ATCTCCCGGA CCCCTGAGGT CACGTGCGTG GTGGTGGACG |
| 781 | TGAGCCACGA AGACCCCGAG GTCCAGTTCA ACTGGTACGT GGACGGCGTG GAGGTGCATA |
| 841 | ATGCCAAGAC AAAGCCACGG GAGGAGCAGT TCAACAGCAC GTTCCGTGTG GTCAGCGTCC |
| 901 | TCACCGTTGT GCACCAGGAC TGGCTGAACG GCAAGGAGTA CAAGTGCAAG GTCTCCAACA |
| 961 | AAGGCCTCCC AGCCCCCATC GAGAAAACCA TCTCCAAAAC CAAAGGGCAG CCCCGAGAAC |
| 1021 | CACAGGTGTA CACCCTGCCC CCATCCCGGG AGGAGATGAC CAAGAACCAG GTCAGCCTGA |
| 1081 | CCTGCCTGGT CAAAGGCTTC TACCCCAGCG ACATCGCCGT GGAGTGGGAG AGCAATGGGC |
| 1141 | AGCCGGAGAA CAACTACAAG ACCACGCCTC CCATGCTGGA CTCCGACGGC TCCTTCTTCC |
| 1201 | TCTACAGCAA GCTCACCGTG GACAAGAGCA GGTGGCAGCA GGGGAACGTC TTCTCATGCT |
| 1261 | CCGTGATGCA TGAGGCTCTG CACAACCACT ACACGCAGAA GAGCCTCTCC CTGTCTCCGG |
| 1321 | GTGCACGTAC GCGCCTGGTG CAGTACCGCG GCGAGGTGCA GGCCATGCTC GGCCAGAGCA |
| 1381 | CCGAGGAGCT GCGGGTGCGC CTCGCCTCCC ACCTGCGCAA GCTGtgaTAT CTCGAGCTAG |
| 1441 | CTGGCCAGAC ATGATAAGAT ACATTGATGA GTTTGGACAA ACCACAACTA GAATGCAGTG |
| 1501 | AAAAAAATGC TTTATTTGTG AAATTTGTGA TGCTATTGCT TTATTTGTAA CCATTATAAG |
| 1561 | CTGCAATAAA CAAGTTAACA ACAACAATTG CATTCATTTT ATGTTTCAGG TTCAGGGGGA |
| 1621 | GGTGTGGGAG GTTTTTTAAA GCAAGTAAAA CCTCTACAAA TGTGGTATGG AATTAATTCT |
| 1681 | AAAATACAGC ATAGCAAAAC TTTAACCTCC AAATCAAGCC TCTACTTGAA TCCTTTTCTG |
| 1741 | AGGGATGAAT AAGGCATAGG CATCAGGGGC TGTTGCCAAT GTGCATTAGC TGTTTGCAGC |
| 1801 | CTCACCTTCT TTCATGGAGT TTAAGATATA GTGTATTTTC CCAAGGTTTG AACTAGCTCT |
| 1861 | TCATTTCTTT ATGTTTTAAA TGCACTGACC TCCCACATTC CCTTTTTAGT AAAATATTCA |
| 1921 | GAAATAATTT AAATACATCA TTGCAATGAA AATAAATGTT TTTTATTAGG CAGAATCCAG |
| 1981 | ATGCTCAAGG CCCTTCATAA TATCCCCCAG TTTAGTAGTT GGACTTAGGG AACAAAGGAA |
| 2041 | CCTTTAATAG AAATTGGACA GCAAGAAAGC GAGCTTCTAG CTTATCCTCA GTCCTGCTCC |
| 2101 | TCTGCCACAA AGTGCACGCA GTTGCCGGCC GGGTCGCGCA GGGCGAACTC CCGCCCCCAC |
| 2161 | GGCTGCTCGC CGATCTCGGT CATGGCCGGC CCGGAGGCGT CCCGGAAGTT CGTGGACACG |
| 2221 | ACCTCCGACC ACTCGGCGTA CAGCTCGTCC AGGCCGCGCA CCCACACCCA GGCCAGGGTG |
| 2281 | TTGTCCGGCA CCACCTGGTC CTGGACCGCG CTGATGAACA GGGTCACGTC GTCCCGGACC |
| 2341 | ACACCGGCGA AGTCGTCCTC CACGAAGTCC CGGGAGAACC CGAGCCGGTC GGTCCAGAAC |
| 2401 | TCGACCGCTC CGGCGACGTC GCGCGCGGTG AGCACCGGAA CGGCACTGGT CAACTTGGCC |
| 2461 | ATGATGGCTC CTCctgtcag gagaggaaag agaagaaggt tagtacaatt gCTATAGTGA |
| 2521 | GTTGTATTAT ACTATGCAGA TATACTATGC CAATGATTAA TTGTCAAACT AGGGCTGCAg |
| 2581 | ggttcatagt gccacttttc ctgcactgcc ccatctcctg cccacccttt cccaggcata |
| 2641 | gacagtcagt gacttacCAA ACTCACAGGA GGGAGAAGGC AGAAGCTTGA GACAGACCCG |
| 2701 | CGGGACCGCC GAACTGCGAG GGGACGTGGC TAGGGCGGCT TCTTTTATGG TGCGCCGGCC |
| 2761 | CTCGGAGGCA GGGCGCTCGG GGAGGCCTAG CGGCCAATCT GCGGTGGCAG GAGGCGGGGC |
| 2821 | CGAAGGCCGT GCCTGACCAA TCCGGAGCAC ATAGGAGTCT CAGCCCCCCG CCCCAAAGCA |
| 2881 | AGGGGAAGTC ACGCGCCTGT AGCGCCAGCG TGTTGTGAAA TGGGGGCTTG GGGGGGTTGG |
| 2941 | GGCCCTGACT AGTCAAAACA AACTCCCATT GACGTCAATG GGGTGGAGAC TTGGAAATCC |
| 3001 | CCGTGAGTCA AACCGCTATC CACGCCCATT GATGTACTGC CAAAACCGCA TCATCATGGT |
| 3061 | AATAGCGATG ACTAATACGT AGATGTACTG CCAAGTAGGA AAGTCCCATA AGGTCATGTA |
| 3121 | CTGGGCATAA TGCCAGGCGG GCCATTTACC GTCATTGACG TCAATAGGGG GCGTACTTGG |
| 3181 | CATATGATAC ACTTGATGTA CTGCCAAGTG GGCAGTTTAC CGTAAATACT CCACCCATTG |
| 3241 | ACGTCAATGG AAAGTCCCTA TTGGCGTTAC TATGGGAACA TACGTCATTA TTGACGTCAA |
| 3301 | TGGGCGGGGG TCGTTGGGCG GTCAGCCAGG CGGGCCATTT ACCGTAAGTT ATGTAACGCC |
| 3361 | TGCAGGTTAA TTAAGAACAT GTGAGCAAAA GGCCAGCAAA AGGCCAGGAA CCGTAAAAAG |
| 3421 | GCCGCGTTGC TGGCGTTTTT CCATAGGCTC CGCCCCCCTG ACGAGCATCA CAAAAATCGA |
| 3481 | CGCTCAAGTC AGAGGTGGCG AAACCCGACA GGACTATAAA GATACCAGGC GTTTCCCCCT |
| 3541 | GGAAGCTCCC TCGTGCGCTC TCCTGTTCCG ACCCTGCCGC TTACCGGATA CCTGTCCGCC |
| 3601 | TTTCTCCCTT CGGGAAGCGT GGCGCTTTCT CATAGCTCAC GCTGTAGGTA TCTCAGTTCG |
| 3661 | GTGTAGGTCG TTCGCTCCAA GCTGGGCTGT GTGCACGAAC CCCCCGTTCA GCCCGACCGC |
| 3721 | TGCGCCTTAT CCGGTAACTA TCGTCTTGAG TCCAACCCGG TAAGACACGA CTTATCGCCA |
| 3781 | CTGGCAGCAG CCACTGGTAA CAGGATTAGC AGAGCGAGGT ATGTAGGCGG TGCTACAGAG |
| 3841 | TTCTTGAAGT GGTGGCCTAA CTACGGCTAC ACTAGAAGAA CAGTATTTGG TATCTGCGCT |
| 3901 | CTGCTGAAGC CAGTTACCTT CGGAAAAAGA GTTGGTAGCT CTTGATCCGG CAAACAAACC |
| 3961 | ACCGCTGGTA GCGGTGGTTT TTTTGTTTGC AAGCAGCAGA TTACGCGCAG AAAAAAAGGA |
| 4021 | TCTCAAGAAG ATCCTTTGAT CTTTTCTACG GGGTCTGACG CTCAGTGGAA CGAAAACTCA |
| 4081 | CGTTAAGGGA TTTTGGTCAT GGCTAGTTAA TTAACATTTA AATCAGCGGC CGCAATAAAA |
| 4141 | TATCTTTATT TTCATTACAT CTGTGTGTTG GTTTTTTGTG TGAATCGTAA CTAACATACG |
| 4201 | CTCTCCATCA AAACAAAACG AAACAAAACA AACTAGCAAA ATAGGCTGTC CCCAGTGCAA |
| 4261 | GTGCAGGTGC CAGAACATTT CTCTATCGAA |
| Amino acid sequence | |
| (SEQ ID NO: 82) | |
| MYRMQLLSCIALSLALVTNSAPLERKSSVECPPCPAPPVAGPSVFLFPP | |
| KPKDTLMISRTPEVTCVVVDVSHEDPEVQFNWYVDGVEVEINAKTKPREEQF | |
| NSTFRVVSVLTVVHQDWLNGKEYKCKVSNKGLPAPIEKTISKTKGQPREPQV | |
| YTLPPSREEMTKNQVSLTCLVKGFYPSDIAVEWESNGQPENNYKTTPPMLDS | |
| DGSFFLYSKLTVDKSRWQQGNVFSCSVMHEALHNHYTQKSLSLSPGARTRL | |
| VQYRGEVQAMLGQSTEELRVRLASHLRKL | |
| 184R pfcn-hg2 ApoE 114-144 R1365 | |
| Nucleic acid sequence | |
| (SEQ ID NO: 83) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCACCGGCGA AGGAGGGCCA CCATGTACAG GATGCAACTC CTGTCTTGCA |
| 601 | TTGCACTAAG TCTTGCACTT GTCACGAATT CGGCACCTCT CGAGCGCAAA TCTAGTGTCG |
| 661 | AGTGCCCACC GTGCCCAGCA CCACCTGTGG CAGGACCGTC AGTCTTCCTC TTCCCCCCAA |
| 721 | AACCCAAGGA CACCCTCATG ATCTCCCGGA CCCCTGAGGT CACGTGCGTG GTGGTGGACG |
| 781 | TGAGCCACGA AGACCCCGAG GTCCAGTTCA ACTGGTACGT GGACGGCGTG GAGGTGCATA |
| 841 | ATGCCAAGAC AAAGCCACGG GAGGAGCAGT TCAACAGCAC GTTCCGTGTG GTCAGCGTCC |
| 901 | TCACCGTTGT GCACCAGGAC TGGCTGAACG GCAAGGAGTA CAAGTGCAAG GTCTCCAACA |
| 961 | AAGGCCTCCC AGCCCCCATC GAGAAAACCA TCTCCAAAAC CAAAGGGCAG CCCCGAGAAC |
| 1021 | CACAGGTGTA CACCCTGCCC CCATCCCGGG AGGAGATGAC CAAGAACCAG GTCAGCCTGA |
| 1081 | CCTGCCTGGT CAAAGGCTTC TACCCCAGCG ACATCGCCGT GGAGTGGGAG AGCAATGGGC |
| 1141 | AGCCGGAGAA CAACTACAAG ACCACGCCTC CCATGCTGGA CTCCGACGGC TCCTTCTTCC |
| 1201 | TCTACAGCAA GCTCACCGTG GACAAGAGCA GGTGGCAGCA GGGGAACGTC TTCTCATGCT |
| 1261 | CCGTGATGCA TGAGGCTCTG CACAACCACT ACACGCAGAA GAGCCTCTCC CTGTCTCCGG |
| 1321 | GTGCACGTAC GCGCCTGGTG CAGTACCGCG GCGAGGTGCA GGCCATGCTC GGCCAGAGCA |
| 1381 | CCGAGGAGCT GCGGGTGaGC CTCGCCTCCC ACCTGCGCAA GCTGtgaTAT CTCGAGCTAG |
| 1441 | CTGGCCAGAC ATGATAAGAT ACATTGATGA GTTTGGACAA ACCACAACTA GAATGCAGTG |
| 1501 | AAAAAAATGC TTTATTTGTG AAATTTGTGA TGCTATTGCT TTATTTGTAA CCATTATAAG |
| 1561 | CTGCAATAAA CAAGTTAACA ACAACAATTG CATTCATTTT ATGTTTCAGG TTCAGGGGGA |
| 1621 | GGTGTGGGAG GTTTTTTAAA GCAAGTAAAA CCTCTACAAA TGTGGTATGG AATTAATTCT |
| 1681 | AAAATACAGC ATAGCAAAAC TTTAACCTCC AAATCAAGCC TCTACTTGAA TCCTTTTCTG |
| 1741 | AGGGATGAAT AAGGCATAGG CATCAGGGGC TGTTGCCAAT GTGCATTAGC TGTTTGCAGC |
| 1801 | CTCACCTTCT TTCATGGAGT TTAAGATATA GTGTATTTTC CCAAGGTTTG AACTAGCTCT |
| 1861 | TCATTTCTTT ATGTTTTAAA TGCACTGACC TCCCACATTC CCTTTTTAGT AAAATATTCA |
| 1921 | GAAATAATTT AAATACATCA TTGCAATGAA AATAAATGTT TTTTATTAGG CAGAATCCAG |
| 1981 | ATGCTCAAGG CCCTTCATAA TATCCCCCAG TTTAGTAGTT GGACTTAGGG AACAAAGGAA |
| 2041 | CCTTTAATAG AAATTGGACA GCAAGAAAGC GAGCTTCTAG CTTATCCTCA GTCCTGCTCC |
| 2101 | TCTGCCACAA AGTGCACGCA GTTGCCGGCC GGGTCGCGCA GGGCGAACTC CCGCCCCCAC |
| 2161 | GGCTGCTCGC CGATCTCGGT CATGGCCGGC CCGGAGGCGT CCCGGAAGTT CGTGGACACG |
| 2221 | ACCTCCGACC ACTCGGCGTA CAGCTCGTCC AGGCCGCGCA CCCACACCCA GGCCAGGGTG |
| 2281 | TTGTCCGGCA CCACCTGGTC CTGGACCGCG CTGATGAACA GGGTCACGTC GTCCCGGACC |
| 2341 | ACACCGGCGA AGTCGTCCTC CACGAAGTCC CGGGAGAACC CGAGCCGGTC GGTCCAGAAC |
| 2401 | TCGACCGCTC CGGCGACGTC GCGCGCGGTG AGCACCGGAA CGGCACTGGT CAACTTGGCC |
| 2461 | ATGATGGCTC CTCctgtcag gagaggaaag agaagaaggt tagtacaatt gCTATAGTGA |
| 2521 | GTTGTATTAT ACTATGCAGA TATACTATGC CAATGATTAA TTGTCAAACT AGGGCTGCAg |
| 2581 | ggttcatagt gccacttttc ctgcactgcc ccatctcctg cccacccttt cccaggcata |
| 2641 | gacagtcagt gacttacCAA ACTCACAGGA GGGAGAAGGC AGAAGCTTGA GACAGACCCG |
| 2701 | CGGGACCGCC GAACTGCGAG GGGACGTGGC TAGGGCGGCT TCTTTTATGG TGCGCCGGCC |
| 2761 | CTCGGAGGCA GGGCGCTCGG GGAGGCCTAG CGGCCAATCT GCGGTGGCAG GAGGCGGGGC |
| 2821 | CGAAGGCCGT GCCTGACCAA TCCGGAGCAC ATAGGAGTCT CAGCCCCCCG CCCCAAAGCA |
| 2881 | AGGGGAAGTC ACGCGCCTGT AGCGCCAGCG TGTTGTGAAA TGGGGGCTTG GGGGGGTTGG |
| 2941 | GGCCCTGACT AGTCAAAACA AACTCCCATT GACGTCAATG GGGTGGAGAC TTGGAAATCC |
| 3001 | CCGTGAGTCA AACCGCTATC CACGCCCATT GATGTACTGC CAAAACCGCA TCATCATGGT |
| 3061 | AATAGCGATG ACTAATACGT AGATGTACTG CCAAGTAGGA AAGTCCCATA AGGTCATGTA |
| 3121 | CTGGGCATAA TGCCAGGCGG GCCATTTACC GTCATTGACG TCAATAGGGG GCGTACTTGG |
| 3181 | CATATGATAC ACTTGATGTA CTGCCAAGTG GGCAGTTTAC CGTAAATACT CCACCCATTG |
| 3241 | ACGTCAATGG AAAGTCCCTA TTGGCGTTAC TATGGGAACA TACGTCATTA TTGACGTCAA |
| 3301 | TGGGCGGGGG TCGTTGGGCG GTCAGCCAGG CGGGCCATTT ACCGTAAGTT ATGTAACGCC |
| 3361 | TGCAGGTTAA TTAAGAACAT GTGAGCAAAA GGCCAGCAAA AGGCCAGGAA CCGTAAAAAG |
| 3421 | GCCGCGTTGC TGGCGTTTTT CCATAGGCTC CGCCCCCCTG ACGAGCATCA CAAAAATCGA |
| 3481 | CGCTCAAGTC AGAGGTGGCG AAACCCGACA GGACTATAAA GATACCAGGC GTTTCCCCCT |
| 3541 | GGAAGCTCCC TCGTGCGCTC TCCTGTTCCG ACCCTGCCGC TTACCGGATA CCTGTCCGCC |
| 3601 | TTTCTCCCTT CGGGAAGCGT GGCGCTTTCT CATAGCTCAC GCTGTAGGTA TCTCAGTTCG |
| 3661 | GTGTAGGTCG TTCGCTCCAA GCTGGGCTGT GTGCACGAAC CCCCCGTTCA GCCCGACCGC |
| 3721 | TGCGCCTTAT CCGGTAACTA TCGTCTTGAG TCCAACCCGG TAAGACACGA CTTATCGCCA |
| 3781 | CTGGCAGCAG CCACTGGTAA CAGGATTAGC AGAGCGAGGT ATGTAGGCGG TGCTACAGAG |
| 3841 | TTCTTGAAGT GGTGGCCTAA CTACGGCTAC ACTAGAAGAA CAGTATTTGG TATCTGCGCT |
| 3901 | CTGCTGAAGC CAGTTACCTT CGGAAAAAGA GTTGGTAGCT CTTGATCCGG CAAACAAACC |
| 3961 | ACCGCTGGTA GCGGTGGTTT TTTTGTTTGC AAGCAGCAGA TTACGCGCAG AAAAAAAGGA |
| 4021 | TCTCAAGAAG ATCCTTTGAT CTTTTCTACG GGGTCTGACG CTCAGTGGAA CGAAAACTCA |
| 4081 | CGTTAAGGGA TTTTGGTCAT GGCTAGTTAA TTAACATTTA AATCAGCGGC CGCAATAAAA |
| 4141 | TATCTTTATT TTCATTACAT CTGTGTGTTG GTTTTTTGTG TGAATCGTAA CTAACATACG |
| 4201 | CTCTCCATCA AAACAAAACG AAACAAAACA AACTAGCAAA ATAGGCTGTC CCCAGTGCAA |
| 4261 | GTGCAGGTGC CAGAACATTT CTCTATCGAA |
| Amino acid sequence | |
| (SEQ ID NO: 84) | |
| MYRMQLLSCIALSLALVTNSAPLERKSSVECPPCPAPPVAGPSVFLFPP | |
| KPKDTLMISRTPEVTCVVVDVSHEDPEVQFNWYVDGVEVEINAKTKPREEQF | |
| NSTFRVVSVLTVVHQDWLNGKEYKCKVSNKGLPAPIEKTISKTKGQPREPQV | |
| YTLPPSREEMTKNQVSLTCLVKGFYPSDIAVEWESNGQPENNYKTTPPMLDS | |
| DGSFFLYSKLTVDKSRWQQGNVFSCSVMHEALHNHYTQKSLSLSPGARTRL | |
| VQYRGEVQAMLGQSTEELRVSLASHLRKL | |
| 197F pfuse hfc2 ApoE 114-144 | |
| Nucleic acid sequence | |
| (SEQ ID NO: 85) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCTG CTTGCTCAAC |
| 481 | TCTACGTCTT TGTTTCGTTT TCTGTTCTGC GCCGTTACAG ATCCAAGCTG TGACCGGCGC |
| 541 | CTACCTGAGA TCAccggcGA AGGAGGGCCA CCATGTACAG GATGCAACTC CTGTCTTGCA |
| 601 | TTGCACTAAG TCTTGCACTT GTCACGAATT CGATaCGCCT GGTGCAGTAC CGCGGCGAGG |
| 661 | TGCAGGCCAT GCTCGGCCAG AGtACtGAGG AGCTGCGGGT GCGCCTCGCC TCCCACCTGC |
| 721 | GCAAGCTGat ATCGGCCATG GTTAGATCTG TGGAGTGCCC ACCTTGCCCA GCACCACCTG |
| 781 | TGGCAGGACC TTCAGTCTTC CTCTTCCCCC CAAAACCCAA GGACACCCTG ATGATCTCCA |
| 841 | GAACCCCTGA GGTCACGTGC GTGGTGGTGG ACGTGAGCCA CGAAGACCCC GAGGTCCAGT |
| 901 | TCAACTGGTA CGTGGACGGC ATGGAGGTGC ATAATGCCAA GACAAAGCCA CGGGAGGAGC |
| 961 | AGTTCAACAG CACGTTCCGT GTGGTCAGCG TCCTCACCGT CGTGCACCAG GACTGGCTGA |
| 1021 | ACGGCAAGGA GTACAAGTGC AAGGTCTCCA ACAAAGGCCT CCCAGCCCCC ATCGAGAAAA |
| 1081 | CCATCTCCAA AACCAAAGGG CAGCCCCGAG AACCACAGGT GTACACCCTG CCCCCATCCC |
| 1141 | GGGAGGAGAT GACCAAGAAC CAGGTCAGCC TGACCTGCCT GGTCAAAGGC TTCTACCCCA |
| 1201 | GCGACATCGC CGTGGAGTGG GAGAGCAATG GGCAGCCGGA GAACAACTAC AAGACCACAC |
| 1261 | CTCCCATGCT GGACTCCGAC GGCTCCTTCT TCCTCTACAG CAAGCTCACC GTGGACAAGA |
| 1321 | GCAGGTGGCA GCAGGGGAAC GTCTTCTCAT GCTCCGTGAT GCATGAGGCT CTGCACAACC |
| 1381 | ACTACACACA GAAGAGCCTC TCCCTGTCTC CGGGTAAATG AgtgccacgG CTAGCTGGCC |
| 1441 | AGACATGATA AGATACATTG ATGAGTTTGG ACAAACCACA ACTAGAATGC AGTGAAAAAA |
| 1501 | ATGCTTTATT TGTGAAATTT GTGATGCTAT TGCTTTATTT GTAACCATTA TAAGCTGCAA |
| 1561 | TAAACAAGTT AACAACAACA ATTGCATTCA TTTTATGTTT CAGGTTCAGG GGGAGGTGTG |
| 1621 | GGAGGTTTTT TAAAGCAAGT AAAACCTCTA CAAATGTGGT ATGGAATTAA TTCTAAAATA |
| 1681 | CAGCATAGCA AAACTTTAAC CTCCAAATCA AGCCTCTACT TGAATCCTTT TCTGAGGGAT |
| 1741 | GAATAAGGCA TAGGCATCAG GGGCTGTTGC CAATGTGCAT TAGCTGTTTG CAGCCTCACC |
| 1801 | TTCTTTCATG GAGTTTAAGA TATAGTGTAT TTTCCCAAGG TTTGAACTAG CTCTTCATTT |
| 1861 | CTTTATGTTT TAAATGCACT GACCTCCCAC ATTCCCTTTT TAGTAAAATA TTCAGAAATA |
| 1921 | ATTTAAATAC ATCATTGCAA TGAAAATAAA TGTTTTTTAT TAGGCAGAAT CCAGATGCTC |
| 1981 | AAGGCCCTTC ATAATATCCC CCAGTTTAGT AGTTGGACTT AGGGAACAAA GGAACCTTTA |
| 2041 | ATAGAAATTG GACAGCAAGA AAGCGAGCTT CTAGCTTATC CTCAGTCCTG CTCCTCTGCC |
| 2101 | ACAAAGTGCA CGCAGTTGCC GGCCGGGTCG CGCAGGGCGA ACTCCCGCCC CCACGGCTGC |
| 2161 | TCGCCGATCT CGGTCATGGC CGGCCCGGAG GCGTCCCGGA AGTTCGTGGA CACGACCTCC |
| 2221 | GACCACTCGG CGTACAGCTC GTCCAGGCCG CGCACCCACA CCCAGGCCAG GGTGTTGTCC |
| 2281 | GGCACCACCT GGTCCTGGAC CGCGCTGATG AACAGGGTCA CGTCGTCCCG GACCACACCG |
| 2341 | GCGAAGTCGT CCTCCACGAA GTCCCGGGAG AACCCGAGCC GGTCGGTCCA GAACTCGACC |
| 2401 | GCTCCGGCGA CGTCGCGCGC GGTGAGCACC GGAACGGCAC TGGTCAACTT GGCCATGATG |
| 2461 | GCTCCTCctg tcaggagagg aaagagaaga aggttagtac aattgCTATA GTGAGTTGTA |
| 2521 | TTATACTATG CAGATATACT ATGCCAATGA TTAATTGTCA AACTAGGGCT GCAgggttca |
| 2581 | tagtgccact tttcctgcac tgccccatct cctgcccacc ctttcccagg catagacagt |
| 2641 | cagtgactta cCAAACTCAC AGGAGGGAGA AGGCAGAAGC TTGAGACAGA CCCGCGGGAC |
| 2701 | CGCCGAACTG CGAGGGGACG TGGCTAGGGC GGCTTCTTTT ATGGTGCGCC GGCCCTCGGA |
| 2761 | GGCAGGGCGC TCGGGGAGGC CTAGCGGCCA ATCTGCGGTG GCAGGAGGCG GGGCCGAAGG |
| 2821 | CCGTGCCTGA CCAATCCGGA GCACATAGGA GTCTCAGCCC CCCGCCCCAA AGCAAGGGGA |
| 2881 | AGTCACGCGC CTGTAGCGCC AGCGTGTTGT GAAATGGGGG CTTGGGGGGG TTGGGGCCCT |
| 2941 | GACTAGTCAA AACAAACTCC CATTGACGTC AATGGGGTGG AGACTTGGAA ATCCCCGTGA |
| 3001 | GTCAAACCGC TATCCACGCC CATTGATGTA CTGCCAAAAC CGCATCATCA TGGTAATAGC |
| 3061 | GATGACTAAT ACGTAGATGT ACTGCCAAGT AGGAAAGTCC CATAAGGTCA TGTACTGGGC |
| 3121 | ATAATGCCAG GCGGGCCATT TACCGTCATT GACGTCAATA GGGGGCGTAC TTGGCATATG |
| 3181 | ATACACTTGA TGTACTGCCA AGTGGGCAGT TTACCGTAAA TACTCCACCC ATTGACGTCA |
| 3241 | ATGGAAAGTC CCTATTGGCG TTACTATGGG AACATACGTC ATTATTGACG TCAATGGGCG |
| 3301 | GGGGTCGTTG GGCGGTCAGC CAGGCGGGCC ATTTACCGTA AGTTATGTAA CGCCTGCAGG |
| 3361 | TTAATTAAGA ACATGTGAGC AAAAGGCCAG CAAAAGGCCA GGAACCGTAA AAAGGCCGCG |
| 3421 | TTGCTGGCGT TTTTCCATAG GCTCCGCCCC CCTGACGAGC ATCACAAAAA TCGACGCTCA |
| 3481 | AGTCAGAGGT GGCGAAACCC GACAGGACTA TAAAGATACC AGGCGTTTCC CCCTGGAAGC |
| 3541 | TCCCTCGTGC GCTCTCCTGT TCCGACCCTG CCGCTTACCG GATACCTGTC CGCCTTTCTC |
| 3601 | CCTTCGGGAA GCGTGGCGCT TTCTCATAGC TCACGCTGTA GGTATCTCAG TTCGGTGTAG |
| 3661 | GTCGTTCGCT CCAAGCTGGG CTGTGTGCAC GAACCCCCCG TTCAGCCCGA CCGCTGCGCC |
| 3721 | TTATCCGGTA ACTATCGTCT TGAGTCCAAC CCGGTAAGAC ACGACTTATC GCCACTGGCA |
| 3781 | GCAGCCACTG GTAACAGGAT TAGCAGAGCG AGGTATGTAG GCGGTGCTAC AGAGTTCTTG |
| 3841 | AAGTGGTGGC CTAACTACGG CTACACTAGA AGAACAGTAT TTGGTATCTG CGCTCTGCTG |
| 3901 | AAGCCAGTTA CCTTCGGAAA AAGAGTTGGT AGCTCTTGAT CCGGCAAACA AACCACCGCT |
| 3961 | GGTAGCGGTG GTTTTTTTGT TTGCAAGCAG CAGATTACGC GCAGAAAAAA AGGATCTCAA |
| 4021 | GAAGATCCTT TGATCTTTTC TACGGGGTCT GACGCTCAGT GGAACGAAAA CTCACGTTAA |
| 4081 | GGGATTTTGG TCATGGCTAG TTAATTAACA TTTAAATCAG CGGCCGCAAT AAAATATCTT |
| 4141 | TATTTTCATT ACATCTGTGT GTTGGTTTTT TGTGTGAATC GTAACTAACA TACGCTCTCC |
| 4201 | ATCAAAACAA AACGAAACAA AACAAACTAG CAAAATAGGC TGTCCCCAGT GCAAGTGCAG |
| 4261 | GTGCCAGAAC ATTTCTCTAT CGAA |
| Amino acid sequence | |
| (SEQ ID NO: 86) | |
| MYRMQLLSCIALSLALVTNSIRLVQYRGEVQAMLGQSTEELRVRLAS | |
| HLRKLISAMVRSVECPPCPAPPVAGPSVFLFPPKPKDTLMISRTPEVTCVVVDV | |
| SHEDPEVQFNWYVDGMEVEINAKTKPREEQFNSTFRVVSVLTVVHQDWLNG | |
| KEYKCKVSNKGLPAPIEKTISKTKGQPREPQVYTLPPSREEMTKNQVSLTCLV | |
| KGFYPSDIAVEWESNGQPENNYKTTPPMLDSDGSFFLYSKLTVDKSRWQQGN | |
| VFSCSVMHEALHNHYTQKSLSLSPGK | |
| 197G pfuse-hfc2 ApoE 114-144 R136S | |
| Nucleic acid sequence | |
| (SEQ ID NO: 87) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCTG CTTGCTCAAC |
| 481 | TCTACGTCTT TGTTTCGTTT TCTGTTCTGC GCCGTTACAG ATCCAAGCTG TGACCGGCGC |
| 541 | CTACCTGAGA TCAccggcGA AGGAGGGCCA CCATGTACAG GATGCAACTC CTGTCTTGCA |
| 601 | TTGCACTAAG TCTTGCACTT GTCACGAATT CGATaCGCCT GGTGCAGTAC CGCGGCGAGG |
| 661 | TGCAGGCCAT GCTCGGCCAG AGtActGAGG AGCTGCGGGT GaGCCTCGCC TCCCACCTGC |
| 721 | GCAAGCTGat ATCGGCCATG GTTAGATCTG TGGAGTGCCC ACCTTGCCCA GCACCACCTG |
| 781 | TGGCAGGACC TTCAGTCTTC CTCTTCCCCC CAAAACCCAA GGACACCCTG ATGATCTCCA |
| 841 | GAACCCCTGA GGTCACGTGC GTGGTGGTGG ACGTGAGCCA CGAAGACCCC GAGGTCCAGT |
| 901 | TCAACTGGTA CGTGGACGGC ATGGAGGTGC ATAATGCCAA GACAAAGCCA CGGGAGGAGC |
| 961 | AGTTCAACAG CACGTTCCGT GTGGTCAGCG TCCTCACCGT CGTGCACCAG GACTGGCTGA |
| 1021 | ACGGCAAGGA GTACAAGTGC AAGGTCTCCA ACAAAGGCCT CCCAGCCCCC ATCGAGAAAA |
| 1081 | CCATCTCCAA AACCAAAGGG CAGCCCCGAG AACCACAGGT GTACACCCTG CCCCCATCCC |
| 1141 | GGGAGGAGAT GACCAAGAAC CAGGTCAGCC TGACCTGCCT GGTCAAAGGC TTCTACCCCA |
| 1201 | GCGACATCGC CGTGGAGTGG GAGAGCAATG GGCAGCCGGA GAACAACTAC AAGACCACAC |
| 1261 | CTCCCATGCT GGACTCCGAC GGCTCCTTCT TCCTCTACAG CAAGCTCACC GTGGACAAGA |
| 1321 | GCAGGTGGCA GCAGGGGAAC GTCTTCTCAT GCTCCGTGAT GCATGAGGCT CTGCACAACC |
| 1381 | ACTACACACA GAAGAGCCTC TCCCTGTCTC CGGGTAAATG AgtgccacgG CTAGCTGGCC |
| 1441 | AGACATGATA AGATACATTG ATGAGTTTGG ACAAACCACA ACTAGAATGC AGTGAAAAAA |
| 1501 | ATGCTTTATT TGTGAAATTT GTGATGCTAT TGCTTTATTT GTAACCATTA TAAGCTGCAA |
| 1561 | TAAACAAGTT AACAACAACA ATTGCATTCA TTTTATGTTT CAGGTTCAGG GGGAGGTGTG |
| 1621 | GGAGGTTTTT TAAAGCAAGT AAAACCTCTA CAAATGTGGT ATGGAATTAA TTCTAAAATA |
| 1681 | CAGCATAGCA AAACTTTAAC CTCCAAATCA AGCCTCTACT TGAATCCTTT TCTGAGGGAT |
| 1741 | GAATAAGGCA TAGGCATCAG GGGCTGTTGC CAATGTGCAT TAGCTGTTTG CAGCCTCACC |
| 1801 | TTCTTTCATG GAGTTTAAGA TATAGTGTAT TTTCCCAAGG TTTGAACTAG CTCTTCATTT |
| 1861 | CTTTATGTTT TAAATGCACT GACCTCCCAC ATTCCCTTTT TAGTAAAATA TTCAGAAATA |
| 1921 | ATTTAAATAC ATCATTGCAA TGAAAATAAA TGTTTTTTAT TAGGCAGAAT CCAGATGCTC |
| 1981 | AAGGCCCTTC ATAATATCCC CCAGTTTAGT AGTTGGACTT AGGGAACAAA GGAACCTTTA |
| 2041 | ATAGAAATTG GACAGCAAGA AAGCGAGCTT CTAGCTTATC CTCAGTCCTG CTCCTCTGCC |
| 2101 | ACAAAGTGCA CGCAGTTGCC GGCCGGGTCG CGCAGGGCGA ACTCCCGCCC CCACGGCTGC |
| 2161 | TCGCCGATCT CGGTCATGGC CGGCCCGGAG GCGTCCCGGA AGTTCGTGGA CACGACCTCC |
| 2221 | GACCACTCGG CGTACAGCTC GTCCAGGCCG CGCACCCACA CCCAGGCCAG GGTGTTGTCC |
| 2281 | GGCACCACCT GGTCCTGGAC CGCGCTGATG AACAGGGTCA CGTCGTCCCG GACCACACCG |
| 2341 | GCGAAGTCGT CCTCCACGAA GTCCCGGGAG AACCCGAGCC GGTCGGTCCA GAACTCGACC |
| 2401 | GCTCCGGCGA CGTCGCGCGC GGTGAGCACC GGAACGGCAC TGGTCAACTT GGCCATGATG |
| 2461 | GCTCCTCctg tcaggagagg aaagagaaga aggttagtac aattgCTATA GTGAGTTGTA |
| 2521 | TTATACTATG CAGATATACT ATGCCAATGA TTAATTGTCA AACTAGGGCT GCAgggttca |
| 2581 | tagtgccact tttcctgcac tgccccatct cctgcccacc ctttcccagg catagacagt |
| 2641 | cagtgactta cCAAACTCAC AGGAGGGAGA AGGCAGAAGC TTGAGACAGA CCCGCGGGAC |
| 2701 | CGCCGAACTG CGAGGGGACG TGGCTAGGGC GGCTTCTTTT ATGGTGCGCC GGCCCTCGGA |
| 2761 | GGCAGGGCGC TCGGGGAGGC CTAGCGGCCA ATCTGCGGTG GCAGGAGGCG GGGCCGAAGG |
| 2821 | CCGTGCCTGA CCAATCCGGA GCACATAGGA GTCTCAGCCC CCCGCCCCAA AGCAAGGGGA |
| 2881 | AGTCACGCGC CTGTAGCGCC AGCGTGTTGT GAAATGGGGG CTTGGGGGGG TTGGGGCCCT |
| 2941 | GACTAGTCAA AACAAACTCC CATTGACGTC AATGGGGTGG AGACTTGGAA ATCCCCGTGA |
| 3001 | GTCAAACCGC TATCCACGCC CATTGATGTA CTGCCAAAAC CGCATCATCA TGGTAATAGC |
| 3061 | GATGACTAAT ACGTAGATGT ACTGCCAAGT AGGAAAGTCC CATAAGGTCA TGTACTGGGC |
| 3121 | ATAATGCCAG GCGGGCCATT TACCGTCATT GACGTCAATA GGGGGCGTAC TTGGCATATG |
| 3181 | ATACACTTGA TGTACTGCCA AGTGGGCAGT TTACCGTAAA TACTCCACCC ATTGACGTCA |
| 3241 | ATGGAAAGTC CCTATTGGCG TTACTATGGG AACATACGTC ATTATTGACG TCAATGGGCG |
| 3301 | GGGGTCGTTG GGCGGTCAGC CAGGCGGGCC ATTTACCGTA AGTTATGTAA CGCCTGCAGG |
| 3361 | TTAATTAAGA ACATGTGAGC AAAAGGCCAG CAAAAGGCCA GGAACCGTAA AAAGGCCGCG |
| 3421 | TTGCTGGCGT TTTTCCATAG GCTCCGCCCC CCTGACGAGC ATCACAAAAA TCGACGCTCA |
| 3481 | AGTCAGAGGT GGCGAAACCC GACAGGACTA TAAAGATACC AGGCGTTTCC CCCTGGAAGC |
| 3541 | TCCCTCGTGC GCTCTCCTGT TCCGACCCTG CCGCTTACCG GATACCTGTC CGCCTTTCTC |
| 3601 | CCTTCGGGAA GCGTGGCGCT TTCTCATAGC TCACGCTGTA GGTATCTCAG TTCGGTGTAG |
| 3661 | GTCGTTCGCT CCAAGCTGGG CTGTGTGCAC GAACCCCCCG TTCAGCCCGA CCGCTGCGCC |
| 3721 | TTATCCGGTA ACTATCGTCT TGAGTCCAAC CCGGTAAGAC ACGACTTATC GCCACTGGCA |
| 3781 | GCAGCCACTG GTAACAGGAT TAGCAGAGCG AGGTATGTAG GCGGTGCTAC AGAGTTCTTG |
| 3841 | AAGTGGTGGC CTAACTACGG CTACACTAGA AGAACAGTAT TTGGTATCTG CGCTCTGCTG |
| 3901 | AAGCCAGTTA CCTTCGGAAA AAGAGTTGGT AGCTCTTGAT CCGGCAAACA AACCACCGCT |
| 3961 | GGTAGCGGTG GTTTTTTTGT TTGCAAGCAG CAGATTACGC GCAGAAAAAA AGGATCTCAA |
| 4021 | GAAGATCCTT TGATCTTTTC TACGGGGTCT GACGCTCAGT GGAACGAAAA CTCACGTTAA |
| 4081 | GGGATTTTGG TCATGGCTAG TTAATTAACA TTTAAATCAG CGGCCGCAAT AAAATATCTT |
| 4141 | TATTTTCATT ACATCTGTGT GTTGGTTTTT TGTGTGAATC GTAACTAACA TACGCTCTCC |
| 4201 | ATCAAAACAA AACGAAACAA AACAAACTAG CAAAATAGGC TGTCCCCAGT GCAAGTGCAG |
| 4261 | GTGCCAGAAC ATTTCTCTAT CGAA |
| Amino acid sequence | |
| (SEQ ID NO: 88) | |
| MYRMQLLSCIALSLALVTNSIRLVQYRGEVQAMLGQSTEELRVSLASH | |
| LRKLISAMVRSVECPPCPAPPVAGPSVFLFPPKPKDTLMISRTPEVTCVVVDVS | |
| HEDPEVQFNWYVDGMEVEINAKTKPREEQFNSTFRVVSVLTVVEIQDWLNGK | |
| EYKCKVSNKGLPAPIEKTISKTKGQPREPQVYTLPPSREEMTKNQVSLTCLVK | |
| GFYPSDIAVEWESNGQPENNYKTTPPMLDSDGSFFLYSKLTVDKSRWQQGNV | |
| FSCSVMHEALHNHYTQKSLSLSPGK | |
| 184U pfcn hg2 ApoE 129-157 | |
| Nucleic acid sequence | |
| (SEQ ID NO: 89) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCACCGGCGA AGGAGGGCCA CCATGTACAG GATGCAACTC CTGTCTTGCA |
| 601 | TTGCACTAAG TCTTGCACTT GTCACGAATT CGGCACCTCT CGAGCGCAAA TCTAGTGTCG |
| 661 | AGTGCCCACC GTGCCCAGCA CCACCTGTGG CAGGACCGTC AGTCTTCCTC TTCCCCCCAA |
| 721 | AACCCAAGGA CACCCTCATG ATCTCCCGGA CCCCTGAGGT CACGTGCGTG GTGGTGGACG |
| 781 | TGAGCCACGA AGACCCCGAG GTCCAGTTCA ACTGGTACGT GGACGGCGTG GAGGTGCATA |
| 841 | ATGCCAAGAC AAAGCCACGG GAGGAGCAGT TCAACAGCAC GTTCCGTGTG GTCAGCGTCC |
| 901 | TCACCGTTGT GCACCAGGAC TGGCTGAACG GCAAGGAGTA CAAGTGCAAG GTCTCCAACA |
| 961 | AAGGCCTCCC AGCCCCCATC GAGAAAACCA TCTCCAAAAC CAAAGGGCAG CCCCGAGAAC |
| 1021 | CACAGGTGTA CACCCTGCCC CCATCCCGGG AGGAGATGAC CAAGAACCAG GTCAGCCTGA |
| 1081 | CCTGCCTGGT CAAAGGCTTC TACCCCAGCG ACATCGCCGT GGAGTGGGAG AGCAATGGGC |
| 1141 | AGCCGGAGAA CAACTACAAG ACCACGCCTC CCATGCTGGA CTCCGACGGC TCCTTCTTCC |
| 1201 | TCTACAGCAA GCTCACCGTG GACAAGAGCA GGTGGCAGCA GGGGAACGTC TTCTCATGCT |
| 1261 | CCGTGATGCA TGAGGCTCTG CACAACCACT ACACGCAGAA GAGCCTCTCC CTGTCTCCGG |
| 1321 | GTGCACGTAC GAGCACCGAG GAGCTGCGGG TGCGCCTCGC CTCCCACCTG CGCAAGCTGC |
| 1381 | GTAAGCGGCT CCTCCGCGAT GCCGATGACC TGCAGAAGtg aTATCTCGAG CTAGCTGGCC |
| 1441 | AGACATGATA AGATACATTG ATGAGTTTGG ACAAACCACA ACTAGAATGC AGTGAAAAAA |
| 1501 | ATGCTTTATT TGTGAAATTT GTGATGCTAT TGCTTTATTT GTAACCATTA TAAGCTGCAA |
| 1561 | TAAACAAGTT AACAACAACA ATTGCATTCA TTTTATGTTT CAGGTTCAGG GGGAGGTGTG |
| 1621 | GGAGGTTTTT TAAAGCAAGT AAAACCTCTA CAAATGTGGT ATGGAATTAA TTCTAAAATA |
| 1681 | CAGCATAGCA AAACTTTAAC CTCCAAATCA AGCCTCTACT TGAATCCTTT TCTGAGGGAT |
| 1741 | GAATAAGGCA TAGGCATCAG GGGCTGTTGC CAATGTGCAT TAGCTGTTTG CAGCCTCACC |
| 1801 | TTCTTTCATG GAGTTTAAGA TATAGTGTAT TTTCCCAAGG TTTGAACTAG CTCTTCATTT |
| 1861 | CTTTATGTTT TAAATGCACT GACCTCCCAC ATTCCCTTTT TAGTAAAATA TTCAGAAATA |
| 1921 | ATTTAAATAC ATCATTGCAA TGAAAATAAA TGTTTTTTAT TAGGCAGAAT CCAGATGCTC |
| 1981 | AAGGCCCTTC ATAATATCCC CCAGTTTAGT AGTTGGACTT AGGGAACAAA GGAACCTTTA |
| 2041 | ATAGAAATTG GACAGCAAGA AAGCGAGCTT CTAGCTTATC CTCAGTCCTG CTCCTCTGCC |
| 2101 | ACAAAGTGCA CGCAGTTGCC GGCCGGGTCG CGCAGGGCGA ACTCCCGCCC CCACGGCTGC |
| 2161 | TCGCCGATCT CGGTCATGGC CGGCCCGGAG GCGTCCCGGA AGTTCGTGGA CACGACCTCC |
| 2221 | GACCACTCGG CGTACAGCTC GTCCAGGCCG CGCACCCACA CCCAGGCCAG GGTGTTGTCC |
| 2281 | GGCACCACCT GGTCCTGGAC CGCGCTGATG AACAGGGTCA CGTCGTCCCG GACCACACCG |
| 2341 | GCGAAGTCGT CCTCCACGAA GTCCCGGGAG AACCCGAGCC GGTCGGTCCA GAACTCGACC |
| 2401 | GCTCCGGCGA CGTCGCGCGC GGTGAGCACC GGAACGGCAC TGGTCAACTT GGCCATGATG |
| 2461 | GCTCCTCctg tcaggagagg aaagagaaga aggttagtac aattgCTATA GTGAGTTGTA |
| 2521 | TTATACTATG CAGATATACT ATGCCAATGA TTAATTGTCA AACTAGGGCT GCAgggttca |
| 2581 | tagtgccact tttcctgcac tgccccatct cctgcccacc ctttcccagg catagacagt |
| 2641 | cagtgactta cCAAACTCAC AGGAGGGAGA AGGCAGAAGC TTGAGACAGA CCCGCGGGAC |
| 2701 | CGCCGAACTG CGAGGGGACG TGGCTAGGGC GGCTTCTTTT ATGGTGCGCC GGCCCTCGGA |
| 2761 | GGCAGGGCGC TCGGGGAGGC CTAGCGGCCA ATCTGCGGTG GCAGGAGGCG GGGCCGAAGG |
| 2821 | CCGTGCCTGA CCAATCCGGA GCACATAGGA GTCTCAGCCC CCCGCCCCAA AGCAAGGGGA |
| 2881 | AGTCACGCGC CTGTAGCGCC AGCGTGTTGT GAAATGGGGG CTTGGGGGGG TTGGGGCCCT |
| 2941 | GACTAGTCAA AACAAACTCC CATTGACGTC AATGGGGTGG AGACTTGGAA ATCCCCGTGA |
| 3001 | GTCAAACCGC TATCCACGCC CATTGATGTA CTGCCAAAAC CGCATCATCA TGGTAATAGC |
| 3061 | GATGACTAAT ACGTAGATGT ACTGCCAAGT AGGAAAGTCC CATAAGGTCA TGTACTGGGC |
| 3121 | ATAATGCCAG GCGGGCCATT TACCGTCATT GACGTCAATA GGGGGCGTAC TTGGCATATG |
| 3181 | ATACACTTGA TGTACTGCCA AGTGGGCAGT TTACCGTAAA TACTCCACCC ATTGACGTCA |
| 3241 | ATGGAAAGTC CCTATTGGCG TTACTATGGG AACATACGTC ATTATTGACG TCAATGGGCG |
| 3301 | GGGGTCGTTG GGCGGTCAGC CAGGCGGGCC ATTTACCGTA AGTTATGTAA CGCCTGCAGG |
| 3361 | TTAATTAAGA ACATGTGAGC AAAAGGCCAG CAAAAGGCCA GGAACCGTAA AAAGGCCGCG |
| 3421 | TTGCTGGCGT TTTTCCATAG GCTCCGCCCC CCTGACGAGC ATCACAAAAA TCGACGCTCA |
| 3481 | AGTCAGAGGT GGCGAAACCC GACAGGACTA TAAAGATACC AGGCGTTTCC CCCTGGAAGC |
| 3541 | TCCCTCGTGC GCTCTCCTGT TCCGACCCTG CCGCTTACCG GATACCTGTC CGCCTTTCTC |
| 3601 | CCTTCGGGAA GCGTGGCGCT TTCTCATAGC TCACGCTGTA GGTATCTCAG TTCGGTGTAG |
| 3661 | GTCGTTCGCT CCAAGCTGGG CTGTGTGCAC GAACCCCCCG TTCAGCCCGA CCGCTGCGCC |
| 3721 | TTATCCGGTA ACTATCGTCT TGAGTCCAAC CCGGTAAGAC ACGACTTATC GCCACTGGCA |
| 3781 | GCAGCCACTG GTAACAGGAT TAGCAGAGCG AGGTATGTAG GCGGTGCTAC AGAGTTCTTG |
| 3841 | AAGTGGTGGC CTAACTACGG CTACACTAGA AGAACAGTAT TTGGTATCTG CGCTCTGCTG |
| 3901 | AAGCCAGTTA CCTTCGGAAA AAGAGTTGGT AGCTCTTGAT CCGGCAAACA AACCACCGCT |
| 3961 | GGTAGCGGTG GTTTTTTTGT TTGCAAGCAG CAGATTACGC GCAGAAAAAA AGGATCTCAA |
| 4021 | GAAGATCCTT TGATCTTTTC TACGGGGTCT GACGCTCAGT GGAACGAAAA CTCACGTTAA |
| 4081 | GGGATTTTGG TCATGGCTAG TTAATTAACA TTTAAATCAG CGGCCGCAAT AAAATATCTT |
| 4141 | TATTTTCATT ACATCTGTGT GTTGGTTTTT TGTGTGAATC GTAACTAACA TACGCTCTCC |
| 4201 | ATCAAAACAA AACGAAACAA AACAAACTAG CAAAATAGGC TGTCCCCAGT GCAAGTGCAG |
| 4261 | GTGCCAGAAC ATTTCTCTAT CGAA |
| Amino acid sequence | |
| (SEQ ID NO: 90) | |
| MYRMQLLSCIALSLALVTNSAPLERKSSVECPPCPAPPVAGPSVFLFPP | |
| KPKDTLMISRTPEVTCVVVDVSHEDPEVQFNWYVDGVEVEINAKTKPREEQF | |
| NSTFRVVSVLTVVHQDWLNGKEYKCKVSNKGLPAPIEKTISKTKGQPREPQV | |
| YTLPPSREEMTKNQVSLTCLVKGFYPSDIAVEWESNGQPENNYKTTPPMLDS | |
| DGSFFLYSKLTVDKSRWQQGNVFSCSVMHEALHNHYTQKSLSLSPGARTSTE | |
| ELRVRLASHLRKLRKRLLRDADDLQK | |
| 184V pfcn hg2 129-157 R136S | |
| Nucleic acid sequence | |
| (SEQ ID NO: 91) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCACCGGCGA AGGAGGGCCA CCATGTACAG GATGCAACTC CTGTCTTGCA |
| 601 | TTGCACTAAG TCTTGCACTT GTCACGAATT CGGCACCTCT CGAGCGCAAA TCTAGTGTCG |
| 661 | AGTGCCCACC GTGCCCAGCA CCACCTGTGG CAGGACCGTC AGTCTTCCTC TTCCCCCCAA |
| 721 | AACCCAAGGA CACCCTCATG ATCTCCCGGA CCCCTGAGGT CACGTGCGTG GTGGTGGACG |
| 781 | TGAGCCACGA AGACCCCGAG GTCCAGTTCA ACTGGTACGT GGACGGCGTG GAGGTGCATA |
| 841 | ATGCCAAGAC AAAGCCACGG GAGGAGCAGT TCAACAGCAC GTTCCGTGTG GTCAGCGTCC |
| 901 | TCACCGTTGT GCACCAGGAC TGGCTGAACG GCAAGGAGTA CAAGTGCAAG GTCTCCAACA |
| 961 | AAGGCCTCCC AGCCCCCATC GAGAAAACCA TCTCCAAAAC CAAAGGGCAG CCCCGAGAAC |
| 1021 | CACAGGTGTA CACCCTGCCC CCATCCCGGG AGGAGATGAC CAAGAACCAG GTCAGCCTGA |
| 1081 | CCTGCCTGGT CAAAGGCTTC TACCCCAGCG ACATCGCCGT GGAGTGGGAG AGCAATGGGC |
| 1141 | AGCCGGAGAA CAACTACAAG ACCACGCCTC CCATGCTGGA CTCCGACGGC TCCTTCTTCC |
| 1201 | TCTACAGCAA GCTCACCGTG GACAAGAGCA GGTGGCAGCA GGGGAACGTC TTCTCATGCT |
| 1261 | CCGTGATGCA TGAGGCTCTG CACAACCACT ACACGCAGAA GAGCCTCTCC CTGTCTCCGG |
| 1321 | GTGCACGTAC GAGCACCGAG GAGCTGCGGG TGaGCCTCGC CTCCCACCTG CGCAAGCTGC |
| 1381 | GTAAGCGGCT CCTCCGCGAT GCCGATGACC TGCAGAAGtg aTATCTCGAG CTAGCTGGCC |
| 1441 | AGACATGATA AGATACATTG ATGAGTTTGG ACAAACCACA ACTAGAATGC AGTGAAAAAA |
| 1501 | ATGCTTTATT TGTGAAATTT GTGATGCTAT TGCTTTATTT GTAACCATTA TAAGCTGCAA |
| 1561 | TAAACAAGTT AACAACAACA ATTGCATTCA TTTTATGTTT CAGGTTCAGG GGGAGGTGTG |
| 1621 | GGAGGTTTTT TAAAGCAAGT AAAACCTCTA CAAATGTGGT ATGGAATTAA TTCTAAAATA |
| 1681 | CAGCATAGCA AAACTTTAAC CTCCAAATCA AGCCTCTACT TGAATCCTTT TCTGAGGGAT |
| 1741 | GAATAAGGCA TAGGCATCAG GGGCTGTTGC CAATGTGCAT TAGCTGTTTG CAGCCTCACC |
| 1801 | TTCTTTCATG GAGTTTAAGA TATAGTGTAT TTTCCCAAGG TTTGAACTAG CTCTTCATTT |
| 1861 | CTTTATGTTT TAAATGCACT GACCTCCCAC ATTCCCTTTT TAGTAAAATA TTCAGAAATA |
| 1921 | ATTTAAATAC ATCATTGCAA TGAAAATAAA TGTTTTTTAT TAGGCAGAAT CCAGATGCTC |
| 1981 | AAGGCCCTTC ATAATATCCC CCAGTTTAGT AGTTGGACTT AGGGAACAAA GGAACCTTTA |
| 2041 | ATAGAAATTG GACAGCAAGA AAGCGAGCTT CTAGCTTATC CTCAGTCCTG CTCCTCTGCC |
| 2101 | ACAAAGTGCA CGCAGTTGCC GGCCGGGTCG CGCAGGGCGA ACTCCCGCCC CCACGGCTGC |
| 2161 | TCGCCGATCT CGGTCATGGC CGGCCCGGAG GCGTCCCGGA AGTTCGTGGA CACGACCTCC |
| 2221 | GACCACTCGG CGTACAGCTC GTCCAGGCCG CGCACCCACA CCCAGGCCAG GGTGTTGTCC |
| 2281 | GGCACCACCT GGTCCTGGAC CGCGCTGATG AACAGGGTCA CGTCGTCCCG GACCACACCG |
| 2341 | GCGAAGTCGT CCTCCACGAA GTCCCGGGAG AACCCGAGCC GGTCGGTCCA GAACTCGACC |
| 2401 | GCTCCGGCGA CGTCGCGCGC GGTGAGCACC GGAACGGCAC TGGTCAACTT GGCCATGATG |
| 2461 | GCTCCTCctg tcaggagagg aaagagaaga aggttagtac aattgCTATA GTGAGTTGTA |
| 2521 | TTATACTATG CAGATATACT ATGCCAATGA TTAATTGTCA AACTAGGGCT GCAgggttca |
| 2581 | tagtgccact tttcctgcac tgccccatct cctgcccacc ctttcccagg catagacagt |
| 2641 | cagtgactta cCAAACTCAC AGGAGGGAGA AGGCAGAAGC TTGAGACAGA CCCGCGGGAC |
| 2701 | CGCCGAACTG CGAGGGGACG TGGCTAGGGC GGCTTCTTTT ATGGTGCGCC GGCCCTCGGA |
| 2761 | GGCAGGGCGC TCGGGGAGGC CTAGCGGCCA ATCTGCGGTG GCAGGAGGCG GGGCCGAAGG |
| 2821 | CCGTGCCTGA CCAATCCGGA GCACATAGGA GTCTCAGCCC CCCGCCCCAA AGCAAGGGGA |
| 2881 | AGTCACGCGC CTGTAGCGCC AGCGTGTTGT GAAATGGGGG CTTGGGGGGG TTGGGGCCCT |
| 2941 | GACTAGTCAA AACAAACTCC CATTGACGTC AATGGGGTGG AGACTTGGAA ATCCCCGTGA |
| 3001 | GTCAAACCGC TATCCACGCC CATTGATGTA CTGCCAAAAC CGCATCATCA TGGTAATAGC |
| 3061 | GATGACTAAT ACGTAGATGT ACTGCCAAGT AGGAAAGTCC CATAAGGTCA TGTACTGGGC |
| 3121 | ATAATGCCAG GCGGGCCATT TACCGTCATT GACGTCAATA GGGGGCGTAC TTGGCATATG |
| 3181 | ATACACTTGA TGTACTGCCA AGTGGGCAGT TTACCGTAAA TACTCCACCC ATTGACGTCA |
| 3241 | ATGGAAAGTC CCTATTGGCG TTACTATGGG AACATACGTC ATTATTGACG TCAATGGGCG |
| 3301 | GGGGTCGTTG GGCGGTCAGC CAGGCGGGCC ATTTACCGTA AGTTATGTAA CGCCTGCAGG |
| 3361 | TTAATTAAGA ACATGTGAGC AAAAGGCCAG CAAAAGGCCA GGAACCGTAA AAAGGCCGCG |
| 3421 | TTGCTGGCGT TTTTCCATAG GCTCCGCCCC CCTGACGAGC ATCACAAAAA TCGACGCTCA |
| 3481 | AGTCAGAGGT GGCGAAACCC GACAGGACTA TAAAGATACC AGGCGTTTCC CCCTGGAAGC |
| 3541 | TCCCTCGTGC GCTCTCCTGT TCCGACCCTG CCGCTTACCG GATACCTGTC CGCCTTTCTC |
| 3601 | CCTTCGGGAA GCGTGGCGCT TTCTCATAGC TCACGCTGTA GGTATCTCAG TTCGGTGTAG |
| 3661 | GTCGTTCGCT CCAAGCTGGG CTGTGTGCAC GAACCCCCCG TTCAGCCCGA CCGCTGCGCC |
| 3721 | TTATCCGGTA ACTATCGTCT TGAGTCCAAC CCGGTAAGAC ACGACTTATC GCCACTGGCA |
| 3781 | GCAGCCACTG GTAACAGGAT TAGCAGAGCG AGGTATGTAG GCGGTGCTAC AGAGTTCTTG |
| 3841 | AAGTGGTGGC CTAACTACGG CTACACTAGA AGAACAGTAT TTGGTATCTG CGCTCTGCTG |
| 3901 | AAGCCAGTTA CCTTCGGAAA AAGAGTTGGT AGCTCTTGAT CCGGCAAACA AACCACCGCT |
| 3961 | GGTAGCGGTG GTTTTTTTGT TTGCAAGCAG CAGATTACGC GCAGAAAAAA AGGATCTCAA |
| 4021 | GAAGATCCTT TGATCTTTTC TACGGGGTCT GACGCTCAGT GGAACGAAAA CTCACGTTAA |
| 4081 | GGGATTTTGG TCATGGCTAG TTAATTAACA TTTAAATCAG CGGCCGCAAT AAAATATCTT |
| 4141 | TATTTTCATT ACATCTGTGT GTTGGTTTTT TGTGTGAATC GTAACTAACA TACGCTCTCC |
| 4201 | ATCAAAACAA AACGAAACAA AACAAACTAG CAAAATAGGC TGTCCCCAGT GCAAGTGCAG |
| 4261 | GTGCCAGAAC ATTTCTCTAT CGAA |
| Amino acid sequence | |
| (SEQ ID NO: 92) | |
| MYRMQLLSCIALSLALVTNSAPLERKSSVECPPCPAPPVAGPSVFLFPP | |
| KPKDTLMISRTPEVTCVVVDVSHEDPEVQFNWYVDGVEVEINAKTKPREEQF | |
| NSTFRVVSVLTVVHQDWLNGKEYKCKVSNKGLPAPIEKTISKTKGQPREPQV | |
| YTLPPSREEMTKNQVSLTCLVKGFYPSDIAVEWESNGQPENNYKTTPPMLDS | |
| DGSFFLYSKLTVDKSRWQQGNVFSCSVMHEALHNHYTQKSLSLSPGARTSTE | |
| ELRVSLASHLRKLRKRLLRDADDLQK | |
| 197H pfuse hfc2 ApoE 129-157 | |
| Nucleic acid sequence | |
| (SEQ ID NO: 93) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCAccggcGA AGGAGGGCCA CCATGTACAG GATGCAACTC CTGTCTTGCA |
| 601 | TTGCACTAAG TCTTGCACTT GTCACGAATT CGATAAGCAC CGAGGAGCTG CGGGTGCGCC |
| 661 | TCGCCTCCCA CCTGCGCAAG CTGCGTAAGC GGCTCCTCCG CGATGCCGAT GACCTGCAGA |
| 721 | AGatatcgGC CATGGTTAGA TCTGTGGAGT GCCCACCTTG CCCAGCACCA CCTGTGGCAG |
| 781 | GACCTTCAGT CTTCCTCTTC CCCCCAAAAC CCAAGGACAC CCTGATGATC TCCAGAACCC |
| 841 | CTGAGGTCAC GTGCGTGGTG GTGGACGTGA GCCACGAAGA CCCCGAGGTC CAGTTCAACT |
| 901 | GGTACGTGGA CGGCATGGAG GTGCATAATG CCAAGACAAA GCCACGGGAG GAGCAGTTCA |
| 961 | ACAGCACGTT CCGTGTGGTC AGCGTCCTCA CCGTCGTGCA CCAGGACTGG CTGAACGGCA |
| 1021 | AGGAGTACAA GTGCAAGGTC TCCAACAAAG GCCTCCCAGC CCCCATCGAG AAAACCATCT |
| 1081 | CCAAAACCAA AGGGCAGCCC CGAGAACCAC AGGTGTACAC CCTGCCCCCA TCCCGGGAGG |
| 1141 | AGATGACCAA GAACCAGGTC AGCCTGACCT GCCTGGTCAA AGGCTTCTAC CCCAGCGACA |
| 1201 | TCGCCGTGGA GTGGGAGAGC AATGGGCAGC CGGAGAACAA CTACAAGACC ACACCTCCCA |
| 1261 | TGCTGGACTC CGACGGCTCC TTCTTCCTCT ACAGCAAGCT CACCGTGGAC AAGAGCAGGT |
| 1321 | GGCAGCAGGG GAACGTCTTC TCATGCTCCG TGATGCATGA GGCTCTGCAC AACCACTACA |
| 1381 | CACAGAAGAG CCTCTCCCTG TCTCCGGGTA AATGAgtgcc acgGCTAGCT GGCCAGACAT |
| 1441 | GATAAGATAC ATTGATGAGT TTGGACAAAC CACAACTAGA ATGCAGTGAA AAAAATGCTT |
| 1501 | TATTTGTGAA ATTTGTGATG CTATTGCTTT ATTTGTAACC ATTATAAGCT GCAATAAACA |
| 1561 | AGTTAACAAC AACAATTGCA TTCATTTTAT GTTTCAGGTT CAGGGGGAGG TGTGGGAGGT |
| 1621 | TTTTTAAAGC AAGTAAAACC TCTACAAATG TGGTATGGAA TTAATTCTAA AATACAGCAT |
| 1681 | AGCAAAACTT TAACCTCCAA ATCAAGCCTC TACTTGAATC CTTTTCTGAG GGATGAATAA |
| 1741 | GGCATAGGCA TCAGGGGCTG TTGCCAATGT GCATTAGCTG TTTGCAGCCT CACCTTCTTT |
| 1801 | CATGGAGTTT AAGATATAGT GTATTTTCCC AAGGTTTGAA CTAGCTCTTC ATTTCTTTAT |
| 1861 | GTTTTAAATG CACTGACCTC CCACATTCCC TTTTTAGTAA AATATTCAGA AATAATTTAA |
| 1921 | ATACATCATT GCAATGAAAA TAAATGTTTT TTATTAGGCA GAATCCAGAT GCTCAAGGCC |
| 1981 | CTTCATAATA TCCCCCAGTT TAGTAGTTGG ACTTAGGGAA CAAAGGAACC TTTAATAGAA |
| 2041 | ATTGGACAGC AAGAAAGCGA GCTTCTAGCT TATCCTCAGT CCTGCTCCTC TGCCACAAAG |
| 2101 | TGCACGCAGT TGCCGGCCGG GTCGCGCAGG GCGAACTCCC GCCCCCACGG CTGCTCGCCG |
| 2161 | ATCTCGGTCA TGGCCGGCCC GGAGGCGTCC CGGAAGTTCG TGGACACGAC CTCCGACCAC |
| 2221 | TCGGCGTACA GCTCGTCCAG GCCGCGCACC CACACCCAGG CCAGGGTGTT GTCCGGCACC |
| 2281 | ACCTGGTCCT GGACCGCGCT GATGAACAGG GTCACGTCGT CCCGGACCAC ACCGGCGAAG |
| 2341 | TCGTCCTCCA CGAAGTCCCG GGAGAACCCG AGCCGGTCGG TCCAGAACTC GACCGCTCCG |
| 2401 | GCGACGTCGC GCGCGGTGAG CACCGGAACG GCACTGGTCA ACTTGGCCAT GATGGCTCCT |
| 2461 | Cctgtcagga gaggaaagag aagaaggtta gtacaattgC TATAGTGAGT TGTATTATAC |
| 2521 | TATGCAGATA TACTATGCCA ATGATTAATT GTCAAACTAG GGCTGCAggg ttcatagtgc |
| 2581 | cacttttcct gcactgcccc atctcctgcc caccctttcc caggcataga cagtcagtga |
| 2641 | cttacCAAAC TCACAGGAGG GAGAAGGCAG AAGCTTGAGA CAGACCCGCG GGACCGCCGA |
| 2701 | ACTGCGAGGG GACGTGGCTA GGGCGGCTTC TTTTATGGTG CGCCGGCCCT CGGAGGCAGG |
| 2761 | GCGCTCGGGG AGGCCTAGCG GCCAATCTGC GGTGGCAGGA GGCGGGGCCG AAGGCCGTGC |
| 2821 | CTGACCAATC CGGAGCACAT AGGAGTCTCA GCCCCCCGCC CCAAAGCAAG GGGAAGTCAC |
| 2881 | GCGCCTGTAG CGCCAGCGTG TTGTGAAATG GGGGCTTGGG GGGGTTGGGG CCCTGACTAG |
| 2941 | TCAAAACAAA CTCCCATTGA CGTCAATGGG GTGGAGACTT GGAAATCCCC GTGAGTCAAA |
| 3001 | CCGCTATCCA CGCCCATTGA TGTACTGCCA AAACCGCATC ATCATGGTAA TAGCGATGAC |
| 3061 | TAATACGTAG ATGTACTGCC AAGTAGGAAA GTCCCATAAG GTCATGTACT GGGCATAATG |
| 3121 | CCAGGCGGGC CATTTACCGT CATTGACGTC AATAGGGGGC GTACTTGGCA TATGATACAC |
| 3181 | TTGATGTACT GCCAAGTGGG CAGTTTACCG TAAATACTCC ACCCATTGAC GTCAATGGAA |
| 3241 | AGTCCCTATT GGCGTTACTA TGGGAACATA CGTCATTATT GACGTCAATG GGCGGGGGTC |
| 3301 | GTTGGGCGGT CAGCCAGGCG GGCCATTTAC CGTAAGTTAT GTAACGCCTG CAGGTTAATT |
| 3361 | AAGAACATGT GAGCAAAAGG CCAGCAAAAG GCCAGGAACC GTAAAAAGGC CGCGTTGCTG |
| 3421 | GCGTTTTTCC ATAGGCTCCG CCCCCCTGAC GAGCATCACA AAAATCGACG CTCAAGTCAG |
| 3481 | AGGTGGCGAA ACCCGACAGG ACTATAAAGA TACCAGGCGT TTCCCCCTGG AAGCTCCCTC |
| 3541 | GTGCGCTCTC CTGTTCCGAC CCTGCCGCTT ACCGGATACC TGTCCGCCTT TCTCCCTTCG |
| 3601 | GGAAGCGTGG CGCTTTCTCA TAGCTCACGC TGTAGGTATC TCAGTTCGGT GTAGGTCGTT |
| 3661 | CGCTCCAAGC TGGGCTGTGT GCACGAACCC CCCGTTCAGC CCGACCGCTG CGCCTTATCC |
| 3721 | GGTAACTATC GTCTTGAGTC CAACCCGGTA AGACACGACT TATCGCCACT GGCAGCAGCC |
| 3781 | ACTGGTAACA GGATTAGCAG AGCGAGGTAT GTAGGCGGTG CTACAGAGTT CTTGAAGTGG |
| 3841 | TGGCCTAACT ACGGCTACAC TAGAAGAACA GTATTTGGTA TCTGCGCTCT GCTGAAGCCA |
| 3901 | GTTACCTTCG GAAAAAGAGT TGGTAGCTCT TGATCCGGCA AACAAACCAC CGCTGGTAGC |
| 3961 | GGTGGTTTTT TTGTTTGCAA GCAGCAGATT ACGCGCAGAA AAAAAGGATC TCAAGAAGAT |
| 4021 | CCTTTGATCT TTTCTACGGG GTCTGACGCT CAGTGGAACG AAAACTCACG TTAAGGGATT |
| 4081 | TTGGTCATGG CTAGTTAATT AACATTTAAA TCAGCGGCCG CAATAAAATA TCTTTATTTT |
| 4141 | CATTACATCT GTGTGTTGGT TTTTTGTGTG AATCGTAACT AACATACGCT CTCCATCAAA |
| 4201 | ACAAAACGAA ACAAAACAAA CTAGCAAAAT AGGCTGTCCC CAGTGCAAGT GCAGGTGCCA |
| 4261 | GAACATTTCT CTATCGAA |
| Amino acid sequence | |
| (SEQ ID NO: 94) | |
| MYRMQLLSCIALSLALVTNSISTEELRVRLASHLRKLRKRLLRDADDL | |
| QKISAMVRSVECPPCPAPPVAGPSVFLFPPKPKDTLMISRTPEVTCVVVDVSHE | |
| DPEVQFNWYVDGMEVHNAKTKPREEQFNSTFRVVSVLTVVEIQDWLNGKEY | |
| KCKVSNKGLPAPIEKTISKTKGQPREPQVYTLPPSREEMTKNQVSLTCLVKGF | |
| YPSDIAVEWESNGQPENNYKTTPPMLDSDGSFFLYSKLTVDKSRWQQGNVFS | |
| CSVMHEALHNHYTQKSLSLSPGK | |
| 1971 pfuse hfc2 ApoE 129-157 R136S | |
| Nucleic acid sequence | |
| (SEQ ID NO: 95) | |
| 1 | GGATCTGCGA TCGCTCCGGT GCCCGTCAGT GGGCAGAGCG CACATCGCCC ACAGTCCCCG |
| 61 | AGAAGTTGGG GGGAGGGGTC GGCAATTGAA CGGGTGCCTA GAGAAGGTGG CGCGGGGTAA |
| 121 | ACTGGGAAAG TGATGTCGTG TACTGGCTCC GCCTTTTTCC CGAGGGTGGG GGAGAACCGT |
| 181 | ATATAAGTGC AGTAGTCGCC GTGAACGTTC TTTTTCGCAA CGGGTTTGCC GCCAGAACAC |
| 241 | AGCTGAAGCT TCGAGGGGCT CGCATCTCTC CTTCACGCGC CCGCCGCCCT ACCTGAGGCC |
| 301 | GCCATCCACG CCGGTTGAGT CGCGTTCTGC CGCCTCCCGC CTGTGGTGCC TCCTGAACTG |
| 361 | CGTCCGCCGT CTAGGTAAGT TTAAAGCTCA GGTCGAGACC GGGCCTTTGT CCGGCGCTCC |
| 421 | CTTGGAGCCT ACCTAGACTC AGCCGGCTCT CCACGCTTTG CCTGACCCtg cttgctcaac |
| 481 | tctacgTCTT TGTTTCGTTT TCTGTTCTGC GCCGTTACAG ATCCAAGCTG TGACCGGCGC |
| 541 | CTACCTGAGA TCAccggcGA AGGAGGGCCA CCATGTACAG GATGCAACTC CTGTCTTGCA |
| 601 | TTGCACTAAG TCTTGCACTT GTCACGAATT CGATAAGCAC CGAGGAGCTG CGGGTGaGCC |
| 661 | TCGCCTCCCA CCTGCGCAAG CTGCGTAAGC GGCTCCTCCG CGATGCCGAT GACCTGCAGA |
| 721 | AGatatcgGC CATGGTTAGA TCTGTGGAGT GCCCACCTTG CCCAGCACCA CCTGTGGCAG |
| 781 | GACCTTCAGT CTTCCTCTTC CCCCCAAAAC CCAAGGACAC CCTGATGATC TCCAGAACCC |
| 841 | CTGAGGTCAC GTGCGTGGTG GTGGACGTGA GCCACGAAGA CCCCGAGGTC CAGTTCAACT |
| 901 | GGTACGTGGA CGGCATGGAG GTGCATAATG CCAAGACAAA GCCACGGGAG GAGCAGTTCA |
| 961 | ACAGCACGTT CCGTGTGGTC AGCGTCCTCA CCGTCGTGCA CCAGGACTGG CTGAACGGCA |
| 1021 | AGGAGTACAA GTGCAAGGTC TCCAACAAAG GCCTCCCAGC CCCCATCGAG AAAACCATCT |
| 1081 | CCAAAACCAA AGGGCAGCCC CGAGAACCAC AGGTGTACAC CCTGCCCCCA TCCCGGGAGG |
| 1141 | AGATGACCAA GAACCAGGTC AGCCTGACCT GCCTGGTCAA AGGCTTCTAC CCCAGCGACA |
| 1201 | TCGCCGTGGA GTGGGAGAGC AATGGGCAGC CGGAGAACAA CTACAAGACC ACACCTCCCA |
| 1261 | TGCTGGACTC CGACGGCTCC TTCTTCCTCT ACAGCAAGCT CACCGTGGAC AAGAGCAGGT |
| 1321 | GGCAGCAGGG GAACGTCTTC TCATGCTCCG TGATGCATGA GGCTCTGCAC AACCACTACA |
| 1381 | CACAGAAGAG CCTCTCCCTG TCTCCGGGTA AATGAgtgcc acgGCTAGCT GGCCAGACAT |
| 1441 | GATAAGATAC ATTGATGAGT TTGGACAAAC CACAACTAGA ATGCAGTGAA AAAAATGCTT |
| 1501 | TATTTGTGAA ATTTGTGATG CTATTGCTTT ATTTGTAACC ATTATAAGCT GCAATAAACA |
| 1561 | AGTTAACAAC AACAATTGCA TTCATTTTAT GTTTCAGGTT CAGGGGGAGG TGTGGGAGGT |
| 1621 | TTTTTAAAGC AAGTAAAACC TCTACAAATG TGGTATGGAA TTAATTCTAA AATACAGCAT |
| 1681 | AGCAAAACTT TAACCTCCAA ATCAAGCCTC TACTTGAATC CTTTTCTGAG GGATGAATAA |
| 1741 | GGCATAGGCA TCAGGGGCTG TTGCCAATGT GCATTAGCTG TTTGCAGCCT CACCTTCTTT |
| 1801 | CATGGAGTTT AAGATATAGT GTATTTTCCC AAGGTTTGAA CTAGCTCTTC ATTTCTTTAT |
| 1861 | GTTTTAAATG CACTGACCTC CCACATTCCC TTTTTAGTAA AATATTCAGA AATAATTTAA |
| 1921 | ATACATCATT GCAATGAAAA TAAATGTTTT TTATTAGGCA GAATCCAGAT GCTCAAGGCC |
| 1981 | CTTCATAATA TCCCCCAGTT TAGTAGTTGG ACTTAGGGAA CAAAGGAACC TTTAATAGAA |
| 2041 | ATTGGACAGC AAGAAAGCGA GCTTCTAGCT TATCCTCAGT CCTGCTCCTC TGCCACAAAG |
| 2101 | TGCACGCAGT TGCCGGCCGG GTCGCGCAGG GCGAACTCCC GCCCCCACGG CTGCTCGCCG |
| 2161 | ATCTCGGTCA TGGCCGGCCC GGAGGCGTCC CGGAAGTTCG TGGACACGAC CTCCGACCAC |
| 2221 | TCGGCGTACA GCTCGTCCAG GCCGCGCACC CACACCCAGG CCAGGGTGTT GTCCGGCACC |
| 2281 | ACCTGGTCCT GGACCGCGCT GATGAACAGG GTCACGTCGT CCCGGACCAC ACCGGCGAAG |
| 2341 | TCGTCCTCCA CGAAGTCCCG GGAGAACCCG AGCCGGTCGG TCCAGAACTC GACCGCTCCG |
| 2401 | GCGACGTCGC GCGCGGTGAG CACCGGAACG GCACTGGTCA ACTTGGCCAT GATGGCTCCT |
| 2461 | Cctgtcagga gaggaaagag aagaaggtta gtacaattgC TATAGTGAGT TGTATTATAC |
| 2521 | TATGCAGATA TACTATGCCA ATGATTAATT GTCAAACTAG GGCTGCAggg ttcatagtgc |
| 2581 | cacttttcct gcactgcccc atctcctgcc caccctttcc caggcataga cagtcagtga |
| 2641 | cttacCAAAC TCACAGGAGG GAGAAGGCAG AAGCTTGAGA CAGACCCGCG GGACCGCCGA |
| 2701 | ACTGCGAGGG GACGTGGCTA GGGCGGCTTC TTTTATGGTG CGCCGGCCCT CGGAGGCAGG |
| 2761 | GCGCTCGGGG AGGCCTAGCG GCCAATCTGC GGTGGCAGGA GGCGGGGCCG AAGGCCGTGC |
| 2821 | CTGACCAATC CGGAGCACAT AGGAGTCTCA GCCCCCCGCC CCAAAGCAAG GGGAAGTCAC |
| 2881 | GCGCCTGTAG CGCCAGCGTG TTGTGAAATG GGGGCTTGGG GGGGTTGGGG CCCTGACTAG |
| 2941 | TCAAAACAAA CTCCCATTGA CGTCAATGGG GTGGAGACTT GGAAATCCCC GTGAGTCAAA |
| 3001 | CCGCTATCCA CGCCCATTGA TGTACTGCCA AAACCGCATC ATCATGGTAA TAGCGATGAC |
| 3061 | TAATACGTAG ATGTACTGCC AAGTAGGAAA GTCCCATAAG GTCATGTACT GGGCATAATG |
| 3121 | CCAGGCGGGC CATTTACCGT CATTGACGTC AATAGGGGGC GTACTTGGCA TATGATACAC |
| 3181 | TTGATGTACT GCCAAGTGGG CAGTTTACCG TAAATACTCC ACCCATTGAC GTCAATGGAA |
| 3241 | AGTCCCTATT GGCGTTACTA TGGGAACATA CGTCATTATT GACGTCAATG GGCGGGGGTC |
| 3301 | GTTGGGCGGT CAGCCAGGCG GGCCATTTAC CGTAAGTTAT GTAACGCCTG CAGGTTAATT |
| 3361 | AAGAACATGT GAGCAAAAGG CCAGCAAAAG GCCAGGAACC GTAAAAAGGC CGCGTTGCTG |
| 3421 | GCGTTTTTCC ATAGGCTCCG CCCCCCTGAC GAGCATCACA AAAATCGACG CTCAAGTCAG |
| 3481 | AGGTGGCGAA ACCCGACAGG ACTATAAAGA TACCAGGCGT TTCCCCCTGG AAGCTCCCTC |
| 3541 | GTGCGCTCTC CTGTTCCGAC CCTGCCGCTT ACCGGATACC TGTCCGCCTT TCTCCCTTCG |
| 3601 | GGAAGCGTGG CGCTTTCTCA TAGCTCACGC TGTAGGTATC TCAGTTCGGT GTAGGTCGTT |
| 3661 | CGCTCCAAGC TGGGCTGTGT GCACGAACCC CCCGTTCAGC CCGACCGCTG CGCCTTATCC |
| 3721 | GGTAACTATC GTCTTGAGTC CAACCCGGTA AGACACGACT TATCGCCACT GGCAGCAGCC |
| 3781 | ACTGGTAACA GGATTAGCAG AGCGAGGTAT GTAGGCGGTG CTACAGAGTT CTTGAAGTGG |
| 3841 | TGGCCTAACT ACGGCTACAC TAGAAGAACA GTATTTGGTA TCTGCGCTCT GCTGAAGCCA |
| 3901 | GTTACCTTCG GAAAAAGAGT TGGTAGCTCT TGATCCGGCA AACAAACCAC CGCTGGTAGC |
| 3961 | GGTGGTTTTT TTGTTTGCAA GCAGCAGATT ACGCGCAGAA AAAAAGGATC TCAAGAAGAT |
| 4021 | CCTTTGATCT TTTCTACGGG GTCTGACGCT CAGTGGAACG AAAACTCACG TTAAGGGATT |
| 4081 | TTGGTCATGG CTAGTTAATT AACATTTAAA TCAGCGGCCG CAATAAAATA TCTTTATTTT |
| 4141 | CATTACATCT GTGTGTTGGT TTTTTGTGTG AATCGTAACT AACATACGCT CTCCATCAAA |
| 4201 | ACAAAACGAA ACAAAACAAA CTAGCAAAAT AGGCTGTCCC CAGTGCAAGT GCAGGTGCCA |
| 4261 | GAACATTTCT CTATCGAA |

Amino acid sequence
(SEQ ID NO: 96)
MYRMQLLSCIALSLALVTNSISTEELRVSLASHLRKLRKRLLRDADDL
QKISAMVRSVECPPCPAPPVAGPSVFLFPPKPKDTLMISRTPEVTCVVVDVSHE
DPEVQFNWYVDGMEVHNAKTKPREEQFNSTFRVVSVLTVVHQDWLNGKEY
KCKVSNKGLPAPIEKTISKTKGQPREPQVYTLPPSREEMTKNQVSLTCLVKGF
YPSDIAVEWESNGQPENNYKTTPPMLDSDGSFFLYSKLTVDKSRWQQGNVFS
CSVMHEALHNHYTQKSLSLSPGK

### Example 5: CRISPR-Cas9 Mediated Editing and Base Editing of APOE

To introduce the R136S mutation in APOE using CRISPR-Cas9, gRNA sequences were designed (Table 7). In one example, a gRNA sequence is cloned into lentiCRISPR v2 using 2 oligos to form a linker containing the 20 base sequence that is cloned into the BsmB1 site downstream of the U6 promoter. To support repair, a template was designed with 2 additional silent mutations. The template has 50 bases flanking the area with the mutations. An exemplary template sequence is as follows:

(SEQ ID NO: 65)
CGCCTGGTGCAGTACCGCGGCGAGGTGCAGGCCATGCTCGGCCAGAGCAC
aGAGGAGCTcCGcGTGaGtCTCGCaagCCACCTGCGCAAGCTGCGTAAGC
GGCTCCTCCGCGATGCCGATGACCTGC

where silent mutations to abolish PAM motifs are double underlined, the codon corresponding to the R136S mutation is bolded, and silent mutation to generate SacI site for cleaving PCR products from clones that received the template is italicized.

TABLE 7
gRNA designs for introducing R136S mutation

| | | SEQ | Distance | ON | OFF |
|---|---|---|---|---|---|
| | | ID | From | target | target |
| Plasmid | gRNA | NO: | DSB | score | score |
| 184O1 | CTTACGCAGCTTGCGCAGGT | 69 | 16 | 61.2 | 90.5 |
| 184O2 | GCTTGCGCAGGTGGGAGGCG | 70 | 6 | 59.5 | 53.9 |
| 184O3 | ACGCAGCTTGCGCAGGTGGG | 71 | 13 | 59.4 | 62.9 |
| 184O4 | CCAGAGCACCGAGGAGCTGC | 72 | 9 | 49.3 | 42.7 |
| Optional 184O5 | GCCAGAGCACCGAGGAGCTG | 73 | 10 | 52.8 | 33.7 |
| Optional 184O6 | GAGGCGCACCCGCAGCTCCT | 74 | 11 | 51.1 | 60.5 |

Without wishing to be bound by theory, a proposed mechanism for the APOE3ch mutation is loss of function (e.g. in binding to HSPG). Accordingly, gRNA sequences were designed to “knockout” APOE using CRISPR-Cas9 (Table 8). The gRNAs are designed to target exon 3 (amino acids 1-61) of ApoE. In an example, a gRNA sequence is cloned into lentiCRISPR v2 by ordering 2 oligos to form a linker containing the 20 base sequence that is cloned into BsmB1 site downstream of the U6 promoter. Repair is done by non-homologous end joining (NHEJ), which is an error-prone process and often results in short insertions or deletions leading to APOE knockout.

TABLE 8
gRNA designs for APOE knockout

| | | SEQ | Break | ON | OFF |
|---|---|---|---|---|---|
| | | ID | at amino | target | target |
| Plasmid | gRNA | NO: | acid | score | score |
| 184Q1 | AGCTGCGCCAGCAGACCGAG | 75 | 18 | 66.3 | 74.4 |
| 184Q2 | CCAGGCCAAGGTGGAGCAAG | 76 | 3 | 65.6 | 49.3 |
| 184Q3 | CACAGGATGCCAGGCCAAGG | 77 | 1 | 65.4 | 44.5 |
| 184Q4 | ACAGTGTCTGCACCCAGCGC | 78 | 38 | 60.6 | 71.4 |
| 184Q5 | GGCCAAGGTGGAGCAAGCGG | 79 | 5 | 59.3 | 71.3 |

A R136H mutation in APOE is predicted to have a similar effect as the R136S mutation. Accordingly, to introduce a R136H mutation in APOE using base editing techniques, the following gRNA was designed (Table 9). The gRNA sequence GAGGCGCACCCGCAGCTCCT is cloned into pLenti sgRNA (addgene 71409), using 2 oligos to form a linker containing the 20 base sequence that is cloned into BsmB1 site downstream of the U6 promoter. Plasmid Addgene base editor plasmid pCMV-BE3 (#73021) is used to produce base editing.

TABLE 9
gRNA design for APOE base editing

| | SEQ | | Base | OFF |
|---|---|---|---|---|
| | ID | R136H | editing | target |
| gRNA | NO: | change | score | score |
| GAGGCGCACCCGCAGCTCCT | 80 | CGC→CAC | 4.6 | 60.5 |

### Example 6: High-Throughput Screening of Molecules that Modify ApoE and Heparin Binding

To screen for molecules that affect ApoE and heparin binding, ApoE proteins are pre-incubated with candidate polypeptides, small molecules, nucleic acids, lipids or carbohydrates. The pre-incubated ApoE proteins are introduced to heparin-coated surfaces (such as plates or columns), allowed to bind to heparin/HSPG/GAG, and unbound ApoE and candidate molecules are washed off. Alternatively, heparin-coated surfaces (such as plates) can be pre-incubated with candidate molecules, before applying ApoE proteins. The levels of ApoE bound to heparin are detected using antibodies, protein assays, or fluorescence, and the effect of the candidate molecules on ApoE/heparin binding are assessed. Candidate molecules that reduce ApoE/heparin binding may represent novel therapeutics for prevention or treatment of cognitive decline associated with dementia and/or mild cognitive impairment in a human subject in need of such treatments. An example of such molecules include EZ-482 (See, e.g. Mondal et al. Biochemistry 55(18):2613-21, 2016. The structure of EZ-482 is shown below.).

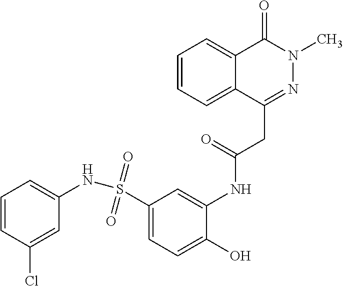 [SMILES: Cn1nc(CC(=O)Nc2cc(S(=O)(=O)Nc3cccc(Cl)c3)ccc2O)c2ccccc2c1=O]

### Example 7: High-Throughput Screening of Molecules that Modify ApoE and Anti-ApoE Antibody Binding

To screen for molecules that affect ApoE and anti-ApoE antibody binding, ApoE proteins are pre-incubated with candidate polypeptides, small molecules, nucleic acids, lipids or carbohydrates. The pre-incubated ApoE proteins are introduced to surfaces (such as plates or columns) coated with antibodies that bind to the HSPG-binding sites of ApoE (any of the anti-ApoE antibodies as described herein), allowed to bind to the antibodies, and unbound ApoE and candidate molecules are washed off. Alternatively, heparin-coated surfaces (such as plates) can be pre-incubated with candidate molecules, before applying ApoE proteins. The levels of ApoE bound to anti-ApoE antibodies are detected using antibodies, protein assays, or fluorescence, and the effect of the candidate molecules on the binding are assessed. Candidate molecules that increase or reduce ApoE/heparin binding may represent novel therapeutics for prevention or treatment of cognitive decline associated with dementia and/or mild cognitive impairment in a human subject in need of such treatments.

### Example 8: Antibody Characterization

Antibodies that bind to wild type ApoE and ApoEch mutant were further evaluated using heparin-affinity chromatography, western blotting, subclones analysis, monoclonal antibody screening, screening for selectivity between huApoE3 and msApoE, and in vivo subretinal injections.

### Methods

### Chromatography Experiments

His-tagged recombinant ApoE peptide (50 μg/mL) was incubated 3 h R.T. with each antibody individually at 1:10 dilution in 20 mM Tris HCl buffer (pH 7.5). Samples were tested for heparin binding using the heparin sepharose column. Briefly, the column was allowed to reach R.T. and washed 5 times with 20 mM Tris-HCL. Protein input was loaded onto the column upon collecting 10 μL for WB experiments. Input was recycled through the column 5 times. Flow-through was collected and the column was subsequently washed 5 times and recovered fractions were labeled as “washes”. 1 mL fractions were retrieved for each 0.05 M step of the NaCl salt gradient from 0 to 1 M. 5 M fraction was also tested to ensure complete release of the protein from the column. WB or ELISA were used to test changes in ApoE binding to heparin.

### Western Blotting

Samples for WB analyses were prepared by diluting 10 μL of each fraction in 4× sample buffer (Laemmli's SDS-Sample buffer, BP-110R, Boston bioproducts), 4 μL DTT (Sigma Aldrich), and 16 μL 1×RIPA buffer. Electrophoresis was performed under denaturing conditions, using a vertical electrophoretic chamber (Biorad). Bands separated on 4-20% precast gels (Biorad) using constant voltage (15′ 70V, 1 h 100V). Transfer of the proteins was performed on nitrocellulose membranes (Millipore) at 70 V constant voltage for 1 h. Membranes were blocked 1 h R.T. using Odyssey blocking buffer (Licor) and membranes were washed 3×10′ with TBS-0.05% Tween 20 (Thermo fisher) prior incubation with primary antibody (Anti-His, rb, 1:5000 Novus biological) and secondary antibody (Donkey anti-rb-800, 1:10000, Licor). Image acquisition was done using Odyssey scanner. Data was analyzed using Image J. Data was normalized by the input and expressed normalized intensities over fraction number (0=input, 1-27=increasing 0.05 M NaCl step gradient in 20 mM Tris HCl pH 7.5, 28=5 M NaCl in 20 mM Tris HCl pH 7.5).

### ELISA

To test selectivity of the antibodies for huApoE3WT, huApoE3ch or msApoE, anti-his ELISA coated plates were incubated with 0.0025 μg/μL of the target protein for 2 h at R.T. under gentle shaking. Wells were washed 5 times with 1× wash buffer (R&D) and subsequently incubated with serial dilutions of Innovagen antibody of interest over night at 4° C. on a shaker (100 μL/well). The following day, wells were washed 5 times with sample buffer and incubated for 45 minutes using Rabbit-anti-mouse HRP-conjugated buffer (1:10,000; 100 μL/well; Abcam). After 5 more washes, plates were incubated with chromogen A+B 1:1 to initiate the colorimetric reaction (100 μL/well). The reaction was stopped with 2 N sulfidric acid (R&D stop solution, 50 μL/well) and absorbance detected at 450 nm spectroscopically.

Antibodies designed against the heparin binding domain of APOE were tested for affinity to APOE3 and APOEch mutant recombinant protein using an ELISA. The Ni-NTA HisSorb Plates (Qiagen) plates were washed 3 times with wash buffer 1 (DY008), the APOE recombinant proteins were suspended in buffer (DY008) to give a final concentration of 0.5 ug/ml. The plates were incubated with 200ul for 2 hours. The plate was then washed 5 times with 1× wash buffer (DY008), the plates were then incubated with antibodies in a serial dilution series from 1:1000 to 1:32000 and incubated overnight at 4° C. The plate is then washed 5 times in 1× wash buffer (DY008). The plates were then incubated with Anti-mouse HRP (Abcam; ab97046) (1:10000) for 45 minutes. The plate was then washed 5 times in 1× wash buffer to ensure complete removal of unbound secondary antibody. The Sulfuric acid from the ELISA reagent kit (DY008) was warmed to 37° C. prior to addition of 100 μl of tetramethylbenzidine (Millipore) initiating the detection phase of the reaction. After a 5-mins incubation, sulfuric acid was added to terminate the reaction. The plate was then read using Synery 2 microplate reader (BioTek Instrument. Inc) and the Gen5 version1.11 software).

### Results

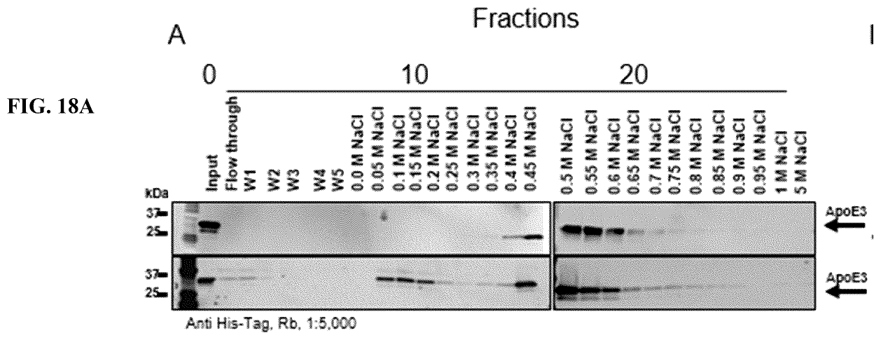

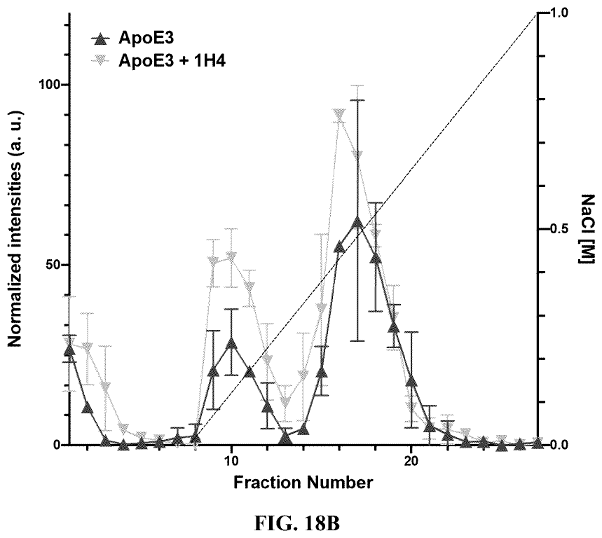

Antibody 1H4 was evaluated using heparin-affinity chromatography and western blotting. ApoE3 was incubated either with negative control (vehicle, top blots) or 1H4 (bottom blots) and each fraction was subjected to heparin-affinity chromatography and western blotting (FIG. 18A). ApoE3 positive bands are indicated by the arrows and detected using the antibody anti his-tag (rb, 1:5,000) that specifically detects the his-tag of the recombinant human APOE. FIG. 18B shows quantification of WB blotting bands detected by the antibody anti hi-tag as shown in FIG. 18A. Intensities were normalized to the input. These results show that ApoE3 binding to heparin was reduced in the presence of the antibody, and that antibody 1H4 competes with ApoE for heparin binding. N=2 independent experiments.

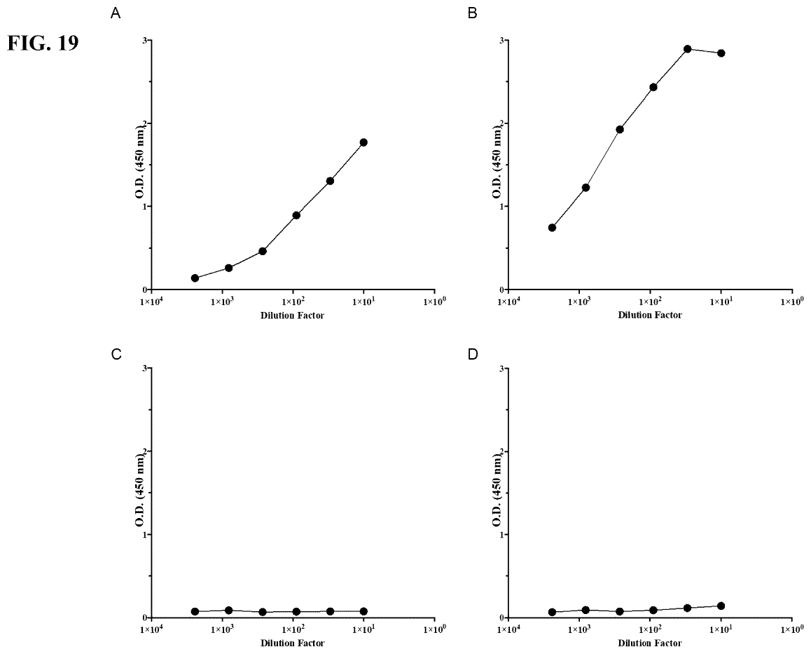

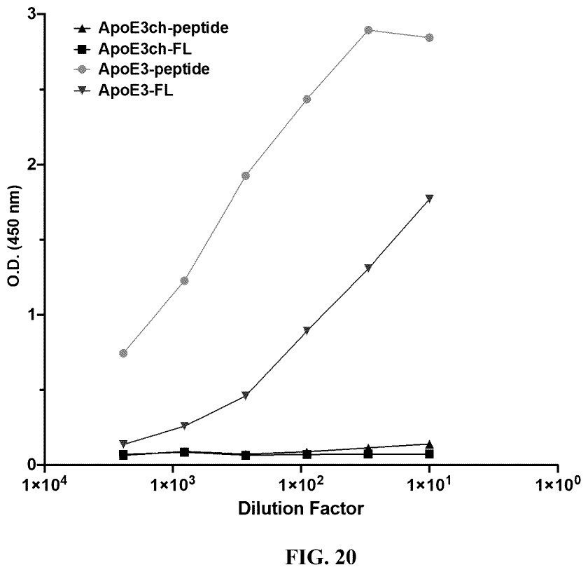

Next, subclone analysis of 1H4 serum was carried out. FIGS. 19A-19D show ELISA results of 1H4-2 serum tested with ApoE3 WT full-length protein (A), ApoE3 WT peptide (B), ApoE3ch full length protein (C), and ApoE3ch peptide (D). The results are expressed as optical density at 450 nm over dilution factor of the serum tested. FIG. 20 is a diagram comparing the results shown in FIGS. 19A-19D.

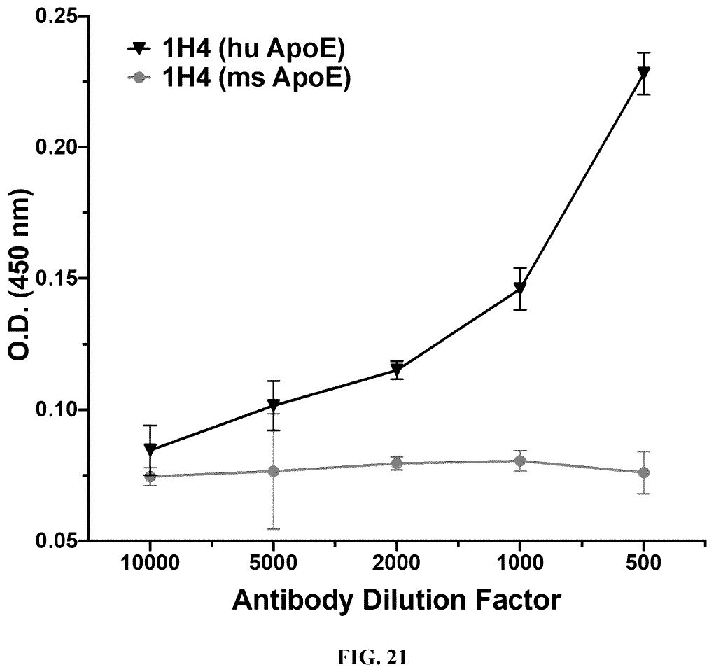

FIG. 21 shows representative ELISA profiles of serial dilutions of the antibody 1H4 incubated either with human recombinant ApoE3 or mouse recombinant ApoE3. The results show that the antibody preferentially binds to the human protein and not to the mouse, and that antibody 1H4 is selective for human ApoE. The results are shown as averaged optical densities detected at 450 nm±s.e.m. (n=2).

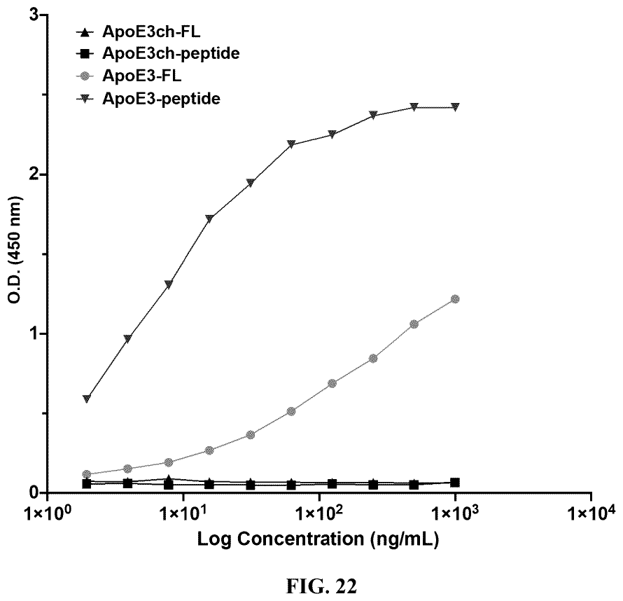

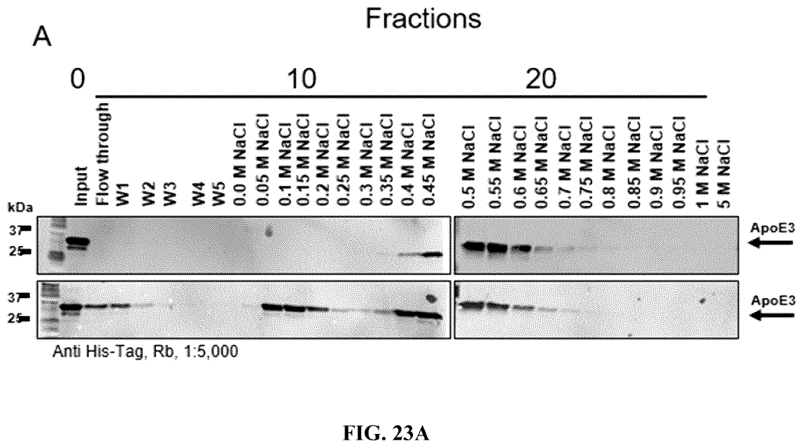

Next, monoclonal 1H4 antibody was purified from cloned hybridoma, and subjected to ELISA evaluation. FIG. 22 shows results from the ELISA experiments.

Antibody 7C11 was evaluated using heparin-affinity chromatography, western blotting, quantitative ELISA for chromatography fractions, and competitive ELISA for binding analyses.

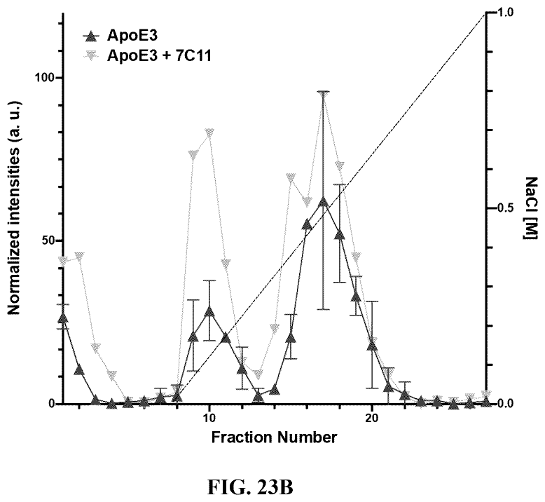

Heparin affinity chromatography fractions of ApoE3 incubated either with negative control (vehicle, top blots) or the antibody 7C11 (bottom blots) and subjected to western blotting (FIG. 23A). ApoE3 positive bands are indicated by the arrows and detected using the antibody anti his-tag (rb, 1:5,000) that specifically detects the his-tag of the recombinant peptide. FIG. 23B shows quantification of the WB blotting bands detected by the antibody anti hi-tag as shown in FIG. 23A. Intensities were normalized to the input. These results show that ApoE3 binding to heparin was reduced in the presence of the antibody, and that 7C11 competes with ApoE for heparin binding.

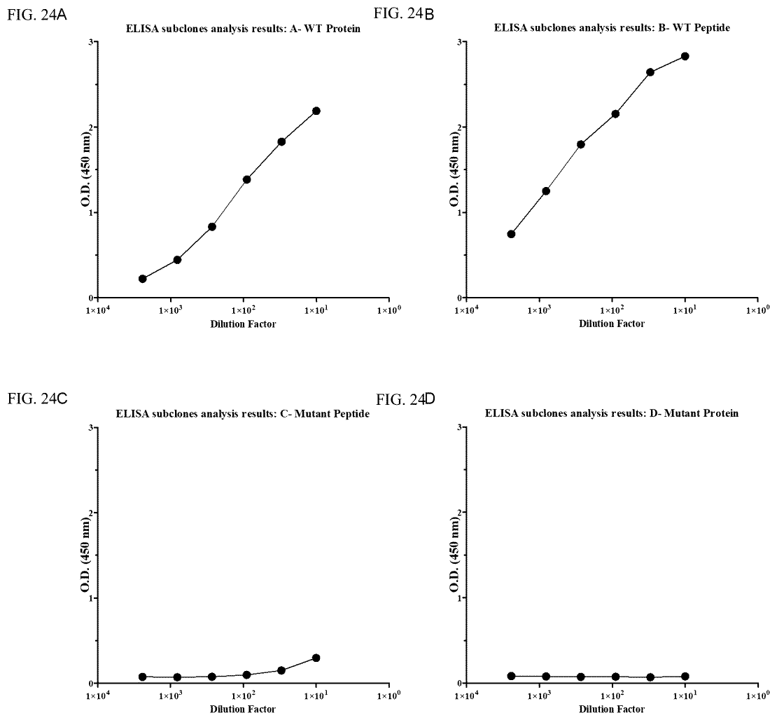

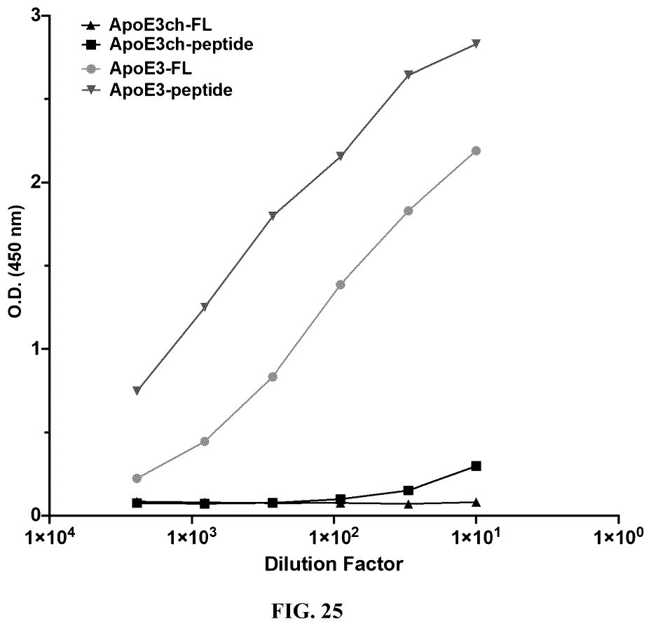

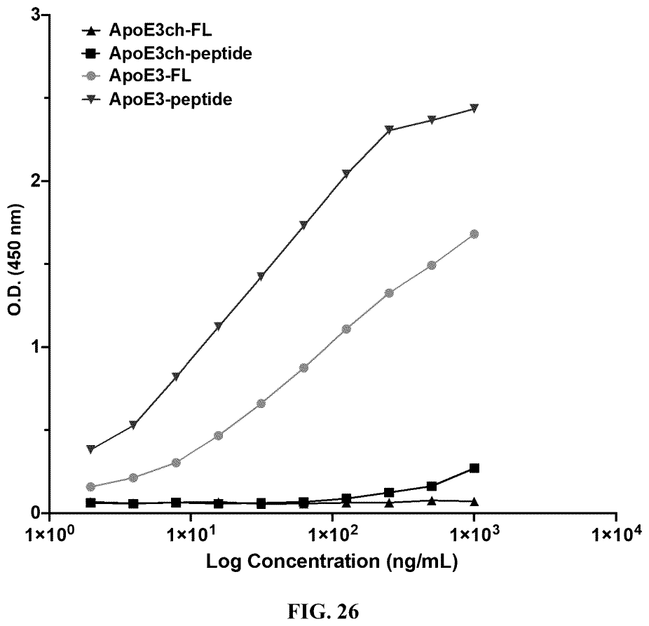

Next, subclone analysis of 7C11-1 serum was carried out. FIGS. 24A-24D show ELISA results from testing the 7C11-1 serum with ApoE3 WT full-length protein (A), ApoE3 WT peptide (B), ApoE3ch full length protein (C), or ApoE3ch peptide (D). The results are shown as optical density at 450 nm over dilution factor of the serum tested. FIG. 25 is a diagram comparing the results shown in FIGS. 19A-19D. Next, monoclonal 7C11-1 antibody was purified from cloned hybridoma, and subjected to ELISA evaluation. FIG. 26 shows results from the ELISA experiments.

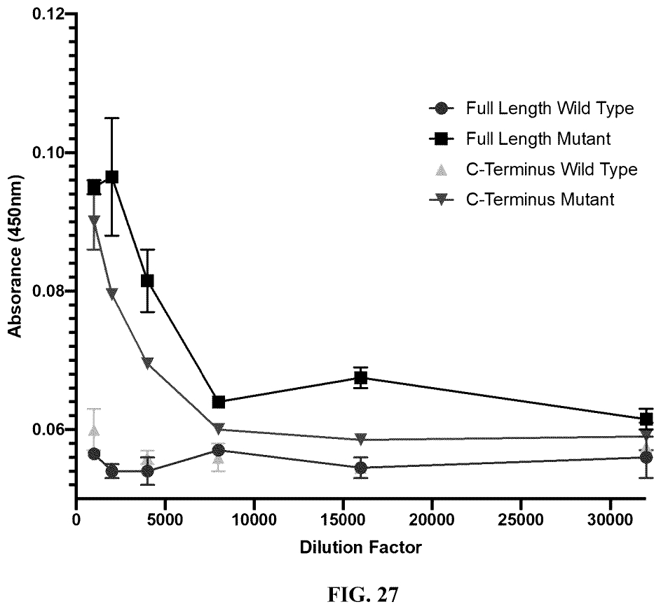

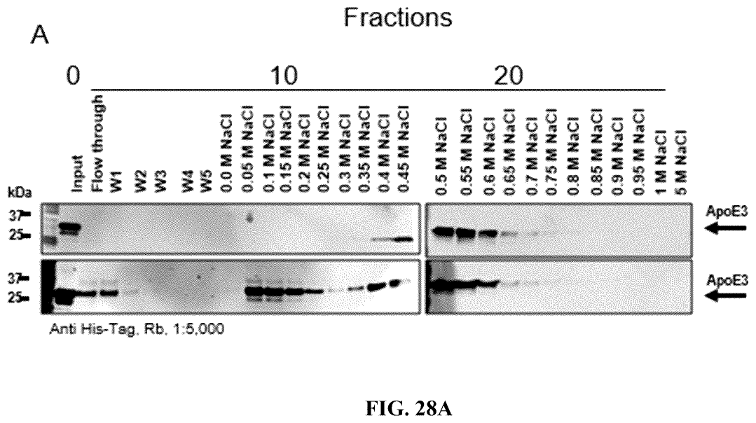

The 19G10-2 antibody was further evaluated using Heparin-Affinity chromatography, Western Blotting, Quantitative ELISA for chromatography fractions, and ELISA for binding analyses. ELISA screening of the 19G10-2 antibody was performed against the heparin binding domain of APOE3 Wild Type (WT) and APOE3ch Mutant recombinant protein. As shown in FIG. 27, the 19G10-2 antibody displays specificity towards both the full length and c-terminal domain of the APOE3ch (amino acids 125 to 299) mutant recombinant protein and some interaction with APOE3 WT.

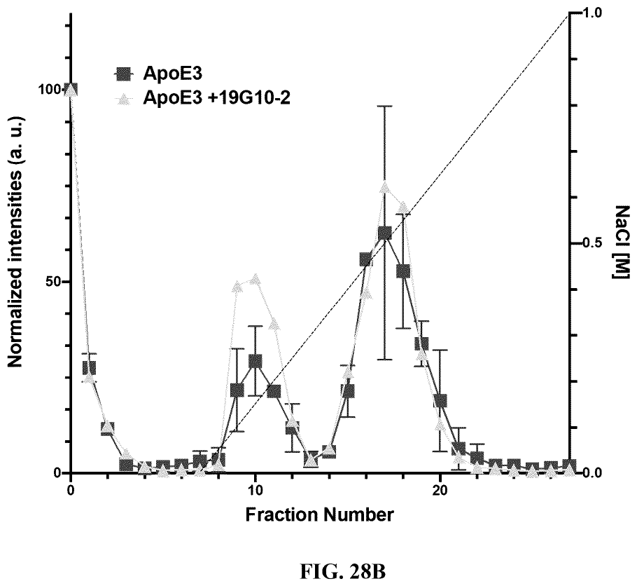

Heparin affinity chromatography fractions of ApoE3 incubated either with negative control (vehicle, top blots) or the antibody 19G10-2 (bottom blots) were subjected to ELISA analysis (FIG. 28A). ApoE3 positive bands are indicated by the arrows and detected using the antibody anti his-tag (rb, 1:5,000) that specifically detects the his-tag of the recombinant peptide. FIG. 28B shows quantification of WB blotting bands detected by the antibody anti hi-tag as shown in FIG. 28A. Intensities were normalized to the input. These results show that despite being designed against the ApoE3ch-HSPG domain, 19G10-2 competes with wild type ApoE for heparin binding and resulted in reduced ApoE3 binding to heparin. Without wishing to be bound by theory, antibody 19G10-2 may recognize and/or stabilize a conformation specific feature of APOE (e.g. an APOE polymer or aggregate) that is less likely to bind heparin/HSPG/GAG.

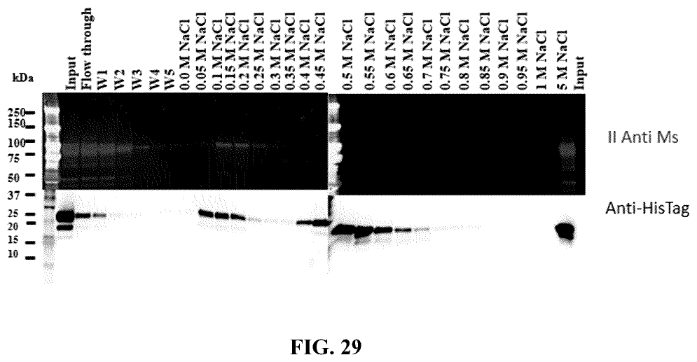

FIG. 29 shows western blotting of ApoE3 WT incubated with 19G10-2 serum antibody. Top blots: membranes probed with secondary anti-mouse to detect 19G10-2 antibody. Bottom membranes were incubated with anti his-tag as described previously to detect ApoE3 positive fractions. This analysis demonstrates that antibody-APOE complexes (left side of the blots; top and bottom) do not bind to heparin, while free APOE bind to heparin with high affinity (right side of the blot; bottom).

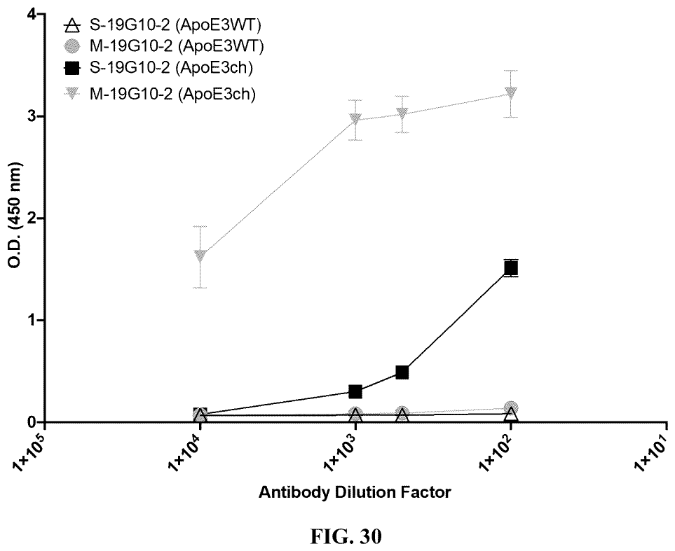

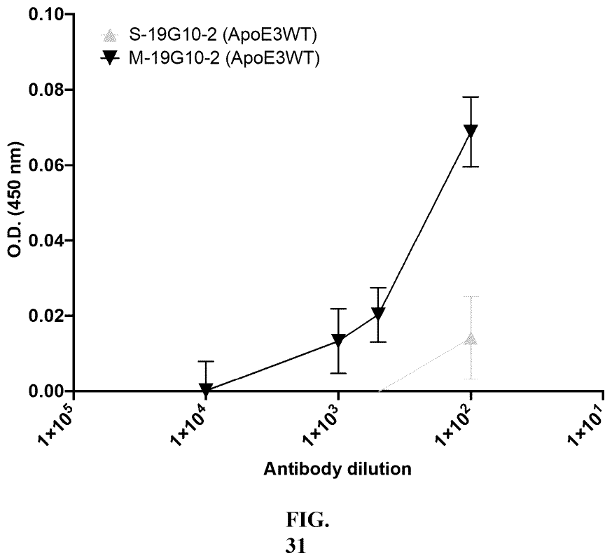

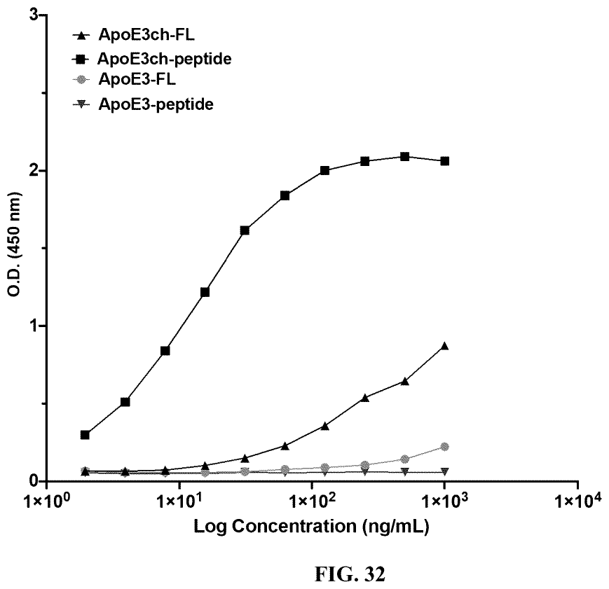

FIG. 30 is representative ELISA showing the differences in binding of both serumand monoclonal antibody hybridoma supernatant 19G10-2 for ApoE3WT or ApoE3ch. Data confirms the preponderant selectivity of this antibody for the ApoE3ch variant. FIG. 31 is an enlargement of the Y axes showing some limited binding profiles of the antibody 19G10-2 (serum, grey profile; monoclonal, black binding profile) in the presence of ApoE3WT. Next, monoclonal 19G10-2 antibody was purified from cloned hybridoma, and subjected to ELISA evaluation. FIG. 32 shows results from the ELISA experiments. A signal of recognition of the full-length WT APOE higher than that of the WT ApoE peptide suggest binding of a conformation-specific feature.

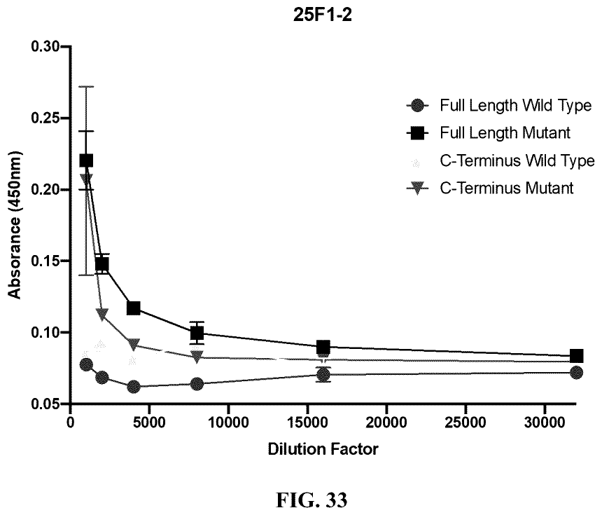

The 25F1-2 antibody was further evaluated using Heparin-Affinity chromatography, Western Blotting, Quantitative ELISA for chromatography fractions, and ELISA for binding analyses. FIG. 33 shows ELISA screening of the 25F1-2 antibody against the heparin binding domain of APOE3 Wild Type (WT) and APOE3ch Mutant recombinant protein. As shown in FIG. 33, the 25F1-2 antibody shows high affinity for the APOE3 mutant full length and c terminal protein, however, it does not appear to have strong binding to the c-terminus of the APOE3WT protein and showed limited interaction with APOE3 WT full length protein. The results are displayed as optical density at 450 nm over dilution factor of the serum tested.

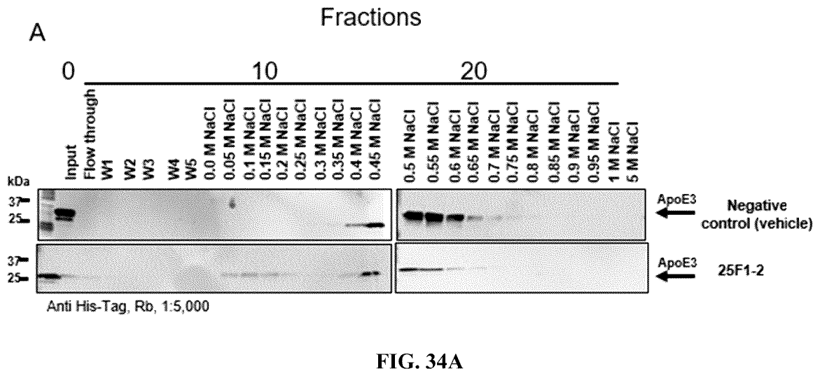

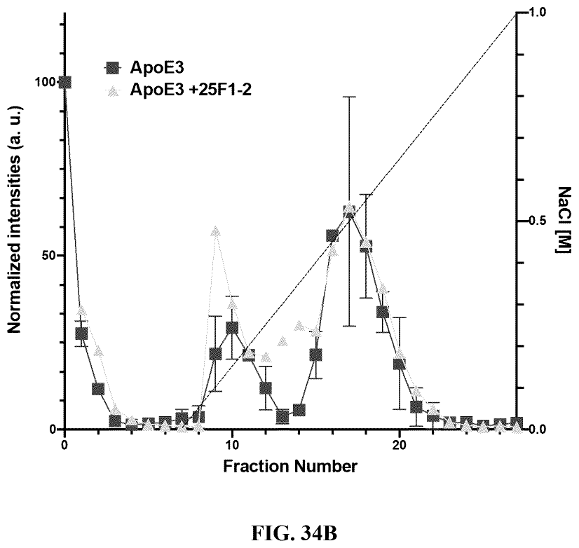

Heparin affinity chromatography fractions of ApoE3 incubated either with negative control (vehicle, top blots) or the antibody 25F1-2 (bottom blots) were subjected to western blotting (FIG. 34A). ApoE3 positive bands are indicated by the arrows and detected using the antibody anti his-tag (rb, 1:5,000) that specifically detects the his-tag of the recombinant peptide. FIG. 34B shows quantification of WB blotting bands detected by the antibody anti hi-tag as shown in FIG. 34A. Intensities were normalized to the input. These results show that despite being designed against the ApoE3ch-HSPG domain, 25F1-2 competes with wild type ApoE for heparin binding and resulted in reduced ApoE3 binding to heparin.

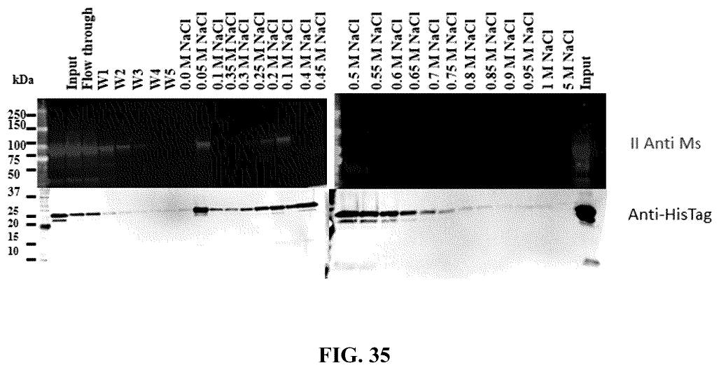

FIG. 35 shows western blotting of ApoE3 WT incubated with the 25F1-2 monoclonal antibody. Top blots: membranes probed with secondary anti-mouse to detect 25F1-2. Bottom membranes were incubated with anti his-tag as described previously to detect ApoE3 positive fractions. This analysis demonstrates that antibody-APOE complexes (left blots; top and bottom) do not bind to heparin, while free ApoE bind to heparin with high affinity (right blot, bottom).

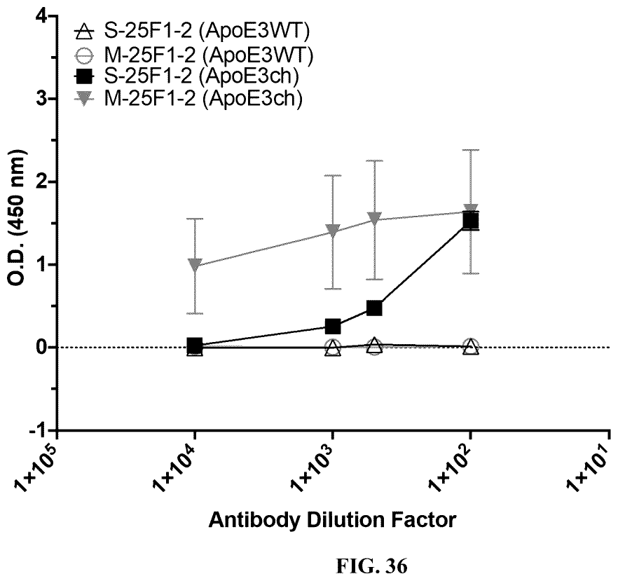

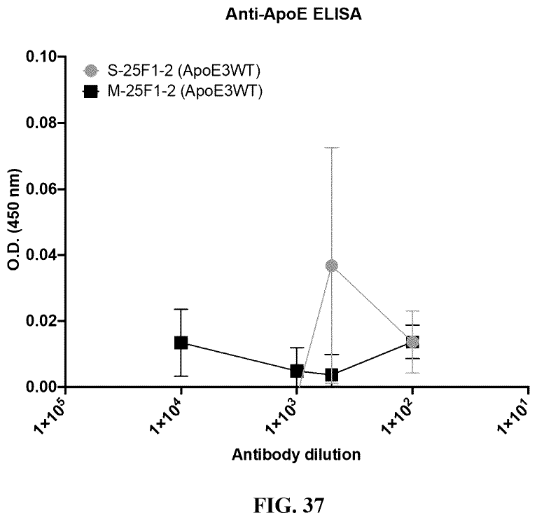

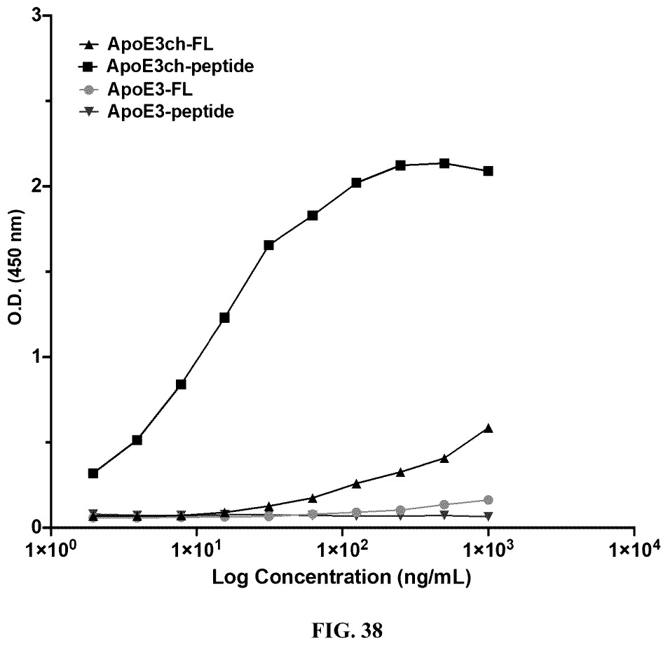

FIG. 36 is representative ELISA showing the differences in binding of both 25F1-2 serum and monoclonal antibody hybridoma supernatant 25F1-2 for ApoE3WT or ApoE3ch. These results confirms the preponderant selectivity of this antibody for the ApoE3ch variant. FIG. 37 is an enlargement of the Y axes of FIG. 36, showing the binding profiles of the antibody 25F1-2 in the presence of ApoE3WT. Next, monoclonal 25F1-2 antibody was purified from cloned hybridoma, and subjected to ELISA evaluation. FIG. 38 shows results from the ELISA experiments. A signal of some recognition of the full-length WT APOE higher than that of the WT ApoE peptide suggest binding of a conformation-specific feature.

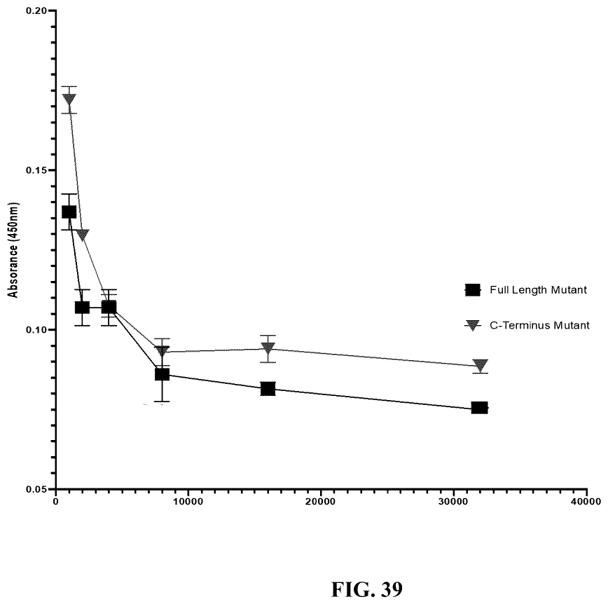

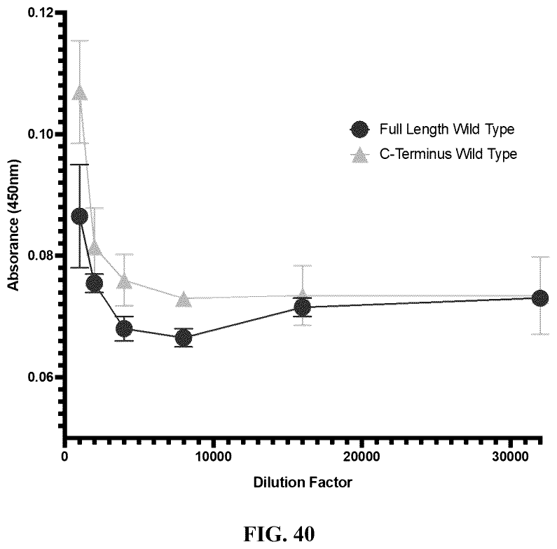

The mouse antibody 1343ab (renamed from 23B2) was evaluated using heparin-affinity chromatography and western blotting, and quantitative ELISA for chromatography fractions. FIGS. 39 and 40 show ELISA screening of the 1343 antibody against the heparin binding domain of APOE3 Wild Type (WT) and APOE3ch Mutant recombinant protein. 1343 displayed reactivity to both the APOE3 WT and APOE3ch mutant C-Terminus and full-length recombinant APOE proteins (mutant refers to Christchurch mutant).

ApoE in protein fractions eluted from heparin columns using an increasing NaCl gradient in the presence or absence of the 1343 was subjected to western blotting (FIG. 41A). Individual blots were cropped between 25 to 50 kDa. Blank spaces separate individual blots. FT=flow through. An ELISA was carried out to quantify differences in the NaCl elution patterns of different ApoE in the presence and absence of 1343 (FIG. 41B). N=3 columns per isoform in independent experiments were analyzed side-by-side twice on different days to quantify differences. Error bars depict standard error of mean.

Upon validation, CDR sequences from mouse antibodies 1H4-2, 7C11-1, 19G10-2, 25F1-2, and 1343ab were grafted into human IgG backbones (IgG2 or IgG4) to generate humanized counterparts.

### Example 9: In Vivo Validation of ApoE Antibodies

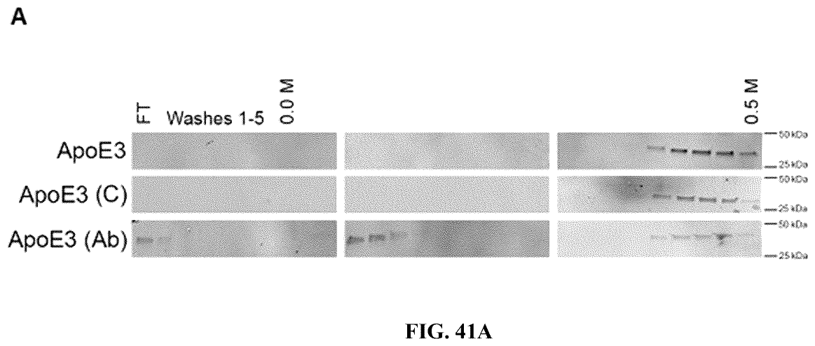

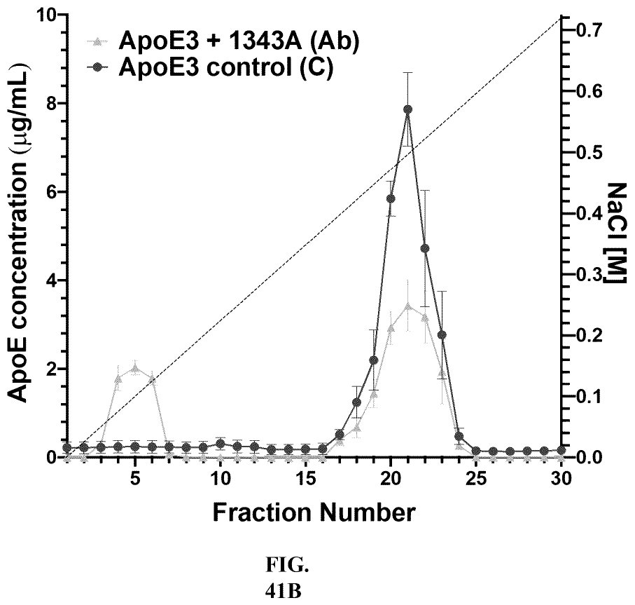

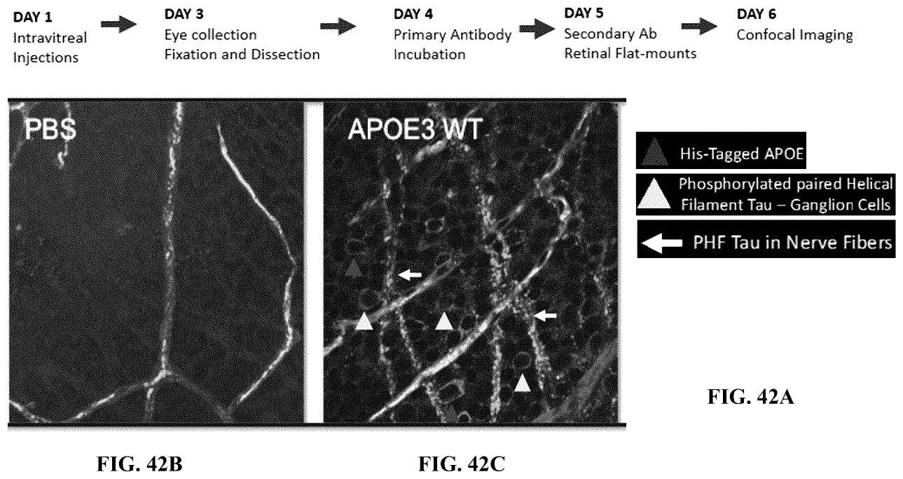

An intraocular model of inducible APOE-dependent Tau hyperphosphorylation (paired helical filament formation) was generated. This model was used to test the ability for the ApoE antibodies to inhibit Tau pathology, which is a marker of neurodegeneration. FIG. 42A shows an exemplary experimental outline.

Briefly, his-tagged recombinant human APOE3 was injected intravitreally into a B6; C3-Tg(Prnp-MAPT*P301S)PS19Vle/J mouse (Jackson lab 008169) (Yoshiyama et al. 53(3):337-51, 2007). This mouse model contains a human tau P301S mutation and is a validated animal model for Alzheimer's disease and other tauopathies such as frontotemporal dementia (See e.g. Bugiani et al. 58(6):667-77, 1999). Mice injected with PBS were used as control.

As shown in FIG. 42B, in the control retina of 6-week old mice, paired helical filaments of phosphorylated tau (PHF) are absent from ganglion cells and their axon fibers (signal inside vessels labeled with isolectin B4 are background signal). In contrast, administration of recombinant human APOE3 (his tagged) triggered robust formation of PHF detected with the AT8 antibody. PHF are robust in ganglion cell axons (arrows) and in the ganglion cell bodies. Human APOE was detected around the ganglion cell bodies using anti-His antibody.

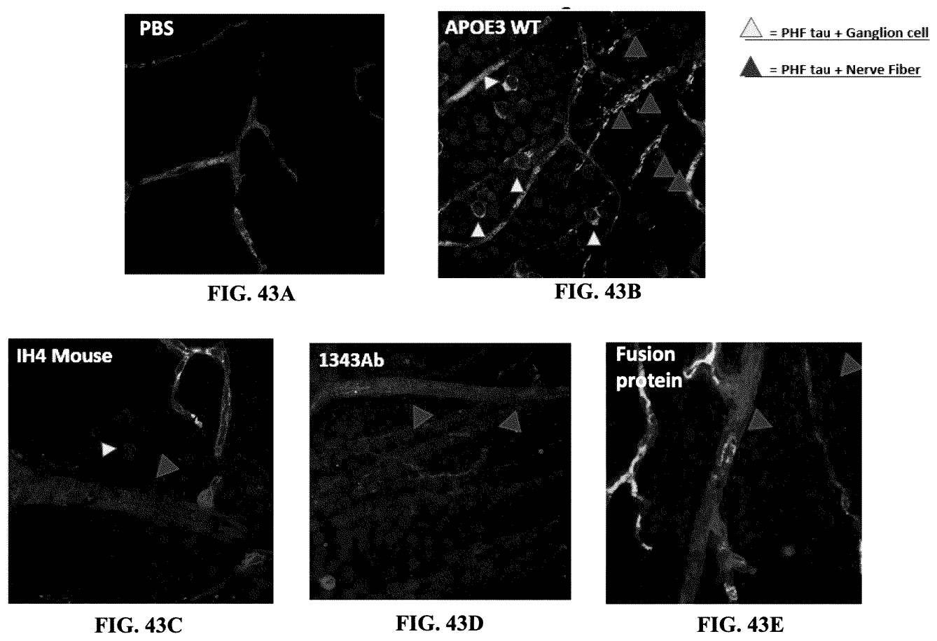

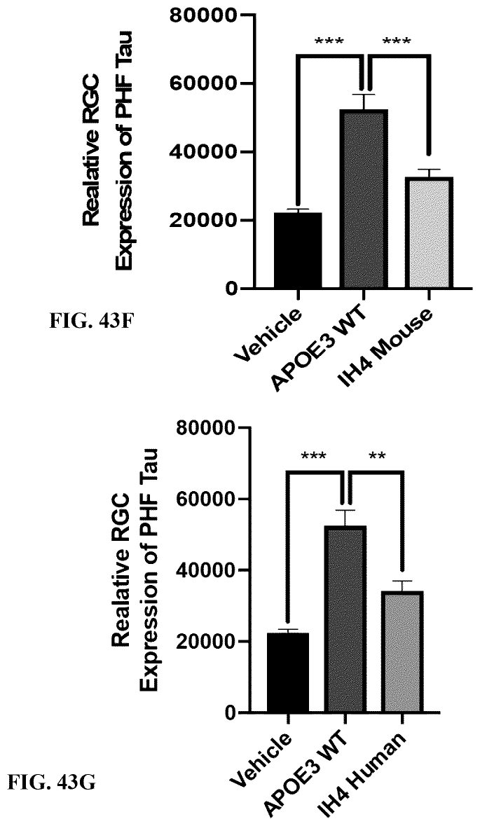

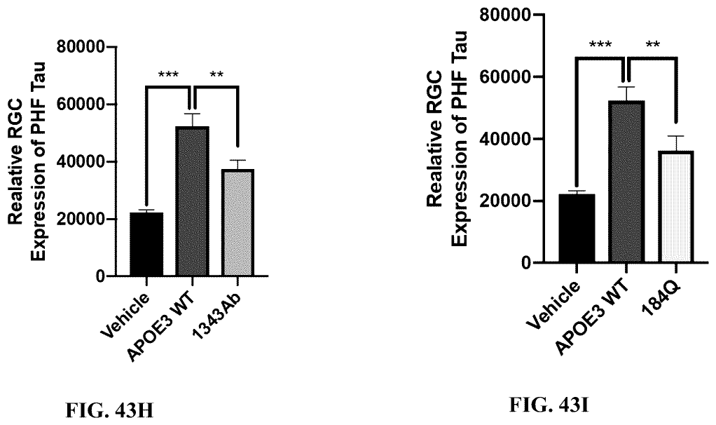

The mouse 1H4-2 antibody and the humanized 1343 Ah antibody were injected intravitreally into the eye of mice from the above mouse model (final volume 2 μL). The animals were sacrificed on day 3 post injection and retinas were dissected and immunolabeled. Retinas were stained with DAPI, Isolectin, and AT8 (pTAU), which recognizes phosphorylated paired helical filament tau (PHF tau). Retinas were imaged using the SP8 confocal microscope. As shown in FIGS. 43B, 43C, 43F and 43G, APOE3 WT resulted in a significant increase in PHF Tau, which is significantly reduced by 1H4-2 (***p<0.001). Similarly, administration of the humanized 1H4-2 IgG2/kappa recombinant monoclonal antibody effectively reduced APOE-dependent induction of PHF tau pathology in vivo (FIG. 43H; **p<0.01, ***p<0.001). As shown in FIGS. 43D and 43H, PHF Tau level was significantly reduced by the humanized 1343 Ah (**p<0.01, ***p<0.001).

The similarity of the efficacy between the mouse monoclonal antibody and the corresponding humanized antibody confirms the affinity of the binding domain for ApoE or ApoEch, and that the binding property is retained during humanization. This shows the binding properties of the CDRs are transferable from the original mouse IgG1 to other proteins including human IgG2.

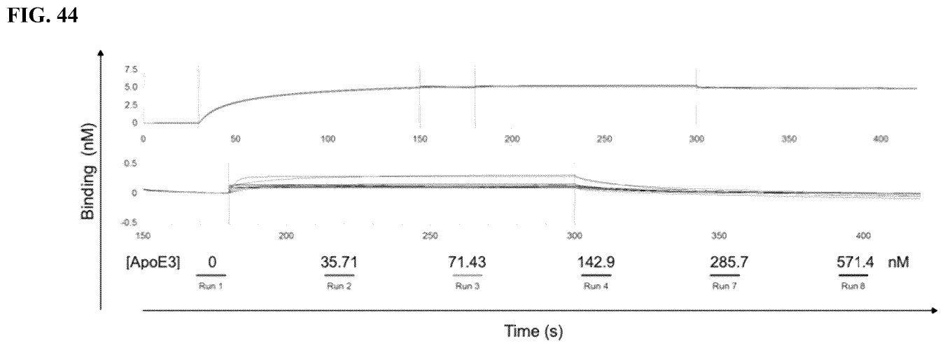

The binding affinity between ApoE3 and the monoclonal antibody 1H4 (mAb 1H4) was determined using the advanced kinetic module of the BLItz system (Blitz Pro, FB-609928, ver. 1.3.1.3). Briefly, a protein A biosensor was loaded with mAb 1H4 and both association and dissociation constants were determined at increasing concentrations of the full length ApoE3 protein (Innovagen) from 0 to 571.4 nM. The following running settings were used: 30 s initial baseline in the experimental media, 120 s loading of the ligand (mAb 1H4) on the biosensors, 30 s new baseline before association (120 s) and dissociation (120 s) steps. A total of 6 runs of 420 s at 2200 rpm and room temperature were conducted to determine the binding parameters of ApoE3. Global fitting and step corrections of the dissociation experiments were performed using the BLItz software (ver. 1.1.0.7). FIG. 44 shows representative binding measurements of increasing concentrations (nM) of ApoE3 protein to 1H4 on protein A biosensor. KD, Ka and Kd were measured (table 10) and calculated using the BLItz system. Top panel is representative of the association steps, the bottom panel is representative of the dissociation steps of the binding kinetic. The X and Y axes depicts time in second and binding in nM, respectively.

| | Con. | | | | | |
|---|---|---|---|---|---|---|
| Run | ApoE3 | KD | ka | | | kd |
| Index | (nM) | (nM) | (1/Ms) | Ka Error | kd (1/s) | Error |
| 1 | 0 | | | | | |
| 2 | 35.71 | 14.28 | 2900000 | 0.04142 | 0.001119 | 0.1543 |
| 3 | 71.43 | 14.28 | 2900000 | 0.04142 | 0.001119 | 0.1425 |
| 4 | 142.9 | 14.28 | 2900000 | 0.04142 | 0.001119 | 0.3063 |
| 7 | 285.7 | 14.28 | 2900000 | 0.04142 | 0.001119 | 0.09633 |
| 8 | 571.4 | 14.28 | 2900000 | 0.04142 | 0.001119 | 0.146 |
| | Con. | | | | | |
| Run | ApoE3 | | Rmax | R | | |
| Index | (nM) | Rmax | Error | Equilibrium | | |
| 1 | 0 | | | | | |
| 2 | 35.71 | 0.1543 | 0.005199 | 0.1102 | | |
| 3 | 71.43 | 0.1425 | 0.003305 | 0.1187 | | |
| 4 | 142.9 | 0.3063 | 0.003587 | 0.2785 | | |
| 7 | 285.7 | 0.09633 | 0.001949 | 0.09174 | | |
| 8 | 571.4 | 0.146 | 0.001883 | 0.1425 | | |

### Example 10: In Vivo Validation of APOE Fragment Fusion Proteins

Next, a fusion protein containing an APOE fragment that includes the HSPG-binding domain, and the Fc region of a human IgG was tested using a similar in vivo model. Briefly, 0.78 μg of recombinant full-length APOE was used to induce tau pathology. 0.14 μg of the fusion protein was injected intravitreally in to the mice. As shown in FIGS. 43E and 43I, the fusion protein diminished APOE-dependent tau pathology in neurons.

### Example 11: Sequences of Chimeric Antibodies

The sequences of the chimeric antibodies where CDRs were transferred from mouse to human IgG2 or IgG4 are shown below:
Normal font=vector
Italicized=Signal peptide
underlined=VL/VH
double underlined=Constant part (human Kappa/IgG4/IgG2)

Sequences of expression vectors for mAb 1H4 IgG2/Kappa:
>p1H4.VL.hk
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCAGACAACGTGCTGACACAGAGCCCTGCCAGCCTGGCTGTTTCTCTGGGACAGAGAG
CCACCATCAGCTGCAAGGCCAGCCAGAGCGTTGACTACGACGGCGACAGCTACATGAACTGG
TATCAGCAGAAGCCCGGCCAGCCACCTAAGGTGTTCATCTACGCCGCCAGCAACCTGGAAAG
CGGCATCCCTGCCAGATTTTCTGGCTCTGGCAGCGGCACCGACTTCACCCTGAATATCCATC
CTGTGGAAGAAGAGGACGCCGCCACCTACTACTGCCAGCAGAGCAATGAGGACCCCTGGACA
TTTGGCGGAGGCACCAAGCTGGAAATCAAGCGTACGGTGGCTGCACCATCTGTCTTCATCTT
CCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAGAGGGAGCTAGCGTGGCAT
CTAGACACTCTCGAGAAGGGTTCGATCCCTACCGGTTAGTAATGAGTTTGATATCTCGACAA
TCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAG
TCGGATCTCCCTTTGGGCCGCCTCCCCGCCTGGAAACGGGGGAGGCTAACTGAAACACGGAA
GGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATAC
CCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACCCTATC
TCGGTCTATTCTTTTGATTTATAAGGGATTTTGGGGATTTCGGCCTATTGGTTAAAAAATGA
GCTGATTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACTCCGCCCAGTTC
CGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAA
AAGCTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAGGCTAT
TCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGAC
TCTGGGGTTCGCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAA
AGTGCCACCTGACGTCGACGGATCGGGAGATCTCCCGATCCCCTATGGTCGACTCTCAGTAC
AATCTGCTCTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGACAATTGCATG
AAGAATCTGCTTAGG
>p1H4.VE.hIgG2
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCGGAAGTGAAGCTGGTGGAAAGCGGCGGAGGTGTTGTTCAGCCTGGCGGATCTCTGA
AGCTGAGCTGTGCCGCCAGCGGCTTCACCTTTAGCAGCTACACAATGAGCTGGGTCCGACAG
ACCCCTGAGAAGAGACTGGAATGGGTCGCCAAGATCCGGAACGGCGGAGGCATCACCTACTA
CCTGGATACCCTGAAGGGCAGATTCACCATCAGCCGGGACAACGCCAAGAACACCCTGTACC
TGCAGATGAGCAGCCTGAAGTCCGAGGACACCGCCATCTACTTTTGCGCCAGACACTACTAC
GGCAGCGAGGACTACTTCGACTATTGGGGCCAGGGCACCACACTGACCGTTAGCTCTGCTAG
CACCAAGGGCCCATCGGTCTTCCCCCTGGCGCCCTGCTCCAGGAGCACCTCCGAGAGCACAG
CGGCCCTGGGCTGCCTGGTCAAGGACTACTTCCCCGAACCGGTGACGGTGTCGTGGAACTCA
GGCGCTCTGACCAGCGGCGTGCACACCTTCCCAGCTGTCCTACAGTCCTCAGGACTCTACTC
CCTCAGCAGCGTGGTGACCGTGCCCTCCAGCAACTTCGGCACCCAGACCTACACCTGCAACG
TAGATCACAAGCCCAGCAACACCAAGGTGGACAAGACAGTTGAGCGCAAATGTTGTGTCGAG
TGCCCACCGTGCCCAGCACCACCTGTGGCAGGACCGTCAGTCTTCCTCTTCCCCCCAAAACC
CAAGGACACCCTCATGATCTCCCGGACCCCTGAGGTCACGTGCGTGGTGGTGGACGTGAGCC
ACGAAGACCCCGAGGTCCAGTTCAACTGGTACGTGGACGGCGTGGAGGTGCATAATGCCAAG
ACAAAGCCACGGGAGGAGCAGTTCAACAGCACGTTCCGTGTGGTCAGCGTCCTCACCGTTGT
GCACCAGGACTGGCTGAACGGCAAGGAGTACAAGTGCAAGGTCTCCAACAAAGGCCTCCCAG
CCCCCATCGAGAAAACCATCTCCAAAACCAAAGGGCAGCCCCGAGAACCACAGGTGTACACC
CTGCCCCCATCCCGGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGGAGAACAACTACA
AGACCACGCCTCCCATGCTGGACTCCGACGGCTCCTTCTTCCTCTACAGCAAGCTCACCGTG
GACAAGAGCAGGTGGCAGCAGGGGAACGTCTTCTCATGCTCCGTGATGCATGAGGCTCTGCA
CAACCACTACACGCAGAAGAGCCTCTCCCTGTCTCCGGGTAAATGACCTAGCGTGGCATCTA
GACACTCTCGAGAAGGGTTCGATCCCTACCGGTTAGTAATGAGTTTGATATCTCGACAATCA
ACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTC
ATTTTCTCCTCCTTGTATAAATCCTGGTTGCTGTCTCTTTATGAGGAGTTGTGGCCCGTTGT
CAGGCAACGTGGCGTGGTGTGCACTGTGTTTGCTGACGCAACCCCCACTGGTTGGGGCATTG
CCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTC
CGTGGTGTTGTCGGGGAAGCTGACGTCCTTTCCATGGCTGCTCGCCTGTGTTGCCACCTGGA
TTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCG
GATCTCCCTTTGGGCCGCCTCCCCGCCTGGAAACGGGGGAGGCTAACTGAAACACGGAAGGA
GACAATACCGGAAGGAACCCGCGCTATGACGGCAATAAAAAGACAGAATAAAACGCACGGGT
GTTGGGTCGTTTGTTCATAAACGCGGGGTTCGGTCCCAGGGCTGGCACTCTGTCGATACCCC
ACCGAGACCCCATTGGGGCCAATACGCCCGCGTTTCTTCCTTTTCCCCACCCCACCCCCCAA
GTTCGGGTGAAGGCCCAGGGCTCGCAGCCAACGTCGGGGCGGCAGGCCCTGCCATAGCAGAT
CTGCGCAGCTGGGGCTCTAGGGGGTATCCCCACGCGCCCTGTAGCGGCGCATTAAGCGCGGC
GGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGG
GGCATCCCTTTAGGGTTCCGATTTAGTGCTTTACGGCACCTCGACCCCAAAAAACTTGATTA
GGGTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCTCG
GTCTATTCTTTTGATTTATAAGGGATTTTGGGGATTTCGGCCTATTGGTTAAAAAATGAGCT
GATTTAACAAAAATTTAACGCGAATTAATTCTGTGGAATGTGTGTCAGTTAGGGTGTGGAAA
GTCCCCAGGCTCCCCAGCAGGCAGAAGTATGCAAAGCATGCATCTCAATTAGTCAGCAACCA
GGTGTGGAAAGTCCCCAGGCTCCCCAGCAGGCAGAAGTATGCAAAGCATGCATCTCAATTAG
TCAGCAACCATAGTCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGCCGCCTCTG
CCTCTGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAAGAGACAGGATGAGGATCGTTTCG
CATGATTGAACAAGATGGATTGCACGCAGGTTCTCCGGCCGCTTGGGTGGAGAGGCTATTCG
GCTATGACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTGCAGGA
CGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTG
TCATCTCACCTTGCTCCTGCCGAGAAAGTATCCATCATGGCTGATGCAATGCGGCGGCTGCA
TACGCTTGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAGGGGCTC
GCGCCAGCCGAACTGTTCGCCAGGCTCAAGGCGCGCATGCCCGACGGCGAGGATCTCGTCGT
GACCCATGGCGATGCCTGCTTGCCGAATATCATGGTGGAAAATGGCCGCTTTTCTGGATTCA
TCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGC
TCCCGATTCGCAGCGCATCGCCTTCTATCGCCTTCTTGACGAGTTCTTCTGAGCGGGACTCT
GGGGTTCGCGAAATGACCGACCAAGCGACGCCCAACCTGCCATCACGAGATTTCGATTCCAC
CGCCGCCTTCTATGAAAGGTTGGGCTTCGGAATCGTTTTCCGGGACGCCGGCTGGATGATCC
TCCAGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGACCT
CTAGCTAGAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGT
GAGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCT
CAATGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGG
TCTGACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCA
GTCTATTAATTGTTGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCA
TACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGT
GCCACCTGACGTCGACGGATCGGGAGATCTCCCGATCCCCTATGGTCGACTCTCAGTACAAT
CTGCTCTGATGCCGCATAGTTAAGCCAGTATCTGCTCCCTGCTTGTGTGTTGGAGGTCGCTG
AGTAGTGCGCGAGCAAAATTTAAGCTACAACAAGGCAAGGCTTGACCGACAATTGCATGAAG
AATCTGCTTAGG
Sequences of expression vectors for mAb 1343Ah IgG2/kappa:
>p1343Ah.VL.hk
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCAGACATCGTGCTGACACAGAGCCCTGCCAGCCTGGCTGTTTCTCTGGGACAGAGAG
CCACCATCAGCTGCAAGGCCAGCCAGAGCGTTGACTACGACGGCGAGAACTACATGAACTGG
TATCAGCAGAAGCCCGGACAGAGCCCCAAGCTGCTGATCTACGTGGCCAGCAATCTGGAAAG
CGGCATCCCCGCCAGATTTTCTGGCAGCGGAAGCGGCACCGACTTCACCCTGAATATCCATC
CTGTGGAAGAAGAGGACGCCGCCACCTACTACTGCCAGCAGTCCAATCTGGACCCCTGGACA
TTTGGCGGAGGCACCAAGCTGGAAATCAAGCGTACGGTGGCTGCACCATCTGTCTTCATCTT
CCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAGAGGGAGCTAGCGTGGCAT
CTAGACACTCTCGAGAAGGGTTCGATCCCTACCGGTTAGTAATGAGTTTGATATCTCGACAA
TCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAG
TCGGATCTCCCTTTGGGCCGCCTCCCCGCCTGGAAACGGGGGAGGCTAACTGAAACACGGAA
GGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATAC
CCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACCCTATC
TCGGTCTATTCTTTTGATTTATAAGGGATTTTGGGGATTTCGGCCTATTGGTTAAAAAATGA
GCTGATTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACTCCGCCCAGTTC
CGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAA
AAGCTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAGGCTAT
TCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGAC
TCTGGGGTTCGCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAA
AGTGCCACCTGACGTCGACGGATCGGGAGATCTCCCGATCCCCTATGGTCGACTCTCAGTAC
AATCTGCTCTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGACAATTGCATG
AAGAATCTGCTTAGG
>p1343Ah.VH.hIgG2
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCGGAGGTCCAGCTGCAGCAGTCTGGCGCCGAACTTGTTAGACCTGGCGCTCTGGTCA
AGCTGAGCTGTAAAGCCAGCGGCTTCAACATCAAGGACTACCATCTGCACTGGGTCAAGCAG
AGGCCTGAGCAGGGACTCGAGTGGATCGGCTGGATCGACCCCGAGAACGGCAACGTGATCTA
CGACCCCAAGTTCCAGGGCAAAGCCACCATGACCGTGGTCACCAGCAGCAACACAGCCTACC
TGCAGCTGAGAAGCCTGACCAGCGAAGATACCGCCGTGTACTTCTGCACCAGAGGCACAGCC
AGAGCCAGCTTCGATTATTGGGGCCAGGGCACCAGCCTGACCGTTTCTTCTGCTAGCACCAA
GGGCCCATCGGTCTTCCCCCTGGCGCCCTGCTCCAGGAGCACCTCCGAGAGCACAGCGGCCC
TGGGCTGCCTGGTCAAGGACTACTTCCCCGAACCGGTGACGGTGTCGTGGAACTCAGGCGCT
CTGACCAGCGGCGTGCACACCTTCCCAGCTGTCCTACAGTCCTCAGGACTCTACTCCCTCAG
CAGCGTGGTGACCGTGCCCTCCAGCAACTTCGGCACCCAGACCTACACCTGCAACGTAGATC
ACAAGCCCAGCAACACCAAGGTGGACAAGACAGTTGAGCGCAAATGTTGTGTCGAGTGCCCA
CCGTGCCCAGCACCACCTGTGGCAGGACCGTCAGTCTTCCTCTTCCCCCCAAAACCCAAGGA
CACCCTCATGATCTCCCGGACCCCTGAGGTCACGTGCGTGGTGGTGGACGTGAGCCACGAAG
ACCCCGAGGTCCAGTTCAACTGGTACGTGGACGGCGTGGAGGTGCATAATGCCAAGACAAAG
CCACGGGAGGAGCAGTTCAACAGCACGTTCCGTGTGGTCAGCGTCCTCACCGTTGTGCACCA
GGACTGGCTGAACGGCAAGGAGTACAAGTGCAAGGTCTCCAACAAAGGCCTCCCAGCCCCCA
TCGAGAAAACCATCTCCAAAACCAAAGGGCAGCCCCGAGAACCACAGGTGTACACCCTGCCC
CCATCCCGGGAGGAGATGACCAAGAACCAGGTCAGCCTGACCTGCCTGGTCAAAGGCTTCTA
CCCCAGCGACATCGCCGTGGAGTGGGAGAGCAATGGGCAGCCGGAGAACAACTACAAGACCA
CGCCTCCCATGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCA
CTACACGCAGAAGAGCCTCTCCCTGTCTCCGGGTAAATGACCTAGCGTGGCATCTAGACACT
CTCGAGAAGGGTTCGATCCCTACCGGTTAGTAATGAGTTTGATATCTCGACAATCAACCTCT
GGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCACCA
CCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGT
GTTGTCGGGGAAGCTGACGTCCTTTCCATGGCTGCTCGCCTGTGTTGCCACCTGGATTCTGC
GCGGGACGTCCTTCTGCTACGTCCCTTCGGCCCTCAATCCAGCGGACCTTCCTTCCCGCGGC
CTGCTGCCGGCTCTGCGGCCTCTTCCGCGTCTTCGCCTTCGCCCTCAGACGAGTCGGATCTC
CCTTTGGGCCGCCTCCCCGCCTGGAAACGGGGGAGGCTAACTGAAACACGGAAGGAGACAAT
ACCGGAAGGAACCCGCGCTATGACGGCAATAAAAAGACAGAATAAAACGCACGGGTGTTGGG
TCGTTTGTTCATAAACGCGGGGTTCGGTCCCAGGGCTGGCACTCTGTCGATACCCCACCGAG
ACCCCATTGGGGCCAATACGCCCGCGTTTCTTCCTTTTCCCCACCCCACCCCCCAAGTTCGG
GTGAAGGCCCAGGGCTCGCAGCCAACGTCGGGGCGGCAGGCCCTGCCATAGCAGATCTGCGC
AGCTGGGGCTCTAGGGGGTATCCCCACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGCATC
CCTTTAGGGTTCCGATTTAGTGCTTTACGGCACCTCGACCCCAAAAAACTTGATTAGGGTGA
TGGTTCACGTAGTGGGCCATCGCCCTGATAGACGGTTTTTCGCCCTTTGACGTTGGAGTCCA
CGTTCTTTAATAGTGGACTCTTGTTCCAAACTGGAACAACACTCAACCCTATCTCGGTCTAT
TCTTTTGATTTATAAGGGATTTTGGGGATTTCGGCCTATTGGTTAAAAAATGAGCTGATTTA
ACAAAAATTTAACGCGAATTAATTCTGTGGAATGTGTGTCAGTTAGGGTGTGGAAAGTCCCC
AGGCTCCCCAGCAGGCAGAAGTATGCAAAGCATGCATCTCAATTAGTCAGCAACCAGGTGTG
GAAAGTCCCCAGGCTCCCCAGCAGGCAGAAGTATGCAAAGCATGCATCTCAATTAGTCAGCA
ACCATAGTCCCGCCCCTAACTCCGCCCATCCCGCCCCTAACTCCGCCCAGTTCCGCCCATTC
TCCGCCCCATGGCTGACTAATTTTTTTTATTTATGCAGAGGCCGAGGCCGCCTCTGCCTCTG
AGCTATTCCAGAAGTAGTGAGGAGGCTTTTTTGGAGGCCTAGGCTTTTGCAAAAAGCTCCCG
GGAGCTTGTATATCCATTTTCGGATCTGATCAAGAGACAGGATGAGGATCGTTTCGCATGAT
TGAACAAGATGGATTGCACGCAGGTTCTCCGGCCGCTTGGGTGGAGAGGCTATTCGGCTATG
ACTGGGCACAACAGACAATCGGCTGCTCTGATGCCGCCGTGTTCCGGCTGTCAGCGCAGGGG
CGCCCGGTTCTTTTTGTCAAGACCGACCTGTCCGGTGCCCTGAATGAACTGCAGGACGAGGC
AGCGCGGCTATCGTGGCTGGCCACGACGGGCGTTCCTTGCGCAGCTGTGCTCGACGTTGTCA
CTGAAGCGGGAAGGGACTGGCTGCTATTGGGCGAAGTGCCGGGGCAGGATCTCCTGTCATCT
CACCTTGCTCCTGCCGAGAAAGTATCCATCATGGCTGATGCAATGCGGCGGCTGCATACGCT
TGATCCGGCTACCTGCCCATTCGACCACCAAGCGAAACATCGCATCGAGCGAGCACGTACTC
GGATGGAAGCCGGTCTTGTCGATCAGGATGATCTGGACGAAGAGCATCAGGGGCTCGCGCCA
GCCGAACTGTTCGCCAGGCTCAAGGCGCGCATGCCCGACGGCGAGGATCTCGTCGTGACCCA
TGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCT
GAAGAGCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAGCGGGACTCTGGGGTT
CGCGAAATGACCGACCAAGCGACGCCCAACCTGCCATCACGAGATTTCGATTCCACCGCCGC
CTTCTATGAAAGGTTGGGCTTCGGAATCGTTTTCCGGGACGCCGGCTGGATGATCCTCCAGC
GCGGGGATCTCATGCTGGAGTTCTTCGCCCACCCCAACTTGTTTATTGCAGCTTATAATGGT
TACAAATAAAGCAATAGCATCACAAATTTCACAAATAAAGCATTTTTTTCACTGCATTCTAG
TTGTGGTTTGTCCAAACTCATCAATGTATCTTATCATGTCTGTATACCGTCGACCTCTAGCT
AGAGCTTGGCGTAATCATGGTCATAGCTGTTTCCTGTGTGAAATTGTTATCCGCTCACAATT
CCACACAACATACGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTCTTCCGCT
TCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCAATGC
TCACGCTGTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGACAGT
ATTTGGTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTACGCGC
AGAAAAAAAGGATCTCAAGAAGATCCTTTGATCTTTTCTACGGGGTCTGACGCTCAGTGGAA
CGAAAACTCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCAGTCTAT
TAATTGTTGCCGGGAAGCTAGAGTAAGTAGTTCGCCAGTTAATAGTTTGCGCAACGTTGTTG
CCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGAGTAC
TCAACCAAGTCATTCTGAGAATAGTGTATGCGGCGACCGAGTTGCTCTTGCCCGGCGTCAAT
ACGGGATAATACCGCGCCACATAGCAGAACTTTAAAAGTGCTCATCATTGGAAAACGTTCTT
CGGGGCGAAAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACC
TGACGTCGACGGATCGGGAGATCTCCCGATCCCCTATGGTCGACTCTCAGTACAATCTGCTC
TGATGCCGCATAGTTAAGCCAGTATCTGCTCCCTGCTTGTGTGTTGGAGGTCGCTGAGTAGT
GCGCGAGCAAAATTTAAGCTACAACAAGGCAAGGCTTGACCGACAATTGCATGAAGAATCTG
CTTAGG
Sequences of expression vectors for mAb 19G10 IgG4/kappa:
>p19G10.VL.hk
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCAGACATCGTGCTGACACAGAGCCCTGCCAGCCTGGCTGTTTCTCTGGGACAGAGAG
CCACCATCAGCTGCAAGGCCAGCCAGAGCGTTGACTACGACGGCGACAGCTACATGAACTGG
TATCAGCAGAAGTCCGGCCAGCCTCCTAAGCTGCTGATCTACGCCGCCAGCAATCTGGAAAG
CGGCATCCCTGCCAGATTTTCCGGCTCTGGCAGCGGCACCGACTTCACCCTGAATATCCATC
CTGTGGAAGAAGAGGACGCCGCCACCTACTACTGCCAGCAGAGCAATGTGGACCCCTGGACA
TTTGGCGGAGGCACCAAGCTGGAAATCAAGCGTACGGTGGCTGCACCATCTGTCTTCATCTT
CCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAGAGGGAGCTAGCGTGGCAT
CTAGACACTCTCGAGAAGGGTTCGATCCCTACCGGTTAGTAATGAGTTTGATATCTCGACAA
TCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAG
TCGGATCTCCCTTTGGGCCGCCTCCCCGCCTGGAAACGGGGGAGGCTAACTGAAACACGGAA
GGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATAC
CCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACCCTATC
TCGGTCTATTCTTTTGATTTATAAGGGATTTTGGGGATTTCGGCCTATTGGTTAAAAAATGA
GCTGATTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACTCCGCCCAGTTC
CGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAA
AAGCTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAGGCTAT
TCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGAC
TCTGGGGTTCGCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAA
AGTGCCACCTGACGTCGACGGATCGGGAGATCTCCCGATCCCCTATGGTCGACTCTCAGTAC
AATCTGCTCTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGACAATTGCATG
AAGAATCTGCTTAGG
>p19G10.VH.hIgG4
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCGGAGGTCCAGCTGCAGCAGTCTGGCGCCGAACTTGTTAGACCTGGCGCTCTGGTCA
AGCTGAGCTGTAAAGCCAGCGGCTTCAACATCAAGGACTACCACATGCACTGGGTCAAAGAG
CGGCCTGAGCAGGGACTCGAGTGGATCGGATGGATCGACCCCGAGAACGGCAACACTATGTA
CGACCCCAAGTTCCAGGGCAAAGCCAGCATCACCGCCGACACCTCTAGCAACACAGCCTACC
TGCAGCTGAGCAGCCTGACCTCTGAAGATACCGCCGTGTACTACTGCGTGCGGGGAACAGCC
AGAGCCAGCTTTGATTATTGGGGCCAGGGCACCACACTGACCGTGTCATCTGCTAGCACCAA
GGGCCCATCGGTCTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATC
ACAAGCCCAGCAACACCAAGGTGGACAAGAGAGTTGAGTCCAAATATGGTCCCCCATGCCCA
TCATGCCCAGCACCTGAGTTCCTGGGGGGACCATCAGTCTTCCTGTTCCCCCCAAAACCCAA
GGACACTCTCATGATCTCCCGGACCCCTGAGGTCACGTGCGTGGTGGTGGACGTGAGCCAGG
AAGACCCCGAGGTCCAGTTCAACTGGTACGTGGATGGCGTGGAGGTGCATAATGCCAAGACA
AAGCCGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCCGTCCT
CCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTT
CTACCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAA
CCACTACACACAGAAGAGCCTCTCCCTGTCTCTGGGTAAATAACCTAGCGTGGCATCTAGAC
ACTCTCGAGAAGGGTTCGATCCCTACCGGTTAGTAATGAGTTTGATATCTCGACAATCAACC
TCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGT
GGTGTTGTCGGGGAAGCTGACGTCCTTTCCATGGCTGCTCGCCTGTGTTGCCACCTGGATTC
TGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGAT
CTCCCTTTGGGCCGCCTCCCCGCCTGGAAACGGGGGAGGCTAACTGAAACACGGAAGGAGAC
AATACCGGAAGGAACCCGCGCTATGACGGCAATAAAAAGACAGAATAAAACGCACGGGTGTT
GGGTCGTTTGTTCATAAACGCGGGGTTCGGTCCCAGGGCTGGCACTCTGTCGATACCCCACC
GAGACCCCATTGGGGCCAATACGCCCGCGTTTCTTCCTTTTCCCCACCCCACCCCCCAAGTT
CGGGTGAAGGCCCAGGGCTCGCAGCCAACGTCGGGGCGGCAGGCCCTGCCATAGCAGATCTG
CGCAGCTGGGGCTCTAGGGGGTATCCCCACGCGCCCTGTAGCGGCGCATTAAGCGCGGCGGG
TGTGGTGGTTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAATCGGGGC
ATCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGT
CCACGTTCTTTAATAGTGGACTCTTGTTCCAAACTGGAACAACACTCAACCCTATCTCGGTC
TATTCTTTTGATTTATAAGGGATTTTGGGGATTTCGGCCTATTGGTTAAAAAATGAGCTGAT
TTAACAAAAATTTAACGCGAATTAATTCTGTGGAATGTGTGTCAGTTAGGGTGTGGAAAGTC
CCCAGGCTCCCCAGCAGGCAGAAGTATGCAAAGCATGCATCTCAATTAGTCAGCAACCAGGT
GTGGAAAGTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGGCCGCCTCTGCCT
CTGAGCTATTCCAGAAGTAGTGAGGAGGCTTTTTTGGAGGCCTAGGCTTTTGCAAAAAGCTC
CCGGGAGCTTGTATATCCATTTTCGGATCTGATCAAGAGACAGGATGAGGATCGTTTCGCAT
GATTGAACAAGATGGATTGCACGCAGGTTCTCCGGCCGCTTGGGTGGAGAGGCTATTCGGCT
ATGACTGGGCACAACAGACAATCGGCTGCTCTGATGCCGCCGTGTTCCGGCTGTCAGCGCAG
GGGCGCCCGGTTCTTTTTGTCAAGACCGACCTGTCCGGTGCCCTGAATGAACTGCAGGACGA
GGCAGCGCGGCTATCGTGGCTGGCCACGACGGGCGTTCCTTGCGCAGCTGTGCTCGACGTTG
TCACTGAAGCGGGAAGGGACTGGCTGCTATTGGGCGAAGTGCCGGGGCAGGATCTCCTGTCA
TCTCACCTTGCTCCTGCCGAGAAAGTATCCATCATGGCTGATGCAATGCGGCGGCTGCATAC
GCTTGATCCGGCTACCTGCCCATTCGACCACCAAGCGAAACATCGCATCGAGCGAGCACGTA
CTCGGATGGAAGCCGGTCTTGTCGATCAGGATGATCTGGACGAAGAGCATCAGGGGCTCGCG
CCAGCCGAACTGTTCGCCAGGCTCAAGGCGCGCATGCCCGACGGCGAGGATCTCGTCGTGAC
CCATGGCGATGCCTGCTTGCCGAATATCATGGTGGAAAATGGCCGCTTTTCTGGATTCATCG
ACTGTGGCCGGCTGGGTGTGGCGGACCGCTATCAGGACATAGCGTTGGCTACCCGTGATATT
GCTGAAGAGCTTGGCGGCGAATGGGCTGACCGCTTCCTCGTGCTTTACGGTATCGCCGCTCC
CGATTCGCAGCGCATCGCCTTCTATCGCCTTCTTGACGAGTTCTTCTGAGCGGGACTCTGGG
GTTCGCGAAATGACCGACCAAGCGACGCCCAACCTGCCATCACGAGATTTCGATTCCACCGC
CGCCTTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTATAAT
GGTTACAAATAAAGCAATAGCATCACAAATTTCACAAATAAAGCATTTTTTTCACTGCATTC
TAGTTGTGGTTTGTCCAAACTCATCAATGTATCTTATCATGTCTGTATACCGTCGACCTCTA
GCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCAA
TGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCT
GACAGTTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAAAC
CAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCC
ACCTGACGTCGACGGATCGGGAGATCTCCCGATCCCCTATGGTCGACTCTCAGTACAATCTG
CTCTGATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATGAAGAAT
CTGCTTAGG
Sequences of expression vectors for mAb 25F1 IgG4/kappa:
>p25F1.VL.hk
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCAGACATCGTGCTGACACAGAGCCCTGCCAGCCTGGCTGTTTCTCTGGGACAGAGAG
CCACCATCAGCTGCAAGGCCAGCCAGAGCGTTGACTACGACGGCGACACCTACATGAACTGG
TATCAGCAGAAGCCCGGCCAGCCACCTAAGCTGCTGATCTACACAGCCAGCAACCTGGAAAG
CGGCATCCCCGCCAGATTTTCTGGCAGCGGAAGCGGCACCGACTTCACCCTGAATATCCATC
CTGTGGAAGAGGTGGACGCCGCCACCTACTACTGCCAGCAGAGCAATGAGGACCCCTGGACA
TTTGGCGGAGGCACCAAGCTGGAAATCAAGCGTACGGTGGCTGCACCATCTGTCTTCATCTT
CCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAGAGGGAGCTAGCGTGGCAT
CTAGACACTCTCGAGAAGGGTTCGATCCCTACCGGTTAGTAATGAGTTTGATATCTCGACAA
TCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAG
TCGGATCTCCCTTTGGGCCGCCTCCCCGCCTGGAAACGGGGGAGGCTAACTGAAACACGGAA
GGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATAC
CCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACCCTATC
TCGGTCTATTCTTTTGATTTATAAGGGATTTTGGGGATTTCGGCCTATTGGTTAAAAAATGA
GCTGATTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACTCCGCCCAGTTC
CGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAA
AAGCTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAGGCTAT
TCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGAC
TCTGGGGTTCGCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAA
AGTGCCACCTGACGTCGACGGATCGGGAGATCTCCCGATCCCCTATGGTCGACTCTCAGTAC
AATCTGCTCTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGACAATTGCATG
AAGAATCTGCTTAGG
>p20F1.VE.hIgG4
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCGGAGGTCCAGCTGCAGCAGTCTGGCGCCGAACTTGTTAGACCTGGCGCTCTGGTCA
AGTGGTCCTGTAAAGCCAGCGGCTTCAACATCAAGGACTACCACATCCACTGGGTCAAGCAG
AGGCCTGAGCAGGGCCTCGATTGGATCGGCTGGATCGACCCCGAGATCGACAAGACCCTGTA
CGACCCCAAGTTCCAGGGCAAAGCCAGAATCACCGCCGACACCAGCAGCAACACAGCCTACC
TGCAACTGAGCAGCCTGACCAGCGAAGATACCGCCGTGTACTACTGCGCCAGAGGAACAGCC
AGAGCCAGCTTCGATTATTGGGGCCAGGGCACCACACTGACCGTGTCATCTGCTAGCACCAA
GGGCCCATCGGTCTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAG
CAGCGTGGTGACCGTGCCCTCCAGCAGCTTGGGCACGAAGACCTACACCTGCAACGTAGATC
ACAAGCCCAGCAACACCAAGGTGGACAAGAGAGTTGAGTCCAAATATGGTCCCCCATGCCCA
TCATGCCCAGCACCTGAGTTCCTGGGGGGACCATCAGTCTTCCTGTTCCCCCCAAAACCCAA
GGACACTCTCATGATCTCCCGGACCCCTGAGGTCACGTGCGTGGTGGTGGACGTGAGCCAGG
AAGACCCCGAGGTCCAGTTCAACTGGTACGTGGATGGCGTGGAGGTGCATAATGCCAAGACA
AAGCCGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCCGTCCT
CCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTT
CTACCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAA
CCACTACACACAGAAGAGCCTCTCCCTGTCTCTGGGTAAATAACCTAGCGTGGCATCTAGAC
ACTCTCGAGAAGGGTTCGATCCCTACCGGTTAGTAATGAGTTTGATATCTCGACAATCAACC
TCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCGTTGTCAG
GCAACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGT
GGTGTTGTCGGGGAAGCTGACGTCCTTTCCATGGCTGCTCGCCTGTGTTGCCACCTGGATTC
TGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGAT
CTCCCTTTGGGCCGCCTCCCCGCCTGGAAACGGGGGAGGCTAACTGAAACACGGAAGGAGAC
AATACCGGAAGGAACCCGCGCTATGACGGCAATAAAAAGACAGAATAAAACGCACGGGTGTT
GGGTCGTTTGTTCATAAACGCGGGGTTCGGTCCCAGGGCTGGCACTCTGTCGATACCCCACC
GAGACCCCATTGGGGCCAATACGCCCGCGTTTCTTCCTTTTCCCCACCCCACCCCCCAAGTT
CGGGTGAAGGCCCAGGGCTCGCAGCCAACGTCGGGGCGGCAGGCCCTGCCATAGCAGATCTG
CGCAGCTGGGGCTCTAGGGGGTATCCCCACGCGCCCTGTAGCGGCGCATTAAGCGCGGCGGG
TGTGGTGGTTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAATCGGGGC
ATCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGT
CCACGTTCTTTAATAGTGGACTCTTGTTCCAAACTGGAACAACACTCAACCCTATCTCGGTC
TATTCTTTTGATTTATAAGGGATTTTGGGGATTTCGGCCTATTGGTTAAAAAATGAGCTGAT
TTAACAAAAATTTAACGCGAATTAATTCTGTGGAATGTGTGTCAGTTAGGGTGTGGAAAGTC
CCCAGGCTCCCCAGCAGGCAGAAGTATGCAAAGCATGCATCTCAATTAGTCAGCAACCAGGT
GTGGAAAGTCCCCAGGCTCCCCAGCAGGCAGAAGTATGCAAAGCATGCATCTCAATTAGTCA
GCAACCATAGTCCCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGGCCGCCTCTGCCT
CTGAGCTATTCCAGAAGTAGTGAGGAGGCTTTTTTGGAGGCCTAGGCTTTTGCAAAAAGCTC
CCGGGAGCTTGTATATCCATTTTCGGATCTGATCAAGAGACAGGATGAGGATCGTTTCGCAT
GATTGAACAAGATGGATTGCACGCAGGTTCTCCGGCCGCTTGGGTGGAGAGGCTATTCGGCT
ATGACTGGGCACAACAGACAATCGGCTGCTCTGATGCCGCCGTGTTCCGGCTGTCAGCGCAG
GGGCGCCCGGTTCTTTTTGTCAAGACCGACCTGTCCGGTGCCCTGAATGAACTGCAGGACGA
GGCAGCGCGGCTATCGTGGCTGGCCACGACGGGCGTTCCTTGCGCAGCTGTGCTCGACGTTG
TCACTGAAGCGGGAAGGGACTGGCTGCTATTGGGCGAAGTGCCGGGGCAGGATCTCCTGTCA
TCTCACCTTGCTCCTGCCGAGAAAGTATCCATCATGGCTGATGCAATGCGGCGGCTGCATAC
GCTTGATCCGGCTACCTGCCCATTCGACCACCAAGCGAAACATCGCATCGAGCGAGCACGTA
CTCGGATGGAAGCCGGTCTTGTCGATCAGGATGATCTGGACGAAGAGCATCAGGGGCTCGCG
CCAGCCGAACTGTTCGCCAGGCTCAAGGCGCGCATGCCCGACGGCGAGGATCTCGTCGTGAC
CCATGGCGATGCCTGCTTGCCGAATATCATGGTGGAAAATGGCCGCTTTTCTGGATTCATCG
ACTGTGGCCGGCTGGGTGTGGCGGACCGCTATCAGGACATAGCGTTGGCTACCCGTGATATT
GCTGAAGAGCTTGGCGGCGAATGGGCTGACCGCTTCCTCGTGCTTTACGGTATCGCCGCTCC
CGATTCGCAGCGCATCGCCTTCTATCGCCTTCTTGACGAGTTCTTCTGAGCGGGACTCTGGG
GTTCGCGAAATGACCGACCAAGCGACGCCCAACCTGCCATCACGAGATTTCGATTCCACCGC
CGCCTTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTATAAT
GGTTACAAATAAAGCAATAGCATCACAAATTTCACAAATAAAGCATTTTTTTCACTGCATTC
TAGTTGTGGTTTGTCCAAACTCATCAATGTATCTTATCATGTCTGTATACCGTCGACCTCTA
GCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCAA
TGCTCACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCT
GACAGTTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAAAC
CAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCC
ACCTGACGTCGACGGATCGGGAGATCTCCCGATCCCCTATGGTCGACTCTCAGTACAATCTG
CTCTGATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATGAAGAAT
CTGCTTAGG
Sequences of expression vectors for mAb 7C11.1 IgG2/kappa:
>p7C11.1.VL.hk
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCAGACAACGTGCTGACACAGAGCCCTGCCAGCCTGGCTGTTTCTCTGGGACAGAGAG
CCACCATCAGCTGCAAGGCCAGCCAGAGCGTTGACTACGACGGCGACAGCTACATGAACTGG
TATCAGCAGAAGCCCGGCCAGCCACCTAAGGTGTTCATCTACGCCGCCAGCAACCTGGAAAG
CGGCATCCCTGCCAGATTTTCTGGCTCTGGCAGCGGCACCAACTTCACCCTGAACATTCACC
CCGTGGAAGAAGAGGACGCCGCCACCTACTACTGCCAGCAGAGCAATGAGGACCCCTGGACA
TTTGGCGGAGGCACCAAGCTGGAAATCAAGCGTACGGTGGCTGCACCATCTGTCTTCATCTT
CCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAGAGGGAGCTAGCGTGGCAT
CTAGACACTCTCGAGAAGGGTTCGATCCCTACCGGTTAGTAATGAGTTTGATATCTCGACAA
TCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGGCACTGACAA
TTCCGTGGTGTTGTCGGGGAAGCTGACGTCCTTTCCATGGCTGCTCGCCTGTGTTGCCACCT
GGATTCTGCGCGGGACGTCCTTCTGCTACGTCCCTTCGGCCCTCAATCCAGCGGACCTTCCT
TCCCGCGGCCTGCTGCCGGCTCTGCGGCCTCTTCCGCGTCTTCGCCTTCGCCCTCAGACGAG
TCGGATCTCCCTTTGGGCCGCCTCCCCGCCTGGAAACGGGGGAGGCTAACTGAAACACGGAA
GGAGACAATACCGGAAGGAACCCGCGCTATGACGGCAATAAAAAGACAGAATAAAACGCACG
GGTGTTGGGTCGTTTGTTCATAAACGCGGGGTTCGGTCCCAGGGCTGGCACTCTGTCGATAC
CCCACCGAGACCCCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGGCGCATTAAGCGC
GGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGA
TTAGGGTGATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCAACCCTATC
TCGGTCTATTCTTTTGATTTATAAGGGATTTTGGGGATTTCGGCCTATTGGTTAAAAAATGA
GCTGATTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACTCCGCCCAGTTC
CGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAA
AAGCTCCCGGGAGCTTGTATATCCATTTTCGGATCTGATCAAGAGACAGGATGAGGATCGTT
TCGCATGATTGAACAAGATGGATTGCACGCAGGTTCTCCGGCCGCTTGGGTGGAGAGGCTAT
TCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAGCGAG
CACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGAT
TCATCGACTGTGGCCGGCTGGGTGTGGCGGACCGCTATCAGGACATAGCGTTGGCTACCCGT
GATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGAC
TCTGGGGTTCGCGAAATGACCGACCAAGCGACGCCCAACCTGCCATCACGAGATTTCGATTC
CACCGCCGCCTTCTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATAC
TCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAA
AGTGCCACCTGACGTCGACGGATCGGGAGATCTCCCGATCCCCTATGGTCGACTCTCAGTAC
AATCTGCTCTGATGCCGCATAGTTAAGCCAGTATCTGCTCCCTGCTTGTGTGTTGGAGGTCG
CTGAGTAGTGCGCGAGCAAAATTTAAGCTACAACAAGGCAAGGCTTGACCGACAATTGCATG
AAGAATCTGCTTAGG
>p7C11.1.VH.hIgG2
GTTAGGCGTTTTGCGCTGCTTCGCGATGTACGGGCCAGATATACGCGTTGACATTGATTATT
GACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAA
TGTCGTAACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTGTTTTG
ACCTCCATAGAAGACACCGGGACCGATCCAGCCTCCGGACTCTAGAGGATCGAACCCTTAAG
CTTGCCACCATGTACAGGATGCAACTCCTGTCTTGCATTGCACTAAGTCTTGCACTTGTCAC
GAATTCGGAAGTGAAGCTGGTGGAAAGCGGCGGAGGACTGGTTCAACCTGGCGGATCTCTGA
AGCTGAGCTGTGCCGCCAGCGGCTTCACCTTCAGCAGATACACAATGAGCTGGGTCCGACAG
ACCCCTGAGAAGAGACTGGAATGGGTCGCCAAGATCAGAAACGTCGGCGGCATCACCTACTA
TCCCGACACCGTGAAGGGCAGATTCACCATCTCCAGAGACAACGCCAAGAACACCCTGTACC
TGCAGATGAGCAGCCTGAAGTCCGAGGACACCGCCATGTACTACTGCGCCAGACACTACTAC
GGCAGCGAGGACTACTTCGACTATTGGGGCCAGGGCACCACACTGACCGTTAGCTCTGCTAG
CACCAAGGGCCCATCGGTCTTCCCCCTGGCGCCCTGCTCCAGGAGCACCTCCGAGAGCACAG
CGGCCCTGGGCTGCCTGGTCAAGGACTACTTCCCCGAACCGGTGACGGTGTCGTGGAACTCA
GGCGCTCTGACCAGCGGCGTGCACACCTTCCCAGCTGTCCTACAGTCCTCAGGACTCTACTC
CCTCAGCAGCGTGGTGACCGTGCCCTCCAGCAACTTCGGCACCCAGACCTACACCTGCAACG
TAGATCACAAGCCCAGCAACACCAAGGTGGACAAGACAGTTGAGCGCAAATGTTGTGTCGAG
TGCCCACCGTGCCCAGCACCACCTGTGGCAGGACCGTCAGTCTTCCTCTTCCCCCCAAAACC
CAAGGACACCCTCATGATCTCCCGGACCCCTGAGGTCACGTGCGTGGTGGTGGACGTGAGCC
ACGAAGACCCCGAGGTCCAGTTCAACTGGTACGTGGACGGCGTGGAGGTGCATAATGCCAAG
ACAAAGCCACGGGAGGAGCAGTTCAACAGCACGTTCCGTGTGGTCAGCGTCCTCACCGTTGT
GCACCAGGACTGGCTGAACGGCAAGGAGTACAAGTGCAAGGTCTCCAACAAAGGCCTCCCAG
CCCCCATCGAGAAAACCATCTCCAAAACCAAAGGGCAGCCCCGAGAACCACAGGTGTACACC
CTGCCCCCATCCCGGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGGAGAACAACTACA
AGACCACGCCTCCCATGCTGGACTCCGACGGCTCCTTCTTCCTCTACAGCAAGCTCACCGTG
GACAAGAGCAGGTGGCAGCAGGGGAACGTCTTCTCATGCTCCGTGATGCATGAGGCTCTGCA
CAACCACTACACGCAGAAGAGCCTCTCCCTGTCTCCGGGTAAATGACCTAGCGTGGCATCTA
GACACTCTCGAGAAGGGTTCGATCCCTACCGGTTAGTAATGAGTTTGATATCTCGACAATCA
ACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTC
ATTTTCTCCTCCTTGTATAAATCCTGGTTGCTGTCTCTTTATGAGGAGTTGTGGCCCGTTGT
CAGGCAACGTGGCGTGGTGTGCACTGTGTTTGCTGACGCAACCCCCACTGGTTGGGGCATTG
CCACCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAATTC
CGTGGTGTTGTCGGGGAAGCTGACGTCCTTTCCATGGCTGCTCGCCTGTGTTGCCACCTGGA
TTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCG
GATCTCCCTTTGGGCCGCCTCCCCGCCTGGAAACGGGGGAGGCTAACTGAAACACGGAAGGA
GACAATACCGGAAGGAACCCGCGCTATGACGGCAATAAAAAGACAGAATAAAACGCACGGGT
GTTGGGTCGTTTGTTCATAAACGCGGGGTTCGGTCCCAGGGCTGGCACTCTGTCGATACCCC
ACCGAGACCCCATTGGGGCCAATACGCCCGCGTTTCTTCCTTTTCCCCACCCCACCCCCCAA
GTTCGGGTGAAGGCCCAGGGCTCGCAGCCAACGTCGGGGCGGCAGGCCCTGCCATAGCAGAT
CTGCGCAGCTGGGGCTCTAGGGGGTATCCCCACGCGCCCTGTAGCGGCGCATTAAGCGCGGC
GGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGG
GGCATCCCTTTAGGGTTCCGATTTAGTGCTTTACGGCACCTCGACCCCAAAAAACTTGATTA
GGGTGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACCCTATCTCG
GTCTATTCTTTTGATTTATAAGGGATTTTGGGGATTTCGGCCTATTGGTTAAAAAATGAGCT
GATTTAACAAAAATTTAACGCGAATTAATTCTGTGGAATGTGTGTCAGTTAGGGTGTGGAAA
GTCCCCAGGCTCCCCAGCAGGCAGAAGTATGCAAAGCATGCATCTCAATTAGTCAGCAACCA
GGTGTGGAAAGTCCCCAGGCTCCCCAGCAGGCAGAAGTATGCAAAGCATGCATCTCAATTAG
TCAGCAACCATAGTCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGCCGCCTCTG
CCTCTGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAAGAGACAGGATGAGGATCGTTTCG
CATGATTGAACAAGATGGATTGCACGCAGGTTCTCCGGCCGCTTGGGTGGAGAGGCTATTCG
GCTATGACTGGGCACAACAGACAATCGGCTGCTCTGATGCCGCCGTGTTCCGGCTGTCAGCG
CAGGGGCGCCCGGTTCTTTTTGTCAAGACCGACCTGTCCGGTGCCCTGAATGAACTGCAGGA
CGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTG
TCATCTCACCTTGCTCCTGCCGAGAAAGTATCCATCATGGCTGATGCAATGCGGCGGCTGCA
TACGCTTGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAGGGGCTC
GCGCCAGCCGAACTGTTCGCCAGGCTCAAGGCGCGCATGCCCGACGGCGAGGATCTCGTCGT
GACCCATGGCGATGCCTGCTTGCCGAATATCATGGTGGAAAATGGCCGCTTTTCTGGATTCA
TCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGC
TCCCGATTCGCAGCGCATCGCCTTCTATCGCCTTCTTGACGAGTTCTTCTGAGCGGGACTCT
GGGGTTCGCGAAATGACCGACCAAGCGACGCCCAACCTGCCATCACGAGATTTCGATTCCAC
CGCCGCCTTCTATGAAAGGTTGGGCTTCGGAATCGTTTTCCGGGACGCCGGCTGGATGATCC
TCCAGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGACCT
CTAGCTAGAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGT
GAGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCT
CAATGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGG
TCTGACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCA
GTCTATTAATTGTTGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCA
TACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGT
GCCACCTGACGTCGACGGATCGGGAGATCTCCCGATCCCCTATGGTCGACTCTCAGTACAAT
CTGCTCTGATGCCGCATAGTTAAGCCAGTATCTGCTCCCTGCTTGTGTGTTGGAGGTCGCTG
AGTAGTGCGCGAGCAAAATTTAAGCTACAACAAGGCAAGGCTTGACCGACAATTGCATGAAG
AATCTGCTTAGG

### REFERENCES

1. Lendon, C. L., et al. E280A PS-1 mutation causes Alzheimer's disease but age of onset is not modified by ApoE alleles. Hum Mutat 10, 186-195 (1997).
2. Aguirre-Acevedo, D. C., et al. [Validity and reliability of the CERAD-Col neuropsychological battery]. Rev Neurol 45, 655-660 (2007).
3. Yesavage, J. A. Opportunities for and obstacles to treatments for dementias. J Am Geriatr Soc 31, 59-60 (1983).
4. Reisberg, B. Functional assessment staging (FAST). Psychopharmacol Bull 24, 653-659 (1988).
5. Mahley, R. W., Huang, Y. & Rall, S. C., Jr. Pathogenesis of type III hyperlipoproteinemia (dysbetalipoproteinemia). Questions, quandaries, and paradoxes. J Lipid Res 40, 1933-1949 (1999).
6. Fisher, S., et al. A scalable, fully automated process for construction of sequence-ready human exome targeted capture libraries. Genome Biol 12, R1 (2011).
7. Lek, M., et al. Analysis of protein-coding genetic variation in 60,706 humans. Nature 536, 285-291 (2016).
8. Li, H. Aligning sequence reads, clone sequences and assembly contigs with BWA-MEM. .arXiv:1303.3997v1 [q-bio.GN] (2013).
9. Kohler, S., et al. The Human Phenotype Ontology project: linking molecular biology and disease through phenotype data. Nucleic Acids Res 42, D966-974 (2014).
10. Smedley, D., et al. A Whole-Genome Analysis Framework for Effective Identification of Pathogenic Regulatory Variants in Mendelian Disease. Am J Hum Genet 99, 595-606 (2016).
11. Chen, J., Li, Q. & Wang, J. Topology of human apolipoprotein E3 uniquely regulates its diverse biological functions. Proc Natl Acad Sci USA 108, 14813-14818 (2011).
12. Rose, A. S., et al. NGL viewer: web-based molecular graphics for large complexes. Bioinformatics 34, 3755-3758 (2018).
13. Zhong, L., et al. A rapid and cost-effective method for genotyping apolipoprotein E gene polymorphism. Mol Neurodegener 11, 2 (2016).
14. Quiroz, Y. T., et al. Association Between Amyloid and Tau Accumulation in Young Adults With Autosomal Dominant Alzheimer Disease. JAMA Neurol (2018).
15. Fleisher, A. S., et al. Associations between biomarkers and age in the presenilin 1 E280A autosomal dominant Alzheimer disease kindred: a cross-sectional study. JAMA Neurol 72, 316-324 (2015).
16. Becker, J. A., et al. Amyloid-associated cortical thinning in clinically normal elderly. Ann Neurol 69, 1032-1042 (2011).
17. Braak, H., Rüb, U., Schultz, C. & Del Tredici, K. Vulnerability of cortical neurons to Alzheimer's and Parkinson's diseases. J Alzheimers Dis 9, 35-44 (2006).
18. Braak, H. & Braak, E. Diagnostic criteria for neuropathologic assessment of Alzheimer's disease. Neurobiol Aging 18, S85-88 (1997).
19. Johnson, K. A., et al. Tau positron emission tomographic imaging in aging and early Alzheimer disease. Ann Neurol 79, 110-119 (2016).
20. Chien, D. T., et al. Early clinical PET imaging results with the novel PHF-tau radioligand [F-18]-T807. J Alzheimers Dis 34, 457-468 (2013).
21. Wang, L., et al. Evaluation of Tau Imaging in Staging Alzheimer Disease and Revealing Interactions Between beta-Amyloid and Tauopathy. JAMA Neurol 73, 1070-1077 (2016).
22. Logan, J., et al. Graphical analysis of reversible radioligand binding from time-activity measurements applied to [N-11C-methyl]-(−)-cocaine PET studies in human subjects. J Cereb Blood Flow Metab 10, 740-747 (1990).
23. Amariglio, R. E., et al. Subjective cognitive concerns, amyloid-β, and neurodegeneration in clinically normal elderly. Neurology 85, 56-62 (2015).
24. Hedden, T., et al. Disruption of functional connectivity in clinically normal older adults harboring amyloid burden. J Neurosci 29, 12686-12694 (2009).
25. Beecham, G. W., et al. Genome-wide association meta-analysis of neuropathologic features of Alzheimer's disease and related dementias. PLOS Genet 10, e1004606 (2014).
26. Hashimoto, T., et al. Apolipoprotein E, especially apolipoprotein E4, increases the oligomerization of amyloid beta peptide. J Neurosci 32, 15181-15192 (2012).
27. Hudry, E., et al. Gene transfer of human Apoe isoforms results in differential modulation of amyloid deposition and neurotoxicity in mouse brain. Sci Transl Med 5, 212ra161 (2013).
28. Lemere, C. A., et al. The E280A presenilin 1 Alzheimer mutation produces increased A beta 42 deposition and severe cerebellar pathology. Nat Med 2, 1146-1150 (1996).
29. Quiroz, Y. T., et al. Association Between Amyloid and Tau Accumulation in Young Adults With Autosomal Dominant Alzheimer Disease. JAMA Neurol (2018).
30. Acosta-Baena, N., et al. Pre-dementia clinical stages in presenilin 1 E280A familial early-onset Alzheimer's disease: a retrospective cohort study. Lancet Neurol 10, 213-220 (2011).
31. Lopera, F., et al. Clinical features of early-onset Alzheimer disease in a large kindred with an E280A presenilin-1 mutation. JAMA 277, 793-799 (1997).
32. Cacace, R., Sleegers, K. & Van Broeckhoven, C. Molecular genetics of early-onset Alzheimer's disease revisited. Alzheimers Dement 12, 733-748 (2016).
33. Albert, M. S., et al. The diagnosis of mild cognitive impairment due to Alzheimer's disease: recommendations from the National Institute on Aging-Alzheimer's Association workgroups on diagnostic guidelines for Alzheimer's disease. Alzheimers Dement 7, 270-279 (2011).
34. Smedley, D., et al. A Whole-Genome Analysis Framework for Effective Identification of Pathogenic Regulatory Variants in Mendelian Disease. Am J Hum Genet 99, 595-606 (2016).
35. Corder, E. H., et al. Protective effect of apolipoprotein E type 2 allele for late onset Alzheimer disease. Nat Genet 7, 180-184 (1994).
36. Corder, E. H., et al. Gene dose of apolipoprotein E type 4 allele and the risk of Alzheimer's disease in late onset families. Science 261, 921-923 (1993).
37. Farrer, L. A., et al. Effects of age, sex, and ethnicity on the association between apolipoprotein E genotype and Alzheimer disease. A meta-analysis. APOE and Alzheimer Disease Meta Analysis Consortium. JAMA 278, 1349-1356 (1997).
38. Wardell, M. R., Brennan, S. O., Janus, E. D., Fraser, R. & Carrell, R. W. Apolipoprotein E2-Christchurch (136 Arg - - - Ser). New variant of human apolipoprotein E in a patient with type III hyperlipoproteinemia. J Clin Invest 80, 483-490 (1987).
39. Candas-Estebanez, B., et al. APOE Variants E2, E3, and E4 Can Be Miscalled By Classical PCR-RFLP When The Christchurch Variant Is Also Present. J Clin Lab Anal 31(2017).
40. Wardell M R, Brennan S O, Janus E D, Fraser R, Carrell R W. Apolipoprotein E2-Christchurch (136 Arg - - - Ser). New variant of human apolipoprotein E in a patient with type III hyperlipoproteinemia. J Clin Invest 1987; 80:483-90.
41. Velez J I, Lopera F, Sepulveda-Falla D, et al. APOE*E2 allele delays age of onset in PSEN1 E280A Alzheimer's disease. Mol Psychiatry 2016; 21:916-24.
42. Hollingworth P, Harold D, Sims R, et al. Common variants at ABCA7, MS4A6A/MS4A4E, EPHA1, CD33 and CD2AP are associated with Alzheimer's disease. Nat Genet 2011; 43:429-35.
43. Wardell, M. R., Brennan, S. O., Janus, E. D., Fraser, R. & Carrell, R. W. Apolipoprotein E2-Christchurch (136 Arg - - - Ser). New variant of human apolipoprotein E in a patient with type III hyperlipoproteinemia. J Clin Invest 80, 483-490 (1987).
44. Mahley, R. W., Huang, Y. & Rall, S. C., Jr. Pathogenesis of type III hyperlipoproteinemia (dysbetalipoproteinemia). Questions, quandaries, and paradoxes. J Lipid Res 40, 1933-1949 (1999).
45. Mahley R W. Apolipoprotein E: from cardiovascular disease to neurodegenerative disorders. J Mol Med (Berl) 2016; 94:739-46.
46. Romeo S, Pennacchio L A, Fu Y, et al. Population-based resequencing of ANGPTLA uncovers variations that reduce triglycerides and increase HDL. Nat Genet 2007; 39:513-6.
47. Mahley, R. W. Apolipoprotein E: from cardiovascular disease to neurodegenerative disorders. J Mol Med (Berl) 94, 739-746 (2016).
48. Hashimoto, T., et al. Apolipoprotein E, especially apolipoprotein E4, increases the oligomerization of amyloid beta peptide. J Neurosci 32, 15181-15192 (2012).
49. Walsh, D. M., et al. Naturally secreted oligomers of amyloid beta protein potently inhibit hippocampal long-term potentiation in vivo. Nature 416, 535-539 (2002).
50. Lalli, M. A., et al. Whole-genome sequencing suggests a chemokine gene cluster that modifies age at onset in familial Alzheimer's disease. Mol Psychiatry 20, 1294-1300 (2015).
51. Huynh, T. V., et al. Age-Dependent Effects of apoE Reduction Using Antisense Oligonucleotides in a Model of beta-amyloidosis. Neuron 96, 1013-1023 e1014 (2017).
52. Huynh, T. V., et al. Age-Dependent Effects of apoE Reduction Using Antisense Oligonucleotides in a Model of beta-amyloidosis. Neuron 96, 1013-1023 e1014 (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cognitive decline associated with dementia and/or mild cognitive impairment in a human subject in need thereof, comprising administering to the human subject a therapeutically effective amount of an antibody or an antigen-binding portion thereof; wherein the antibody or an antigen-binding portion thereof comprises:
(a) a heavy chain variable region (VH) that comprises:
(i) a heavy chain CDR1 (HCDR1) sequence according to any one of SEQ ID NOs: 7, 17, 27, 37 and 47,
(ii) a HCDR2 sequence according to any one of SEQ ID NOs: 8, 18, 28, 38 and 48, and
(iii) a HCDR3 sequence according to any one of SEQ ID NOs: 9 and 49; and
(b) a light chain variable region (VL) that comprises:
(i) a light chain CDR1 (LCDR1) sequence according to any one of SEQ ID NOs: 4, 34 and 44,
(ii) a LCDR2 sequence according to any one of SEQ ID NOs: 5, 35 and 45, and
(iii) a LCDR3 sequence according to any one of SEQ ID NOs: 6, 26, and 46.

2. The method of claim 1, wherein the antibody is a humanized or chimeric antibody.

3. The method of claim 1, wherein the antibody is a monoclonal antibody, or wherein the antigen binding fragment thereof is a single-chain Fv (scFv)nanobody, a Fab, a Fab′ or a F(ab′)2.

4. The method of claim 1, wherein the antibody is a monoclonal antibody that comprises (i) a mouse IgG1, IgG2a, IgG2b, IgG2c, or IgG3 heavy chain constant region, or an Fc-modified variant thereof with reduced effector function, or (ii) a human IgG1, IgG2, IgG3, or IgG4 heavy chain constant region, or an Fc-modified variant thereof with reduced effector function.

5. The method of claim 1, wherein the antibody or antigen-binding portion thereof comprises:
(a) a VH sequence according to:
(i) amino acids 20-139 of SEQ ID NO: 13;
(ii) amino acids 20-139 of SEQ ID NO: 22;
(iii) amino acids 20-137 of SEQ ID NO: 33;
(iv) amino acids 20-137 of SEQ ID NO: 43; or
(v) amino acids 1-118 of SEQ ID NO: 53; and
(b) a VL sequence according to:
(i) amino acids 21-131 of SEQ ID NO: 12;
(ii) amino acids 21-131 of SEQ ID NO: 23;
(iii) amino acids 21-131 of SEQ ID NO: 32;
(iv) amino acids 21-131 of SEQ ID NO: 42; or
(v) amino acids 1-111 of SEQ ID NO: 52.

6. The method of claim 1, wherein the antibody or an antigen-binding portion thereof comprises:
(a) a VH that comprises:
(i) a HCDR1 sequence according to SEQ ID NO: 7,
(ii) a HCDR2 sequence according to SEQ ID NO: 8, and
(iii) a HCDR3 sequence according to SEQ ID NO: 9; and
(b) a VL that comprises:
(i) a LCDR1 sequence according to SEQ ID NO: 4,
(ii) a LCDR2 sequence according to SEQ ID NO: 5, and
(iii) a LCDR3 sequence according to SEQ ID NO: 6.

7. The method of claim 1, wherein the antibody or an antigen-binding portion thereof comprises:
(a) a VH that comprises:
(i) a HCDR1 sequence according to SEQ ID NO: 17,
(ii) a HCDR2 sequence according to SEQ ID NO: 18, and
(iii) a HCDR3 sequence according to SEQ ID NO: 9; and
(b) a VL that comprises:
(i) a LCDR1 sequence according to SEQ ID NO: 4,
(ii) a LCDR2 sequence according to SEQ ID NO: 5, and
(iii) a LCDR3 sequence according to SEQ ID NO: 6.

8. The method of claim 1, wherein the antibody or an antigen-binding portion thereof comprises:
(a) a VH that comprises:
(i) a HCDR1 sequence according to SEQ ID NO: 27,
(ii) a HCDR2 sequence according to SEQ ID NO: 28, and
(iii) a HCDR3 sequence according to SEQ ID NO: 49; and
(b) a VL that comprises:
(i) a LCDR1 sequence according to SEQ ID NO: 4,
(ii) a LCDR2 sequence according to SEQ ID NO: 5, and
(iii) a LCDR3 sequence according to SEQ ID NO: 26.

9. The method of claim 1, wherein the antibody or an antigen-binding portion thereof comprises:
(a) a VH that comprises:
(i) a HCDR1 sequence according to SEQ ID NO: 37,
(ii) a HCDR2 sequence according to SEQ ID NO: 38, and
(iii) a HCDR3 sequence according to SEQ ID NO: 49; and
(b) a VL that comprises:
(i) a LCDR1 sequence according to SEQ ID NO: 34,
(ii) a LCDR2 sequence according to SEQ ID NO: 35, and
(iii) a LCDR3 sequence according to SEQ ID NO: 6.

10. The method of claim 1, wherein the antibody or an antigen-binding portion thereof comprises:
(a) a VH that comprises:
(i) a HCDR1 sequence according to SEQ ID NO: 47,
(ii) a HCDR2 sequence according to SEQ ID NO: 48, and
(iii) a HCDR3 sequence according to SEQ ID NO: 49; and
(b) a VL that comprises:
(i) a LCDR1 sequence according to SEQ ID NO: 44,
(ii) a LCDR2 sequence according to SEQ ID NO: 45, and
(iii) a LCDR3 sequence according to SEQ ID NO: 46.

11. The method of claim 1, wherein the antibody or an antigen-binding portion thereof comprises:
(a) a VH sequence with at least 80% sequence identity to amino acids 20-139 of SEQ ID NO: 13; and
(b) a VL sequence with at least 80% sequence identity to amino acids 21-131 of SEQ ID NO: 12.

12. The method of claim 1, wherein the antibody or an antigen-binding portion thereof comprises:
(I) (a) a VH sequence with at least 80% sequence identity to amino acids 20-139 of SEQ ID NO: 33; and
(b) a VL sequence with at least 80% sequence identity to amino acids 21-131 of SEQ ID NO: 32;
(II) (a) a VH sequence with at least 80% sequence identity to amino acids 20-139 of SEQ ID NO: 43; and
(b) a VL sequence with at least 80% sequence identity to amino acids 21-131 of SEQ ID NO: 42; or
(III) (a) a VH sequence with at least 80% sequence identity to amino acids 20-139 of SEQ ID NO: 53; and
(b) a VL sequence with at least 80% sequence identity to amino acids 21-131 of SEQ ID NO: 52.

13. The method of claim 1, wherein the antibody or an antigen-binding portion thereof comprises:
(a) a VH sequence with at least 80% sequence identity to amino acids 20-139 of SEQ ID NO: 22; and
(b) a VL sequence with at least 80% sequence identity to amino acids 21-131 of SEQ ID NO: 23.

14. The method of claim 1, wherein the antibody or an antigen-binding portion thereof exhibits at least one of the following properties:
(i) binds to an ApoE3 protein comprising the amino acid sequence set forth in SEQ ID NO:3 with a K_{D} of 20 nM or less, wherein the K_{D} is measured by surface plasmon resonance (Biacore) analysis;
(ii) binds to an ApoE3 protein comprising the amino acid sequence set forth in SEQ ID NO:3 with an on rate (Kon) of 3×10⁵ l/Ms or more, wherein the on rate (Kon) is measured by surface plasmon resonance (Biacore) analysis; and
(iii) binds to an ApoE3 protein comprising the amino acid sequence set forth in SEQ ID NO:3 with an off rate (koff) of about 1×10⁴ l/s or less, wherein the off rate (koff) is measured by surface plasmon resonance (Biacore) analysis.

15. The method of claim 1, wherein the subject has a disease, condition or disorder selected from the group consisting of: Alzheimer's disease, dementia, vascular cognitive impairment, vascular dementia, cerebral autosomal dominant arteriopathy with subcortical infarcts and leukoencephalopathy (CADASIL), cerebral autosomal recessive arteriopathy with subcortical infarcts and leukoencephalopathy (CARASIL), Lewy body dementia, frontotemporal dementia, amyotrophic lateral sclerosis, multiple sclerosis, Parkinson's disease, Huntington's disease, a neurodegenerative disease, a cerebrovascular disease, brain injury, chronic traumatic encephalopathy, a tauopathy, an amyloidopathy, a synucleinopathy, Creutzfeldt-Jakob disease, retinal degeneration, age-related macular degeneration, glaucoma, retinal injury, nerve degeneration, and aging.

16. The method of claim 15, wherein the subject has Alzheimer's disease.

17. The method of claim 1, wherein the subject has a genetic variation in a gene selected from the group consisting of APOE4, APOE3, PSEN1, PSEN2, APP, MAPT, and any combination thereof.

18. The method of claim 16, wherein the human subject:
(a) has an APOE gene with a first allele encoding an ApoE3 protein with an R136S mutation and a second allele encoding an ApoE3 protein without an R136S mutation,
(b) has an APOE gene with a first allele encoding an ApoE3 without an R136S mutation and a second allele encoding an ApoE3 protein without an R136S mutation, or
(c) does not have an APOE gene with a first allele encoding an ApoE3 protein without an R136S mutation and a second allele encoding an ApoE3 protein without an R136S mutation.

19. A method of method of treating cognitive decline associated with dementia and/or mild cognitive impairment in a human subject in need thereof, comprising administering to the human subject a therapeutically effective amount of an antibody or an antigen-binding portion thereof; wherein the antibody or an antigen-binding portion thereof comprises:
(I) (a) a heavy chain variable region (VH) that comprises:
(i) a HCDR1 sequence according to the sequence X₁YX₃X₄X₅, wherein X₁ is S, R or D, X₃ is T or H, X₄ is I or M, X₅ is S or H,
(ii) a HCDR2 sequence according to:
(A) the sequence KIRNX₅GGITYYX₁₂DTX₁₅ KG, wherein X₅ is G or V, X₁₁ is L or P, and X₁₅ is V or L,
(B) SEQ ID NO: 48, or
(C) the sequence WIDPEX₆X₇X₈TX₁₀YDPKFQG, wherein X₆ is N or I, X₇ is G or D, and X₈ is N or K, and X₁₀ is M or L, and
(iii) a HCDR3 sequence according to any one of SEQ ID NOs: 9 and 49; and
(b) a light chain variable region (VL) that comprises:
(i) a light chain CDR1 (LCDR1) sequence according to the sequence KASQSVDYDGX₁₁X₁₂YMN, wherein X₁₁ is D or E, and X₁₂ is T, N or S,
(ii) a LCDR2 sequence according to the sequence X₁ASNLES, wherein X₁ is V, A or T, and
iii) a LCDR3 sequence according to the sequence QQSNX₅DPWT, wherein X₅ is E, V or L.

20. The method of claim 19, wherein the antibody or an antigen-binding portion thereof comprises:
(a) a VH that comprises:
(i) a HCDR1 sequence according to the sequence X₁YX₃X₄X₅, wherein X₁ is S, R or D, X₃ is T or H, X₄ is I or M, X₅ is S or H,
(ii) a HCDR2 sequence according to the sequence KIRNX₅GGITYYX₁₂DTX₁₅ KG, wherein X₅ is G or V, X₁₁ is L or P, and X₁₅ is V or L, and
(iii) a HCDR3 sequence according to SEQ ID NO: 9; and
(b) a VL that comprises:
(i) a light chain CDR1 (LCDR1) sequence according to the sequence KASQSVDYDGX₁₁X₁₂YMN, wherein X₁₁ is D or E, and X₁₂ is T, N or S,
(ii) a LCDR2 sequence according to the sequence X₁ASNLES, wherein X₁ is V, A or T, and
(iii) a LCDR3 sequence according to the sequence QQSNX₅DPWT, wherein X₅ is E, V or L.

21. The method of claim 19, wherein the antibody or an antigen-binding portion thereof comprises:
(a) a VH that comprises:
(i) a HCDR1 sequence according to the sequence X₁YX₃X₄X₅, wherein X₁ is S, R or D, X₃ is T or H, X₄ is I or M, X₅ is S or H,
(ii) a HCDR2 sequence according to SEQ ID NO: 48 or according to the sequence WIDPEX₆X₇X₈TX₁₀YDPKFQG, wherein X₆ is N or I, X₇ is G or D, and X₈ is Nor K, and X₁₀ is M or L, and
(iii) a HCDR3 sequence according to SEQ ID NO: 49; and
(b) a VL that comprises:
(i) a LCDR1 sequence according to the sequence KASQSVDYDGX₁₁X₁₂YMN, wherein X₁₁ is D or E, and X₁₂ is T, N or S,
(ii) a LCDR2 sequence according to the sequence X₁ASNLES, wherein X₁ is V, A or T, and
(iii) a LCDR3 sequence according to the sequence QQSNX₅DPWT, wherein X₅ is E, V or L.

22. The method of claim 19, wherein the antibody or an antigen-binding portion thereof comprises:
(a) a VH that comprises:
(i) a HCDR1 sequence according to any one of SEQ ID NOs: 7, 17, 27, 37 and 47,
(ii) a HCDR2 sequence according to any one of SEQ ID NOs: 8, 18, 28, 38 and 48, and
(iii) a HCDR3 sequence according to any one of SEQ ID NOs: 9 and 49; and
(b) a VL that comprises:
(i) a LCDR1 sequence according to any one of SEQ ID NOs: 4, 34 and 44,
(ii) a LCDR2 sequence according to any one of SEQ ID NOs: 5, 35 and 45, and
(iii) a LCDR3 sequence according to any one of SEQ ID NOs: 6, 26 and 46.

23. The method of claim 22, wherein the antibody or an antigen-binding portion thereof comprises:
(a) a VH that comprises:
(i) a HCDR1 sequence according to SEQ ID NO: 17,
(ii) a HCDR2 sequence according to SEQ ID NO: 18, and
(iii) a HCDR3 sequence according to SEQ ID NO: 9; and
(b) a VL that comprises:
(i) a LCDR1 sequence according to SEQ ID NO: 4,
(ii) a LCDR2 sequence according to SEQ ID NO: 5, and
(iii) a LCDR3 sequence according to SEQ ID NO: 6.

24. The method of claim 19, wherein the antibody or an antigen-binding portion thereof comprises:
(a) a VH sequence with at least 80% sequence identity to SEQ ID NO: 22; and
(b) a VL sequence with at least 80% sequence identity to SEQ ID NO: 23.

25. The method of claim 19, wherein the antibody is a monoclonal antibody, or wherein the antigen binding fragment thereof is a single-chain Fv (scFv)nanobody, a Fab, a Fab′ or a F(ab′)2.

26. The method of claim 19, wherein the antibody is a monoclonal antibody that comprises (i) a mouse IgG1, IgG2a, IgG2b, IgG2c, or IgG3 heavy chain constant region, or an Fc-modified variant thereof with reduced effector function, or (ii) a human IgG1, IgG2, IgG3, or IgG4 heavy chain constant region, or an Fc-modified variant thereof with reduced effector function.

27. The method of claim 19, wherein the antibody or an antigen-binding portion thereof exhibits at least one of the following properties:
(i) binds to an ApoE3 comprising the amino acid sequence set forth in SEQ ID NO:3 with a K_{D} of 20 nM or less, wherein the K_{D} is measured by surface plasmon resonance (Biacore) analysis;
(ii) binds to an ApoE3 comprising the amino acid sequence set forth in SEQ ID NO:3 with an on rate (Kon) of 3×10⁵ l/Ms or more, wherein the on rate (Kon) is measured by surface plasmon resonance (Biacore) analysis; and
(iii) binds to an ApoE3 comprising the amino acid sequence set forth in SEQ ID NO:3 with an off rate (koff) of about 1×10⁻⁴ l/s or less, wherein the off rate (koff) is measured by surface plasmon resonance (Biacore) analysis.

28. The method of claim 19, wherein the subject has a disease, condition or disorder selected from the group consisting of: Alzheimer's disease, dementia, vascular cognitive impairment, vascular dementia, cerebral autosomal dominant arteriopathy with subcortical infarcts and leukoencephalopathy (CADASIL), cerebral autosomal recessive arteriopathy with subcortical infarcts and leukoencephalopathy (CARASIL), Lewy body dementia, frontotemporal dementia, amyotrophic lateral sclerosis, multiple sclerosis, Parkinson's disease, Huntington's disease, a neurodegenerative disease, a cerebrovascular disease, brain injury, chronic traumatic encephalopathy, a tauopathy, an amyloidopathy, a synucleinopathy, Creutzfeldt-Jakob disease, retinal degeneration, age-related macular degeneration, glaucoma, retinal injury, nerve degeneration, and aging.

29. The method of claim 19, wherein the subject has a genetic variation in a gene selected from the group consisting of APOE4, APOE3, PSEN1, PSEN2, APP, MAPT and any combination thereof.

30. A method of inhibiting or preventing binding of a heparan sulfate proteoglycan (HSPG) or heparin to Apolipoprotein E (ApoE) comprising contacting a polypeptide with an anti-Apolipoprotein E (ApoE) domain to an ApoE comprising the amino acid sequence set forth in SEQ ID NO:3, wherein the polypeptide binds to the ApoE comprising the amino acid sequence set forth in SEQ ID NO: 3 with higher affinity than to an ApoE comprising the amino acid sequence set forth in SEQ ID NO: 2; thereby inhibiting or preventing binding of heparan sulfate proteoglycan (HSPG) or heparin to the ApoE comprising the amino acid sequence set forth in SEQ ID NO: 3.
